SEQUENCE LISTING (548 sequences)

SEQ ID NO: 1 - protein, 106 aa, synthetic construct
  REGION 1..106: note = Synthetic
DIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQIT

SEQ ID NO: 2 - protein, 121 aa, synthetic construct
  REGION 1..121: note = Synthetic
QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSS

SEQ ID NO: 3 - protein, 247 aa, synthetic construct
  REGION 1..247: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 4 - protein, 106 aa, synthetic construct
  REGION 1..106: note = Synthetic
GQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 5 - protein, 106 aa, synthetic construct
  REGION 1..106: note = Synthetic
GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 6 - protein, 110 aa, synthetic construct
  REGION 1..110: note = Synthetic
APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAK

SEQ ID NO: 7 - protein, 114 aa, synthetic construct
  REGION 1..114: note = Synthetic
NPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTE

SEQ ID NO: 8 - protein, 115 aa, synthetic construct [sequence omitted]

SEQ ID NO: 9 - protein, 114 aa, synthetic construct
  REGION 1..114: note = Synthetic
NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTE

SEQ ID NO: 10 - protein, 115 aa, synthetic construct
  REGION 1..115: note = Synthetic
AFTVTVPKDLYVVEYGSNMTIECKFPVEKQLDLAALQVFWMMEDKNIIQFVHGEEDLKVQHSSYRQRARLLKDQLSLGNAALQITDVKLQDAGVYTCLIAYKGADYKRITVKVNA

SEQ ID NO: 11 - protein, 114 aa, synthetic construct
  REGION 1..114: note = Synthetic
NPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTE

SEQ ID NO: 12 - protein, 106 aa, synthetic construct [sequence omitted]

SEQ ID NO: 13 - protein, 27 aa, synthetic construct
  REGION 1..27: note = Synthetic
EFATMRPTWAWWLFLVLLLALWAPARG

SEQ ID NO: 14 - protein, 217 aa, Homo sapiens
  REGION 1..217: note = misc_feature - Human IgG1 Fc sequence 231-447 (EU-numbering)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 10 aa, synthetic construct
  REGION 1..10: note = Synthetic
EAAAKEAAAK

SEQ ID NO: 16 - protein, 5 aa, synthetic construct
  REGION 1..5: note = Synthetic
EAAAK

SEQ ID NO: 17 - protein, 4 aa, synthetic construct
  REGION 1..4: note = Synthetic
PPPP

SEQ ID NO: 18 - skipped

SEQ ID NO: 19 - protein, 5 aa, synthetic construct
  REGION 1..5: note = Synthetic
GGGGS

SEQ ID NO: 20 - protein, 15 aa, synthetic construct
  REGION 1..15: note = Synthetic
EAAAKEAAAKEAAAK

SEQ ID NO: 21 - protein, 118 aa, synthetic construct [sequence omitted]

SEQ ID NO: 22 - protein, 107 aa, synthetic construct [sequence omitted]

SEQ ID NO: 23 - protein, 113 aa, synthetic construct [sequence omitted]

SEQ ID NO: 24 - protein, 107 aa, synthetic construct [sequence omitted]

SEQ ID NO: 25 - protein, 119 aa, synthetic construct
  REGION 1..119: note = Synthetic
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVS

SEQ ID NO: 26 - protein, 106 aa, synthetic construct
  REGION 1..106: note = Synthetic
DIQMTQSPSTLSASVGDRVTITCKSQLSVGYMHWYQQKPGKAPKLLIYDTSKLASGVPSRFSGSGSGTEFTLTISSLQPDDFATYYCFQGSGYPFTFGGGTKLEIK

SEQ ID NO: 27-28 - skipped (2 entries)

SEQ ID NO: 29 - protein, 217 aa, Homo sapiens; identical to SEQ ID NO: 14
  REGION 1..217: note = misc_feature - Human IgG1 Fc sequence 231-447 (EU-numbering)

SEQ ID NO: 30-49 - skipped (20 entries)

SEQ ID NO: 50 - protein, 15 aa, synthetic construct
  REGION 1..15: note = Synthetic
EPKSCDKTHTCPPCP

SEQ ID NO: 51-101 - skipped (51 entries)

SEQ ID NO: 102 - protein, 213 aa, synthetic construct
  REGION 1..213: note = Synthetic
DIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 106 aa, synthetic construct; identical to SEQ ID NO: 1
  REGION 1..106: note = Synthetic

SEQ ID NO: 104 - skipped

SEQ ID NO: 105 - protein, 7 aa, synthetic construct
  REGION 1..7: note = Synthetic
DSSKLAS

SEQ ID NO: 106 - protein, 9 aa, synthetic construct
  REGION 1..9: note = Synthetic
QQWSRNPPT

SEQ ID NO: 107-108 - skipped (2 entries)

SEQ ID NO: 109 - protein, 5 aa, synthetic construct
  REGION 1..5: note = Synthetic
RSTMH

SEQ ID NO: 110 - protein, 17 aa, synthetic construct
  REGION 1..17: note = Synthetic
YINPSSAYTNYNQKFKD

SEQ ID NO: 111 - protein, 12 aa, synthetic construct
  REGION 1..12: note = Synthetic
PQVHYDYNGFPY

SEQ ID NO: 112-115 - skipped (4 entries)

SEQ ID NO: 116 - protein, 114 aa, synthetic construct; identical to SEQ ID NO: 9
  REGION 1..114: note = Synthetic

SEQ ID NO: 117-118 - skipped (2 entries)

SEQ ID NO: 119 - protein, 478 aa, synthetic construct
  REGION 1..478: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 120 - protein, 120 aa, synthetic construct [sequence omitted]

SEQ ID NO: 121 - protein, 5 aa, synthetic construct
  REGION 1..5: note = Synthetic
DTYIH

SEQ ID NO: 122 - protein, 17 aa, synthetic construct
  REGION 1..17: note = Synthetic
RIYPTNGYTRYADSVKG

SEQ ID NO: 123 - protein, 11 aa, synthetic construct
  REGION 1..11: note = Synthetic
WGGDGFYAMDY

SEQ ID NO: 124 - protein, 107 aa, synthetic construct [sequence omitted]

SEQ ID NO: 125 - protein, 11 aa, synthetic construct
  REGION 1..11: note = Synthetic
RASQDVNTAVA

SEQ ID NO: 126 - protein, 7 aa, synthetic construct
  REGION 1..7: note = Synthetic
SASFLYS

SEQ ID NO: 127 - protein, 9 aa, synthetic construct
  REGION 1..9: note = Synthetic
QQHYTTPPT

SEQ ID NO: 128-140 - skipped (13 entries)

SEQ ID NO: 141 - protein, 574 aa, synthetic construct
  REGION 1..574: note = Synthetic
NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEEAAAKEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 142-149 - skipped (8 entries)

SEQ ID NO: 150 - protein, 698 aa, synthetic construct
  REGION 1..698: note = Synthetic
DIQMTQSPSSLSASVGDRVTITCSASSSVSYMHWYQQKSGKAPKLLIYDTSKLASGVPSRFSGSGSGTDFTLTISSLQPEDFATYYCQQWSGYPLTFGQGTKLEIKGGGGSGGGGSGGGGSGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSGGGGSGEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 151 - protein, 602 aa, synthetic construct
  REGION 1..602: note = Synthetic
DIQMTQSPSSLSASVGDRVTITCSASSSVSYMHWYQQKSGKAPKLLIYDTSKLASGVPSRFSGSGSGTDFTLTISSLQPEDFATYYCQQWSGYPLTFGQGTKLEIKGGGGSGGGGSGGGGSGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGGGGGSGDRPWNPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTE

SEQ ID NO: 152 - protein, 598 aa, synthetic construct
  REGION 1..598: note = Synthetic
NPPTFSPALLVVTEGDNATFTCSFSNTSESFVLNWYRMSPSNQTDKLAAFPEDRSQPGQDARFRVTQLPNGRDFHMSVVRARRNDSGTYLCGAISLAPKLQIKESLRAELRVTEGGGGSGDIQMTQSPSSLSASVGDRVTITCSASSSVSYMHWYQQKSGKAPKLLIYDTSKLASGVPSRFSGSGSGTDFTLTISSLQPEDFATYYCQQWSGYPLTFGQGTKLEIKGGGGSGGGGSGGGGSGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 153 - protein, 602 aa, synthetic construct
  REGION 1..602: note = Synthetic
DIQMTQSPSSLSASVGDRVTITCSASSSVSYMHWYQQKSGKAPKLLIYDTSKLASGVPSRFSGSGSGTDFTLTISSLQPEDFATYYCQQWSGYPLTFGQGTKLEIKGGGGSGGGGSGGGGSGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGGGGGSGDRPWNPPTFSPALLVVTEGDNATFTCSFSNTSESFVLNWYRMSPSNQTDKLAAFPEDRSQPGQDARFRVTQLPNGRDFHMSVVRARRNDSGTYLCGAISLAPKLQIKESLRAELRVTE

SEQ ID NO: 154-189 - skipped (36 entries)

SEQ ID NO: 190 - protein, 574 aa, synthetic construct
  REGION 1..574: note = Synthetic
NPPTFSPALLVVTEGDNATFTCSFSNTSESFVLNWYRMSPSNQTDALAAFPEDRSQPGQDSRFRVTQLPNGRDFHMSVVRARRNDSGTYLCGAASLAPKAQIKESLRAELRVTEEAAAKEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 191 - protein, 114 aa, synthetic construct
  REGION 1..114: note = Synthetic
NPPTFSPALLVVTEGDNATFTCSFSNTSESFVLNWYRMSPSNQTDALAAFPEDRSQPGQDSRFRVTQLPNGRDFHMSVVRARRNDSGTYLCGAASLAPKAQIKESLRAELRVTE

SEQ ID NO: 192-198 - skipped (7 entries)

SEQ ID NO: 199 - protein, 7 aa, synthetic construct
  REGION 1..7: note = Synthetic
RSYQRPS

SEQ ID NO: 200 - protein, 11 aa, synthetic construct
  REGION 1..11: note = Synthetic
ATWDDSLDGWV

SEQ ID NO: 201-205 - skipped (5 entries)

SEQ ID NO: 206 - protein, 118 aa, synthetic construct
  REGION 1..118: note = Synthetic
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSS

SEQ ID NO: 207 - protein, 5 aa, synthetic construct
  REGION 1..5: note = Synthetic
RSTMH

SEQ ID NO: 208 - protein, 17 aa, synthetic construct
  REGION 1..17: note = Synthetic
YINPSSAYTNYNQKFKD

SEQ ID NO: 209 - protein, 12 aa, synthetic construct
  REGION 1..12: note = Synthetic
PQVHYDYNGFPY

SEQ ID NO: 210 - protein, 109 aa, synthetic construct
  REGION 1..109: note = Synthetic
NFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVL

SEQ ID NO: 211 - protein, 10 aa, synthetic construct
  REGION 1..10: note = Synthetic
SASSSVSYMN

SEQ ID NO: 212 - protein, 7 aa, synthetic construct
  REGION 1..7: note = Synthetic
DSSKLAS

SEQ ID NO: 213 - skipped

SEQ ID NO: 214 - protein, 9 aa, synthetic construct
  REGION 1..9: note = Synthetic
QQWSRNPPT

SEQ ID NO: 215 - protein, 118 aa, synthetic construct
  REGION 1..118: note = Synthetic
EVQLVESGGGLVQPGGSLRLSCAASGVTFNYYGMSWIRQAPGKGLEWVASITRSGGRIYYPDSVKGRFTISRENTQKTLYLQMNSLRAEDTAVYYCTLDGRDGWVAYWGQGTLVTVSS

SEQ ID NO: 216 - skipped

SEQ ID NO: 217 - protein, 17 aa, synthetic construct
  REGION 1..17: note = Synthetic
SITRSGGRIYYPDSVKG

SEQ ID NO: 218 - skipped

SEQ ID NO: 219 - protein, 107 aa, synthetic construct
  REGION 1..107: note = Synthetic
NFMLTQPSSVSGVPGQRVTISCTGNTGNIGSNYVNWYQQLPGTAPKLLIYRDDKRPSGVPDRFSGSKSGTSASLAITGFQAEDEADYYCQSYSSGFIFGGGTKLTVL

SEQ ID NO: 220 - protein, 13 aa, synthetic construct
  REGION 1..13: note = Synthetic
TGNTGNIGSNYVN

SEQ ID NO: 221 - protein, 7 aa, synthetic construct
  REGION 1..7: note = Synthetic
RDDKRPS

SEQ ID NO: 222 - skipped

SEQ ID NO: 223 - protein, 125 aa, synthetic construct
  REGION 1..125: note = Synthetic
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 224 - protein, 5 aa, synthetic construct
  REGION 1..5: note = Synthetic
KYAMN

SEQ ID NO: 225 - protein, 19 aa, synthetic construct
  REGION 1..19: note = Synthetic
RIRSKYNNYATYYADSVKD

SEQ ID NO: 226 - protein, 14 aa, synthetic construct
  REGION 1..14: note = Synthetic
HGNFGNSYISYWAY

SEQ ID NO: 227 - protein, 109 aa, synthetic construct
  REGION 1..109: note = Synthetic
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 228 - protein, 14 aa, synthetic construct
  REGION 1..14: note = Synthetic
GSSTGAVTSGNYPN

SEQ ID NO: 229 - protein, 7 aa, synthetic construct
  REGION 1..7: note = Synthetic
GTKFLAP

SEQ ID NO: 230 - protein, 9 aa, synthetic construct
  REGION 1..9: note = Synthetic
VLWYSNRWV

SEQ ID NO: 231 - protein, 125 aa, synthetic construct
  REGION 1..125: note = Synthetic
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 232 - protein, 5 aa, synthetic construct
  REGION 1..5: note = Synthetic
TYAMN

SEQ ID NO: 233 - protein, 19 aa, synthetic construct
  REGION 1..19: note = Synthetic
RIRSKYNNYATYYADSVKG

SEQ ID NO: 234 - protein, 14 aa, synthetic construct
  REGION 1..14: note = Synthetic
HGNFGNSYVSWFAY

SEQ ID NO: 235 - protein, 109 aa, synthetic construct
  REGION 1..109: note = Synthetic
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 236 - protein, 14 aa, synthetic construct
  REGION 1..14: note = Synthetic
GSSTGAVTTSNYAN

SEQ ID NO: 237 - protein, 7 aa, synthetic construct
  REGION 1..7: note = Synthetic
GTNKRAP

SEQ ID NO: 238 - protein, 9 aa, synthetic construct
  REGION 1..9: note = Synthetic
ALWYSNLWV

SEQ ID NO: 239 - protein, 121 aa, synthetic construct
  REGION 1..121: note = Synthetic
QVQLVQSGAEVKKPGASVKVSCKASGYTFTRSTMHWVRQAPGQGLEWIGYINPSSAYTNYNQKFKDRVTITADKSTSTAYMELSSLRSEDTAVYYCASPQVHYDYNGFPYWGQGTLVTVSS

SEQ ID NO: 240-242 - skipped (3 entries)

SEQ ID NO: 243 - protein, 106 aa, synthetic construct
  REGION 1..106: note = Synthetic
DIQMTQSPSSLSASVGDRVTITCSASSSVSYMNWYQQKPGKAPKRLIYDSSKLASGVPSRFSGSGSGTEFTLTISSLQPEDFATYYCQQWSRNPPTFGGGTKVEIK

SEQ ID NO: 244-249 - skipped (6 entries)

SEQ ID NO: 250 - protein, 448 aa, synthetic construct
  REGION 1..448: note = Synthetic
QVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 251 - protein, 213 aa, synthetic construct
  REGION 1..213: note = Synthetic
DIQMTQSPSSLSASVGDRVTITCSASSSVSYMHWYQQKSGKAPKLLIYDTSKLASGVPSRFSGSGSGTDFTLTISSLQPEDFATYYCQQWSGYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 252 - protein, 448 aa, synthetic construct
  REGION 1..448: note = Synthetic
QVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 253 - protein, 213 aa, synthetic construct; identical to SEQ ID NO: 102
  REGION 1..213: note = Synthetic

SEQ ID NO: 254 - protein, 215 aa, synthetic construct
  REGION 1..215: note = Synthetic
NFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 255 - protein, 574 aa, synthetic construct; identical to SEQ ID NO: 141
  REGION 1..574: note = Synthetic

SEQ ID NO: 256 - protein, 347 aa, synthetic construct
  REGION 1..347: note = Synthetic
NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 257 - protein, 213 aa, synthetic construct
  REGION 1..213: note = Synthetic
DIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 258 - protein, 574 aa, synthetic construct; identical to SEQ ID NO: 190
  REGION 1..574: note = Synthetic

SEQ ID NO: 259 - protein, 478 aa, synthetic construct
  REGION 1..478: note = Synthetic
DIQMTQSPSSLSASVGDRVTITCSASSSVSYMHWYQQKSGKAPKLLIYDTSKLASGVPSRFSGSGSGTDFTLTISSLQPEDFATYYCQQWSGYPLTFGQGTKLEIKGGGGSGGGGSGGGGSGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 260 - protein, 480 aa, synthetic construct
  REGION 1..480: note = Synthetic
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSGGGGSGGGGSGGGGSGGGGSNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 261 - protein, 571 aa, synthetic construct
  REGION 1..571: note = Synthetic
NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEEAAAKEAAAKEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 262 - protein, 567 aa, synthetic construct
  REGION 1..567: note = Synthetic
NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEGGGGSGEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 263 - protein, 335 aa, synthetic construct
  REGION 1..335: note = Synthetic
NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEGGGGSGNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 264 - protein, 339 aa, synthetic construct
  REGION 1..339: note = Synthetic
NFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGDRPWNPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTE

SEQ ID NO: 265 - protein, 571 aa, synthetic construct
  REGION 1..571: note = Synthetic
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGGGSGDRPWNPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTE

SEQ ID NO: 266 - protein, 698 aa, synthetic construct
  REGION 1..698: note = Synthetic
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGGGSGDIQMTQSPSSLSASVGDRVTITCSASSSVSYMHWYQQKSGKAPKLLIYDTSKLASGVPSRFSGSGSGTDFTLTISSLQPEDFATYYCQQWSGYPLTFGQGTKLEIKGGGGSGGGGSGGGGSGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSS

SEQ ID NO: 267 - protein, 598 aa, synthetic construct
  REGION 1..598: note = Synthetic
NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEGGGGSGDIQMTQSPSSLSASVGDRVTITCSASSSVSYMHWYQQKSGKAPKLLIYDTSKLASGVPSRFSGSGSGTDFTLTISSLQPEDFATYYCQQWSGYPLTFGQGTKLEIKGGGGSGGGGSGGGGSGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 268 - protein, 598 aa, synthetic construct
  REGION 1..598: note = Synthetic
NPPTFSPALLVVTEGDNATFTCSFSNTSESFVLNWYRMSPSNQTDALAAFPEDRSQPGQDARFRVTQLPNGRDFHMSVVRARRNDSGTYLCGAASLAPKAQIKESLRAELRVTEGGGGSGDIQMTQSPSSLSASVGDRVTITCSASSSVSYMHWYQQKSGKAPKLLIYDTSKLASGVPSRFSGSGSGTDFTLTISSLQPEDFATYYCQQWSGYPLTFGQGTKLEIKGGGGSGGGGSGGGGSGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 269 - protein, 602 aa, synthetic construct; identical to SEQ ID NO: 151
  REGION 1..602: note = Synthetic

SEQ ID NO: 270 - protein, 478 aa, synthetic construct
  REGION 1..478: note = Synthetic
DIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIKGGGGSGGGGSGGGGSGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 271 - protein, 568 aa, synthetic construct
  REGION 1..568: note = Synthetic
NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEGGGGSGQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 272 - protein, 698 aa, synthetic construct
  REGION 1..698: note = Synthetic
DIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIKGGGGSGGGGSGGGGSGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSGGGGSGEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 273 - protein, 698 aa, synthetic construct; identical to SEQ ID NO: 150
  REGION 1..698: note = Synthetic

SEQ ID NO: 274 - skipped

SEQ ID NO: 275 - protein, 9 aa, synthetic construct
  REGION 1..9: note = Synthetic
DGRDGWVAY

SEQ ID NO: 276 - protein, 106 aa, synthetic construct
  REGION 1..106: note = Synthetic
DIQMTQSPSSLSASVGDRVTITCSASSSVSYMHWYQQKSGKAPKLLIYDTSKLASGVPSRFSGSGSGTDFTLTISSLQPEDFATYYCQQWSGYPLTFGQGTKLEIK

SEQ ID NO: 277 - protein, 106 aa, synthetic construct
  REGION 1..106: note = Synthetic
DIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIK

SEQ ID NO: 278 - protein, 106 aa, synthetic construct; identical to SEQ ID NO: 1
  REGION 1..106: note = Synthetic

SEQ ID NO: 279 - protein, 7 aa, synthetic construct
  REGION 1..7: note = Synthetic
DTSKLAS

SEQ ID NO: 280 - skipped

SEQ ID NO: 281 - protein, 118 aa, synthetic construct; identical to SEQ ID NO: 206
  REGION 1..118: note = Synthetic

SEQ ID NO: 282 - protein, 6 aa, synthetic construct
  REGION 1..6: note = Synthetic
GGGGSG

SEQ ID NO: 283 - protein, 10 aa, synthetic construct
  REGION 1..10: note = Synthetic
GGGGSGDRPW

SEQ ID NO: 284 - protein, 20 aa, synthetic construct
  REGION 1..20: note = Synthetic
GGGGSGGGGSGGGGSGGGGS

SEQ ID NO: 285 - protein, 10 aa, synthetic construct
  REGION 1..10: note = Synthetic
GGYDGRGFDY

SEQ ID NO: 286 - protein, 5 aa, synthetic construct
  REGION 1..5: note = Synthetic
GYTMN

SEQ ID NO: 287 - protein, 13 aa, synthetic construct
  REGION 1..13: note = Synthetic
KRNTGNIGSNYVN

SEQ ID NO: 288 - protein, 17 aa, synthetic construct
  REGION 1..17: note = Synthetic
LITPYNGASSYNQKFRG

SEQ ID NO: 289 - protein, 109 aa, synthetic construct; identical to SEQ ID NO: 210
  REGION 1..109: note = Synthetic

SEQ ID NO: 290 - protein, 114 aa, synthetic construct; identical to SEQ ID NO: 9
  REGION 1..114: note = Synthetic

SEQ ID NO: 291 - protein, 114 aa, synthetic construct
  REGION 1..114: note = Synthetic
NPPTFSPALLVVTEGDNATFTCSFSNTSESFVLNWYRMSPSNQTDALAAFPEDRSQPGQDARFRVTQLPNGRDFHMSVVRARRNDSGTYLCGAASLAPKAQIKESLRAELRVTE

SEQ ID NO: 292 - protein, 114 aa, synthetic construct; identical to SEQ ID NO: 191
  REGION 1..114: note = Synthetic

SEQ ID NO: 293 - skipped

SEQ ID NO: 294 - protein, 9 aa, synthetic construct
  REGION 1..9: note = Synthetic
QQWSGYPLT

SEQ ID NO: 295 - protein, 8 aa, synthetic construct
  REGION 1..8: note = Synthetic
QSYSSGFI

SEQ ID NO: 296 - skipped

SEQ ID NO: 297 - protein, 119 aa, synthetic construct
  REGION 1..119: note = Synthetic
QVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSS

SEQ ID NO: 298 - protein, 7 aa, synthetic construct
  REGION 1..7: note = Synthetic
RNDKRPD

SEQ ID NO: 299 - skipped

SEQ ID NO: 300 - protein, 10 aa, synthetic construct
  REGION 1..10: note = Synthetic
SASSSVSYMH

SEQ ID NO: 301 - protein, 17 aa, synthetic construct
  REGION 1..17: note = Synthetic
SITSSGGRIYYPDSVKG

SEQ ID NO: 302 - protein, 5 aa, synthetic construct
  REGION 1..5: note = Synthetic
YYGMS

SEQ ID NO: 303 - protein, 5 aa, synthetic construct
  REGION 1..5: note = Synthetic
YYGMS

SEQ ID NO: 304-309 - skipped (6 entries)

SEQ ID NO: 310 - protein, 5 aa, synthetic construct
  REGION 1..5: note = Synthetic
SNPMI

SEQ ID NO: 311 - protein, 16 aa, synthetic construct
  REGION 1..16: note = Synthetic
IIDTDGSTYYADWAKG

SEQ ID NO: 312 - protein, 13 aa, synthetic construct
  REGION 1..13: note = Synthetic
RLHGSSNGYYDDL

SEQ ID NO: 313 - protein, 11 aa, synthetic construct
  REGION 1..11: note = Synthetic
QASQSIYSYLS

SEQ ID NO: 314 - protein, 7 aa, synthetic construct
  REGION 1..7: note = Synthetic
KASTLAS

SEQ ID NO: 315 - protein, 13 aa, synthetic construct
  REGION 1..13: note = Synthetic
QQGYTVTNVDKNT

SEQ ID NO: 316 - protein, 119 aa, synthetic construct
  REGION 1..119: note = Synthetic
QVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSS

SEQ ID NO: 317 - protein, 111 aa, synthetic construct
  REGION 1..111: note = Synthetic
DGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIK

SEQ ID NO: 318 - protein, 483 aa, synthetic construct
  REGION 1..483: note = Synthetic
QVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSGGGGSGGGGSGGGGSGGGGSDGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 319 - protein, 571 aa, synthetic construct
  REGION 1..571: note = Synthetic
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGGGSGDRPWNPPTFSPALLVVTEGDNATFTCSFSNTSESFVLNWYRMSPSNQTDKLAAFPEDRSQPGQDARFRVTQLPNGRDFHMSVVRARRNDSGTYLCGAISLAPKLQIKESLRAELRVTE

SEQ ID NO: 320 - protein, 571 aa, synthetic construct; identical to SEQ ID NO: 265
  REGION 1..571: note = Synthetic

SEQ ID NO: 321 - protein, 567 aa, synthetic construct; identical to SEQ ID NO: 262
  REGION 1..567: note = Synthetic

SEQ ID NO: 322 - protein, 567 aa, synthetic construct
  REGION 1..567: note = Synthetic
NPPTFSPALLVVTEGDNATFTCSFSNTSESFVLNWYRMSPSNQTDKLAAFPEDRSQPGQDARFRVTQLPNGRDFHMSVVRARRNDSGTYLCGAISLAPKLQIKESLRAELRVTEGGGGSGEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 323 - protein, 703 aa, synthetic construct
  REGION 1..703: note = Synthetic
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGGGSGQVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSGGGGSGGGGSGGGGSGGGGSDGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIK

SEQ ID NO: 324 - protein, 603 aa, synthetic construct
  REGION 1..603: note = Synthetic
NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEGGGGSGQVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSGGGGSGGGGSGGGGSGGGGSDGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 325 - protein, 603 aa, synthetic construct
  REGION 1..603: note = Synthetic
NPPTFSPALLVVTEGDNATFTCSFSNTSESFVLNWYRMSPSNQTDKLAAFPEDRSQPGQDARFRVTQLPNGRDFHMSVVRARRNDSGTYLCGAISLAPKLQIKESLRAELRVTEGGGGSGQVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSGGGGSGGGGSGGGGSGGGGSDGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 326 - protein, 607 aa, synthetic construct
  REGION 1..607: note = Synthetic
QVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSGGGGSGGGGSGGGGSGGGGSDGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGGGGGSGDRPWNPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTE

SEQ ID NO: 327 - protein, 607 aa, synthetic construct
  REGION 1..607: note = Synthetic
QVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSGGGGSGGGGSGGGGSGGGGSDGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGGGGGSGDRPWNPPTFSPALLVVTEGDNATFTCSFSNTSESFVLNWYRMSPSNQTDKLAAFPEDRSQPGQDARFRVTQLPNGRDFHMSVVRARRNDSGTYLCGAISLAPKLQIKESLRAELRVTE

SEQ ID NO: 328-399 - skipped (72 entries)

SEQ ID NO: 400 - protein, 571 aa, synthetic construct
  REGION 1..571: note = Synthetic
NPPTFSPALLVVTEGDNATFTCSFSNTSESFVLNWYRMSPSNQTDALAAFPEDRSQPGQDARFRVTQLPNGRDFHMSVVRARRNDSGTYLCGAASLAPKAQIKESLRAELRVTEEAAAKEAAAKEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 401 - protein, 571 aa, synthetic construct
  REGION 1..571: note = Synthetic
NPPTFSPALLVVTEGDNATFTCSFSNTSESFVLNWYRMSPSNQTDKLAAFPEDRSQPGQDARFRVTQLPNGRDFHMSVVRARRNDSGTYLCGAISLAPKAQIKESLRAELRVTEEAAAKEAAAKEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 402 - protein, 571 aa, synthetic construct
  REGION 1..571: note = Synthetic
NPPTFSPALLVVTEGDNATFTCSFSNTSESFVLNWYRMSPSNQTDKLAAFPEDRSQPGQDARFRVTQLPNGRDFHMSVVRARRNDSGTYLCGAISLAPKLQIKESLRAELRVTEEAAAKEAAAKEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 403 - protein, 571 aa, synthetic construct
  REGION 1..571: note = Synthetic
NPPTFSPALLVVTEGDNATFTCSFSNTSESFRLVWHRESPGYETDTLASFPEDRSTPLPDARFRVTQLPNGRDFHMSVVRARRNDSGTYVCGAIAFHPVIQIKESLRAELRVTEEAAAKEAAAKEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 404 - protein, 568 aa, synthetic construct
  REGION 1..568: note = Synthetic
NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEGGGGSGQVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 405 - protein, 218 aa, synthetic construct
  REGION 1..218: note = Synthetic
DGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 406 - protein, 703 aa, synthetic construct
  REGION 1..703: note = Synthetic
QVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSGGGGSGGGGSGGGGSGGGGSDGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIKGGGGSGEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 407 - protein, 483 aa, synthetic construct; identical to SEQ ID NO: 318
  REGION 1..483: note = Synthetic

SEQ ID NO: 408 - protein, 447 aa, synthetic construct
  REGION 1..447: note = Synthetic
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 409 - protein, 567 aa, synthetic construct
  REGION 1..567: note = Synthetic
NPPTFSPALLVVTEGDNATFTCSFSNTSESFVLNWYRMSPSNQTDALAAFPEDRSQPGQDARFRVTQLPNGRDFHMSVVRARRNDSGTYLCGAASLAPKAQIKESLRAELRVTEGGGGSGEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 410 - protein, 568 aa, synthetic construct
  REGION 1..568: note = Synthetic
NPPTFSPALLVVTEGDNATFTCSFSNTSESFVLNWYRMSPSNQTDALAAFPEDRSQPGQDARFRVTQLPNGRDFHMSVVRARRNDSGTYLCGAASLAPKAQIKESLRAELRVTEGGGGSGQVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 411 - protein, 571 aa, synthetic construct
  REGION 1..571: note = Synthetic
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGGGSGDRPWNPPTFSPALLVVTEGDNATFTCSFSNTSESFVLNWYRMSPSNQTDALAAFPEDRSQPGQDARFRVTQLPNGRDFHMSVVRARRNDSGTYLCGAASLAPKAQIKESLRAELRVTE

SEQ ID NO: 412 - protein, 335 aa, synthetic construct
  REGION 1..335: note = Synthetic
NPPTFSPALLVVTEGDNATFTCSFSNTSESFVLNWYRMSPSNQTDALAAFPEDRSQPGQDARFRVTQLPNGRDFHMSVVRARRNDSGTYLCGAASLAPKAQIKESLRAELRVTEGGGGSGNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 413 - protein, 567 aa, synthetic construct
  REGION 1..567: note = Synthetic
NPPTFSPALLVVTEGDNATFTCSFSNTSESFRLVWHRESPGYETDTLASFPEDRSTPLPDARFRVTQLPNGRDFHMSVVRARRNDSGTYVCGAIAFHPVIQIKESLRAELRVTEGGGGSGEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 414 - protein, 567 aa, synthetic construct
  REGION 1..567: note = Synthetic
NPPTFSPALLVVTEGDNATFTCSFSNTSESFVLNWYRMSPSNQTDKLAAFPEDRSQPGQDARFRVTQLPNGRDFHMSVVRARRNDSGTYLCGAISLAPKAQIKESLRAELRVTEGGGGSGEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 415 - protein, 568 aa, synthetic construct
  REGION 1..568: note = Synthetic
NPPTFSPALLVVTEGDNATFTCSFSNTSESFRLVWHRESPGYETDTLASFPEDRSTPLPDARFRVTQLPNGRDFHMSVVRARRNDSGTYVCGAIAFHPVIQIKESLRAELRVTEGGGGSGQVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 416 - protein, 218 aa, synthetic construct; identical to SEQ ID NO: 405
  REGION 1..218: note = Synthetic

SEQ ID NO: 417 - protein, 568 aa, synthetic construct
  REGION 1..568: note = Synthetic
NPPTFSPALLVVTEGDNATFTCSFSNTSESFVLNWYRMSPSNQTDKLAAFPEDRSQPGQDARFRVTQLPNGRDFHMSVVRARRNDSGTYLCGAISLAPKLQIKESLRAELRVTEGGGGSGQVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 418 - protein, 568 aa, synthetic construct
  REGION 1..568: note = Synthetic
NPPTFSPALLVVTEGDNATFTCSFSNTSESFVLNWYRMSPSNQTDKLAAFPEDRSQPGQDARFRVTQLPNGRDFHMSVVRARRNDSGTYLCGAISLAPKAQIKESLRAELRVTEGGGGSGQVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 419 - protein, 571 aa, synthetic construct
  REGION 1..571: note = Synthetic
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGGGSGDRPWNPPTFSPALLVVTEGDNATFTCSFSNTSESFRLVWHRESPGYETDTLASFPEDRSTPLPDARFRVTQLPNGRDFHMSVVRARRNDSGTYVCGAIAFHPVIQIKESLRAELRVTE

SEQ ID NO: 420 - protein, 571 aa, synthetic construct
  REGION 1..571: note = Synthetic
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGGGSGDRPWNPPTFSPALLVVTEGDNATFTCSFSNTSESFVLNWYRMSPSNQTDKLAAFPEDRSQPGQDARFRVTQLPNGRDFHMSVVRARRNDSGTYLCGAISLAPKAQIKESLRAELRVTE

SEQ ID NO: 421 - protein, 335 aa, synthetic construct
  REGION 1..335: note = Synthetic
NPPTFSPALLVVTEGDNATFTCSFSNTSESFRLVWHRESPGYETDTLASFPEDRSTPLPDARFRVTQLPNGRDFHMSVVRARRNDSGTYVCGAIAFHPVIQIKESLRAELRVTEGGGGSGNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 422 - protein, 335 aa, synthetic construct
  REGION 1..335: note = Synthetic
NPPTFSPALLVVTEGDNATFTCSFSNTSESFVLNWYRMSPSNQTDKLAAFPEDRSQPGQDARFRVTQLPNGRDFHMSVVRARRNDSGTYLCGAISLAPKLQIKESLRAELRVTEGGGGSGNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 423 - protein, 335 aa, synthetic construct
  REGION 1..335: note = Synthetic
NPPTFSPALLVVTEGDNATFTCSFSNTSESFVLNWYRMSPSNQTDKLAAFPEDRSQPGQDARFRVTQLPNGRDFHMSVVRARRNDSGTYLCGAISLAPKAQIKESLRAELRVTEGGGGSGNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 424 - protein, 598 aa, synthetic construct
  REGION 1..598: note = Synthetic
NPPTFSPALLVVTEGDNATFTCSFSNTSESFVLNWYRMSPSNQTDKLAAFPEDRSQPGQDARFRVTQLPNGRDFHMSVVRARRNDSGTYLCGAISLAPKAQIKESLRAELRVTEGGGGSGDIQMTQSPSSLSASVGDRVTITCSASSSVSYMHWYQQKSGKAPKLLIYDTSKLASGVPSRFSGSGSGTDFTLTISSLQPEDFATYYCQQWSGYPLTFGQGTKLEIKGGGGSGGGGSGGGGSGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 425 - protein, 603 aa, synthetic construct
  REGION 1..603: note = Synthetic
NPPTFSPALLVVTEGDNATFTCSFSNTSESFVLNWYRMSPSNQTDALAAFPEDRSQPGQDARFRVTQLPNGRDFHMSVVRARRNDSGTYLCGAASLAPKAQIKESLRAELRVTEGGGGSGQVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSGGGGSGGGGSGGGGSGGGGSDGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 426 - protein, 607 aa, synthetic construct
  REGION 1..607: note = Synthetic
QVQLVESGGGLVQPGGSLRLSCSVSGIDLSSNPMIWVRQAPGKGLQYIGIIDTDGSTYYADWAKGRFTISKDSTTVYLQINSPRAEDTAVYYCARRLHGSSNGYYDDLWGQGTLVTVSSGGGGSGGGGSGGGGSGGGGSDGQMTQSPSSVSASVGDRVTITCQASQSIYSYLSWYQQKPGQRPKLLIYKASTLASGVPSRFSGSGSGTDFTLTISSVQPEDFATYYCQQGYTVTNVDKNTFGGGTKVEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGGGGGSGDRPWNPPTFSPALLVVTEGDNATFTCSFSNTSESFVLNWYRMSPSNQTDALAAFPEDRSQPGQDARFRVTQLPNGRDFHMSVVRARRNDSGTYLCGAASLAPKAQIKESLRAELRVTE

SEQ ID NO: 427 - protein, 450 aa, synthetic construct
  REGION 1..450: note = Synthetic
QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 428 - protein, 574 aa, synthetic construct
  REGION 1..574: note = Synthetic
NPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTEEAAAKEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 429 - protein, 574 aa, synthetic construct
  REGION 1..574: note = Synthetic
NPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKLQIKESLRAELRVTEEAAAKEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 430 - protein, 574 aa, synthetic construct
  REGION 1..574: note = Synthetic
NPPTFSPALLVVTEGDNATFTCSFSNTSESFRLVWHRESPGYETDTLASFPEDRSTPLPDARFRVTQLPNGRDFHMSVVRARRNDSGTYVCGAIAFHPVIQIKESLRAELRVTEEAAAKEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 431 - protein, 602 aa, synthetic construct
  REGION 1..602: note = Synthetic
DIQMTQSPSSLSASVGDRVTITCSASSSVSYMHWYQQKSGKAPKLLIYDTSKLASGVPSRFSGSGSGTDFTLTISSLQPEDFATYYCQQWSGYPLTFGQGTKLEIKGGGGSGGGGSGGGGSGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGGGGGSGDRPWNPPTFSPALLVVTEGDNATFTCSFSNTSESFVLNWYRMSPSNQTDALAAFPEDRSQPGQDARFRVTQLPNGRDFHMSVVRARRNDSGTYLCGAASLAPKAQIKESLRAELRVTE

SEQ ID NO: 432 - protein, 347 aa, synthetic construct
  REGION 1..347: note = Synthetic
NPPTFSPALLVVTEGDNATFTCSFSNTSESFRLVWHRESPGYETDTLASFPEDRSTPLPDARFRVTQLPNGRDFHMSVVRARRNDSGTYVCGAIAFHPVIQIKESLRAELRVTEAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 433 - protein, 347 aa, synthetic construct
  REGION 1..347: note = Synthetic
NPPTFSPALLVVTEGDNATFTCSFSNTSESFVLNWYRMSPSNQTDKLAAFPEDRSQPGQDARFRVTQLPNGRDFHMSVVRARRNDSGTYLCGAISLAPKLQIKESLRAELRVTEAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 434 - protein, 347 aa, synthetic construct
  REGION 1..347: note = Synthetic
NPPTFSPALLVVTEGDNATFTCSFSNTSESFVLNWYRMSPSNQTDKLAAFPEDRSQPGQDARFRVTQLPNGRDFHMSVVRARRNDSGTYLCGAISLAPKAQIKESLRAELRVTEAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 435 - protein, 598 aa, synthetic construct
  REGION 1..598: note = Synthetic
NPPTFSPALLVVTEGDNATFTCSFSNTSESFRLVWHRESPGYETDTLASFPEDRSTPLPDARFRVTQLPNGRDFHMSVVRARRNDSGTYVCGAIAFHPVIQIKESLRAELRVTEGGGGSGDIQMTQSPSSLSASVGDRVTITCSASSSVSYMHWYQQKSGKAPKLLIYDTSKLASGVPSRFSGSGSGTDFTLTISSLQPEDFATYYCQQWSGYPLTFGQGTKLEIKGGGGSGGGGSGGGGSGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 436 - protein, 602 aa, synthetic construct
  REGION 1..602: note = Synthetic
DIQMTQSPSSLSASVGDRVTITCSASSSVSYMHWYQQKSGKAPKLLIYDTSKLASGVPSRFSGSGSGTDFTLTISSLQPEDFATYYCQQWSGYPLTFGQGTKLEIKGGGGSGGGGSGGGGSGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGGGGGSGDRPWNPPTFSPALLVVTEGDNATFTCSFSNTSESFRLVWHRESPGYETDTLASFPEDRSTPLPDARFRVTQLPNGRDFHMSVVRARRNDSGTYVCGAIAFHPVIQIKESLRAELRVTE

SEQ ID NO: 437 - protein, 602 aa, synthetic construct
  REGION 1..602: note = Synthetic
DIQMTQSPSSLSASVGDRVTITCSASSSVSYMHWYQQKSGKAPKLLIYDTSKLASGVPSRFSGSGSGTDFTLTISSLQPEDFATYYCQQWSGYPLTFGQGTKLEIKGGGGSGGGGSGGGGSGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGGGGGSGDRPWNPPTFSPALLVVTEGDNATFTCSFSNTSESFVLNWYRMSPSNQTDKLAAFPEDRSQPGQDARFRVTQLPNGRDFHMSVVRARRNDSGTYLCGAISLAPKAQIKESLRAELRVTE

SEQ ID NO: 438 - protein, 339 aa, synthetic construct
  REGION 1..339: note = Synthetic
NFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGDRPWNPPTFSPALLVVTEGDNATFTCSFSNTSESFVLNWYRMSPSNQTDALAAFPEDRSQPGQDARFRVTQLPNGRDFHMSVVRARRNDSGTYLCGAASLAPKAQIKESLRAELRVTE

SEQ ID NO: 439 - protein, 697 aa, synthetic construct
  REGION 1..697: note = Synthetic
DIQMTQSPSSLSASVGDRVTITCSASSSVSYMHWYQQKSGKAPKLLIYDTSKLASGVPSRFSGSGSGTDFTLTISSLQPEDFATYYCQQWSGYPLTFGQGTKLEIKGGGGSGGGGSGGGGSGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSGGGGSEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 440 - protein, 231 aa, synthetic construct
  REGION 1..231: note = Synthetic
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 441 - protein, 574 aa, synthetic construct
  REGION 1..574: note = Synthetic
NPPTFSPALLVVTEGDNATFTCSFSNTSESFRLVWHRESPSYQTDTLAAFPEDRSQPGQDARFRVTQLPNGRDFHMSVVRARRNDSGTYVCGAISLAPKIQIKESLRAELRVTEEAAAKEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 442-499 - skipped (58 entries)

SEQ ID NO: 500 - protein, 118 aa, synthetic construct
  REGION 1..118: note = Synthetic
QSLEESGGRLVTPGTPLTLTCTVSGIDLSSNPMIWVRQAPGKGLQYIGIIDTDGSTYYASWAKGRFTGSKTSTTVDLKITSPTTEDTATYFCARRLHGSSNGYYDDLWGQGTLVTISS

SEQ ID NO: 501 - protein, 111 aa, synthetic construct
  REGION 1..111: note = Synthetic
DGVMTQTPSSVSAAVGGTVTIKCQASQSIYSYLSWYQQKPGQRPKLLIYKASTLASGVPSRFKGSGSGTEFTLTISGVQCDDAATYYCQQGYTVTNVDKNTFGGGTEVVVK

SEQ ID NO: 502 - protein, 121 aa, synthetic construct
  REGION 1..121: note = Synthetic
QVQLVESGGGLVQPGGSLRLSCSVSGIDLSSNPMIWVRQAPGKGLEYIGIIDTDGSTYYADWAKGRFTISKDSSKNTVYLQMNSLRAEDTAVYYCARRLHGSSNGYYDDLWGQGTLVTVSS

SEQ ID NO: 503 - protein, 121 aa, synthetic construct
  REGION 1..121: note = Synthetic
QVQLVESGGGLVQPGGSLRLSCSVSGIDLSSNPMIWVRQAPGKGLQYIGIIDTDGSTYYADWAKGRFTISKDSSKNTVYLQMNSLRAEDTAVYYCARRLHGSSNGYYDDLWGQGTLVTVSS

SEQ ID NO: 504 - protein, 119 aa, synthetic construct
  REGION 1..119: note = Synthetic
QVQLVESGGGLVQPGGSLRLSCSVSGIDLSSNPMIWVRQAPGKGLEYIGIIDTDGSTYYADWAKGRFTISKDSTTVYLQMNSLRAEDTAVYYCARRLHGSSNGYYDDLWGQGTLVTVSS

SEQ ID NO: 505 - protein, 119 aa, synthetic construct; identical to SEQ ID NO: 316
  REGION 1..119: note = Synthetic

SEQ ID NO: 506 - protein, 111 aa, synthetic construct
  REGION 1..111: note = Synthetic
DGQMTQSPSSVSASVGDRVTITCQASQSIYSYLSWYQQKPGQRPKLLIYKASTLASGVPSRFSGSGSGTDFTLTISSVQPEDAATYYCQQGYTVTNVDKNTFGGGTKVEVK

SEQ ID NO: 507 - protein, 111 aa, synthetic construct; identical to SEQ ID NO: 317
  REGION 1..111: note = Synthetic

SEQ ID NO: 508 - protein, 261 aa, Homo sapiens
  REGION 1..261: note = misc_feature - Human CLDN18.2
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 509 - protein, 261 aa, Macaca fascicularis
  REGION 1..261: note = misc_feature - Cynomolgus monkey CLDN18
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKTYDGGAHTEDELQSYPSKHDYV

SEQ ID NO: 510 - protein, 264 aa, Mus musculus
  REGION 1..264: note = misc_feature - Mouse CLDN18 A2.1
MSVTACQGLGFVVSLIGFAGIIAATCMDQWSTQDLYNNPVTAVFNYQGLWRSCVRESSGFTECRGYFTLLGLPAMLQAVRALMIVGIVLGVIGILVSIFALKCIRIGSMDDSAKAKMTLTSGILFIISGICAIIGVSVFANMLVTNFWMSTANMYSGMGGMGGMVQTVQTRYTFGAALFVGWVAGGLTLIGGVMMCIACRGLTPDDSNFKAVSYHASGQNVAYRPGGFKASTGFGSNTRNKKIYDGGARTEDDEQSHPTKYDYV

SEQ ID NO: 511 - protein, 261 aa, Homo sapiens
  REGION 1..261: note = misc_feature - Human CLDN18.1
MSTTTCQVVAFLLSILGLAGCIAATGMDMWSTQDLYDNPVTSVFQYEGLWRSCVRQSSGFTECRPYFTILGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 512-529 - skipped (18 entries)

SEQ ID NO: 530 - protein, 114 aa, synthetic construct
  REGION 1..114: note = Synthetic
NPPTFSPALLVVTEGDNATFTCSFSNTSESFRLVWHRESPGYETDTLASFPEDRSTPLPDARFRVTQLPNGRDFHMSVVRARRNDSGTYVCGAIAFHPVIQIKESLRAELRVTE

SEQ ID NO: 531 - protein, 114 aa, synthetic construct
  REGION 1..114: note = Synthetic
NPPTFSPALLVVTEGDNATFTCSFSNTSESFRLVWHRESPSYQTDTLAAFPEDRSQPGQDARFRVTQLPNGRDFHMSVVRARRNDSGTYVCGAISLAPKIQIKESLRAELRVTE

SEQ ID NO: 532 - protein, 25 aa, synthetic construct
  REGION 1..25: note = Synthetic
  VARIANT 6..25: note = EAAAK may or may not be present
EAAAKEAAAKEAAAKEAAAKEAAAK

SEQ ID NO: 533 - protein, 5 aa, synthetic construct
  REGION 1..5: note = Synthetic
GPPPG

SEQ ID NO: 534 - protein, 7 aa, synthetic construct
  REGION 1..7: note = Synthetic
GGPPPGG

SEQ ID NO: 535 - protein, 6 aa, synthetic construct
  REGION 1..6: note = Synthetic
GPPPPG

SEQ ID NO: 536 - protein, 25 aa, synthetic construct
  REGION 1..25: note = Synthetic
  VARIANT 5..20: note = GGGS may or may not be present
GGGSGGGSGGGSGGGSGGGSGGGGS

SEQ ID NO: 537 - protein, 29 aa, synthetic construct
  REGION 1..29: note = Synthetic
  VARIANT 10..29: note = GGGGS may or may not be present
GGGSGGGGSGGGGSGGGGSGGGGSGGGGS

SEQ ID NO: 538 - protein, 45 aa, synthetic construct
  REGION 1..45: note = Synthetic
  VARIANT 5..20: note = GGGS may or may not be present
  VARIANT 26..45: note = GGGGS may or may not be present
GGGSGGGSGGGSGGGSGGGSGGGGSGGGGSGGGGSGGGGSGGGGS

SEQ ID NO: 539 - protein, 25 aa, synthetic construct
  REGION 1..25: note = Synthetic
  VARIANT 6..25: note = GGGGS may or may not be present
GGGGSGGGGSGGGGSGGGGSGGGGS

SEQ ID NO: 540 - protein, 80 aa, synthetic construct
  REGION 1..80: note = Synthetic
  VARIANT 1..80: note = GGGS may or may not be present
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 541 - protein, 5 aa, synthetic construct
  REGION 1..5: note = Synthetic
PPPPP

SEQ ID NO: 542 - protein, 20 aa, synthetic construct
  REGION 1..20: note = Synthetic
  VARIANT 1..20: note = May be present or absent
PPPPPPPPPPPPPPPPPPPP

SEQ ID NO: 543 - protein, 22 aa, synthetic construct
  REGION 1..22: note = Synthetic
  VARIANT 2..21: note = P may be present or absent
GPPPPPPPPPPPPPPPPPPPPG

SEQ ID NO: 544 - protein, 24 aa, synthetic construct
  REGION 1..24: note = Synthetic
  VARIANT 3..22: note = P may be present or absent
GGPPPPPPPPPPPPPPPPPPPPGG

SEQ ID NO: 545 - protein, 442 aa, synthetic construct
  REGION 1..442: note = Synthetic
  VARIANT 1..20: note = G may or may not be present
  VARIANT 21..440: note = May be present or absent
GGGGGGGGGGGGGGGGGGGGGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGG

SEQ ID NO: 546 - protein, 420 aa, synthetic construct
  REGION 1..420: note = Synthetic
  VARIANT 1..420: note = May or may not be present
SGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGG

SEQ ID NO: 547 - protein, 440 aa, synthetic construct
  REGION 1..440: note = Synthetic
  VARIANT 1..440: note = May be present or absent
SEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGG

SEQ ID NO: 548 - protein, 10 aa, synthetic construct
  REGION 1..10: note = Synthetic
EAAAKEAAAK

(19) United States
(12) Patent Application Publication
(10) Pub. No.: US 2025/0019440 A1
(43) Pub. Date: Jan. 16, 2025

(54) IMMUNOMODULATORY TRISPECIFIC T CELL ENGAGER FUSION PROTEINS

(71) Applicant: Zymeworks BC Inc., Vancouver (CA)

(72) Inventors: Surjit Bhimarao Dixit, Vancouver (CA); Eric ESCOBAR-CABRERA, Vancouver (CA); Florian Heinkel, Vancouver (CA); Anna Von Rossum, Vancouver (CA); Harsh Pratap, Vancouver (CA); Maya Poffenberger, Vancouver (CA); Chayne L. Piscitelli, Vancouver (CA); Meghan Marie Verstraete, Vancouver (CA)

(21) Appl. No.: 18/783,028

(22) Filed: Jul. 24, 2024

Related U.S. Application Data
(63) Continuation of application No. PCT/CA2023/050103, filed on Jan. 26, 2023.
(60) Provisional application No. 63/417,655, filed on Oct. 19, 2022.
(60) Provisional application No. 63/303,481, filed on Jan. 26, 2022.

(51) Int. Cl. C07K 16/28; A61K 39/00; A61P 35/00; C07K 16/32

(52) U.S. Cl. CPC C07K 16/2809; A61P 35/00; C07K 16/2818; C07K 16/2827; C07K 16/32; A61K 2039/505; C07K 2317/31; C07K 2317/51; C07K 2317/524; C07K 2317/526; C07K 2317/55; C07K 2317/565; C07K 2317/92

(57) ABSTRACT

The present disclosure provides fusion proteins with a multifunctional biologic design for programmed target engagement. In certain embodiments, the fusion proteins described herein provide for concurrent T cell and target antigen engagement and immune checkpoint targeting.

### CROSS-REFERENCE

This application claims the benefit of U.S. Provisional Application No. 63/303,481, filed Jan. 26, 2022, and U.S. Provisional Application No. 63/417,655, filed Oct. 19, 2022, which are incorporated herein by reference in their entireties for all purposes.

### BACKGROUND

Checkpoint inhibitors such as PD-1 and PD-L1 inhibitors used in the treatment of cancer exert their effect by blocking the interaction of immune checkpoint proteins expressed on T cells and cancer cells, thereby ameliorating immune suppression that would otherwise occur. Several anti-PD-1 antibodies (e.g., pembrolizumab and nivolumab) and anti-PD-L1 antibodies (e.g., atezolizumab) have been approved as therapeutics for certain types of cancer. These checkpoint inhibitors may be administered alone, or in conjunction with another antibody that is directed against an antigen that is overexpressed on a cancer cell, for example, in combination with an anti-HER2 antibody.

### SUMMARY

In certain embodiments, described herein is a trispecific fusion protein comprising: (i) a first binding domain capable of binding CD3 on the surface of a cytotoxic effector cell; (ii) a second binding domain capable of binding a tumor-associated antigen (TAA) on the surface of a tumor cell; (iii) a third binding domain capable of binding PD-L1 on the surface of a tumor cell; and (iv) a scaffold, wherein the first binding domain, the second binding domain and the third binding domain are operably linked to the scaffold. Such trispecific fusion protein can be trivalent or tetravalent, i.e., it can comprise another binding domain, for example, a second anti-TAA binding domain.

In some embodiments, described herein is a tetravalent and trispecific fusion protein comprising: (i) a first binding domain capable of binding CD3 on the surface of a cytotoxic effector cell; (ii) a second binding domain and a third binding domain capable of binding a tumor-associated antigen (TAA) on the surface of a tumor cell; (iii) a fourth binding domain capable of binding PD-L1 on the surface of a tumor cell; and (iv) a scaffold, wherein the first binding domain, the second binding domain, the third binding domain and the fourth binding domain are operably linked to the scaffold.

As further described herein, the trispecific fusion proteins of the present disclosure can have a variety of different formats, and such fusion proteins can comprise one or more peptide linker moieties capable of interconnecting the various polypeptides and domains that a fusion protein can be comprised of.

In some embodiments, provided herein are trispecific fusion proteins comprising an anti-CD3 binding domain comprising a VH region and a VL region that specifically binds a CD3 (Cluster of Differentiation 3) antigen on the surface of a T cell; one or two anti-TAA binding domains comprising a VH region and a VL region that specifically binds a TAA (tumor associated antigen) on the surface of a tumor cell; a PD-1 polypeptide capable of binding to a PD-L1 receptor on the surface of a tumor cell; wherein the anti-CD3 domain, the anti-TAA domain(s) and the PD-1 polypeptide are operably linked to a scaffold. The scaffold may be an antibody, or portion thereof, and may have, comprise or consist of a heterodimeric immunoglobulin (Ig) Fc region.

In certain embodiments, the anti-CD3 binding domain has a Fab format, the anti-TAA binding domain(s) has an scFv format, and the PD-1 polypeptide is fused to the N-terminus of the VH of the anti-CD3 binding domain Fab via a peptidic linker. In certain embodiments, the anti-CD3 binding domain has a Fab format, the anti-TAA binding domain has an scFv format, and the PD-1 polypeptide is fused to the N-terminus of the VL of the anti-CD3 binding domain Fab via a peptidic linker.

In certain embodiments, the PD-1 polypeptide used in the fusion proteins described herein is a wildtype PD-1. In certain embodiments, the PD-1 polypeptide comprises one or more mutations that increase or decrease its binding affinity for PD-L1, wherein such one or more mutations (e.g., amino acid substitution(s)) are relative to a certain wildtype or unmutated PD-1 polypeptide amino acid sequence. In certain embodiments, the PD-1 polypeptide of a fusion protein herein has an affinity for PD-L1 of between about 100 UM and about 10 pM, or between about 10 μM and about 150 pM, or between about 100 nM and about 150 pM.

In certain embodiments, the trispecific fusion proteins described herein comprise an anti-CD3 binding domain comprising a VH region and a VL region that specifically binds a CD3 (Cluster of Differentiation 3) antigen on the surface of a T cell, one or two anti-TAA binding domains comprising a VH region and a VL region that specifically binds a TAA (tumor associated antigen) on the surface of a tumor cell; and a PD-1 polypeptide capable of binding to a PD-L1 receptor on the surface of a tumor cell; wherein the fusion protein has a format of one of the formats exemplified in FIGS. 3A-3LL herein.

In some embodiments, the TAA may be any of mesothelin (MSLN), Claudin18.2 (Cldn18.2), GPC3, DLL3, PSMA, MUC17, LIV1, ROR1 and EGFRvIII.

In some embodiments, the peptidic linker is selected from SEQ ID NOS: 15, 16, 17, 18, 19 and 20.

In some embodiments, the trispecific fusion protein reduces or inhibits the binding of a PD-1 polypeptide located on a T cell to a PD-L1 polypeptide located on a cancer cell.

Also provided herein are pharmaceutical formulations/compositions of the trispecific fusion proteins described herein.

Also provided herein is a method of treating cancer in a subject in need thereof comprising administering the trispecific fusion protein to the subject.

Also provided herein is a method of overcoming or preventing the exhaustion of a T cell comprising exposing the T cell to the trispecific fusion proteins described herein, wherein the T cell can be located in a subject, such as a human or a rodent.

### BRIEF DESCRIPTION OF FIGURES

These and other features, aspects, and advantages of the present invention will become better understood with regard to the following description, and accompanying drawings (also “Figure” or “FIG.” herein), where:

FIG. 1A shows a cartoon of a possible mechanism by which trispecific fusion proteins described herein (PD-1/anti-CD3/anti-TAA) can elicit unique activity as T cell engager therapeutics. The molecule is thought to co-engage the Tumor Associated Antigen (TAA) and PD-L1 on a cancer cell, while also bringing a T cell into close proximity to the cancer cell by binding to CD3 on the T cell surface. This blocks the PD-L1/PD-1 interaction leading to checkpoint inhibition and T cell activation, wherein such interaction may otherwise prevent T cells from being activated.

FIG. 1B shows a spatial model of an antibody (PDB 1HZH) with the CH/CL of one arm representing the size of an scFv domain that would interact with the HER2 protein as TAA (PDB 1N8Z). The Fab interacts with CD3 (PDB 6JXR) and has a model helical linker connecting with a PD-1 polypeptide, which interacts with PD-L1 (PDB: 3BIK). This model shows that from a spatial point of view the co-interactions of the various binding domains of a fusion protein with their respective targets are spatially feasible.

FIG. 2 is a schematic drawing of an exemplary trispecific PD-1/anti-CD3/anti-TAA fusion protein in which the TAA is HER2, according to certain embodiments of the present disclosure.

FIGS. 3A through 3LL show a series of cartoons illustrating various non-limiting examples of possible formats for a PD-1/anti-CD3/anti-TAA trispecific fusion protein described herein, according to certain embodiments of the present disclosure. As illustrated in these cartoons, the PD-1 domain may be fused via a linker to the N-terminus or C-terminus of the anti-CD3 heavy chain or light chain, or it may be linked to a C-terminus of the Fc domain. The PD-1 domain may be fused via a linker to the N-terminus or C-terminus of the anti-TAA heavy chain or light chain. The VH and VL domains of an antigen-binding domain may be in, e.g., a Fab format, a hybrid format with one Fab and one scFv, or a dual scFv. There may be more than one anti-TAA antigen binding domain both comprising a VH and a VL with affinity for the TAA.

FIGS. 4A through 4D show the in vivo tumor growth of animals treated with bispecific or trispecific antibody fusion proteins. FIG. 4A shows tumor growth over time of animals treated either with 1 mg/kg or 0.5 mg/kg of the bispecific antibody fusion protein (v32497, black lines, n=8 each dose) or the trispecific antibody fusion protein (v31929, gray lines, n=8 each dose) once a week for four weeks. Days of test article injection are denoted by vertical dotted lines with arrows on top. FIG. 4B shows tumor growth over time of animals treated with a combination of antibodies designed to independently target the same epitopes as the Trispecific antibody fusion protein (v31929). The Bispecific v32497 was administered at 1 mg/kg once a week, and an anti PD-L1 antibody (Atezolizumab, v33449) was administered at 5 mg/kg twice a week for four weeks (n=8). Days of Trispecific or Bispecific test article injection are denoted by vertical dotted lines with arrows on top. FIG. 4C shows the frequency of durable response for each treatment regimen based upon animals whose tumor regressed to 0 mm³ and remained so for the duration of the study (n/a=not applicable). FIG. 4D shows results from the same study as shown in FIGS. 4A-4C, but with a different donor used to reconstitute the animals, demonstrating that the trispecific fusion protein, v31929, is able to elicit a significantly stronger T cell mediated anti-tumor response when compared to a bispecific construct either alone or in combination with a checkpoint inhibitor, leading to 6 out of 8 full responses (compared to no responses for the control cohorts and control/benchmark constructs).

FIG. 5 shows the levels of expression of the exhaustion markers PD-1, LAG-3 and TIM-3 increasing with sequential CD3/CD28 stimulations. T cells were stimulated with CD3/CD28 Dynabeads every two days for a total of 8 days. T cells were sampled on day 0 and days 2, 4, 6 and 8. Naïve T cells were used as a control. Surface expression of PD-1, LAG-3, and TIM-3 was determined by flow cytometry. Shown are bar graphs representing % of cells expressing the markers as determined by gating marker-positive cells based on FMO controls out of the live/CD3+ gate (A).

FIGS. 6A through 6C show the level of the inflammatory cytokines IFNgamma (“IFNγ” or “IFNg”, FIG. 6A), IL-2 (FIG. 6B) and TNFalpha (“TNFα”, FIG. 6C) in supernatants taken from cell cultures of CD3/CD28-stimulated T cells throughout eight days of culture.

FIG. 7 is a bar graph showing the proliferation of exhausted T cells 5 days after exposure to JIMT-1 tumor cells in the presence of various concentrations of a trispecific PD-1/anti-CD3/anti-HER2 fusion protein (v31929) and control constructs.

FIG. 8A shows the level of the inflammatory cytokines IFNgamma and FIG. 8B shows the level of IL-2 produced by exhausted T cells 3 days after exposure to JIMT-1 tumor cells in the presence of various concentrations of a trispecific PD-1/anti-CD3/anti-HER2 fusion protein (v31929) and control constructs.

FIG. 9 and FIG. 10 are graphs showing the percent survival of JIMT-1 target cells after 2 days of culture with exhausted T cells in the presence of trispecific PD-1/anti-CD3/anti-HER2 fusion protein and controls.

FIG. 11 shows on-cell binding of a trispecific PD-1/anti-CD3/anti-Her2 fusion protein (v31929) on naïve (top graph) and exhausted (bottom graph) T cells. Binding of Atezolizumab (anti-PD-L1) and a CD3 bispecific benchmark, v35923, are also shown as a comparison.

FIG. 12 depicts the potency of fusion protein constructs. The top graph shows the percent survival of H292 target tumor cells after 72 hours of co-culture with T cells in the presence of different trispecific and tri- or tetravalent PD-1/anti-MSLN/anti-CD3 fusion protein constructs with varying formats. Example graphs comparing the potency of two trispecific and tetravalent formats, v38449 (middle graph) and v38450 (bottom graph), against the anti-MSLN MH6T TriTAC benchmark and format-matched bispecific controls are also shown.

FIG. 13 shows graphs of the percent survival of SNU-216, H292, HCT-116, SKOV-3, and OVCAR-3 target tumor cells after 72 hours of co-culture with T cells in the presence of the trispecific and trivalent PD-1/anti-MSLN/anti-CD3 fusion protein v38344 and a control construct v38345, which consists of a format-matched anti-CD3xMSLN bispecific plus a PD-1 moiety with attenuated affinity to PD-L1 relative to the PD-L1 polypeptide used in v38344.

FIG. 14 shows graphs and a table showing the MSLN and PD-L1 surface expression receptor quantification for tumor cell lines SNU-216, H292, HCT-116, SKOV-3, and OVCAR-3. Surface expression receptor quantification of PD-L1 after 24 hours incubation with 20 ng/ml IFNg is also shown.

FIG. 15 shows results from a hybrid PD-1/PD-L1 reporter gene assay probing cross-linking of T cells and HCT-116 tumor cells and blockade of PD-1/PD-L1 checkpoint engagement. Results are shown for the trispecific and tetravalent PD-1/anti-CD3/anti-MSLN fusion protein (v38449), a format-matched bispecific control with an attenuated (KO) PD-1 moiety (v38454), and combination of bispecific control (v38454) with atezolizumab (v33449).

FIG. 16 shows percent survival of SNU-620 target tumor cells after 96 hours of co-culture with T cells in the presence of different trivalent or tetravalent trispecific PD-1/anti-CLDN18.2/anti-CD3 fusion proteins and control constructs (top graph). Example graphs comparing the potency of two trivalent and trispecific formats (v38408 (bottom left graph) and v38410 (bottom right graph)) against AMG-910 and format-matched bispecific controls are also shown.

FIG. 17 shows percent survival of SNU-620 target tumor cells after 72 hours of co-culture with T cells in the presence of different tetravalent and trispecific PD-1/anti-CLDN18.2/anti-CD3 fusion proteins and control constructs (top graph). Example graphs comparing the potency of two tetravalent and trispecific formats (v38999 (bottom left graph) and v39003 (bottom right graph)) against AMG-910 and format-matched bispecific controls are also shown.

FIG. 18 shows percent survival of SNU-620 (top graph), KATO-III (middle graph) and SNU-601 (bottom graph) target tumor cells after 96 or 120 hours of co-culture with T cells using varying T cell-to-tumor cell ratios and in the presence of the trivalent and trispecific PD-1/anti-CLDN18.2/anti-CD3 fusion protein v38410 or control constructs.

FIG. 19 shows graphs and a table showing the CLDN and PD-L1 surface expression receptor quantification for tumor cell lines SNU-620, SNU-601, and KATO-III. Surface expression receptor quantification of PD-L1 after 24 hours incubation with 20 ng/ml IFNg is also shown.

FIG. 20 shows results from a hybrid PD-1/PD-L1 reporter gene assay probing cross-linking of T cells and SNU-620 tumor cells and blockade of PD-1/PD-L1 checkpoint engagement. Results are shown for three trispecific PD-1/anti-CD3/anti-Cldn18.2 variants and their respective format-matched bispecific controls with attenuated (KO) PD-1 moieties, and a combination of bispecific control with atezolizumab (v38408, top graph; v38410, bottom left graph; v39007, bottom right graph).

FIG. 21 shows percent survival of HCT-116 and H292 target tumor cells after 72 hours of culture with T cells in the presence of trispecific PD-1/anti-MSLN/anti-CD3 fusion proteins with an affinity modulated or wildtype (WT) PD-1 moiety, as further described herein. Results for respective format-matched bispecific controls are also shown.

FIG. 22 shows percent survival of SNU-620 target tumor cells after 72 hours of culture with T cells in the presence of trispecific PD-1/anti-Cldn18.2/anti-CD3 fusion proteins with affinity modulated or wildtype (WT) PD-1 moiety, as further described herein. Results for respective format-matched bispecific controls are also shown.

FIG. 23 shows results from a hybrid PD-1/PD-L1 reporter gene assay probing cross-linking of T cells and HCT-116 tumor cells and blockade of PD-1/PD-L1 checkpoint engagement. Results are shown for three trispecific PD-1/anti-CD3/anti-MSLN variants with affinity modulated or wildtype (WT) PD-1 (v38910, top left graph; v38449, top right graph; v38450, bottom graph). Results for a respective format-matched bispecific control with attenuated (KO) PD-1 (v38454, v38455), and a combination of bispecific control with atezolizumab are also shown. The control variant v38354 is a “true” anti-MSLN/anti-CD3 bispecific construct that does not contain a PD-1 moiety.

FIG. 24 shows results from a hybrid PD-1/PD-L1 reporter gene assay probing cross-linking of T cells and SNU-620 tumor cells and blockade of PD-1/PD-L1 checkpoint engagement. Results are shown for three trispecific PD-1/anti-CD3/anti-Cldn18.2 formats using variants with affinity modulated or wildtype (WT) PD-1. Respective format-matched bispecific control with attenuated (KO) PD-1, and combination of bispecific control with atezolizumab are also shown.

FIG. 25 shows cytokine production following a 72-hour TDCC using H292 tumor cells. Tumor cells and T cells were treated with three trispecific PD-1/anti-CD3/anti-MSLN variants with affinity modulated or wildtype (WT) PD-1. Results for a format-matched bispecific control with attenuated (KO) PD-1 is also shown. TNFα, IL-2 and IFNγ production were measured using an MSD assay.

FIG. 26 shows flow cytometry results of the DC differentiation protocol confirming upregulation of DC surface markers CD11c, CD80, CD86, PD-L1 and downregulation of monocyte marker CD14 by day 7 after exposure to the trispecific fusion protein v31929.

FIG. 27 shows the results of the autologous DC-T cell co-culture assay confirming that the trispecific fusion protein (v31929) can co-engage PD-L1-expressing APCs and T cells resulting in high levels of T cell activation as measured by CD25 upregulation and proliferation of both CD4 and CD8 T cells.

FIG. 28 shows assessment of dendritic cell (DC)-T cell co-engagement by trispecific PD-1/anti-CD3/anti-Her2 fusion proteins comprising affinity modulated or WT PD-1 moieties. The trispecific construct with high-affinity PD-1 (v31929) shows superior capacity to activate T cells than the format-matched variants with lower PD-1 affinities.

### DETAILED DESCRIPTION

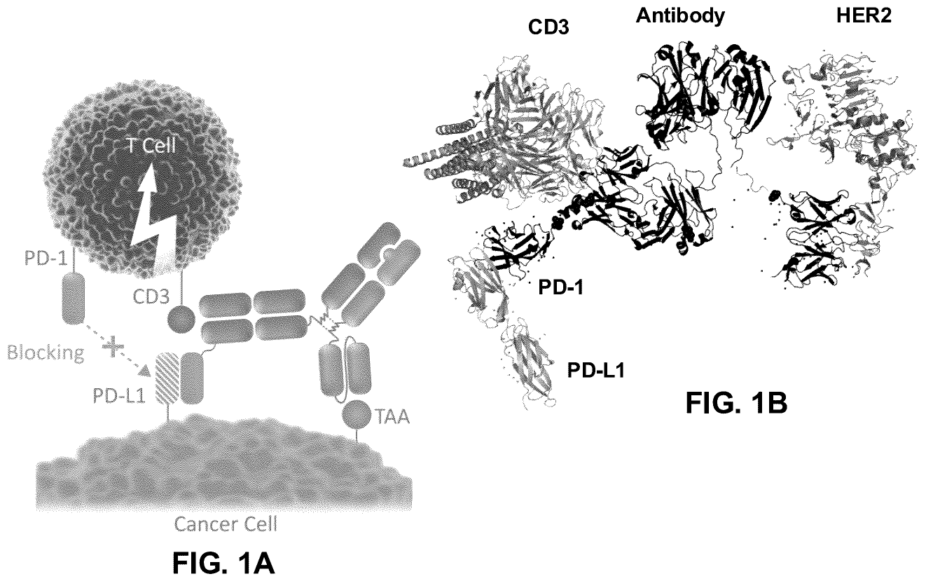

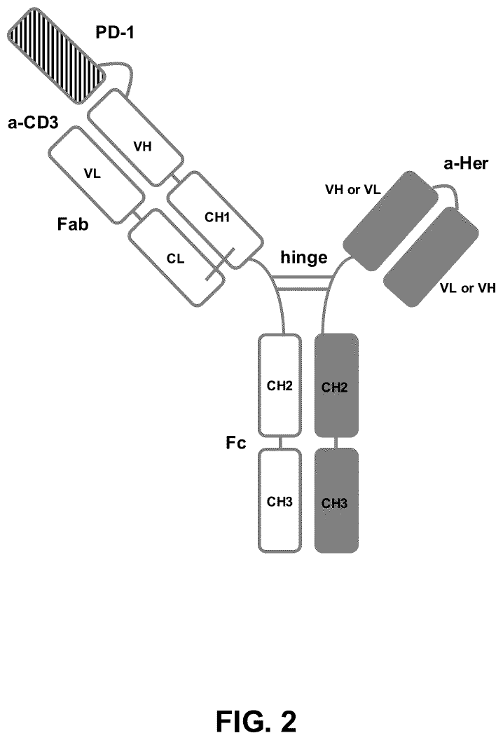

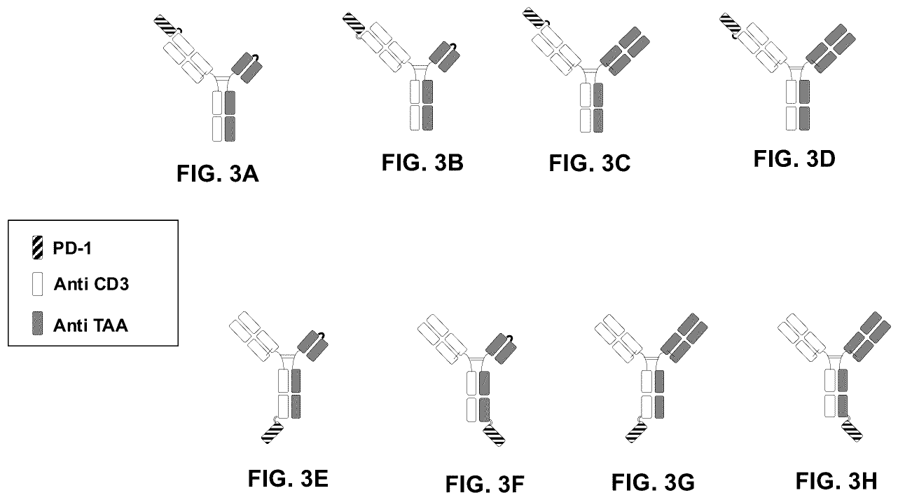

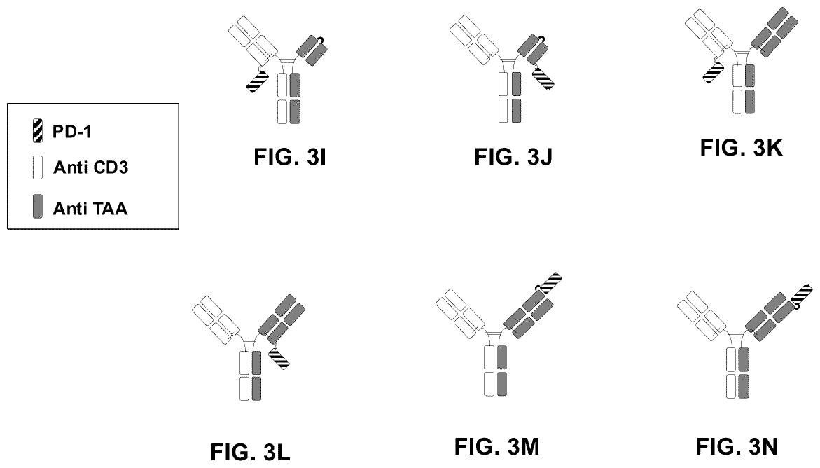

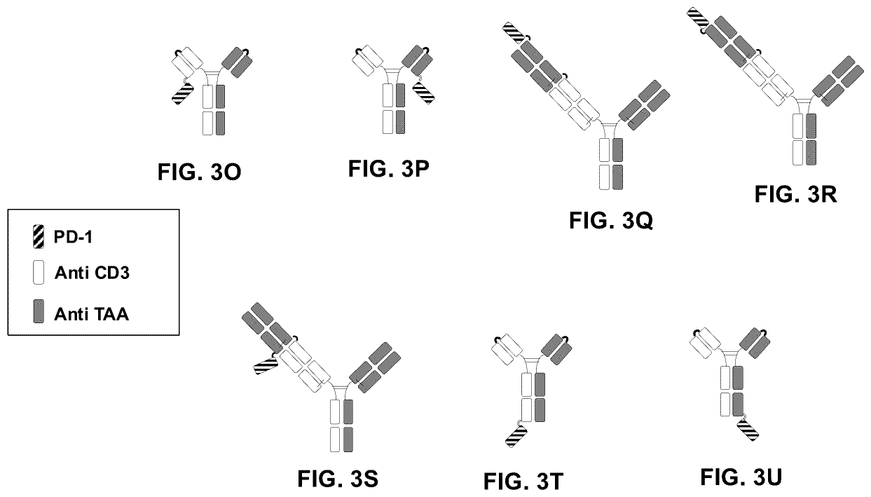

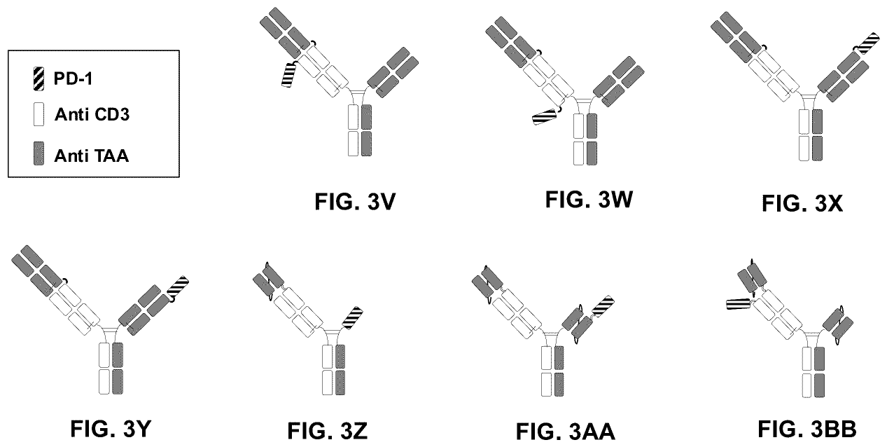

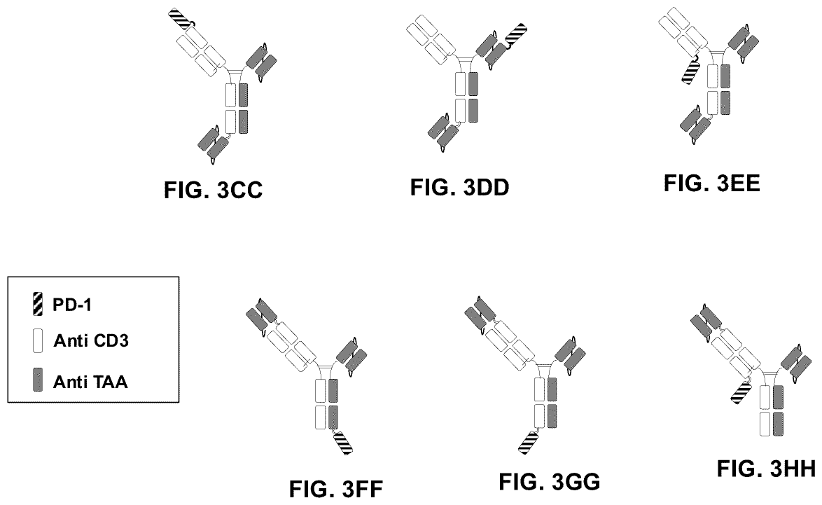

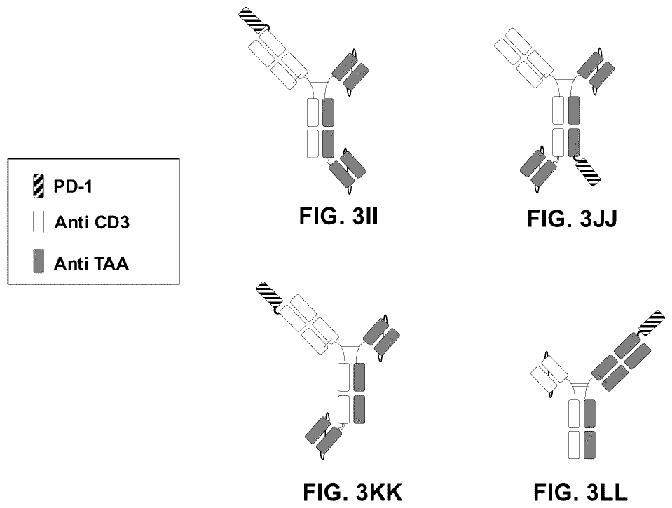

In various embodiments, disclosed herein are trispecific fusion proteins comprising a PD-1 domain derived from the extracellular IgV domain of a PD-1 polypeptide, an anti-CD3 binding domain and an anti-TAA (tumor associated antigen) binding domain fused to a scaffold. Also disclosed are pharmaceutical compositions comprising the trispecific fusion proteins disclosed herein, as well as methods of using them. Schematic drawings of exemplary PD-1/anti-CD3/anti-TAA fusion proteins with varying formats and geometries are shown in FIG. 3A-3LL. A schematic drawing of a specific embodiment of a trispecific fusion protein in which the TAA is HER2 is shown in FIG. 2. FIG. 1A shows a schematic model of a trispecific fusion protein's co-engagement with a T cell and a cancer cell. While bound via the TAA to the cancer cell, the anti-CD3 domain directs T cells to form a close synapse (e.g., a TCR-independent immune synapse), causing the T cell's activation for cancer cell killing. Coincidentally, the PD-1 domain can bind to PD-L1 on the cancer cell (in some cases, this cancer cell can be different to the one that is bound by the fusion protein via the anti-TAA binding domain) and as such acts as a checkpoint inhibitor. The trispecific fusion protein is a trifunctional molecule that combines anti-TAA, anti-CD3 and anti-PD-L1 activity. It is possible, in certain embodiments, that the trifunctional format and geometry of the fusion protein may promote binding to all three targets in the immunological synapse in close proximity, and the antigen binding may benefit from avidity and local concentration effects. FIG. 1B provides a hypothetical three-dimensional model for a trispecific fusion protein interacting with CD3, HER2 (i.e., the TAA) and PD-L1.

### I. Definitions

The terms used in the claims and specification are defined briefly here and, in more detail, below.

The term, “fusion protein,” as used herein, refers to a protein that comprises more than one (e.g., 2, 3, 4, or more) polypeptide regions, chains or domains linked to each other, e.g., by peptide bonds or other covalent bonds (e.g., disulfide bonds). Accordingly, “fused” as used herein in the context of a single fusion polypeptide chain, refers to polypeptide sequences linked to one another through a peptide bond. Examples include antibodies or scaffolds fused to immunomodulatory ligands, antigen binding domains, immunomodulatory receptors, antibodies or antibody fragments. Fusion proteins described herein, which comprise two or more polypeptide chains, are sometimes referred to as “variants” or “constructs”.

The term “biologically functional protein,” as used herein, broadly refers to a polypeptide or protein that has a biological function, e.g., an antibody, or portion thereof, e.g., a dimeric Fc or Fab domain.

The term “antibody,” as used herein, generally refers to immunoglobulin (Ig) and Ig-derived polypeptide constructs, which can include naturally occurring Igs from various species (e.g., human IgG, IgA, IgE, rodents, camelids, sharks, etc.), as well as non-naturally occurring Ig-like molecules, such as the fusion proteins described in this disclosure.

The term “Ligand-receptor pair,” as used herein, refers to a receptor polypeptide and a ligand polypeptide that specifically bind to one another. Examples include the PD-1-PD-L1 pair.

An “immunomodulatory” molecule, as used herein, generally refers to a molecule having the ability either directly or indirectly to modulate an immune response, e.g., upregulation or downregulation of an immune response in a subject, and/or immune cell activity.

The term “peptidic linker,” as used herein, refers to a peptide that joins, couples or links other peptides or polypeptides. Such term can be used interchangeably with the terms “peptide linker” or “peptide-based linker” and generally refers to a linker moiety that comprises at least one amino acid residue. In many embodiments, a linker herein comprises or consists of a consecutive sequence of two or more amino acid residues. In some embodiments, a linker of a trispecific fusion protein herein comprises or consists of a consecutive sequence of about 2, 5, 10, 15, 20, 25, 30, 40 or about 50 amino acid residues.

The terms “Fc region,” “Fc” and “Fc domain” are used interchangeably herein and refer to a C-terminal region of an immunoglobulin heavy chain that contains at least a portion of a constant region.

The term “bispecific,” as used herein in the context of a bispecific fusion protein or antibody, generally refers to a biologically functional protein (e.g., a fusion protein described herein) that can bind specifically two distinct epitopes, wherein the epitopes can be on the same or on different antigens. In some embodiments herein, the term “bispecific” can be used for a fusion protein which comprises three binding domains directed to three different antigens (i.e., may be considered trispecific), with one of such binding domains, however, being a knock-out (KO) binding domain with attenuated, or in some cases, no significant binding to the respective epitope or antigen. As an example, fusion proteins herein that contain a KO PD-1 domain (e.g., comprising one or more knock-out mutations) can be referred to as “bispecific,” and which can be used as a control construct with identical format but reduced PD-L1 binding.

The term “trispecific,” as used herein in the context of a trispecific fusion protein or antibody, generally refers to a biologically functional protein (e.g., a fusion protein described herein) that can bind specifically three distinct epitopes, wherein the epitopes can be on the same or on different antigens.

The term “multispecific,” as used herein in the context of a multispecific fusion protein or antibody, generally refers to a biologically functional protein (e.g., a fusion protein described herein) that can bind specifically to two or more distinct target molecules or epitopes, wherein the epitopes can be on the same or on different antigens.

The “valency” of a fusion protein described herein refers to the total number of antigen binding domains (e.g., scFv, Fab, or other polypeptide moieties) that a fusion protein comprises. In one example, and as described in various embodiments herein, a fusion protein herein can be trivalent, thus comprising three antigen binding domains. In other embodiments, a fusion protein herein is tetravalent, i.e., comprising four antigen binding domains. Such three or four antigen binding domains of a trivalent or tetravalent fusion protein, respectively, can be specific for the same or for different antigens and/or epitopes. As described in various embodiments herein, a fusion protein of the present disclosure can be trispecific, wherein such trispecific fusion protein is at least trivalent as it comprises three antigen binding domains, with each binding domain having a binding specificity for a different epitope and/or antigen. In some embodiments, and as further described herein, a trispecific fusion protein of this disclosure can be tetravalent and comprises four binding domains, wherein three of such four binding domains have binding specificities for three different epitopes and/or antigens, and two of such four binding domains have a binding specificity for the same epitope and antigen.

The term “immune checkpoint,” as used herein, generally refers to a regulatory pathway of the immune system that regulates the immune system activation.

The term “specifically binds,” (and grammatical variations thereof), when referring to binding of a fusion protein herein to a particular antigen, epitope, ligand or receptor, means binding that is measurably different from a non-specific interaction.

As described in more detail below, “mammal” includes both humans and non-humans and include, but is not limited to, humans, non-human primates, canines, felines, murines, bovines, equines, and porcines.

It must be noted that, as used in the specification and the appended claims, the singular forms “a,” “an” and “the” include plural referents unless the context clearly dictates otherwise.

Abbreviations used in this application include the following: PD-1 (Programmed Cell Death Protein 1); PD-L1 (Programmed death-ligand 1); CD3 (Cluster of Differentiation 3); and CD28 (Cluster of Differentiation 28).

As used herein, the term “about” refers to an approximately +/−10% variation from a given value. It is to be understood that such a variation is always included in any given value provided herein, whether or not it is specifically referred to.

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a fusion protein, composition, use or method, denotes that additional elements and/or method steps may be present, but that these additions do not materially affect the manner in which the recited fusion protein, composition, method or use functions. The term “consisting of” when used herein in connection with a fusion protein, composition, use or method, excludes the presence of additional elements and/or method steps. A fusion protein, composition, use or method described herein as comprising certain elements and/or steps may also, in certain embodiments consist essentially of those elements and/or steps, and in other embodiments consist of those elements and/or steps, whether or not these embodiments are specifically referred to.

It is contemplated that any embodiment discussed herein can be implemented with respect to any method, use, fusion protein, or composition disclosed herein, and vice versa.

It is also to be understood that the positive recitation of a feature in one embodiment, serves as a basis for excluding the feature in another embodiment. In particular, where a list of options is presented for a given embodiment or claim, it is to be understood that one or more option can be deleted from the list and the shortened list can form an alternative embodiment, whether or not such an alternative embodiment is specifically referred to.

Various amino acid sequences and sequences of clones and variants referred to herein are found in, e.g., Table AA Parts 1, 2 and 3, and Table BB.

### II. Trispecific Fusion Proteins

In certain embodiments, the present disclosure describes trispecific fusion proteins, such trispecific fusion proteins comprising: (i) a first binding domain capable of binding an antigen on a cytotoxic effector cell; (ii) a second binding domain capable of binding a TAA on a tumor cell; (iii) a third binding domain capable of binding PD-L1 on a tumor cell; and (iv) a scaffold, wherein the first binding domain, the second binding domain and the third binding domain are each operably linked to the scaffold, either directly or via one or more different linkers, e.g., peptidic linkers. In certain embodiments, the antigen on the cytotoxic effector cell is CD3 and the third binding domain is a wildtype or variant PD-1 polypeptide.

In various embodiments, the present disclosure describes a trispecific fusion protein comprising: (i) a first binding domain capable of binding CD3 on the surface of a cytotoxic effector cell (e.g., T cell); (ii) a second binding domain capable of binding a TAA on the surface of a tumor cell; (iii) a PD-1 polypeptide capable of binding PD-L1 on the surface of a tumor cell; and (iv) a scaffold, wherein the first binding domain, the second binding domain and the PD-1 polypeptide are operably linked to the scaffold.

In some embodiments, such trispecific fusion protein is trivalent, i.e., the trispecific fusion protein comprises three binding domains capable of binding to three different epitopes and/or antigens.

In some embodiments, the cytotoxic effector cell is an immune cell. The immune cell can be a T cell. In some embodiments, the TAA and PD-L1 that the fusion protein engages with and binds to are located on the surface of the same tumor cell. In other embodiments, the TAA and PD-L1 are located on the surface of two different tumor cells.

In various embodiments, the first binding domain of a trispecific fusion protein is a Fab domain, the Fab domain comprising a heavy chain comprising a VH sequence and a CH1 sequence and a light chain comprising a VL sequence and CL sequence, as further described and defined herein. Thus, in some embodiments, described herein is a trispecific fusion protein comprising (i) a Fab domain (first binding domain) capable of binding CD3 on the surface of a cytotoxic effector cell; (ii) a second binding domain capable of binding a TAA on the surface of a tumor cell; (iii) a PD-1 polypeptide capable of binding PD-L1 on the surface of a tumor cell; and (iv) a scaffold, wherein the Fab domain, the second binding domain and the PD-1 polypeptide are operably linked to the scaffold.

In various embodiments, the second binding domain of a trispecific fusion protein herein is an scFv domain comprising a VL sequence linked to a VH sequence. The order or connectivity of these two scFv domain sequences can be, from N- to C-terminus, such that the VH sequence is linked to a VL sequence, or the VL sequence is linked to a VH sequence. The VH and VL sequences can be linked to via a peptidic linker, e.g., in the context of an scFv domain referred to herein as linker^{scFv}. Such linker^{scFv} can be a peptide or peptidic linker comprising or consisting of a consecutive sequence of amino acid residues, e.g., about 2, 5, 10, 15, 20, 25, 30 or more consecutive amino acid residues.

In some embodiments, described herein is a trispecific fusion protein comprising (i) a first binding domain capable of binding CD3 on the surface of a cytotoxic effector cell; (ii) an scFv domain (second binding domain) capable of binding a TAA on the surface of a tumor cell; (iii) a PD-1 polypeptide capable of binding PD-L1 on the surface of a tumor cell; and (iv) a scaffold, wherein the first binding domain, the scFv domain and the PD-1 polypeptide are operably linked to the scaffold.

In certain embodiments, described herein is a trispecific fusion protein comprising (i) a Fab domain (first binding domain) capable of binding CD3 on the surface of a cytotoxic effector cell; (ii) an scFv domain (second binding domain) capable of binding a TAA on the surface of a tumor cell; (iii) a PD-1 polypeptide capable of binding PD-L1 on the surface of a tumor cell; and (iv) a scaffold, wherein the first binding domain, the scFv domain and the PD-1 polypeptide are operably linked to the scaffold.

In any of these embodiments, the scaffold is a moiety to which the three binding domains are operably linked, either directly or via one or more different peptidic linkers as further described herein. The term “operably linked,” as used herein in the context of a trispecific fusion protein, generally refers to a direct or indirect connection of a first domain of the trispecific fusion protein, e.g., a first binding domain, to a second domain of the trispecific fusion protein, e.g., a scaffold, wherein each operably linked domains of the fusion protein are capable of performing their function(s) (e.g., biological, physiological and/or chemical function(s)) in a way similar or identical to respective domains that are isolated and not operably linked as part of the trispecific fusion protein. Furthermore, and in instances in which a first domain of a trispecific fusion protein is directly and operably linked to a second domain of the trispecific fusion protein, such first domain is linked to the second domain without a linker and via a direct covalent bond, e.g., a peptide bond. On the other hand, a first domain of a trispecific fusion protein can be linked indirectly and operably to a second domain of the trispecific fusion protein in several ways, e.g., (i) through a linker moiety, and/or (ii) via another domain that may be located in between the first and second domains, e.g., in fusion polypeptide chain in which an scFv domain is linked to an Fc polypeptide via a Fab heavy chain.

In various embodiments, the scaffold of a trispecific fusion protein herein is an Fc domain, the Fc domain comprising a first Fc polypeptide and a second Fc polypeptide. At least one of the first or second Fc polypeptide can comprise a CH2 domain and/or a CH3 domain sequence. In certain embodiments, both the first and second Fc polypeptides each comprise a CH2 domain sequence and a CH3 domain sequence. In some embodiments, both the first Fc polypeptide and the second Fc polypeptide comprise or consist of an identical amino acid sequence. Such Fc domain which comprises Fc polypeptides with identical amino acid sequences can be referred to as a homodimeric Fc domain. In yet other embodiments, a trispecific fusion protein of the present disclosure comprises a heterodimeric Fc domain, the heterodimeric Fc domain comprising a first Fc polypeptide and a second Fc polypeptide, wherein the amino acid sequences of the first and second Fc polypeptides comprise or consist of amino acid sequences that differ in at least one amino acid residue. Thus, the first and second Fc polypeptides of a heterodimeric Fc domain can comprise amino acid sequences that share about 99%, 98%, 97%, 96%, or about 95% sequence identity. In some embodiments, the first and second Fc polypeptides of a heterodimeric Fc domain of the trispecific constructs described herein each comprise one or more asymmetric amino acid modifications (e.g., substitutions) that promote preferential pairing of the first and second Fc polypeptides to form the heterodimeric Fc domain, compared to the formation of a homodimeric Fc domains and relative to an Fc domain that does not comprise such one or more asymmetric modifications.

In some embodiments, described herein is a trispecific fusion protein comprising: (i) a first binding domain capable of binding CD3 on the surface of a cytotoxic effector cell; (ii) a second binding domain capable of binding a tumor-associated antigen (TAA) on the surface of a tumor cell; (iii) a third binding domain capable of binding PD-L1 on the surface of a tumor cell; an; and (iv) a scaffold, wherein the first binding domain, the second binding domain and the third binding domain are operably linked to the scaffold. In some embodiments, the scaffold comprises a dimeric Fc domain comprising a first Fc polypeptide and a second Fc polypeptide. The dimeric Fc domain can be a heterodimeric Fc domain, wherein the amino acid sequence of the first Fc polypeptide differs in at least one amino acid residue from the amino acid sequence of the second Fc polypeptide.

In some embodiments, the first binding domain is linked to the N-terminus of the first Fc polypeptide and the second binding domain is linked to the N-terminus of the second binding domain.

In some embodiments, the first binding domain and the second binding domain are each independently a Fab or an scFv. Thus, in some embodiments, a) the first binding domain is a Fab and the second binding domain is an scFv; or b) the first binding domain is an scFv and the second binding domain is a Fab; or c) the first binding domain is a Fab and the second binding domain is a Fab; or d) the first binding domain is an scFv and the second binding domain is an scFv.

In some embodiments, the first binding domain is linked to the first Fc polypeptide via a first linker^{Fc}. In some embodiments, the second binding domain is linked to the second Fc polypeptide via a second linker^{Fc}. In some of these embodiments, the first linker^{Fc}, the second linker^{Fc}, or both, comprise or consist of an IgG hinge region, or a portion or variant thereof. In some embodiments, the first linker^{Fc}, the second linker^{Fc}, or both, comprise or consist of an amino acid sequence having at least 80%, 90%, or 95% sequence identity to the amino acid sequence set forth in SEQ ID NO: 50 or a fragment thereof.

In some embodiments, the first binding domain is a Fab and is linked to the N-terminus of the first Fc polypeptide via the C-terminus of the Fab. In certain embodiments, wherein the second binding domain is an scFv comprising, from N- to C-terminus, a VH domain linked to a VL domain or a VL domain linked to a VH domain and is linked via its C-terminus to the N-terminus of the second Fc polypeptide.

In various embodiments herein, wherein a trispecific fusion protein herein comprises a first immunoglobulin G heavy chain, a second immunoglobulin G heavy chain and an immunoglobulin light chain, wherein the first heavy chain comprises, from N- to C-terminus, a Fab VH and CH1 domains linked to the first Fc polypeptide, the second heavy chain comprises, from N- to C-terminus, an scFv VH and VL or VL and VH domains linked to the second Fc polypeptide, and the light chain comprises, from N- to C-terminus, the Fab VL and CL domains.

In some embodiments, the third binding domain is linked to (i) the first binding domain, (ii) the second binding domain, or (iii) the scaffold. In some embodiments, the third binding domain is linked to (i) the N-terminus of the Fab VH domain, (ii) the N-terminus of the Fab VL domain, (iii) the C-terminus of the CL domain, (iv) the N-terminus of the scFv VH or VL domain, (v) the C-terminus of the first Fc polypeptide, or (vi) the C-terminus of the second Fc polypeptide.

In some embodiments, the third binding domain comprises a PD-1 polypeptide. In certain embodiments, the third binding domain consists of a PD-1 polypeptide. In some embodiments, the PD-1 polypeptide is a wildtype PD-1 polypeptide comprising or consisting of the amino acid sequence set forth in SEQ ID NO: 7, or a portion of fragment thereof. In other embodiments, the PD-1 polypeptide comprises one or more amino acid modifications compared to a corresponding wildtype PD-1 polypeptide that increase or decrease the binding affinity of the PD-1 polypeptide to PD-L1 when compared to the binding affinity of the corresponding wildtype PD-1 polypeptide to PD-L1. In some of these embodiments, the one or more amino acid modifications comprise one or more amino acid substitutions. In some embodiments, the PD-1 polypeptide used in a trispecific fusion protein has a binding affinity for PD-L1 of from about 100 μM to about 10 pM, from about 10 μM to about 150 pM, or from about 100 nM and 150 pM. In some embodiments, the PD-1 polypeptide comprises or consists of an amino acid sequence having at least about 80%, 90%, 95%, 99%, or 100% sequence identity to the amino acid sequence set forth in SEQ ID NO: 9, as further described herein.

In some embodiments, the fusion protein described herein is trispecific and trivalent, i.e., comprising three antigen binding domains, one anti-CD3 domain, one anti-TAA domain, and one anti-PD-L1 domain. In other embodiments herein, the fusion protein is trispecific and tetravalent, i.e., comprising four antigen binding domains, one anti-CD3 domain, two anti-TAA domain, and one anti-PD-L1 domain.

In certain embodiments, the trivalent and trispecific fusion protein is not v31929.

In certain embodiments, a trivalent and trispecific fusion protein or a tetravalent and trispecific fusion protein herein is capable of binding a TAA other than HER2, i.e., the TAA is not HER2.

In some embodiments, the trispecific fusion protein is tetravalent. Hence, in such embodiments, the trispecific fusion protein comprises a fourth binding domain, and wherein the fourth binding domain is linked either directly or via a linker to the first binding domain, the second binding domain, the third binding domain or the scaffold. Such fourth binding domain can be capable of binding the same TAA, such that the tetravalent fusion protein is capable of binding the TAA via two binding domains. In some embodiments, both the second binding domain and the fourth binding domain that are capable of binding the TAA are scFv domains. In such embodiments, the second binding domain and the fourth binding domain that are capable of binding the TAA each comprise or consist of the same anti-TAA VH and VL sequences.

In some embodiments, described herein is a tetravalent and trispecific fusion protein comprising: (i) a first binding domain capable of binding CD3 on the surface of a cytotoxic effector cell; (ii) a second binding domain and a third binding domain capable of binding a tumor-associated antigen (TAA) on the surface of a first tumor cell; (iii) a fourth binding domain capable of binding PD-L1 on the surface of a second tumor cell; and (iv) a scaffold, wherein the first binding domain, the second binding domain, the third binding domain and the fourth binding domain are operably linked to the scaffold.

In some embodiments, described herein is a trispecific fusion protein comprising: (i) a first binding domain capable of binding CD3 on the surface of a cytotoxic effector cell; (ii) a second binding domain capable of binding a TAA on the surface of a first tumor cell; (iii) a third binding domain capable of binding PD-L1 on the surface of a second tumor cell; and (iv) a scaffold, wherein the first binding domain, the second binding domain and the third binding domain are operably linked to the scaffold, and wherein the trispecific fusion protein is not v31929.

In some embodiments, described herein is a trispecific fusion protein comprising: (i) a first binding domain capable of binding CD3 on the surface of a cytotoxic effector cell; (ii) a second binding domain capable of binding a TAA on the surface of a first tumor cell; (iii) a third binding domain capable of binding PD-L1 on the surface of a second tumor cell; and (iv) a scaffold, wherein the first binding domain, the second binding domain and the third binding domain are operably linked to the scaffold, and wherein the TAA is not HER2.

In some embodiments, described herein is a trispecific fusion protein comprising: (i) a first binding domain capable of binding CD3 on the surface of a cytotoxic effector cell; (ii) a second binding domain capable of binding a tumor-associated antigen (TAA) on the surface of a first tumor cell; (iii) a third binding domain capable of binding PD-L1 on the surface of a second tumor cell; and (iv) a scaffold, wherein the first binding domain, the second binding domain and the third binding domain are operably linked to the scaffold, and wherein the third binding domain is linked to the second binding domain.

In some embodiments, described herein is a trispecific fusion protein comprising: (i) a first binding domain capable of binding CD3 on the surface of a cytotoxic effector cell; (ii) a second binding domain capable of binding a tumor-associated antigen (TAA) on the surface of a first tumor cell; (iii) a third binding domain capable of binding PD-L1 on the surface of a second tumor cell; and (iv) a scaffold, wherein the first binding domain, the second binding domain and the third binding domain are operably linked to the scaffold, and wherein the third binding domain is linked to the scaffold.

In some embodiments, described herein is a trispecific fusion protein comprising: (i) a first binding domain capable of binding CD3 on the surface of a cytotoxic effector cell; (ii) a second binding domain capable of binding a tumor-associated antigen (TAA) on the surface of a first tumor cell; (iii) a third binding domain capable of binding PD-L1 on the surface of a second tumor cell; and (iv) a scaffold, wherein the first binding domain, the second binding domain and the third binding domain are operably linked to the scaffold, and wherein the third binding domain is linked to (i) the second binding domain or (ii) the scaffold.

In some embodiments, described herein is a trispecific fusion protein comprising: (i) a first binding domain capable of binding CD3 on the surface of a cytotoxic effector cell, wherein the first binding domain is a Fab domain comprising a Fab heavy chain and a Fab light chain; (ii) a second binding domain capable of binding a tumor-associated antigen (TAA) on the surface of a first tumor cell; (iii) a third binding domain capable of binding PD-L1 on the surface of a second tumor cell; and (iv) a scaffold, wherein the first binding domain, the second binding domain and the third binding domain are operably linked to the scaffold, and wherein the third binding domain is linked to (i) the N- or C-terminus of the Fab light chain, (ii) second binding domain, or (iii) the scaffold.

In various embodiments, and as further described herein, upon binding to CD3 on the cytotoxic effector cell, the TAA on a tumor cell and PD-L1 on a tumor cell, the trispecific fusion protein forms a TCR-independent immune synapse capable of inducing effector cell mediated cytotoxicity against the tumor cell.

In certain embodiments, the trispecific fusion protein binds the TAA and PD-L1 on the same tumor cell. In other embodiments, the trispecific fusion protein binds the TAA and PD-L1 on different tumor cells.

### III. PD-1 Domains

In some embodiments, described herein are trispecific fusion proteins, with each fusion protein comprising a PD-1 domain (i.e., as the third binding domain) which is capable of binding to its cognate or naturally occurring ligand, PD-L1, on a tumor cell. PD-1 and PD-L1 belong to the Immunoglobulin Superfamily (IgSF) (Natarajan, Kannan; Mage, Michael G; and Margulies, David H (April 2015) Immunoglobulin Superfamily. In: eLS. John Wiley & Sons, Ltd: Chichester., A F Williams 1, A N Barclay (1988) The Immunoglobulin Superfamily—Domains for Cell Surface Recognition Annu Rev Immunol 6:381-405).

The Immunoglobulin Superfamily (IgSF) classifies a commonly found domain in proteins that is based on the core Immunoglobulin (Ig) fold. This Ig-fold consists of a beta-sandwich that is made up of a total of 7 antiparallel beta-strands that are arranged in two beta-sheets of 3 and 4 strands. The two beta-sandwiches are interconnected via a disulfide bridge between strands B and F. A structural motif commonly identified in Ig-folds is the “Greek Key” motif. Common sub-groups of the IgSF are IgV, IgC1 and IgC2 domains. Members are identified based on common structural features and the arrangement of the beta-strands. While IgC domains comprise 7 beta-strands arranged in two sheets of 3 and 4 strands, IgV domains comprise 9 beta-strands arranged in two sheets of 4 and 5 strands. IgC1 and IgC2 differ in the structural arrangement of the strands. IgSF domains can be found in a wide variety of biologically important proteins including antigen receptors, immunoglobulins and immunomodulatory receptors. Surface exposed residues of the core beta sandwich as well as the loops connecting the beta strands can serve as interaction interfaces for antigen recognition, other structural domains in a tertiary/quaternary assembly or a receptor/ligand pair. As the antigen recognition site of immunoglobulins (the VH-VL pair in an antibody such as IgG1) comprises a dimer of two IgV domains, a dimer of either IgSF or IgV domains is structurally compatible to form a steric mask for that antigen recognition site if attached covalently to the N-termini of the antibody.

The ligand-receptor pairs may be immunomodulatory, e.g., is an immune checkpoint, causes immune cell effector function modulation, modulation of T-cell receptor signaling, modulates interactions between antigen-presenting cells and effector cells, or combinations thereof. In certain embodiments, the ligand-receptor pair comprises an extracellular portion of an IgSF receptor and its cognate ligand, or a receptor-binding fragment thereof. A receptor-binding fragment refers to any polypeptide that binds specifically to the receptor of the ligand-receptor pair and can be naturally occurring or non-naturally occurring. “Naturally occurring,” as used herein and as applied to an object herein such as a polypeptide, refers to the fact that the object, e.g., polypeptide, can be found in nature. For example, a polypeptide or polynucleotide sequence that is present in an organism (e.g., mammal or plant) and which can be isolated from the source in nature, and which has not been intentionally modified by man in the laboratory, is naturally occurring. In certain embodiments, the ligand-receptor pairs may be two interacting protein domains that belong to the immunoglobulin domain superfamily and comprise a wildtype PD-1 polypeptide. “Non-naturally occurring”, as used herein, refers to an engineered polypeptide sequence with structural similarity to, e.g., IgSF, such as a mutant of a naturally occurring protein. Examples of immunomodulatory pairs of ligand-receptor domains belonging to the Immunoglobulin Superfamily include, but are not limited to, pairs of the B7/CD28 families (such as PD1-PDL1, PD1-PDL2, CTLA4-CD80, CD28-CD80, CD28-CD86, CTLA4-CD86, PDL1-CD80, and ICOS-ICOSL, NCR3LG1-NKp30, HHLA2-CD28H and CD47-SIRPα). CD80 (also known as B7-1), CD86 (B7-2), PDL1 (B7-H1), ICOSL (B7-H2), PDL2 (B7-DC), CD276 (B7-H3), VTCN1 (B7-H4), VISTA (B7-H5), NCR3LG1 (B7-H6), HHLA2 (B7-H7) belong to the B7 family. The B7 family of proteins is typically considered the ligand and pair with members of the CD28 family which comprises CD28, CTLA4, CD28H, NKp30, PD1 and ICOS. (S. M. West and X. A. Deng. Considering B7-CD28 as a family through sequence and structure. Exp Biol Med (Maywood) 2019; 244 (17): 1577-1583; doi: 10.1177/1535370219855970).

In some embodiments, the PD-1 domain can be the IgV domain of PD-1 (Uniprot ID Q15116, 18-132). The terms PD-1 domain” and “PD-1 polypeptide” are used interchangeably herein. In certain embodiments, the PD-1 domain can be a fragment of PD-1 which retains the ability to bind PD-L1. In certain embodiments described herein, the affinity of the PD-1 domain of the trispecific fusion protein to PD-L1 is altered (e.g., increased or decreased) as compared to the wild-type PD-1. In certain embodiments, the PD-1 domain is engineered to comprise sequences that are distinct from the wild-type PD-1.

In certain embodiments, the PD-1 domain of a fusion protein herein comprises one or more mutations that increase binding affinity of the PD-1 to its cognate binding partner, PD-L1. In certain embodiments, the relative binding affinity of a modified PD-1 polypeptide herein is greater than about 1, 1.5, 2, 2.5 3, 5, 10, 20, 30, 40, 50, 100, 500, 1000, 5,000, 10,000, 50,000 or about 100,000-fold than that of the corresponding wild-type PD-1 to its naturally occurring, cognate binding partner PD-L1.

In certain embodiments, the PD-1 domain of a fusion protein herein comprises one or more mutations that decrease its binding affinity for PD-L1. In certain embodiments, the relative binding affinity of the PD-1 compared to a wild-type PD-L1 is greater than 1, 1.5, 2, 2.5 3, 5, 10, 20, 30, 40, 50, 100, 500, 1000, 5,000, 10,000, 50,000 or 100,000-fold lower than that of the wild-type PD-1 ligand to its naturally occurring, cognate binding partner PD-L1.

In some embodiments, the PD-1 of a fusion protein herein has an amino acid sequence corresponding to SEQ ID NO: 7 or 11. In certain embodiments, the PD-1 has an amino acid sequence that is substantially identical to SEQ ID NO: 7 or 11. In certain embodiments, the PD-1 has an amino acid sequence that is about 80%, about 85%, about 90%, or about 95% identical to SEQ ID NO: 7 or 11. In certain embodiments, the PD-1 has an amino acid sequence that is about 96%, 97%, 98%, or 99% identical to SEQ ID NO: 7 or 11. Any PD-1 variant, e.g., high affinity variants, known in the art can also be used, for example, those provided in R. L. Maute et al., Engineering high-affinity PD-1 variants for optimized immunotherapy and immuno-PET imaging. Proc Natl Acad Sci USA 112, E6506-6514 (2015), WO2016/022994A2 or E. Lazar-Molnar et al., Structure-guided development of a high affinity human Programmed Cell Death-1: Implications for tumor immunotherapy EBIOMedicine 17. 30-44 (2017) and WO2019/241758A1.

In certain embodiments, the PD-1 polypeptide of a fusion protein herein has an affinity for PD-L1 of between about 100 μM and about 10 pM. In certain embodiments, the PD-1 polypeptide has an affinity for PD-L1 of between about 10 μM and about 150 pM. In certain embodiments, the PD-1 polypeptide has an affinity for PD-L1 of between about 100 nM and about 150 pM. In certain embodiments, the PD-1 polypeptide has an affinity for PD-L1 of about 10 pM, 50 pM, 100 pM, 150 pM, 200 pM, 400 pM, 500 pM, 600 pM, 700 pM, 800 pM, 900 pM or about 1 nM. In certain embodiments, the PD-1 polypeptide has an affinity for PD-L1 of about 1 nM, 5 nM, 10 nM, 20 nM, 30 nM, 40 nM, 50 nM, 60 nM, 70 nM, 80 nM, 90 nM or about 100 nM.

In some embodiments, a PD-1 polypeptide of a trispecific fusion protein herein has a binding affinity for PD-L1 from about 1 nM to about 200 nM, from about 10 nM to about 100 nM, from about 20 nM to about 75 nM, or from about 30 nM to about 50 nM.

### IV. Fusion Protein Formats

The trispecific fusion proteins described herein can be in numerous different formats, e.g., as shown in FIG. 3A-3LL. The fusion proteins described herein can be considered to have a modular architecture comprised of several IgG-derived domains, such as scFv, Fab, Fc domains, etc., that are operably linked to one another, e.g., via a scaffold, and that further includes at least a PD-1 domain which is fused to the biologically functional protein (e.g., an antibody or portion thereof), either directly or via a peptidic linker, to form a trispecific fusion protein as described herein. The biologically functional protein, e.g., the Ig-like moiety, in turn comprises at least a first and a second polypeptide chain. For example, either the N-terminus or C-terminus of the PD-1 domain can be fused to the first or second polypeptide of the biologically functional protein, e.g., via a peptidic linker. Various non-limiting formats for trispecific fusion proteins described herein are illustrated in FIG. 3A-3LL. For example, in some embodiments, the PD-1 domain may be fused via a peptidic linker to the N-terminus of the anti-CD3 light chain of the fusion protein. In some embodiments, the PD-1 domain may be fused via a peptidic linker to the N-terminus of the anti-CD3 heavy chain of the fusion protein. In some embodiments, the PD-1 domain may be fused via a peptidic linker to the C-terminus of the anti-CD3 light chain of the fusion protein. In some embodiments, the PD-1 domain may be fused via a peptidic linker to the C-terminus of the anti-TAA light chain of the fusion protein.

In certain embodiments, the VH and VL of the anti-CD3 binding domain are in a Fab format. In certain embodiments, the VH and VL of the anti-CD3 binding domain are in an scFv format. In certain embodiments, the VH and VL of the anti-TAA binding domain are in a Fab format. In certain embodiments, the VH and VL of the anti-TAA binding domain are in an scFv format. In certain embodiments, the VH and VL of the anti-CD3 binding domain are in a Fab format and the VH and VL of the anti-TAA binding domain are in an scFv format. In some embodiments, the VH and VL of the CD3 antigen-binding domain are in a Fab format and the VH and VL of the anti-TAA binding domain are in a Fab format. In certain embodiments, the fusion protein comprises more than one anti-TAA antigen binding domain comprising a VH and a VL which may be in a Fab format or an scFv format. In certain embodiments, the two anti-TAA binding domains are in an scFv format, as further described herein.

In the embodiments described below and illustrated in FIG. 3A-3LL, the scFv domains comprise a VH and a VL and may be in either of two orientations, (1) from N-terminus to C-terminus VH, VL, or (2) from N-terminus to C-terminus VL, VH. The VH region and VL region can be linked by a peptidic linker, for example, GGGGSGGGGSGGGGSGGGGS.

In some embodiments, e.g., as illustrated in FIG. 3A, the fusion protein comprises three polypeptides (1, light chain (LC)), (2, heavy chain (HC)) and (3, heavy chain (HC)), (from left to right), which, from N-terminus to C-terminus, comprise (1) anti-CD3 VL, anti-CD3 CL; (2) PD1 polypeptide, anti-CD3 VH, anti-CD3 CH1, CH2, CH3; and (3) anti-TAA scFv, CH2, CH3.

In some embodiments, e.g., as illustrated in FIG. 3B, the fusion protein comprises three polypeptides (1, LC), (2, HC) and (3, HC) (from left to right), which, from N-terminus to C-terminus, comprise (1) PD1 polypeptide, anti-CD3 VL, anti-CD3 CL; (2) anti-CD3 VH, anti-CD3 CH1, CH2, CH3; and (3) anti-TAA scFv, CH2, CH3.

In some embodiments, e.g., as illustrated in FIG. 3C, the fusion protein comprises four polypeptides (1, LC), (2, HC), (3, HC) and (4, LC) (from left to right), which, from N-terminus to C-terminus, comprise (1) anti-CD3 VL, anti-CD3 CL; (2) PD1 polypeptide, anti-CD3 VH, anti-CD3 CH1, CH2, CH3; (3) anti-TAA VH, CH1, CH2, CH3; and (4) anti TAA VL, CL.

In some embodiments, e.g., as illustrated in FIG. 3D, the fusion protein comprises four polypeptides (1, LC), (2, HC), (3, HC) and (4, LC) (from left to right), which, from N-terminus to C-terminus, comprise (1) PD1 polypeptide, anti-CD3 VL, anti-CD3 CL; (2) anti-CD3 VH, anti-CD3 CH1, CH2, CH3; (3) anti-TAA VH, CH1, CH2, CH3; and (4) anti-TAA VL, CL.

In some embodiments, e.g., as illustrated in FIG. 3E, the fusion protein comprises three polypeptides (1, LC), (2, HC) and (3, HC) (from left to right), which, from N-terminus to C-terminus, comprise (1) anti-CD3 VL, anti-CD3 CL; (2) anti-CD3 VH, anti-CD3 CH1, CH2, CH3, PD-1 polypeptide; and (3) anti-TAA scFv, CH2, CH3.

In some embodiments, e.g., as illustrated in FIG. 3F, the fusion protein comprises three polypeptides (1, LC), (2, HC) and (3, HC) (from left to right), which, from N-terminus to C-terminus, comprise (1) anti-CD3 VL, anti-CD3 CL; (2) anti-CD3 VH, anti-CD3 CH1, CH2, CH3; and (3) anti-TAA scFv, CH2, CH3, PD-1 polypeptide.

In some embodiments, e.g., as illustrated in FIG. 3G, the fusion protein comprises four polypeptides (1, LC), (2, HC), (3, HC) and (4, LC) (from left to right), which, from N-terminus to C-terminus, comprise (1) anti-CD3 VL, anti-CD3 CL; (2) anti-CD3 VH, anti-CD3 CH1, CH2, CH3, PD-1 polypeptide; (3) anti TAA VH, CH1, CH2, CH3; and (4) anti-TAA VL, CL.

In some embodiments, e.g., as illustrated in FIG. 3H, the fusion protein comprises four polypeptides (1, LC), (2, HC), (3, HC) and (4, LC) (from left to right), which, from N-terminus to C-terminus, comprise (1) anti-CD3 VL, anti-CD3 CL; (2) anti-CD3 VH, anti-CD3 CH1, CH2, CH3; (3) anti TAA VH, CH1, CH2, CH3 PD-1 polypeptide; and (4) anti-TAA VL, CL.

In some embodiments, e.g., as illustrated in FIG. 3I, the fusion protein comprises three polypeptides (1, LC), (2, HC) and (3, HC) (from left to right), which, from N-terminus to C-terminus, comprise (1) anti-CD3 VL, anti-CD3 CL, PD-1 polypeptide; (2) anti-CD3 VH, anti-CD3 CH1, CH2, CH3; and (3) anti-TAA scFv, CH2, CH3.

In some embodiments, e.g., as illustrated in FIG. 3J, the fusion protein comprises three polypeptides (1, LC), (2, HC) and (3, HC) (from left to right), which, from N-terminus to C-terminus, comprise (1) anti-CD3 VL, anti-CD3 CL; (2) anti-CD3 VH, anti-CD3 CH1, CH2, CH3; and (3) PD1 polypeptide, anti-TAA scFv, CH2, CH3.

In some embodiments, e.g., as illustrated in FIG. 3K, the fusion protein comprises four polypeptides (1, LC), (2, HC), (3, HC) and (4, LC) (from left to right), which, from N-terminus to C-terminus, comprise (1) anti-CD3 VL, anti-CD3 CL, PD-1 polypeptide; (2) anti-CD3 VH, anti-CD3 CH1, CH2, CH3; (3) anti TAA VH, CH1, CH2, CH3; and (4) anti-TAA VL, CL.

In some embodiments, e.g., as illustrated in FIG. 3L, the fusion protein comprises four polypeptides (1, LC), (2, HC), (3, HC) and (4, LC) (from left to right), which, from N-terminus to C-terminus, comprise (1) anti-CD3 VL, anti-CD3 CL; (2) anti-CD3 VH, anti-CD3 CH1, CH2, CH3; (3) anti-TAA VH, CH1, CH2, CH3; and (4) anti-TAA VL, CL, PD-1 polypeptide.

In some embodiments, e.g., as illustrated in FIG. 3M, the fusion protein comprises four polypeptides (1, LC), (2, HC), (3, HC) and (4) (from left to right), which, from N-terminus to C-terminus, comprise (1) anti-CD3 VL, anti-CD3 CL; (2) anti-CD3 VH, anti-CD3 CH1, CH2, CH3; (3) PD-1 polypeptide, anti-TAA VH, CH1, CH2, CH3; and (4) anti-TAA VL, CL.

In some embodiments, e.g., as illustrated in FIG. 3N, the fusion protein comprises four polypeptides (1, LC), (2, HC), (3, HC) and (4, LC) (from left to right), which, from N-terminus to C-terminus, comprise (1) anti-CD3 VL, anti-CD3 CL; (2) anti-CD3 VH, anti-CD3 CH1, CH2, CH3; (3) anti-TAA VH, CH1, CH2, CH3; and (4) PD-1 polypeptide, anti-TAA VL, CL.

In some embodiments, e.g., as illustrated in FIG. 3O, the fusion protein comprises two polypeptides (1, HC) and (2, HC) (from left to right), which, from N-terminus to C-terminus, comprise (1) PD-1 polypeptide, anti-CD3 scFv, CH2, CH3; and (2) anti-TAA scFv, CH2, CH3.

In some embodiments, e.g., as illustrated in FIG. 3P, the fusion protein comprises two polypeptides (1, HC) and (2, HC) (from left to right), which, from N-terminus to C-terminus, comprise (1) anti-CD3 scFv, CH2, CH3; and (2) PD1-polypeptide, anti-TAA scFv, CH2, CH3.

In some embodiments, e.g., as illustrated in FIG. 3Q, the fusion protein comprises four polypeptides (1, LC), (2, HC), (3, HC) and (4, LC) (from left to right), which, from N-terminus to C-terminus, comprise (1)) anti-TAA VL, CL, anti-CD3 VL, anti-CD3 CL; (2) PD-1 polypeptide, anti-TAA VH, CH1, anti-CD3 VH, CH1, CH2, CH3; (3) anti-TAA VH, CH1, CH2, CH3; and (4) anti-TAA VL, CL.

In some embodiments, e.g., as illustrated in FIG. 3R, the fusion protein comprises four polypeptides (1) (2) (3) and (4) (from left to right), which, from N-terminus to C-terminus, comprise (1) PD-1 polypeptide, anti-TAA VL, CL, anti-CD3 VL, anti-CD3 CL; (2) anti-TAA VH, CH1, anti-CD3 VH, CH1, CH2, CH3; (3) anti-TAA VH, CH1, CH2, CH3; and (4) anti-TAA VL, CL.

In some embodiments, e.g., as illustrated in FIG. 3S, the fusion protein comprises five polypeptides (1, LC), (2, LC), (3, HC), (4, HC) and (5, LC) (from left to right), which, from N-terminus to C-terminus, comprise (1) anti-TAA VL, CLI, PD-1 polypeptide, (2) anti-CD3 VL, anti-CD3 CL; (3) anti-TAA VH, CH1, anti-CD3 VH, CH1, CH2, CH3; (4) anti-TAA VH, CH1, CH2, CH3; and (5) anti-TAA VL, CL.

In some embodiments, e.g., as illustrated in FIG. 3T, the fusion protein comprises two polypeptides (1, HC) and (2, HC) (from left to right), which, from N-terminus to C-terminus, comprise (1) anti-CD3 scFv, CH2, CH3, PD-1 polypeptide; and (2) anti-TAA scFv, CH2, CH3.

In some embodiments, e.g., as illustrated in FIG. 3U, the fusion protein comprises two polypeptides (1, HC) and (2, HC) (from left to right), which, from N-terminus to C-terminus, comprise (1) anti-CD3 scFv, CH2, CH3; and (2) anti-TAA scFv, CH2, CH3, PD-1 polypeptide.

In some embodiments, e.g., as illustrated in FIG. 3V, the fusion protein comprises five polypeptides (1, LC), (2, LC), (3, HC), (4, HC) and (5, LC) (from left to right), which, from N-terminus to C-terminus, comprise (1) anti-TAA VL, CL; (2) PD-1 polypeptide, anti-CD3 VL, CL; (3) anti-TAA VH, CH1, anti-CD3 VH, CH1, CH2, CH3; (4) anti-TAA VH, CH1, CH2, CH3, and (5) anti-TAA VL, CL.

In some embodiments, e.g., as illustrated in FIG. 3W, the fusion protein comprises four polypeptides (1, LC), (2, HC), (3, HC) and (4, LC) (from left to right), which, from N-terminus to C-terminus, comprise (1) anti-TAA VL, CL, anti-CD3 VL, CL, PD-1 polypeptide; (2) anti-TAA VH, CH1, anti-CD3 VH, CH1, CH2, CH3; (3) anti-TAA VH, CH1, CH2, CH3 and (4) anti-TAA VL, CL.

In some embodiments, e.g., as illustrated in FIG. 3X, the fusion protein comprises four polypeptides (1, LC), (2, HC), (3, HC) and (4, LC) (from left to right), which, from N-terminus to C-terminus, comprise (1) anti-TAA VL, CL, anti-CD3 VL, CL: (2) anti-TAA VH, CH1, anti-CD3 VH, CH1, CH2, CH3; (3) PD-1 polypeptide, anti-TAA VH, CH1, CH2, CH3 and (4) anti-TAA VL, CL.

In some embodiments, e.g., as illustrated in FIG. 3Y, the fusion protein comprises four polypeptides (1, LC), (2, HC), (3, HC) and (4, LC) (from left to right), which, from N-terminus to C-terminus, comprise (1) anti-TAA VL, CL, anti-CD3 VL, CL: (2) anti-TAA VH, CH1, anti-CD3 VH, CH1, CH2, CH3; (3) anti-TAA VH, CH1, CH2, CH3 and (4) PD-1 polypeptide, anti-TAA VL, CL.

In some embodiments, e.g., as illustrated in FIG. 3Z, the fusion protein comprises three polypeptides (1, LC), (2, HC) and (3, HC) (from left to right), which, from N-terminus to C-terminus, comprise (1) anti-CD3 VL, CL: (2) anti-TAA scFv, anti-CD3 VH, CH1, CH2, CH3; (3) PD-1 polypeptide, anti-TAA CH2, CH3.

In some embodiments, e.g., as illustrated in FIG. 3AA, the fusion protein comprises three polypeptides (1, LC), (2, HC) and (3, HC) (from left to right), which, from N-terminus to C-terminus, comprise (1) anti-CD3 VL, CL: (2) anti-TAA scFv, anti-CD3 VH, CH1, CH2, CH3; (3) PD-1 polypeptide, anti-TAA scFv, anti-TAA CH2, CH3.

In some embodiments, e.g., as illustrated in FIG. 3BB, the fusion protein comprises three polypeptides (1, LC), (2, HC) and (3, HC) (from left to right), which, from N-terminus to C-terminus, comprise (1) PD-1 polypeptide, anti-CD3 VL, CL; (2) Anti-TAA scFv, anti-CD3 VH, CH1, CH2, CH3, (3) anti-TAA scFv, CH2, CH3.

In some embodiments, e.g., as illustrated in FIG. 3CC, the fusion protein comprises three polypeptides (1) (2) and (3) (from left to right), which, from N-terminus to C-terminus, comprise (1) anti-CD3 VL, CL; (2) PD-1 polypeptide, anti-CD3 VH, CH1, CH2, CH3, anti-TAA scFv; (3) anti-TAA scFv, CH2, CH3.

In some embodiments, e.g., as illustrated in FIG. 3DD, the fusion protein comprises three polypeptides (1, LC), (2, HC) and (3, HC) (from left to right), which, from N-terminus to C-terminus, comprise (1) anti-CD3 VL, CL; (2) anti-CD3 VH, CH1, CH2, CH3, anti-TAA scFv; (3) PD-1 polypeptide, anti-TAA scFv, CH2, CH3.

In some embodiments, e.g., as illustrated in FIG. 3EE, the fusion protein comprises three polypeptides (1, LC), (2, HC) and (3, HC) (from left to right), which, from N-terminus to C-terminus, comprise (1) anti-CD3 VL, CL; PD-1 polypeptide; (2) anti-CD3 VH, CH1, CH2, CH3, anti-TAA scFv; (3) anti-TAA scFv, CH2, CH3.

In some embodiments, e.g., as illustrated in FIG. 3FF, the fusion protein comprises three polypeptides (1, LC), (2, HC) and (3, HC) (from left to right), which, from N-terminus to C-terminus, comprise (1) anti-CD3 VL, CL; (2) anti-TAA scFv, anti-CD3 VH, CH1, CH2, CH3; (3) anti-TAA scFv, CH2, CH3, PD-1 polypeptide.

In some embodiments, e.g., as illustrated in FIG. 3GG, the fusion protein comprises three polypeptides (1, LC), (2, HC) and (3, HC) (from left to right), which, from N-terminus to C-terminus, comprise (1) anti-CD3 VL, CL; (2) anti-TAA scFv, anti-CD3 VH, CH1, CH2, CH3, PD-1 polypeptide; (3) anti-TAA scFv, CH2, CH3.

In some embodiments, e.g., as illustrated in FIG. 3HH, the fusion protein comprises three polypeptides (1, LC), (2, HC) and (3, HC) (from left to right), which, from N-terminus to C-terminus, comprise (1) anti-CD3 VL, CL, PD-1 polypeptide; (2) anti-TAA scFv, anti-CD3 VH, CH1, CH2, CH3; (3) anti-TAA scFv, CH2, CH3.

In some embodiments, e.g., as illustrated in FIG. 3II, the fusion protein comprises three polypeptides (1, LC), (2, HC) and (3, HC) (from left to right), which, from N-terminus to C-terminus, comprise (1) anti-CD3 VL, CL; (2) PD-1 polypeptide, anti-CD3 VH, CH1, CH2, CH3; (3) anti-TAA scFv, CH2, CH3, anti-CD3 scFv.

In some embodiments, e.g., as illustrated in FIG. 3JJ, the fusion protein comprises three polypeptides (1, LC), (2, HC) and (3, HC) (from left to right), which, from N-terminus to C-terminus, comprise (1) anti-CD3 VL, CL; (2) anti-CD3 VH, CH1, CH2, CH3, anti-TAA scFv; (3) anti-TAA scFv, CH2, CH3, PD-1 polypeptide.

In some embodiments, e.g., as illustrated in FIG. 3KK, the fusion protein comprises three polypeptides (1, LC), (2, HC) and (3, HC) (from left to right), which, from N-terminus to C-terminus, comprise (1) PD-1 polypeptide, anti-CD3 VL, CL; (2) anti-CD3 VH, CH1, CH2, CH3, anti-TAA scFv; (3) anti-TAA scFv, CH2, CH3.

In some embodiments, e.g., as illustrated in FIG. 3LL, the fusion protein comprises three polypeptides (1, LC), (2, HC) and (3, HC) (from left to right), which, from N-terminus to C-terminus, comprise (1) anti-CD3 scFv, CH2, CH3 (2) PD-1 polypeptide, anti-TAA VH, CH1, CH2, CH3; (3) anti-TAA VL, CL.

In some embodiments, the fusion protein is trivalent and trispecific and the TAA is HER2.

In various embodiments herein, an amino acid sequence of a trispecific fusion protein can be identified by its clone number which is further identified and defined herein.

In some embodiments, the trispecific fusion protein comprises (i) a first heavy chain having at least 90%, 95%, 97%, 99% or 100% sequence identity to the clone sequences set forth in 22080 and 23734, (ii) a second heavy chain having at least 90%, 95%, 97%, 99% or 100% sequence identity to the clone sequence set forth in 21490, and (iii) a light chain having at least 90%, 95%, 97%, 99% or 100% sequence identity to the clone sequence set forth in 12985.

In some embodiments, the trispecific fusion protein comprises (i) a first heavy chain having at least 90%, 95%, 97%, 99% or 100% sequence identity to the clone sequence set forth in 22080, (ii) a second heavy chain having at least 90%, 95%, 97%, 99% or 100% sequence identity to the clone sequence set forth in 21490, and (iii) a light chain having at least 90%, 95%, 97%, 99% or 100% sequence identity to the clone sequence set forth in 12985.

In some embodiments, the trispecific fusion protein comprises (i) a first heavy chain having at least 90%, 95%, 97%, 99% or 100% sequence identity to the clone sequence set forth in 23734, (ii) a second heavy chain having at least 90%, 95%, 97%, 99% or 100% sequence identity to the clone sequence set forth in 21490, and (iii) a light chain having at least 90%, 95%, 97%, 99% or 100% sequence identity to the clone sequence set forth in 12985.

In some embodiments, the fusion protein is trivalent and trispecific and the TAA is MSLN.

In some embodiments, the trispecific fusion protein comprises (i) a first heavy chain having at least 90%, 95%, 97%, 99% or 100% sequence identity to the clone sequences set forth in 29207, 29208, 29276, 29238, 29282, 22080 or 23734, (ii) a second heavy chain having at least 90%, 95%, 97%, 99% or 100% sequence identity to the clone sequences set forth in 23867, 23270, 29275 or 25095 and (iii) a light chain having at least 90%, 95%, 97%, 99% or 100% sequence identity to the clone sequences set forth in 16412, 23570 or 12985.

In some embodiments, the trispecific fusion protein comprises (i) a first heavy chain having at least 90%, 95%, 97%, 99% or 100% sequence identity to the clone sequence set forth in 29207, (ii) a second heavy chain having at least 90%, 95%, 97%, 99% or 100% sequence identity to the clone sequence set forth in 29275, and (iii) a light chain having at least 90%, 95%, 97%, 99% or 100% sequence identity to the clone sequence set forth in 16412.

In some embodiments, the trispecific fusion protein comprises (i) a first heavy chain having at least 90%, 95%, 97%, 99% or 100% sequence identity to the clone sequence set forth in 29208, (ii) a second heavy chain having at least 90%, 95%, 97%, 99% or 100% sequence identity to the clone sequence set forth in v29275, and (iii) a light chain having at least 90%, 95%, 97%, 99% or 100% sequence identity to the clone sequence set forth in 16412.

In some embodiments, the trispecific fusion protein comprises (i) a first heavy chain having at least 90%, 95%, 97%, 99% or 100% sequence identity to the clone sequence set forth in 29276, (ii) a second heavy chain having at least 90%, 95%, 97%, 99% or 100% sequence identity to the clone sequence set forth in 25095, and (iii) a light chain having at least 90%, 95%, 97%, 99% or 100% sequence identity to the clone sequence set forth in 23570.

In some embodiments, the trispecific fusion protein comprises (i) a first heavy chain having at least 90%, 95%, 97%, 99% or 100% sequence identity to the clone sequence set forth in 29238, (ii) a second heavy chain having at least 90%, 95%, 97%, 99% or 100% sequence identity to the clone sequence set forth in 29275, and (iii) a light chain having at least 90%, 95%, 97%, 99% or 100% sequence identity to the clone sequence set forth in 16412.

In some embodiments, the trispecific fusion protein comprises (i) a first heavy chain having at least 90%, 95%, 97%, 99% or 100% sequence identity to the clone sequence set forth in 22080, (ii) a second heavy chain having at least 90%, 95%, 97%, 99% or 100% sequence identity to the clone sequence set forth in 23867, and (iii) a light chain having at least 90%, 95%, 97%, 99% or 100% sequence identity to the clone sequence set forth in 12985.

In some embodiments, the trispecific fusion protein comprises (i) a first heavy chain having at least 90%, 95%, 97%, 99% or 100% sequence identity to the clone sequence set forth in 23734, (ii) a second heavy chain having at least 90%, 95%, 97%, 99% or 100% sequence identity to the clone sequence set forth in 23867, and (iii) a light chain having at least 90%, 95%, 97%, 99% or 100% sequence identity to the clone sequence set forth in 12985.

In some embodiments, the trispecific fusion protein comprises (i) a first heavy chain having at least 90%, 95%, 97%, 99% or 100% sequence identity to the clone sequence set forth in 29282, (ii) a second heavy chain having at least 90%, 95%, 97%, 99% or 100% sequence identity to the clone sequence set forth in 23270, and (iii) a light chain having at least 90%, 95%, 97%, 99% or 100% sequence identity to the clone sequence set forth in 16412.

In some embodiments, the fusion protein is trivalent and trispecific and the TAA is Cldn18.2.

In some embodiments, the trispecific fusion protein comprises (i) a first heavy chain having at least 90%, 95%, 97%, 99% or 100% sequence identity to the clone sequences set forth in 29241, 29238, 29208 or 29211, (ii) a second heavy chain having at least 90%, 95%, 97%, 99% or 100% sequence identity to the clone sequences set forth in 29264, 29261, 29267 or 28373 and (iii) a light chain having at least 90%, 95%, 97%, 99% or 100% sequence identity to the clone sequence set forth in 16412.

In some embodiments, the trispecific fusion protein comprises (i) a first heavy chain having at least 90%, 95%, 97%, 99% or 100% sequence identity to the clone sequence set forth in 29241, (ii) a second heavy chain having at least 90%, 95%, 97%, 99% or 100% sequence identity to the clone sequence set forth in 28373, and (iii) a light chain having at least 90%, 95%, 97%, 99% or 100% sequence identity to the clone sequence set forth in 16412.

In some embodiments, the trispecific fusion protein comprises (i) a first heavy chain having at least 90%, 95%, 97%, 99% or 100% sequence identity to the clone sequence set forth in 29238, (ii) a second heavy chain having at least 90%, 95%, 97%, 99% or 100% sequence identity to the clone sequence set forth in 28373, and (iii) a light chain having at least 90%, 95%, 97%, 99% or 100% sequence identity to the clone sequence set forth in 16412.

In some embodiments, the trispecific fusion protein comprises (i) a first heavy chain having at least 90%, 95%, 97%, 99% or 100% sequence identity to the clone sequence set forth in 29208, (ii) a second heavy chain having at least 90%, 95%, 97%, 99% or 100% sequence identity to the clone sequence set forth in 28373, and (iii) a light chain having at least 90%, 95%, 97%, 99% or 100% sequence identity to the clone sequence set forth in 16412.

In some embodiments, the trispecific fusion protein comprises (i) a first heavy chain having at least 90%, 95%, 97%, 99% or 100% sequence identity to the clone sequence set forth in 29211, (ii) a second heavy chain having at least 90%, 95%, 97%, 99% or 100% sequence identity to the clone sequence set forth in 28373, and (iii) a light chain having at least 90%, 95%, 97%, 99% or 100% sequence identity to the clone sequence set forth in 16412.

In some embodiments, the trispecific fusion protein is tetravalent and the TAA is MSLN.

In some embodiments, the trispecific fusion protein comprises (i) a first heavy chain having at least 90%, 95%, 97%, 99% or 100% sequence identity to the clone sequences set forth in 29257 or 29283, (ii) a second heavy chain having at least 90%, 95%, 97%, 99% or 100% sequence identity to the clone sequences set forth in 23867, 29258, 29264, 23867, 29263, 29267 or 29261 and (iii) a light chain having at least 90%, 95%, 97%, 99% or 100% sequence identity to the clone sequences set forth in 16412, 29226 or 29220.

In some embodiments, the trispecific fusion protein comprises (i) a first heavy chain having at least 90%, 95%, 97%, 99% or 100% sequence identity to the clone sequence set forth in 29257, (ii) a second heavy chain having at least 90%, 95%, 97%, 99% or 100% sequence identity to the clone sequence set forth in 23867, and (iii) a light chain having at least 90%, 95%, 97%, 99% or 100% sequence identity to the clone sequence set forth in 29220.

In some embodiments, wherein the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 29258, and (iii) a light chain having at least 90%, 95%, 97%, 99% or 100% sequence identity to the clone sequence set forth in 16412.

In some embodiments, the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 29264, and (iii) a light chain having at least 90%, 95%, 97%, 99% or 100% sequence identity to the clone sequence set forth in 16412.

In some embodiments, the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 23867, and (iii) a light chain having at least 90%, 95%, 97%, 99% or 100% sequence identity to the clone sequence set forth in 29220.

In some embodiments, the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 23867, and (iii) a light chain having at least 90%, 95%, 97%, 99% or 100% sequence identity to the clone sequence set forth in 29226.

In some embodiments, the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 29263, and (iii) a light chain having at least 90%, 95%, 97%, 99% or 100% sequence identity to the clone sequence set forth in 16412.

In some embodiments, the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 29264, and (iii) a light chain having at least 90%, 95%, 97%, 99% or 100% sequence identity to the clone sequence set forth in 16412.

In some embodiments, the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 29261, and (iii) a light chain having at least 90%, 95%, 97%, 99% or 100% sequence identity to the clone sequence set forth in 16412.

In some embodiments, the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 29267, and (iii) a light chain having at least 90%, 95%, 97%, 99% or 100% sequence identity to the clone sequence set forth in 16412.

In some embodiments, the trispecific fusion protein is tetravalent and the TAA is Cldn18.2.

In some embodiments, the trispecific fusion protein comprises (i) a first heavy chain having at least 90%, 95%, 97%, 99% or 100% sequence identity to the clone sequence set forth in 29244, (ii) a second heavy chain having at least 90%, 95%, 97%, 99% or 100% sequence identity to the clone sequences set forth in 29245, 29248, 29251 or 29254 and (iii) a light chain having at least 90%, 95%, 97%, 99% or 100% sequence identity to the clone sequence set forth in 16412.

In some embodiments, the trispecific fusion protein comprises (i) a first heavy chain having at least 90%, 95%, 97%, 99% or 100% sequence identity to the clone sequence set forth in 29244, (ii) a second heavy chain having at least 90%, 95%, 97%, 99% or 100% sequence identity to the clone sequence set forth in 29245, and (iii) a light chain having at least 90%, 95%, 97%, 99% or 100% sequence identity to the clone sequence set forth in 16412.

In some embodiments, the trispecific fusion protein comprises (i) a first heavy chain having at least 90%, 95%, 97%, 99% or 100% sequence identity to the clone sequence set forth in 29244, (ii) a second heavy chain having at least 90%, 95%, 97%, 99% or 100% sequence identity to the clone sequence set forth in 29248, and (iii) a light chain having at least 90%, 95%, 97%, 99% or 100% sequence identity to the clone sequence set forth in 16412.

In some embodiments, the trispecific fusion protein comprises (i) a first heavy chain having at least 90%, 95%, 97%, 99% or 100% sequence identity to the clone sequence set forth in 29244, (ii) a second heavy chain having at least 90%, 95%, 97%, 99% or 100% sequence identity to the clone sequence set forth in 29251, and (iii) a light chain having at least 90%, 95%, 97%, 99% or 100% sequence identity to the clone sequence set forth in v16412.

In some embodiments, the trispecific fusion protein comprises (i) a first heavy chain having at least 90%, 95%, 97%, 99% or 100% sequence identity to the clone sequence set forth in 29244, (ii) a second heavy chain having at least 90%, 95%, 97%, 99% or 100% sequence identity to the clone sequence set forth in 29254, and (iii) a light chain having at least 90%, 95%, 97%, 99% or 100% sequence identity to the clone sequence set forth in 16412.

In some embodiments, a trispecific fusion protein of the present disclosure does not comprise a first binding domain that is a Fab capable of binding CD3, a second binding domain that is a scFv capable of binding to HER2, and a PD-1 polypeptide in which (i) the PD-1 polypeptide is linked to the N-terminus of the anti-CD3 Fab domain, (ii) the anti-CD3 Fab domain linked to the N-terminus of a first Fc polypeptide of a heterodimeric Fc domain and (iii) the anti-HER2 scFv domain is liked to a second Fc polypeptide of the heterodimeric Fc domain.

In some embodiments, the trispecific fusion protein is not v31929, which consists of the three polypeptide chain clones 22080 (heavy chain (H) 1), 12985 (light chain (L) 1) and 21490 (heavy chain, H2) comprising the amino acid sequences set forth in SEQ ID NO: 141, SEQ ID NO: 102 and SEQ ID NO: 119, respectively.

In some embodiments, the trispecific fusion protein is v38449, i.e., is tetravalent, and comprises or consists of a first heavy chain comprising the clone sequence set forth in 29283, (ii) a second heavy chain comprising the clone sequence set forth in 29258, and (iii) a light chain comprising the clone sequence set forth in 16412.

In some embodiments, the trispecific fusion protein is v38917, i.e., is tetravalent, and comprises or consists of a first heavy chain comprising the clone sequence set forth in 29283, (ii) a second heavy chain comprising the clone sequence set forth in 29261, and (iii) a light chain comprising the clone sequence set forth in 16412.

In some embodiments, the trispecific fusion protein is v38410, i.e., is trivalent, and comprises or consists of a first heavy chain comprising the clone sequence set forth in 29238, (ii) a second heavy chain comprising the clone sequence set forth in 28373, and (iii) a light chain comprising the clone sequence set forth in 16412.

In some embodiments, the trispecific fusion protein is v38729, i.e., is trivalent, and comprises or consists of a first heavy chain comprising the clone sequence set forth in 29209, (ii) a second heavy chain comprising the clone sequence set forth in 28373, and (iii) a light chain comprising the clone sequence set forth in 16412.

In some embodiments, the trispecific fusion protein is v38999, i.e., is tetravalent, and comprises or consists of a first heavy chain comprising the clone sequence set forth in 29244, (ii) a second heavy chain comprising the clone sequence set forth in 29245, and (iii) a light chain comprising the clone sequence set forth in 16412.

In some embodiments, the trispecific fusion protein is v39000, i.e., is tetravalent, and comprises or consists of a first heavy chain comprising the clone sequence set forth in 29244, (ii) a second heavy chain comprising the clone sequence set forth in 29248, and (iii) a light chain comprising the clone sequence set forth in 16412.

In some embodiments, the trispecific fusion protein is v39003, i.e., is tetravalent, and comprises or consists of a first heavy chain comprising the clone sequence set forth in 29244, (ii) a second heavy chain comprising the clone sequence set forth in 29251, and (iii) a light chain comprising the clone sequence set forth in 16412.

In some embodiments, the trispecific fusion protein is v39004, i.e., is tetravalent, and comprises or consists of a first heavy chain comprising the clone sequence set forth in 29244, (ii) a second heavy chain comprising the clone sequence set forth in 29254, and (iii) a light chain comprising the clone sequence set forth in 16412.

In some embodiments, a trispecific fusion protein herein is trivalent, capable of binding HER2, and is selected from the group consisting of v38400, v38403, v38404, v38405, v31927, v31928 and v38407.

In some embodiments, a trispecific fusion protein herein is trivalent, capable of binding MSLN, and is selected from the group consisting of v38520, v38440, v38441, v38442, v38443, v38344, v38345, v38910, v38911, v38913 and v38914. In other embodiments, a trispecific fusion protein herein is tetravalent, capable of binding MSLN, and is selected from the group consisting of v38448, v38449, v38450, v38451, v38452, v38915, v38919, v38921 and v38922.

In some embodiments, a trispecific fusion protein herein is trivalent, capable of binding Cldn18.2, and is selected from the group consisting of v38408, v38409, v38410, v38411, v38522, v38729, v38731, v38732, v38733, v38735, v38736, v38737, v38739 and v38740. In other embodiments, a trispecific fusion protein herein is tetravalent, capable of binding Cldn18.2, and is selected from the group consisting of v38412, v38413, v38414, v38415, v38416, v38741, v38743, v38744, v38917, v38918, v38999, v39000, v39003, v39004 and v39007.

In some embodiments, a trispecific fusion protein of the present disclosure has an EC₅₀ value for killing tumor cells in a TDCC assay of from about 0.01 pM to about 3 pM, from about 0.02 pM to about 2.5 pM, from about 0.0.5 pM to about 1 pM, or from about 0.02 pM to about 1 pM. In some embodiments, a trispecific fusion protein of the present disclosure has an EC₅₀ value that is from about 1,500-fold to about 180,000-fold higher than that of a corresponding bispecific construct with the same format but that does not comprise an anti-PD-L1 moiety (e.g., a PD-1 polypeptide as described herein). In some embodiments, the trispecific fusion protein is trivalent or tetravalent and comprises a PD-1 polypeptide having an amino acid sequence having at least about 95%, 97%, 99% or 100% sequence identity to the sequence set forth in SEQ ID NO: 9 or 10.

The respective polypeptide chain (i.e., clone) sequences that some of the fusion proteins herein can comprise or consist of are shown in Table X1 as well as the sequence tables herein.

In certain embodiments, the fusion protein is conjugated to another therapeutic and/or diagnostic moiety, for example, a chemotherapeutic agent, or a radioisotope.

### V. Biologically Functional Proteins

In certain embodiments, the biologically functional proteins of the trispecific fusion proteins described herein comprise at least one antigen-binding domain. The binding domains can be, for example, immunoglobulin-based binding domains or non-immunoglobulin-based antibody mimetics, or other polypeptides or small molecules capable of specifically binding to their target, for example, a natural or engineered ligand. Non-immunoglobulin-based antibody mimetic formats include, for example, anticalins, fynomers, affimers, alphabodies, DARPins, and avimers.

The fusion proteins described herein can include or consist of a biologically functional protein. Examples of biologically functional proteins include but are not limited to antibodies and molecules derived therefrom, e.g., polypeptides with antigen binding domains, and polypeptide scaffolds, e.g., a dimeric Fc domain. Thus, in certain embodiments, one or more polypeptide chains, e.g., the first and second polypeptides of the biologically functional proteins described herein are polypeptides comprising variable and/or constant domains of antibodies, or other domains conferring an antigen binding function or a scaffolding function to the fusion protein.

### Antibodies

In certain embodiments, the biologically functional protein is an antibody, i.e., immunoglobin. Antibodies according to the present disclosure can take on various formats as described herein, including antibody fragments, multivalent and/or multispecific antibodies, etc. Thus, in certain embodiments, the biologically functional protein is a multivalent and multispecific antibody. The terms “antibody” and “immunoglobulin” are used interchangeably herein to refer to a polypeptide encoded by an immunoglobulin gene or genes, or a modified version of an immunoglobulin gene, which polypeptide, or a portion thereof, specifically binds to an antigen.

Specific binding of the fusion proteins described herein can be measured, for example, through an enzyme-linked immunosorbent assay (ELISA), a surface plasmon resonance (SPR) technique (employing, for example, a BIAcore instrument) (Liljeblad et al., 2000, Glyco J, 17:323-329), or a traditional binding assay (Hecley, 2002, Endocr Res, 28:217-229). In certain embodiments, specific binding is defined as the extent of binding to an unrelated protein being less than about 10% of the binding to the target antigen as measured by SPR, for example. In certain embodiments, specific binding of an antibody or antibody fragment for a particular antigen or an epitope is defined by a dissociation constant (K_{D}) of ≤1 μM, for example, ≤100 nM, ≤10 nM, ≤1 nM, ≤0.1 nM, ≤0.01 nM, or ≤0.001 nM. In certain embodiments, specific binding of an antibody or antibody fragment for a particular antigen or an epitope is defined by a dissociation constant (K_{D}) of about 10⁻⁶ M or less, for example, about 10⁻⁷ M or less, or about 10⁻⁸ M or less. In some embodiments, specific binding of an antibody or antibody fragment for a particular antigen or an epitope is defined by a dissociation constant (K_{D}) between about 10⁻⁶ M and about 10⁻¹³ M, for example, between about 10⁻⁷ M and about 10⁻¹³ M, between about 10⁻⁸ M and about 10⁻¹³ M, or between about 10⁻⁹ M and about 10⁻¹³ M.

A traditional immunoglobulin structural unit is typically composed of two pairs of polypeptide chains, each pair having one “light” chain (about 25 kilodalton (kD)) and one “heavy” chain (about 50-70 kD). Light chains are classified as either kappa or lambda. The “class” of an immunoglobulin refers to the type of constant domain possessed by its heavy chain. There are five major classes of antibodies: IgA, IgD, IgE, IgG and IgM, and several of these can be further divided into subclasses (isotypes), for example, IgG1, IgG2, IgG3, IgG4, IgA1 and IgA2. The heavy chain constant domains that correspond to the different classes of immunoglobulins are called alpha (a), delta (8), epsilon (¿), gamma (γ) and mu (u), respectively.

In certain embodiments, the antibodies described herein are based on an IgG class immunoglobulin, for example, an IgG1, IgG2, IgG3 or IgG4 immunoglobulin. In some embodiments, the antibodies described herein are based on an IgG1, IgG2 or IgG4 immunoglobulin. In some embodiments, the antibodies described herein are based on an IgG1 immunoglobulin. In the context of the present disclosure, when an antibody is based on a specified immunoglobulin isotype, it is meant that the antibody comprises all or a portion of the constant region of the specified immunoglobulin isotype. It is to be understood that the antibody can also comprise hybrids of isotypes and/or subclasses in some embodiments.

The N-terminal domain of each polypeptide chain of an immunoglobulin defines a variable region of about 100 to 110 or more amino acids in length that is primarily responsible for antigen recognition. The terms variable light chain (VL) and variable heavy chain (VH) refer to these domains in the light and heavy chain respectively.

Accordingly, it can be seen that immunoglobulins can comprise different domains within the heavy and light chains. Such domains can be overlapping and include, the Fc domain (or Fc region), the CH1 domain, the CH2 domain, the CH3 domain, the hinge domain, the heavy constant domain (CH1-hinge-Fc or CH1-hinge-CH2-CH3), the variable heavy domain (VH), the variable light domain (VL) and the light constant domain (CL). The “Fc domain” includes the CH2 and CH3 domains, and optionally a hinge domain (or hinge region).

In each of the VH and VL domains of an immunoglobulin are three loops which are hypervariable in sequence and form an antigen-binding site. Each of these loops is referred to as a “hypervariable region” or “HVR.” The terms hypervariable region (HVR) and complementarity determining region (CDR) are used herein interchangeably in reference to the portions of the variable region that form the antigen-binding domain. With the exception of CDR1 in VH, CDRs generally comprise the amino acid residues that form the hypervariable loops. The VH and VL domains consist of relatively invariant stretches called framework regions (FRs) of between about 15 to 30 amino acids in length separated by the shorter CDRs, which are each typically between about 5 and 15 amino acids in length, although can occasionally be longer or shorter. The three CDRs and four FRs that make up each VH and VL domain are arranged from N- to C-terminus as follows: FR1-CDR1-FR2-CDR2-FR3-CDR3-FR4.

A number of different definitions of the CDR regions are in common use, including those described by Kabat et al. (1983, Sequences of Proteins of Immunological Interest, NIH Publication No. 369-847, Bethesda, MD), by Chothia et al. (1987, J Mol Biol, 196:901-917), as well as the IMGT, AbM and Contact definitions. These different definitions include overlapping or subsets of amino acid residues when compared against each other. By way of example, CDR definitions according to Kabat, Chothia, IMGT, AbM and Contact are provided in Table 1 below. Accordingly, as would be readily apparent to one skilled in the art, the exact numbering and placement of CDRs can differ based on the numbering system employed. However, it is to be understood that the disclosure herein of a variable heavy domain (VH) includes the disclosure of the associated (inherent) heavy chain CDRs (HCDRs) as defined by any of the known numbering systems. Similarly, disclosure herein of a variable light domain (VL) includes the disclosure of the associated (inherent) heavy chain CDRs (HCDRs) as defined by any of the known numbering systems.

TABLE 1
Common CDR Definitions¹

| | Heavy Chain | | | Light Chain | | |
|---|---|---|---|---|---|---|
| Definition | CDR1² | CDR2 | CDR3 | CDR1 | CDR2 | CDR3 |
| Kabat | H31-H35B | H50-H65 | H95-H102 | L24-L34 | L50-L56 | L89-L97 |
| Chothia | H26-H32, | H52-H56 | H95-H102 | L24-L34 | L50-L56 | L89-L97 |
| | H33 or | | | | | |
| | H34 | | | | | |
| IMGT | H26-H33, | H51-H57 | H93-102 | L27-L32 | L50-L52 | L89-L97 |
| | H34, H35, | | | | | |
| | H35A or | | | | | |
| | H35B | | | | | |
| AbM | H26-H35B | H50-H58 | H95-H102 | L24-L34 | L50-L56 | L89-L97 |
| Contact | H30-H35B | H47-H58 | H95-H101 | L30-L36 | L46-L55 | L89-L96 |

¹Either the Kabat or Chothia numbering system can be used for HCDR2, HCDR3 and the light chain CDRs for all definitions except Contact, which uses Chothia numbering
²Using Kabat numbering. The position in the Kabat numbering scheme that demarcates the end of the Chothia and IMGT CDR-H1 loop varies depending on the length of the loop due to the placement of insertions outside of those CDR definitions at positions 35A and 35B in Kabat. The IMGT and Chothia CDR-H1 loop can be unambiguously defined using Chothia numbering. CDR-H1 definitions using Chothia numbering are: Kabat H31-H35, Chothia H26-H32, AbM H26-H35, IMGT H26-H33, Contact H30-H35.

One skilled in the art will appreciate that a limited number of amino acid substitutions can be introduced into the CDR sequences or to the VH or VL sequences of known antibodies or antibody fragments without the antibody or fragment losing its ability to bind its target. Candidate amino acid substitutions can be identified by computer modeling or by techniques such as alanine scanning as described above, with the resulting variants being tested for binding activity by standard techniques. For example, in certain embodiments, the EGFR binding domain comprised by the fusion protein comprises a set of CDRs (i.e., heavy chain CDR1, CDR2 and CDR3, and light chain CDR1, CDR2 and CDR3) that have 90% or greater, 95% or greater, 98% or greater, 99% or greater, or 100% sequence identity to a set of CDRs from cetuximab or panitumumab, wherein the binding domain retains the ability to bind EGFR. In certain embodiments, the EGFR binding domain comprised by the fusion protein comprises a variant of these CDR sequences comprising between 1 and 10 amino acid substitutions across the three CDRs (that is, the CDRs can be modified by including up to 10 amino acid substitutions with any combination of CDRs being modified), for example, between 1 and 7 amino acid substitutions, between 1 and 5 amino acid substitutions, between 1 and 4 amino acid substitutions, between 1 and 3 amino acid substitutions, between 1 and 2 amino acid substitutions, or 1 amino acid substitution, across the CDRs, wherein the variant retains the ability to bind EGFR. Typically, in some embodiments, such amino acid substitutions will be conservative amino acid substitutions such as those outlined in Column 1 or Column 2 of Table 4 below.

In certain embodiments, the antibodies, e.g., trispecific fusion proteins described herein, comprise at least one immunoglobulin domain from a mammalian immunoglobulin, such as a bovine immunoglobulin, a human immunoglobulin, a camelid immunoglobulin, a rat immunoglobulin or a mouse immunoglobulin. In some embodiments, a biologically functional protein, e.g., trispecific fusion proteins described herein, can be a chimeric antibody and comprises two or more immunoglobulin domains, in which at least one domain is from a first mammalian immunoglobulin, for example a human immunoglobulin, and at least a second domain is from a second mammalian immunoglobulin, for example, a mouse or rat immunoglobulin. In some embodiments, the biologically functional protein, e.g., trispecific fusion proteins described herein, comprises at least one immunoglobulin constant domain from a human immunoglobulin.

One skilled in the art will understand that these antibody domains can be combined in various ways to provide an antibody having different formats, including multispecific antibodies of different formats, e.g., those described in FIG. 3A-3LL. In some embodiments, these formats are based generally on antibody formats known in the art (see, for example, review by Brinkmann & Kontermann, 2017, MABS, 9 (2): 182-212, and Müller & Kontermann, “Bispecific Antibodies” in Handbook of Therapeutic Antibodies, Wiley-VCH Verlag GmbH & Co. (2014)).

The antibodies of the biologically functional proteins described herein, e.g., the trispecific fusion proteins described herein, can have different valencies. In certain embodiments, the biologically functional protein described herein comprises a single antigen binding domain. In certain embodiments, the biologically functional protein described herein comprises two or more antigen binding domains, e.g., three antigen binding domains. In certain embodiments, the biologically functional protein comprises an antibody that has different valencies and specificities. A “bispecific antibody” as used herein, comprises two binding domains. In certain embodiments, each of the two binding domains has a unique binding specificity for an epitope on an antigen. A “multispecific antibody” as used herein, comprises two or more binding domains, e.g., three or four binding domains. In certain embodiments, each of the two or more binding domains has a unique binding specificity for an epitope on an antigen. In some embodiments, at least two of the two or more binding domains have unique binding specificities for two epitopes, which can be on the same or on different antigens. For example, the antibody used herein can be bivalent and bispecific, or can be bivalent and have a single specificity, e.g., like an unmodified and naturally occurring IgG antibody. Alternatively, the antibody used herein can be trivalent and bispecific, that is the antibody comprises three binding domains, wherein two of the three binding domains have specificities for different epitopes, e.g., on the same or different antigens. The antibody can also be bispecific and tetravalent, that is the antibody comprises two pairs of binding domains (i.e., four binding domains) with each pair specifically binding a certain epitope. Other valencies are also possible as further described herein.

When the antibody comprises two binding domains that bind to the same target molecule, the binding domains can bind to the same epitope on the target molecule, or they can bind to different epitopes on the target molecule. In some embodiments, the antibody comprises two binding domains that bind to the same epitopes on the same target molecule (e.g., an antigen such as HER2, MSLN, etc.). In some embodiments, the antibody comprises two binding domains that bind to different epitopes on the target molecule. The term “biparatopic” can be used to refer to an antibody which comprises two binding domains that bind to different epitopes on the same target molecule (e.g., an antigen such as HER2, MSLN, etc.). A biparatopic antibody can bind to a single antigen molecule through the two different epitopes, or it can bind to two separate antigen molecules, each through a different epitope.

In certain embodiments, the antibody described herein is biparatopic for a first antigen and monoparatopic for a second antigen, e.g., trivalent and trispecific in total, in that it comprises a first binding domain and a second binding domain, each of which binds to a different epitope on the first target molecule (biparatopic binding), and a third binding domain that binds to the second target molecule (monoparatopic binding). Alternatively, a tetraspecific and biparatopic antibody can comprise a first binding domain and a second binding domain, each binding to a different epitope on the first target molecule (biparatopic binding), and a third binding domain and a fourth binding domain, each binding to a different epitope on the second target molecule (biparatopic binding).

In some embodiments, the antibody described herein further comprises a scaffold and the binding domains are operably linked to the scaffold. “Operably linked,” as used herein, means that the components described are in a relationship permitting each of them to function in their intended manner. The binding domains can be directly or indirectly linked to the scaffold. By indirectly linked, it is meant that a given binding domain is linked to the scaffold via another component, for example, a linker or one of the other binding domains. Various formats for fusion proteins that comprise a scaffold are described in more detail below.

### VI. Antigen Binding Domain Formats

In some embodiments, the fusion proteins described herein include an antibody having at least one antigen binding domain that is an antibody fragment, such as a Fab, a Fab′, a single chain Fab (scFab), a single chain Fv (scFv), a single domain antibody (sdAb), or combinations thereof. In various embodiments, a trispecific fusion protein of the present disclosure comprises three binding domains, wherein at least one of such domains is a Fab domain, and at least one other domain is an scFv domain, as described herein.

A “Fab” or “Fab fragment,” as used herein, contains the constant domain (CL) of the light chain and the first constant domain (CH1) of the heavy chain along with the variable domains VL and VH on the light and heavy chains, respectively, which comprise the CDRs. A Fab′ or Fab′ fragment differs from a Fab fragment by the addition of a few amino acid residues at the C-terminus of the heavy chain CH1 domain, including one or more cysteine residues from the hinge region.

A Fab fragment can comprise or consist of two separate polypeptide chains (a light chain and a heavy chain) or it can be a single chain Fab. A single chain Fab is a Fab molecule in which the Fab light chain and the Fab heavy chain are connected by a peptide linker to form a single peptide chain. Typically, the C-terminus of the Fab light chain is connected to the N-terminus of the Fab heavy chain in the single-chain Fab molecule, however, other formats are also possible. In various embodiments of this disclosure, the Fab of a trispecific fusion protein is not a single chain Fab and comprises a light (CL+VL sequences) and a heavy chain (CH1+VH sequences) as described herein.

An “scFv” or “scFv domain,” as used herein, includes a heavy chain variable domain (VH) and a light chain variable domain (VL) of an antibody in a single polypeptide chain. The scFv of a trispecific fusion protein can optionally comprise a polypeptide linker between the VH and VL domains which can assist the scFv in forming a desired structure for antigen binding. An scFv can include a VL connected from its C-terminus to the N-terminus of a VH by a linker, i.e., VL-Linker-VH (from N- to C-terminus), or alternatively, an scFv can comprise a VH connected through its C-terminus to the N-terminus of a VL by a linker, i.e., VH-Linker-VL (from N- to C-terminus). For a review of scFvs see Pluckthun in The Pharmacology of Monoclonal Antibodies, vol. 113, Rosenburg and Moore eds., Springer-Verlag, New York, pp. 269-315 (1994). In some embodiments, the linker that is connecting the VL and VH sequences of an scFv used in the fusion proteins herein can be a peptide linker. Such peptide linker can comprise or consist of a sequence of about 5, 10, 15, 20, 25, or more consecutive amino acid residues. In some embodiments, such linker can comprise or consist of the sequence (GₐS_{b})x, and wherein a, b, and x are independently integers from 1 to 5. In some embodiments, such linker comprises or consists of the amino acid sequence (G₄S)x, and wherein x is an integer from 1 to 5. In some embodiments, such linker comprises or consists of the amino acid sequence set forth in SEQ ID NO: 284.

The term “sdAb” refers to a single immunoglobulin domain. An sdAb can be, for example, of camelid origin. Camelid antibodies lack light chains, and their antigen-binding sites consist of a single domain, termed a “VHH.” An sdAb comprises three CDR/hypervariable loops that form the antigen-binding site: CDR1, CDR2 and CDR3. sdAbs are fairly stable and easy to express, for example, as a fusion with the Fc chain of an antibody (see, for example, Harmsen & De Haard, 2007, Appl. Microbiol Biotechnol. 77 (1): 13-22).

In some embodiments, one or more of the binding domains comprised by the antibody and trispecific fusion protein described herein can be a natural (e.g., a wildtype amino acid sequence) or engineered (e.g., a sequence having one or more amino acid modifications relative to a wildtype amino acid sequence) ligand or binding domain for the target receptor (e.g., antigen), or a functional fragment of such a ligand, i.e., a fragment capable of specifically binding to the target receptor (e.g., antigen).

In some embodiments, the antigen binding domains of the trispecific fusion proteins described herein can be in the form of combinations of individual scFvs, Fabs, sdAbs, etc. For example, when the binding domains are in the form of scFvs, formats such as tandem scFv ((scFv) 2 or taFv) or triplebody (3 scFvs) can be constructed, in which the scFvs are connected together by a flexible linker. ScFvs can also be used to construct diabody, triabody and tetrabody (tandem diabodies or TandAbs) formats, which comprise 2, 3 and 4 scFvs, respectively, connected by a short linker. The restricted length of the linker (usually about 5 amino acids in length) results in dimerization of the scFvs in a head-to-tail manner. In any of the preceding formats, the scFvs can be further stabilized by inclusion of an interdomain disulfide bond. For example, a disulfide bond can be introduced between VL and VH through introduction of an additional cysteine residue in each chain (for example, at position 44 in VH and 100 in VL) (see, for example, Fitzgerald et al., 1997, Protein Engineering, 10:1221-1225) or a disulfide bond can be introduced between two VHs to provide an antigen binding domain having a DART format (see, for example, Johnson et al., 2010, J Mol. Biol., 399:436-449).

Similarly, fusion protein formats comprising two or more sdAbs, such as VHs or VHHs, connected together through a suitable linker can be used for the biologically functional protein. Other examples of antibody formats that lack a scaffold include those based on Fab fragments, for example, Fab₂, F(ab′)₂ and F(ab′)₃ formats, in which the Fab fragments are connected through a linker or an IgG hinge region.

Combinations of antigen binding domains in different forms can also be employed to generate alternative formats. For example, an scFv or a sdAb can be fused to the C-terminus of either or both of the light and heavy chain of a Fab fragment resulting in a bivalent (Fab-scFv) or (Fab-sdAb) or trivalent (Fab-(scFv)₂ or Fab-(sdAb)₂). Similarly, one or two scFvs or sdAbs can be fused at the hinge region of a F(ab′) fragment to produce a tri- or tetravalent F(ab′)₂-scFv/sdAb. The binding domains can be in one or a combination of the forms described above (for example, scFvs, Fabs and/or sdAbs, or ligand-based binding domains).

In certain specific embodiments, the biologically functional protein of a trispecific fusion protein herein comprises a bi-specific antibody that binds an immune cell antigen, e.g., CD3, and a tumor associated antigen (TAA), e.g., HER2. In certain more specific embodiments, the biologically functional protein comprises a bispecific antibody with a Fab-scFv format wherein the Fab binds an immune cell antigen and the scFv binds a TAA. In certain more specific embodiments, the biologically functional protein comprises a bispecific antibody with a Fab-scFv format wherein the Fab binds CD3 and the scFv binds HER2. In some embodiments, the biologically functional protein comprises a bispecific antibody with a Fab-Fab format wherein one Fab binds CD3 and the other Fab binds HER2.

In certain embodiments, the biologically functional protein comprises two or more antigen binding domains operably linked to a heterodimeric Fc. In this context, the biologically functional protein can be bivalent, trivalent or tetravalent. Non-limiting examples of formats are described below and further herein, e.g., in FIG. 3A-3LL. Other configurations are known in the art (see, for example, Spiess et al., 2015, Mol Immunol., 67:95-106).

Exemplary configurations for a biologically functional protein herein comprising two binding domains operably linked to a heterodimeric Fc, i.e., a bivalent antibody, include, but are not limited to: a) mAb format in which the first binding domain is a Fab that is operably linked to the N-terminus of the first Fc polypeptide of the heterodimeric Fc and the second binding domain is a Fab that is operably linked to the N-terminus of the second Fc polypeptide; b) hybrid format in which the first binding domain is an scFv that is operably linked to the N-terminus of one Fc polypeptide of the heterodimeric Fc and the second binding domain is a Fab that is operably linked to the N-terminus of the other Fc polypeptide, and c) dual scFv format in which the first binding domain is an scFv that is operably linked to the N-terminus of the first Fc polypeptide of the heterodimeric Fc and the second binding domain is an scFv that is operably linked to the N-terminus of the second Fc polypeptide.

Other examples include antibodies comprising one binding domain (either first or second) as a Fab or an scFv operably linked to the N-terminus of the first Fc polypeptide and the other binding domain as a Fab or an scFv operably linked to the C-terminus of the second Fc polypeptide.

Exemplary configurations for a multispecific antibody (e.g., fusion protein) comprising three binding domains operably linked to a heterodimeric Fc (i.e., a trivalent antibody which can be mono-, bi- or trispecific) include, but are not limited to: A) mAb-Fv format in which the first binding domain is a Fab that is operably linked to the N-terminus of the first Fc polypeptide of the heterodimeric Fc and the second binding domain is a Fab that is operably linked to the N-terminus of the second Fc polypeptide, with the third binding domain being made up of a VH domain attached to the C-terminus of one Fc polypeptide and a VL domain attached to the C-terminus of the other Fc polypeptide; B) mAb-scFv format in which the first binding domain is a Fab that is operably linked to the N-terminus of the first Fc polypeptide of the heterodimeric Fc, the second binding domain is a Fab that is operably linked to the N-terminus of the second Fc polypeptide and the third binding domain is an scFv operably linked to the C-terminus of either the first or the second Fc polypeptide; C) scFv-mAb format in which the first binding domain is a Fab that is operably linked to the N-terminus of the first Fc polypeptide of the heterodimeric Fc, the second binding domain is a Fab that is operably linked to the N-terminus of the second Fc polypeptide and the third binding domain is an scFv operably linked to the N-terminus of either the first or the second binding domain; D) central scFv format in which the first binding domain is an scFv that is operably linked to the N-terminus of one Fc polypeptide of the heterodimeric Fc, the second binding domain is a Fab that is operably linked to the N-terminus of the other Fc polypeptide, and the third binding domain is a Fab that is operably linked to the first binding domain (scFv); E) Fab-hybrid format in which the first binding domain is an scFv that is operably linked to the N-terminus of one Fc polypeptide of the heterodimeric Fc, the second binding domain is a Fab that is operably linked to the N-terminus of the other Fc polypeptide, and the third binding domain is a Fab that is operably linked to the N-terminus of the first or second binding domain; F) scFv-hybrid format in which the first binding domain is an scFv that is operably linked to the N-terminus of one Fc polypeptide of the heterodimeric Fc, the second binding domain is a Fab that is operably linked to the N-terminus of the other Fc polypeptide, and the third binding domain is an scFv that is operably linked to the N-terminus of the first or second binding domain; G) hybrid-scFv format in which the first binding domain is an scFv that is operably linked to the N-terminus of one Fc polypeptide of the heterodimeric Fc, the second binding domain is a Fab that is operably linked to the N-terminus of the other Fc polypeptide, and the third binding domain is an scFv that is operably linked to the C-terminus of either the first or the second Fc polypeptide; H) hybrid-Fab format in which the first binding domain is an scFv that is operably linked to the N-terminus of one Fc polypeptide of the heterodimeric Fc, the second binding domain is a Fab that is operably linked to the N-terminus of the other Fc polypeptide, and the third binding domain is a Fab that is operably linked to the C-terminus of either the first or the second Fc polypeptide; and I) Fab-mAb format in which the first binding domain is a Fab that is operably linked to the N-terminus of the first Fc polypeptide of the heterodimeric Fc, the second binding domain is a Fab that is operably linked to the N-terminus of the second Fc polypeptide and the third binding domain is a Fab operably linked to the N-terminus of either the first or the second binding domain.

In various embodiments, a trivalent fusion protein of the present disclosure is trispecific. Such trivalent and trispecific fusion protein herein can comprise a first binding domain (e.g., a Fab or scFv domain) capable of binding an antigen (e.g., CD3) on a cytotoxic effector cell (e.g., an immune cell such as a T cell), a second binding (e.g., a Fab or scFv domain) capable of binding a TAA on a tumor cell, and a third binding domain (e.g., PD-1 polypeptide) capable of binding PD-L1 on a tumor cell.

Exemplary configurations for a multispecific antibody (e.g., fusion protein) comprising four binding domains operably linked to a heterodimeric Fc, i.e., a tetravalent antibody which can be mono-, bi-, tri- or tetraspecific, include, but are not limited to: i) central-scFv2 format in which the first binding domain is an scFv that is operably linked to the N-terminus of one Fc polypeptide of the heterodimeric Fc, the second binding domain is an scFv that is operably linked to the N-terminus of the other Fc polypeptide, the third binding domain is a Fab that is operably linked to one of the scFvs and the fourth binding domain is a Fab that is operably linked to the other scFv, and ii) dual variable domain format in which the first binding domain is a Fab that is operably linked to the N-terminus of one Fc polypeptide of the heterodimeric Fc, the second binding domain is a Fab that is operably linked to the N-terminus of the other Fc polypeptide, the third binding domain is an scFv that is operably linked to one of the Fabs and the fourth binding domain is an scFv that is operably linked to the other Fab. In various other embodiments herein, a tetravalent fusion protein comprises a first binding domain which is a Fab domain, a second binding domain which is a first scFv domain, a third binding domain which is a second scFv domain, and a fourth binding domain. In some embodiments, the fourth binding domain is a Fab or scFv domain. In other embodiments, the fourth binding domain has a non-Fab or non-scFv structure and can be a polypeptide chain having a specificity for a certain antigen. In various embodiments, the fourth binding domain is a PD-1 polypeptide, either a wildtype PD-1 protein or a modified (e.g., mutated and/or truncated) variant thereof.

The antibodies of the biologically functional proteins, e.g., fusion proteins, described herein can comprise a label, a drug, or combinations thereof. Any label known in the art suitable for detection of the fusion proteins described herein can be used. Antibody drug conjugates are described in more detail below.

In certain embodiments, the antigen binding domains of the antibodies of the biologically functional protein described herein bind to the same antigen on the same cell. In certain embodiments, the antigen binding domains bind to more than one antigen on the same cell. In certain embodiments, the antigen binding domains bind to more than one antigen, wherein at least one antigen is on a different cell than another antigen. In certain embodiments, the antigen binding domain(s) of the antibody bind to a tumor cell or an immune cell. In certain embodiments, the antigen binding domains of the antibody bind to a tumor cell and an immune cell.

### Chimeric, Humanized and Variant Antibodies

In some embodiments, the antibodies of the fusion proteins described herein can be derived from immunoglobulins that are from different species, for example, the antibody can be a chimeric antibody or a humanized antibody, as described herein. A “chimeric antibody” refers to an antibody that typically comprises at least one variable domain from a rodent antibody (usually a murine antibody) and at least one constant domain from a human antibody. A “humanized antibody” is a type of chimeric antibody that contains minimal sequence derived from a non-human antibody.

The human constant domain of a chimeric antibody need not be of the same isotype as the non-human constant domain it replaces. Chimeric antibodies are discussed, for example, in Morrison et al., 1984, Proc. Natl. Acad. Sci. USA, 81:6851-55, and U.S. Pat. No. 4,816,567. Generally, humanized antibodies are human immunoglobulins (recipient antibody) in which residues from a hypervariable region of the recipient are replaced by residues from a hypervariable region of a non-human species (donor antibody), such as mouse, rat, rabbit, or non-human primate, having the desired specificity and affinity for a target antigen. This technique for creating humanized antibodies is often referred to as “CDR grafting.” “Chimeric antibody” and “humanized antibody” both refer generally to antibodies that combine immunoglobulin regions or domains from more than one species.

In some instances, additional modifications are made to further refine antibody performance. For example, framework region (FR) residues of the human immunoglobulin are replaced by corresponding non-human residues, or the humanized antibodies can comprise residues that are not found in either the recipient antibody or the donor antibody. In general, a variable domain in a humanized antibody will comprise all or substantially all of the hypervariable regions from a non-human immunoglobulin and all or substantially all of the FRs from a human immunoglobulin sequence. Humanized antibodies are described in more detail in Jones, et al., 1986, Nature, 321:522-525; Riechmann, et al., 1988, Nature, 332:323-329 and Presta, 1992, Curr. Op. Struct. Biol., 2:593-596, for example.

A number of approaches are known in the art for selecting the most appropriate human frameworks in which to graft the non-human CDRs. Early approaches used a limited subset of well-characterized human antibodies, irrespective of the sequence identity to the non-human antibody providing the CDRs (the “fixed frameworks” approach). More recent approaches have employed variable regions with high amino acid sequence identity to the variable regions of the non-human antibody providing the CDRs (“homology matching” or “best-fit” approach). An alternative approach is to select fragments of the framework sequences within each light or heavy chain variable region from several different human antibodies. CDR-grafting can in some cases result in a partial or complete loss of affinity of the grafted molecule for its target antigen. In such cases, affinity can be restored by back-mutating some of the residues of human origin to the corresponding non-human ones. Methods for preparing humanized antibodies by these approaches are well-known in the art (see, for example, Tsurushita & Vasquez, 2004, Humanization of Monoclonal Antibodies, Molecular Biology of B Cells, 533-545, Elsevier Science (USA); Jones et al., 1986, Nature, 321:522-525; Riechmann et al., 1988, Nature, 332:323-329; Presta et al., 1997, Cancer Res, 57 (20): 4593-4599).

Alternatively, or in addition to these traditional approaches, more recent technologies can be employed to further reduce the immunogenicity of a CDR-grafted humanized antibody. For example, frameworks based on human germline sequences or consensus sequences can be employed as acceptor human frameworks rather than human frameworks with somatic mutation(s). Another technique that aims to reduce the potential immunogenicity of non-human CDRs is to graft only specificity-determining residues (SDRs). In this approach, only the minimum CDR residues required for antigen-binding activity (the “SDRs”) are grafted into a human germline framework. This method improves the “humanness” (i.e., the similarity to human germline sequence) of the humanized antibody and thus helps reduce the risk of immunogenicity of the variable region. These techniques have been described in various publications (see, for example, Almagro & Fransson, 2008, Front Biosci, 13:1619-1633; Tan, et al., 2002, J Immunol, 169:1119-1125; Hwang, et al., 2005, Methods, 36:35-42; Pelat, et al., 2008, J Mol Biol, 384:1400-1407; Tamura, et al., 2000, J Immunol, 164:1432-1441; Gonzales, et al., 2004, Mol Immunol, 1:863-872, and Kashmiri, et al., 2005, Methods, 36:25-34).

In certain embodiments, the antibody of a fusion protein herein comprises humanized antibody sequences, for example, one or more humanized variable domains. In some embodiments, the antibody of a fusion protein herein is a humanized antibody.

In certain embodiments, an antigen binding domain comprised by the fusion protein is a substitutional variant of a known antibody that comprises one or more amino acid substitutions in the CDRs of the parent antibody. In certain embodiments, the substitution variant has modifications (for example, improvements) in certain biological properties relative to the parent antibody. For example, the substitution variant can have increased or even decreased affinity for the target protein or it can have reduced immunogenicity. In some embodiments, the substitution variant substantially retains certain biological properties of the parent antibody.

CDR hotspots are residues encoded by codons that undergo mutation at high frequency during the somatic maturation process (see, for example, Chowdhury, 2008, Methods Mol. Biol., 207:179-196). Affinity maturation by constructing and reselecting from secondary libraries has been described (see, for example., Hoogenboom et al. in Methods in Molecular Biology, 178:1-37, O'Brien et al., ed., Human Press, Totowa, N.J. (2001)).

Methods of affinity maturation are well known in the art. For example, diversity can be introduced into the variable genes chosen for maturation by various techniques including, for example, error-prone PCR, chain shuffling or oligonucleotide-directed mutagenesis. A secondary library is then created, and this library is screened to identify any antibody variants with the desired affinity. Another method to introduce diversity involves CDR-directed approaches, in which several CDR residues (for example, 2, 3, 4 or more residues at a time) are randomized. CDR3 of either or both of the heavy or light chain is often targeted for CDR-directed approaches. CDR residues involved in antigen binding can be identified for example using alanine scanning mutagenesis (see, for example, Cunningham and Wells, 1989, Science, 244:1081-1085) or by computer modeling using a crystal structure of an antigen-antibody complex to identify contact points between the antibody and antigen.

In certain embodiments, a substitution variant comprises one or more substitutions within one or more CDRs provided that the substitutions do not substantially reduce the ability of the binding domain to bind its target antigen. For example, a substitution variant can comprise one or more conservative substitutions as described herein within one or more CDRs that do not substantially reduce binding affinity. In some embodiments, a substitution variant comprises one or more amino acid substitutions within the CDRs that do not involve the antigen-contacting amino acids. In some embodiments, a substitution variant comprises a variant VH or VL sequence in which each CDR either is unaltered or contains no more than one, two or three amino acid substitutions.

### Glycosylation Variants

In certain embodiments, the fusion proteins described herein comprise a biologically functional protein based on an IgG Fc in which native glycosylation has been modified. As is known in the art, glycosylation of an Fc can be modified to increase or decrease effector function.

For example, mutation of the conserved asparagine residue at position 297 to alanine, glutamine, lysine or histidine (i.e., N297A, Q, K or H) results in an aglycoslated Fc that lacks all effector function (Bolt et al., 1993, Eur. J. Immunol., 23:403-411; Tao & Morrison, 1989, J. Immunol., 143:2595-2601).

Conversely, removal of fucose from heavy chain N297-linked oligosaccharides has been shown to enhance ADCC, based on improved binding to FcγRIIla (see, for example, Shields et al., 2002, J Biol Chem., 277:26733-26740, and Niwa et al., 2005, J. Immunol. Methods, 306:151-160). Such low fucose antibodies can be produced, for example in knockout Chinese hamster ovary (CHO) cells lacking fucosyltransferase (FUT8) (Yamane-Ohnuki et al., 2004, Biotechnol. Bioeng., 87:614-622), in the variant CHO cell line, Lec 13, that has a reduced ability to attach fucose to N297-linked carbohydrates (International Publication No. WO 03/035835), or in other cells that generate afucosylated antibodies (see, for example, Li et al., 2006, Nat Biotechnol, 24:210-215; Shields et al., 2002, ibid, and Shinkawa et al., 2003, J. Biol. Chem., 278:3466-3473). In addition, International Publication No. WO 2009/135181 describes the addition of fucose analogs to culture medium during antibody production to inhibit incorporation of fucose into the carbohydrate on the antibody.

Other methods of producing antibodies with little or no fucose on the Fc glycosylation site (N297) are well known in the art. For example, the GlymaX® technology (ProBioGen AG) (scc von Horsten et al., 2010, Glycobiology, 20 (12): 1607-1618 and U.S. Pat. No. 8,409,572).

Other glycosylation variants include those with bisected oligosaccharides, for example, variants in which a biantennary oligosaccharide attached to the Fc region of the antibody is bisected by N-acetylglucosamine (GlcNAc). Such glycosylation variants can have reduced fucosylation and/or improved ADCC function. See, for example, International Publication No. WO 2003/011878, U.S. Pat. No. 6,602,684 and US Patent Application Publication No. US 2005/0123546. Useful glycosylation variants also include those having at least one galactose residue in the oligosaccharide attached to the Fc region, which can have improved CDC function (see, for example, International Publication Nos. WO 1997/030087, WO 1998/58964 and WO 1999/22764).

### Polypeptide Scaffolds

In certain embodiments, the biologically functional protein of the fusion proteins described herein comprises a polypeptide scaffold, which can function, e.g., to stabilize or extend the in vivo half-life of the ligand receptor pair.

In certain embodiments, the biologically functional protein comprises or consists of a dimeric Fc region. In certain embodiments, the first and second polypeptide of the biologically functional protein comprises or consists of a dimeric Fc, wherein the first polypeptide consists of a first Fc polypeptide and the second polypeptide consists of a second Fc polypeptide, the first and second Fc polypeptides forming a dimeric Fc region. In certain embodiments, the dimeric Fc region is a heterodimeric Fc. Heterodimeric Fc regions are described in more detail herein.

In certain embodiments, the polypeptide scaffolds are comprised of a first and second polypeptide. In certain embodiments, the PD-1 domain is fused via a peptidic linker to the first polypeptide (e.g., a first heavy chain) or the second polypeptide (e.g., a second heavy chain). Thus, in certain embodiments, the PD-1 domain is fused to the N-terminus of a first polypeptide or to the N-terminus of a second polypeptide via a peptidic linker. Conversely, in certain embodiments, the PD-1 domain is fused to the C-terminus of a first polypeptide or a second polypeptide via a peptidic linker.

In certain embodiments, the biologically functional protein comprises a polypeptide scaffold that consists of a dimeric Fc. In some embodiments, the polypeptide scaffold consisting of a heterodimeric Fc comprises a modified CH3 and/or CH2 domain of Table 2 and/or Table 3, respectively.

### Fc Domains

In certain embodiments, the trispecific fusion proteins described herein include biologically functional proteins, e.g., antibodies and/or polypeptide scaffolds, comprising a dimeric immunoglobulin Fc region. The term “Fc region” includes native sequence Fc regions and variant Fc regions. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD (1991). An “Fc polypeptide” of a dimeric Fc refers to one of the two polypeptides forming the dimeric Fc region, that is a polypeptide comprising C-terminal constant regions of an immunoglobulin heavy chain that is capable of stable self-association.

An Fc region can comprise either a CH3 domain or a CH3 and a CH2 domain. The CH3 domain comprises two CH3 sequences, each comprised by one of the two Fc polypeptides of the dimeric Fc. Similarly, the CH2 domain comprises two CH2 sequences, each comprised by one of the two Fc polypeptides of the dimeric Fc.

In certain embodiments, the fusion proteins described herein comprise an Fc based on a human IgG Fc. In some embodiments, the fusion proteins comprise an Fc based on a human IgG1 Fc. In some embodiments, the fusion proteins comprise an Fc based on a heterodimeric Fc comprising two different Fc polypeptides.

In certain embodiments, the fusion proteins described herein comprise an Fc based on a modified IgG Fc in which the CH3 domain comprises one or more amino acid modifications. In some embodiments, the fusion protein comprises an Fc based on a modified IgG Fc in which the CH2 domain comprises one or more amino acid modifications. In some embodiments, the fusion protein comprises an Fc based on a modified IgG Fc in which the CH3 domain comprises one or more amino acid modifications and the CH2 domain comprises one or more amino acid modifications.

### Modified Fc CH3 Domains

In certain embodiments, the fusion proteins herein comprise a heterodimeric immunoglobulin Fc comprising a modified CH3 domain, wherein the modified CH3 domain comprises one or more asymmetric amino acid modifications. As used herein, an “asymmetric amino acid modification” refers to a modification in which an amino acid at a specific position on the first Fc polypeptide is different to the amino acid at the corresponding position on the second Fc polypeptide. These asymmetric amino acid modifications can comprise modification of only one of the two amino acids at the corresponding position on each Fc polypeptide, or they can comprise modifications of both amino acids at the corresponding positions on each of the first and second Fc polypeptides.

In certain embodiments, the fusion protein comprises a heterodimeric Fc comprising a modified CH3 domain, wherein the modified CH3 domain comprises one or more asymmetric amino acid modifications that promote formation of the heterodimeric Fc over formation of a homodimeric Fc. Amino acid modifications that can be made to the CH3 domain of an Fc in order to promote formation of a heterodimeric Fc are known in the art and include, for example, those described in International Publication No. WO 96/027011 (“knobs into holes”), Gunasckaran et al., 2010, J Biol Chem, 285, 19637-46 (“electrostatic steering”), Davis et al., 2010, Prot Eng Des Sel, 23 (4): 195-202 (strand exchange engineered domain (SEED) technology) and Labrijn et al., 2013, Proc Natl Acad Sci USA, 110 (13): 5145-50 (Fab-arm exchange). Other examples include approaches combining positive and negative design strategies to produce stable asymmetrically modified Fc regions as described in International Publication Nos. WO 2012/058768 and WO 2013/063702.

In certain embodiments, the fusion protein comprises a heterodimeric Fc having a modified CH3 domain as described in International Publication No. WO 2012/058768 or International Patent Publication No. WO 2013/063702.

In some embodiments, the fusion protein comprises a heterodimeric human IgG1 Fc having a modified CH3 domain. Table 2 below provides the amino acid sequence of the human IgG1 Fc sequence, corresponding to amino acids 231 to 447 of the full-length human IgG1 heavy chain. The CH2 domain is typically defined as comprising amino acids 231-340 of the full-length human IgG1 heavy chain and the CH3 domain is typically defined as comprising amino acids 341-447 of the full-length human IgG1 heavy chain.

In certain embodiments, the fusion protein herein comprises a heterodimeric Fc having a modified CH3 domain comprising one or more asymmetric amino acid modifications that promote formation of the heterodimeric Fc over formation of a homodimeric Fc, in which the modified CH3 domain comprises a first Fc polypeptide including amino acid modifications at positions F405 and Y407, and a second Fc polypeptide including amino acid modifications at positions T366 and T394. In some embodiments, the amino acid modification at position F405 of the first Fc polypeptide of the modified CH3 domain is F405A, F405I, F405M, F405S, F405T or F405V. In some embodiments, the amino acid modification at position Y407 of the first Fc polypeptide of the modified CH3 domain is Y407I or Y407V. In some embodiments, the amino acid modification at position T366 of the second Fc polypeptide of the modified CH3 domain is T366I, T366L or T366M. In some embodiments, the amino acid modification at position T394 of the second Fc polypeptide of the modified CH3 domain is T394W. In some embodiments, the first Fc polypeptide of the modified CH3 domain further includes an amino acid modification at position L351. In some embodiments, the amino acid modification at position L351 in the first Fc polypeptide of the modified CH3 domain is L351Y. In some embodiments, the second Fc polypeptide of the modified CH3 domain further includes an amino acid modification at position K392. In some embodiments, the amino acid modification at position K392 in the second Fc polypeptide of the modified CH3 domain is K392F, K392L or K392M. In some embodiments, one or both of the first and second Fc polypeptides of the modified CH3 domain further comprises the amino acid modification T350V.

In certain embodiments, the fusion protein herein comprises a heterodimeric Fc having a modified CH3 domain comprising one or more asymmetric amino acid modifications that promote formation of the heterodimeric Fc over formation of a homodimeric Fc, in which the modified CH3 domain comprises a first Fc polypeptide including the amino acid modification F405A, F405I, F405M, F405S, F405T or F405V together with the amino acid modification Y407I or Y407V, and a second Fc polypeptide including the amino acid modification T366I, T366L or T366M, together with the amino acid modification T394W. In some embodiments, the first Fc polypeptide of the modified CH3 domain further includes the amino acid modification L351Y. In some embodiments, the second Fc polypeptide of the modified CH3 domain further includes the amino acid modification K392F, K392L or K392M. In some embodiments, one or both of the first and second Fc polypeptides of the modified CH3 domain further comprises the amino acid modification T350V.

In certain embodiments, the fusion protein herein comprises a heterodimeric Fc comprising a modified CH3 domain having a first Fc polypeptide that comprises amino acid modifications at positions F405 and Y407, and optionally further comprises an amino acid modification at position L351, and a second Fc polypeptide that comprises amino acid modifications at positions T366 and T394, and optionally further comprises an amino acid modification at position K392, as described above, and the first Fc polypeptide further comprises an amino acid modification at one or both of positions S400 or Q347 and/or the second Fc polypeptide further comprises an amino acid modification at one or both of positions K360 or N390, where the amino acid modification at position S400 is S400E, S400D, S400R or S400K; the amino acid modification at position Q347 is Q347R, Q347E or Q347K; the amino acid modification at position K360 is K360D or K360E, and the amino acid modification at position N390 is N390R, N390K or N390D.

In certain embodiments, the fusion protein herein comprises a heterodimeric Fc comprising a modified CH3 domain comprising the modifications of any one of Variant 1, Variant 2, Variant 3, Variant 4 or Variant 5, as shown in Table 2. In certain embodiments, the CH3 domain has an amino acid sequence corresponding to SEQ ID NO: 4 or SEQ ID NO: 5. In certain embodiments, the CH3 has an amino acid sequence that is substantially identical to SEQ ID NO: 4 or SEQ ID NO: 5. In certain embodiments, the CH3 domain has an amino acid sequence that is about 80%, about 85%, about 90%, or about 95% identical to SEQ ID NO: 4 or SEQ ID NO: 5.

TABLE 2
Human IgG1 Fc Sequences and Variants

| Human IgG1 | APELLGGPSVFLFPPKPKDTL | |
|---|---|---|
| Fc | MISRTPEVTCVVVDVSHEDP | |
| sequence | EVKFNWYVDGVEVHNAKTKP | |
| 231-447 | REEQYNSTYRVVSVLTVLHQ | |
| (EU- | DWLNGKEYKCKVSNKALPAP | |
| numbering) | IEKTISKAKGQPREPQVYTL | |
| | PPSRDELTKNQVSLTCLVKG | |
| | FYPSDIAVEWESNGQPENNY | |
| | KTTPPVLDSDGSFFLYSKLT | |
| | VDKSRWQQGNVFSCSVMHEA | |
| | LHNHYTQKSLSLSPGK | |
| | (SEQ ID NO: 29) | |
| Variant # | Chain | Mutations |
| 1 | A | L351Y_F405A_Y407V |
| | B | T366L_K392M_T394W |
| 2 | A | L351Y_F405A_Y407V |
| | B | T366L_K392L_T394W |
| 3 | A | T350V_L351Y_F405A_Y407V |
| | B | T350V_T366L_K392L_T394W |
| 4 | A | T350V_L351Y_F405A_Y407V |
| | B | T350V_T366L_K392M_T394W |
| 5 | A | T350V_L351Y_S400E_F405A_Y407V |
| | B | T350V_T366L_N390R_K392M_T394W |

### Modified Fc CH2 Domains

In certain embodiments, the fusion protein herein comprises an Fc based on an IgG Fc having a modified CH2 domain. In some embodiments, the fusion protein comprises an Fc based on an IgG Fc having a modified CH2 domain, wherein the modification of the CH2 domain results in altered binding to one or more Fc receptors (FcRs) such as receptors of the FcγRI, FcγRII and FcγRIII subclasses.

A number of amino acid modifications to the CH2 domain that selectively alter the affinity of the Fc for different Fcγ receptors are known in the art. Amino acid modifications that result in increased binding and amino acid modifications that result in decreased binding can both be useful in certain indications. For example, increasing binding affinity of an Fc for FcγRIIIa (an activating receptor) results in increased antibody dependent cell-mediated cytotoxicity (ADCC), which in turn results in increased lysis of the target cell. Decreased binding to FcγRIIb (an inhibitory receptor) likewise can be beneficial in some circumstances. In certain indications, a decrease in, or elimination of, ADCC and complement-mediated cytotoxicity (CDC) can be desirable. In such cases, modified CH2 domains comprising amino acid modifications that result in increased binding to FcγRIIb or amino acid modifications that decrease or eliminate binding of the Fc region to all of the Fcγ receptors (“knock-out” variants) can be useful.

Examples of amino acid modifications to the CH2 domain that alter binding of the Fc by Fcγ receptors include, but are not limited to, the following: S298A/E333A/K334A and S298A/E333A/K334A/K326A (increased affinity for FcγRIIIa) (Lu, et al., 2011, J Immunol Methods, 365 (1-2): 132-41); F243L/R292P/Y300L/V305I/P396L (increased affinity for FcγRIIIa) (Stavenhagen, et al., 2007, Cancer Res, 67 (18): 8882-90); F243L/R292P/Y300L/L235V/P396L (increased affinity for FcγRIIla) (Nordstrom J L, et al., 2011, Breast Cancer Res, 13 (6): R123); F243L (increased affinity for FcγRIIIa) (Stewart, et al., 2011, Protein Eng Des Sel., 24 (9): 671-8); S298A/E333A/K334A (increased affinity for FcγRIIla) (Shields, et al., 2001, J Biol Chem, 276 (9): 6591-604); S239D/1332E/A330L and S239D/1332E (increased affinity for FcγRIIIa) (Lazar, et al., 2006, Proc Natl Acad Sci USA, 103 (11): 4005-10), and S239D/S267E and S267E/L328F (increased affinity for FcγRIIb) (Chu, et al., 2008, Mol Immunol, 45 (15): 3926-33).

Additional modifications that affect Fc binding to Fcγ receptors are described in Therapeutic Antibody Engineering (Strohl & Strohl, Woodhead Publishing series in Biomedicine No 11, ISBN 1 907568 37 9, October 2012, page 283).

In certain embodiments, the fusion protein comprises an Fc based on an IgG Fc having a modified CH2 domain, in which the modified CH2 domain comprises one or more amino acid modifications that result in decreased or eliminated binding of the Fc region to all of the Fcγ receptors (i.e., a “knock-out” or “KO” variant).

Various publications describe strategies that have been used to engineer antibodies to produce “knock-out” variants (see, for example, Strohl, 2009, Curr Opin Biotech 20:685-691, and Strohl & Strohl, “Antibody Fc engineering for optimal antibody performance” In Therapeutic Antibody Engineering, Cambridge: Woodhead Publishing, 2012, pp 225-249). These strategies include reduction of effector function through modification of glycosylation (described in more detail below), use of IgG2/IgG4 scaffolds, or the introduction of mutations in the hinge or CH2 domain of the Fc (see also, U.S. Patent Publication No. 2011/0212087, International Publication No. WO 2006/105338, U.S. Patent Publication No. 2012/0225058, U.S. Patent Publication No. 2012/0251531 and Strop et al., 2012, J. Mol. Biol., 420:204-219).

Specific, non-limiting examples of known amino acid modifications to reduce FcgR and/or complement binding to the Fc include those identified in Table 3.

TABLE 3
Modifications to Reduce Fcg Receptor or Complement Binding to the Fc

| Company | Mutations |
|---|---|
| GSK | N297A |
| Ortho Biotech | L234A/L235A |
| Protein Design labs | IgG2 V234A/G237A |
| Wellcome Labs | IgG4 L235A/G237A/E318A |
| GSK | IgG4 S228P/L236E |
| Merck | IgG2 H268Q/V309L/A330S/A331S |
| Bristol-Myers | C220S/C226S/C229S/P238S |
| Seattle Genetics | C226S/C229S/E3233P/L235V/L235A |
| Medimmune | L234F/L235E/P331S |

Additional examples include Fc regions engineered to include the amino acid modifications L235A/L236A/D265S. In addition, asymmetric amino acid modifications in the CH2 domain that decrease binding of the Fc to all Fcγ receptors are described in International Publication No. WO 2014/190441.

In certain embodiments, the CH2 domain has an amino acid sequence corresponding to SEQ ID NO: 6. In certain embodiments, the CH2 has an amino acid sequence that is substantially identical to SEQ ID NO: 6. In certain embodiments, the CH2 domain has an amino acid sequence that is about 80%, about 85%, about 90%, or about 95% identical to SEQ ID NO: 6.

### Antibody Drug Conjugates

Certain embodiments of the fusion proteins described herein comprise biologically functional proteins that are an antibody conjugated to a drug, i.e., an antibody drug conjugate (ADC). The drug of an ADC can be any therapeutic molecule, e.g., a toxin, a chemotherapeutic agent, a small molecule inhibitor. The ADC can be conjugated to the drug via a linker, which may be a cleavable linker or a non-cleavable linker. A cleavable linker can be susceptible to cleavage under intracellular conditions, for example, through lysosomal processes. Examples of cleavable linkers include linkers that are protease-sensitive, acid-sensitive, reduction-sensitive or photolabile. Conjugation of the drug can be performed by any method known in the art including, but not limited to, lysine or cysteine conjugation, bis-thiol linkers, conjugation using glycosylation sites of antibodies, ultraviolet light conjugation, and use of unnatural amino acids.

### VII. Peptidic Linkers

The trispecific fusion proteins described herein can comprise at least one peptidic linker. A peptidic linker is a peptide that joins or links other peptides or polypeptides of the fusion protein. In certain embodiments, the peptidic linker fuses a polypeptide of the biologically functional protein, e.g., an anti-CD3/anti-TAA antibody moiety, to the PD-1 domain (i.e., third binding domain).

In some embodiments, the peptidic linker linking the PD-1 domain to the remainder of the fusion protein is of sufficient length to allow the antigen-binding domains of the fusion protein to bind to their epitopes. In addition to providing a spacing function, a peptidic linker can provide flexibility or rigidity suitable for properly orienting the one or more domains of the fusion proteins herein, both within the fusion protein and between or among the fusion proteins and their target(s). Further, a peptidic linker can support expression of a full-length fusion protein and stability of the purified protein both in vitro and in vivo following administration to a subject in need thereof, such as a human, and is preferably non-immunogenic or poorly immunogenic in those same subjects. In certain embodiments, a peptidic linker can comprise part or all of a human immunoglobulin hinge, a stalk region of C-type lectins, a family of type II membrane proteins, or combinations thereof.

In certain embodiments, the one or more peptidic linker(s) of a fusion protein is of sufficient length to allow the other components of the fusion protein to (e.g., the anti-CD3 and anti-TAA binding domains) to bind to their respective epitopes and is of about 2 to about 150 amino acids. In certain embodiments, peptidic linkers range in length from about 3 to about 50 amino acids, or about 5 to about 20 amino acids, or about 10 to about 50 amino acids, or about 2 to about 40 amino acids, or about 8 to about 20 amino acids, about 10 to about 60 amino acids, about 10 to about 30 amino acids, or about 15 to about 25 amino acids. In some embodiments, the peptidic linker is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids.

In certain embodiments, the fusion protein comprising the peptidic linker(s) described herein comprises at least two heterologous polypeptides, a first polypeptide located amino (N) terminally to the peptidic linker and a second polypeptide located carboxyl (C) terminally to the peptidic linker, the two heterologous polypeptides thus separated by the peptidic linker.

In certain embodiments, a peptidic linker of a fusion protein comprises an amino acid sequence (EAAAK)n where n is an integer of 1 to 5. In some embodiments, the peptidic linker is EAAAK. In some embodiments, the peptidic linker is EAAAKEAAAK. In some embodiments, the peptidic linker comprises a polyproline linker, optionally having an amino acid sequence of PPP (SEQ ID NO: 41) or PPPP (SEQ ID NO: 40). In certain embodiments, the linker is glycine (G)-proline (P) polypeptide linker, optionally GPPPG, GGPPPGG, GPPPPG or GGPPPGG. In certain embodiments, the peptidic linker is a GlyₙSer linker. In certain embodiments, the peptidic linker comprises an amino acid sequence of (Gly3Ser)ₙ(Gly4Ser)1, (Gly3Ser)1(Gly4Ser)ₙ, (Gly3Ser)ₙ(Gly4Ser)ₙ, or (Gly4Ser)ₙ, wherein n is an integer of 1 to 5. In certain embodiments, the peptidic linkers are suitable for connecting the different domains include sequences comprising glycine-serine linkers, for example, but not limited to, EAAAKEAAAKEAAAK, (G3S)ₙ, PPPPP, Pₙ, GPₙG, GGPₙGG, Gₙ(GₘS)ₙ-GG, (SGₙ)ₘ, (SEGₙ)ₘ, wherein m and n are integers between 0-20, or from 0 to about 20. Numerous peptidic linkers are known in the art, for example, in Chen et al. Adv Drug Deliv Rev. 2013 Oct. 15; 65 (10): 1357-1369.

In certain embodiments, a peptidic linker is an amino acid sequence obtained, derived, or designed from an antibody hinge region sequence, a sequence linking a binding domain to a receptor, or a sequence linking a binding domain to a cell surface transmembrane region or membrane anchor. In some embodiments, a peptidic linker has at least one cysteine capable of participating in at least one disulfide bond under physiological conditions or other standard peptide conditions (e.g., peptide purification conditions, conditions for peptide storage). In certain embodiments, a peptidic linker corresponding or similar to an immunoglobulin hinge peptide retains a cysteine that corresponds to the hinge cysteine disposed toward the amino-terminus of that hinge. In further embodiments, a peptidic linker is from an IgG1 hinge and has been modified to remove any cysteine residues or is an IgG1 hinge that has one cysteine or two cysteines corresponding to hinge cysteines.

In certain embodiments, a peptidic linker for use in a fusion protein herein can comprise an “altered wild-type immunoglobulin hinge region” or “altered immunoglobulin hinge region”. Such altered hinge regions refers to (a) a wild-type immunoglobulin hinge region with up to 30 percent amino acid changes (e.g., up to 25 percent, 20 percent, 15 percent, 10 percent, or 5 percent amino acid substitutions or deletions), (b) a portion of a wild-type immunoglobulin hinge region that is at least 10 amino acids (e.g., at least 12, 13, 14 or 15 amino acids) in length with up to 30 percent amino acid changes (e.g., up to 25 percent, 20 percent, 15 percent, 10 percent, or 5 percent amino acid substitutions or deletions), or (c) a portion of a wild-type immunoglobulin hinge region that comprises the core hinge region (which portion can be 4, 5, 6, 7, 8, 9, 10, 11, 12, 13, 14, or 15, or at least 4, 5, 6, 7, 8, 9, 10, 11, 12, 13, 14, or 15 amino acids in length). In certain embodiments, one or more cysteine residues in a wild-type immunoglobulin hinge region, such as an IgG1 hinge comprising the upper and core regions, can be substituted by one or more other amino acid residues (e.g., one or more serine residues). An altered immunoglobulin hinge region can alternatively or additionally have a proline residue of a wild-type immunoglobulin hinge region, such as an IgG1 hinge comprising the upper and core regions, substituted by another amino acid residue (e.g., a serine residue).

Alternative hinge and linker sequences that can be used as connecting regions can be crafted from portions of cell surface receptors that connect IgV-like or IgC-like domains. Regions between IgV-like domains where the cell surface receptor contains multiple IgV-like domains in tandem and between IgC-like domains where the cell surface receptor contains multiple tandem IgC-like regions could also be used as connecting regions or linker peptides. In certain embodiments, hinge and linker sequences are from 5 to 60 amino acids long, and can be primarily flexible, but can also provide more rigid characteristics, can contain primarily a helical structure with minimal beta sheet structure.

### VII. Targets and Tumor-Associated Antigens

In some embodiments, an antigen-binding domain of the fusion proteins described herein specifically binds to a cell surface molecule. In certain embodiments, an antigen-binding domain of the fusion protein specifically binds to a tumor-associated antigen (TAA). The TAA is any antigenic substance expressed on a tumor cell surface. In some embodiments, an antigen-binding domain specifically and binds to a TAA selected from Fibroblast activation protein alpha (FAPa), Trophoblast glycoprotein (5T4), Tumor-associated calcium signal transducer 2 (Trop2), Fibronectin EDB (EDB-FN), fibronectin F.IIIB domain, CGS-2, EpCAM, EGER, HER-2, HER-3, cMet, CEA, and FOLR1, EpCAM, EGFR, HER-2, HER-3, cMet, CEA, and FOLR1, EpCAM, EGFR, HER-2, HER-3, c-Met, FOLR1, PSMA, CD38, BCMA, and CEA. 5T4, AFP, B7-H3, Cadherin-6, CAIX, CD117, CD123, CD138, CD166, CD19, CD20, CD205, CD22, CD30, CD33, CD40, CD352, CD37, CD44, CD52, CD56, CD70, CD71, CD74, CD79b, DLL3, DR5, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin (MSLN), Mucl, Muc16, NaPi2b, Nectin-4, P-cadherin, NY-ESO-1, PRLR, PSCA, PTK7, ROR1, SLC44A4, SLTRK5, SLTRK6, STEAPI, TIM1, tissue factor (TF), Trop2, WT1. In certain embodiments an antigen-binding domain specifically and binds to a TAA selected from mesothelin, Claudin18.2 (Cldn18.2), GPC3, DLL3, PSMA, MUC17, LIV1, ROR1 and EGFRvIII.

In some embodiments, an antigen-binding domain specifically binds to an antigen expressed on a virally infected cell, bacterially infected cell, damaged red blood cell, arterial plaque cell, inflamed or fibrotic tissue cell.

In certain embodiments, an antigen-binding domain specifically binds a cytokine receptor. Examples of cytokine receptors include, but are not limited to, Type I cytokine receptors, such as GM-CSF receptor, G-CSF receptor, Type I IL receptors, Epo receptor, LIF receptor, CNTF receptor, TPO receptor; Type II Cytokine receptors, such as IFN-alpha receptor (IFNAR1, IFNAR2), IFB-beta receptor, IFN-gamma receptor (IFNGR1, IFNGR2), Type II IF receptors; chemokine receptors, such as CC chemokine receptors, CXC chemokine receptors, CX3C chemokine receptors, XC chemokine receptors; tumor necrosis receptor superfamily receptors, such as TNFRSF5/CD40, TNFRSF8/CD30, TNFRSF7/CD27, TNFRSF1A/TNFR1/CD120a, TNFRSF1B/TNFR2/CD120b; TGF-beta receptors, such as TGF-beta receptor 1, TGF-beta receptor 2; Ig super family receptors, such as IF-1 receptors, CSF-1R, PDGFR (PDGFRA, PDGFRB), SCFR.

In certain embodiments, the antigen-binding domains of the fusion proteins described herein specifically bind to at least one molecule or target of interest in vivo. In certain embodiments, the target of interest is: Cluster of Differentiation 3 (CD3), Human Epidermal Growth Factor Receptor 2 (HER2), Epidermal Growth Factor Receptor (EGFR), Mesothelin (MSLN), Tissue Factor (TF), Cluster of Differentiation 19 (CD19), tyrosine-protein kinase Met (c-Met), Cluster of Differentiation 40 (CD40), Cadherin 3 (CDH3), or combinations thereof. In certain embodiments, the fusion protein comprises an antibody and at least one antigen binding domain of the antibody binds to an epitope on CD3, HER2, EGFR, MSLN, TF, CD19, c-Met, CD40, CDH3, or combinations thereof.

In some embodiments, the target of interest is HER2. In such embodiments, the anti-HER2 binding domain of the fusion protein has a VH having an amino acid sequence corresponding to SEQ ID NO: 120 and a VL having an amino acid sequence corresponding to SEQ ID NO: 124. In certain embodiments, the anti-HER2 paratope has a VH amino acid sequence that is substantially identical to SEQ ID NO: 120 and a VL amino acid sequence that is substantially identical to SEQ ID NO: 124. In certain embodiments, the anti-HER2 paratope has a VH amino acid sequence that is about 80%, about 85%, about 90%, or about 95% identical to SEQ ID NO: 120 and a VL amino acid sequence that is about 80%, about 85%, about 90%, or about 95% identical to SEQ ID NO: 124. In certain embodiments, the anti-HER2 paratope has a VH amino acid sequence that is about 96%, about 97%, about 98%, or about 99% identical to SEQ ID NO: 120 and a VL amino acid sequence that is about 96%, about 97%, about 98%, or about 99% identical to SEQ ID NO: 124. In some embodiments, the anti-HER2 paratope comprises an scFv having an amino acid sequence corresponding to SEQ ID NO: 3. In some embodiments, the anti-HER2 has a VH having 3 CDRS, HCDR1, HDR2 and HCDR3 having amino acid sequences corresponding to SEQ ID NOS: 121, 122 and 123 respectively, and a VL having 3 CDRs LCDR1, LCDR2 and LCDR3 having amino acid sequences corresponding to SEQ ID NOS: 125, 126 and 127 respectively.

In some embodiments, the TAA is HER2 and the anti-HER2 VH sequence of the second binding comprises a HCDR1 sequence comprising DTYIH (SEQ ID NO: 121), a HCDR2 sequence comprising RIYPTNGYTRYADSVKG (SEQ ID NO: 122), and a HCDR3 sequence comprising WGGDGFYAMDY (SEQ ID NO: 123), and the anti-HER2 VL sequence of the second binding comprises an LCDR1 sequence comprising RASQDVNTAVA (SEQ ID NO: 125), an LCDR2 sequence comprising SASFLYS (SEQ ID NO: 126), and an LCDR3 sequence comprising QQHYTTPPT (SEQ ID NO: 127). In some embodiments, the anti-HER2 binding domain comprises a VH domain comprising an amino acid sequence having at least about 90%, 95%, 97%, 99%, or 100% sequence identity to the sequence set forth in SEQ ID NO: 120, and 231, and a VL domain comprising an amino acid sequence having at least about 90%, 95%, 97%, 99%, or 100% sequence identity to the sequence set forth in SEQ ID NO: 124.

In certain embodiments, an antigen-binding domain of the fusion protein binds specifically to a molecule, e.g., a polypeptide, on an immune cell. In certain embodiments, the fusion protein comprises an antigen-binding domain that binds specifically to both a TAA and an antigen-binding domain that specifically binds to a molecule, e.g., a polypeptide, on an immune cell such as CD3. Thus, in certain embodiments, the fusion protein binds to both a tumor cell and an immune cell. In certain embodiments, the immune cell is a T cell. In certain embodiments the immune cell is a macrophage, a dendritic cell, a neutrophil, a B-cell or an NK cell. In certain embodiments the fusion protein binds to a CD3 antigen on a T cell and one or more TAAs on a tumor cell.

In certain embodiments, a trispecific fusion protein of the present disclosure does not bind HER2, i.e., it comprises a binding domain capable of binding a TAA other than HER2.

In some embodiments, a fusion protein herein is capable of binding MSLN. In certain embodiments, such fusion protein comprises a binding domain capable of binding MSLN. Thus, in some embodiments, the TAA is MSLN and the anti-MSLN VH sequence of the second binding domain comprises a HCDR1 sequence comprising GYTMN (SEQ ID NO: 286), a HCDR2 sequence comprising LITPYNGASSYNQKFRG (SEQ ID NO: 288) and a HCDR3 sequence comprising GGYDGRGFDY (SEQ ID NO: 285), and the anti-MSLN VL sequence of the second binding domain comprises an LCDR1 sequence comprising SASSSVSYMH (SEQ ID NO: 300), an LCDR2 sequence comprising DTSKLAS (SEQ ID NO: 279) and an LCDR3 sequence comprising QQWSGYPLT (SEQ ID NO: 294).

In some embodiments, the anti-MSLN VH sequence of the second binding domain comprises an amino acid sequence having at least about 90%, 95%, 97%, 99%, or 100% sequence identity to the sequence set forth in SEQ ID NO: 297, and the anti-MSLN VL sequence of the second binding domain comprises an amino acid sequence having at least about 90%, 95%, 97%, 99%, or 100% sequence identity to the sequence set forth in SEQ ID NOs: 276 or 277. In embodiments describing a trispecific and tetravalent fusion protein, the second and third binding domains can both the capable of binding MSLN, as further described herein. In some of these embodiments, the second and third binding domains each comprise or consist of the same anti-MSLN VH and VL sequences and thus bind to the same epitope on the MSLN target protein.

In some embodiments, a fusion protein described herein comprises a binding domain capable of binding Cldn18.2.

In some embodiments, the TAA is Cldn18.2 and the anti-Cldn18.2 VH sequence of the second binding domain comprises a HCDR1 sequence comprising SNPMI (SEQ ID NO: 310), a HCDR2 sequence comprising IIDTDGSTYYADWAKG (SEQ ID NO: 311) and a HCDR3 sequence comprising RLHGSSNGYYDDL (SEQ ID NO: 312), and the anti-MSLN VL sequence of the second binding domain comprises an LCDR1 sequence comprising QASQSIYSYLS (SEQ ID NO: 313), an LCDR2 sequence comprising KASTLAS (SEQ ID NO: 314) and an LCDR3 sequence comprising QQGYTVTNVDKNT (SEQ ID NO: 315).

In some embodiments, the anti-Cldn18.2 VH sequence of the second binding domain comprises an amino acid sequence having at least about 90%, 95%, 97%, 99%, or 100% sequence identity to the sequence set forth in SEQ ID NO: 316, and the anti-Cldn18.2 VL sequence of the second binding domain comprises an amino acid sequence having at least about 90%, 95%, 97%, 99%, or 100% sequence identity to the sequence set forth in SEQ ID NO: 317.

In embodiments describing a trispecific and tetravalent fusion protein, the second and third binding domains can both the capable of binding Cldn18.2, as further described herein. In some of these embodiments, the second and third binding domains each comprise or consist of the same anti-Cldn18.2 VH and VL sequences and thus bind to the same epitope on the Cldn18.2 target protein.

### T Cell Engagers

A T cell engager (TCE) is a polypeptide construct, often a bispecific antibody, that simultaneously binds a TAA on a tumor cell and CD3 epitope on a T-cell to form a TCR-independent artificial immune synapse. This causes the T cell to become activated and to exert a cytotoxic effect on the tumor cell. Bispecific antibodies capable of targeting T cells to tumor cells have been identified and tested for their efficacy in the treatment of cancers. Blinatumomab is an example of a bi-specific anti-CD3-CD19 antibody in a format called BiTE™ (Bi-specific T-cell Engager) that has been identified for the treatment of B-cell diseases such as relapsed B-cell non-Hodgkin lymphoma and chronic lymphocytic leukemia (Bacuerle et al (2009) Cancer Research 12:4941-4944) and is FDA approved. T cell engagers directed against other tumor-associated target antigens have also been made, and several have entered clinical trials: AMG110/MT110 EpCAM for lung cancer, gastric cancer and colorectal cancer; AMG211/MEDI565 CEA for gastrointestinal adenocarcinoma; and AMG 212/BAY2010112 PSMA for prostate cancer (see Suruadevara, C. M. et al, Oncoimmunology. 2015 June; 4 (6): e1008339). While these studies showed promising clinical efficacy, they were also hampered by severe dose-limiting toxicities primarily due to cytokine release syndrome (CRS). This resulted in narrow therapeutic windows. The use of T cell-binding paratopes and fusion proteins which are more specifically activated primarily in a tumor microenvironment (e.g., which exert a more TAA-dependent T cell cytotoxicity) might reduce the toxicity of TCEs.

In certain embodiments, a fusion protein described herein binds a CD3 antigen on a T cell with one of its binding domains, and both a TAA and a IgSF ligand on a tumor cell with a second and a third binding domain, respectively. In certain embodiments, the binding of the IgSF ligand (e.g., PD-L1) on the tumor cell prevents the binding of its IgSF receptor (e.g., PD-1) on the T cell, thus blocking checkpoint inhibition as illustrated in, e.g., FIGS. 1A and 1B.

In certain embodiments, the fusion proteins comprise an anti-CD3 paratope VH and a VL substantially identical to those of the paratopes shown in Table BB. In certain embodiments, the CD3 paratope comprises VH and VL amino acid sequences of:
(a) a VH comprising an amino acid sequence corresponding to SEQ ID NO: 2 and a VL comprising an amino acid sequence according to SEQ ID NO: 1;
(b) a VH comprising an amino acid sequence corresponding to SEQ ID NO: 206 and a VL comprising an amino acid sequence according to SEQ ID NO: 210;
(c) a VH comprising an amino acid sequence corresponding to SEQ ID NO: 215 and a VL comprising an amino acid sequence according to SEQ ID NO: 219;
(d) a VH comprising an amino acid sequence corresponding to SEQ ID NO: 223 and a VL comprising an amino acid sequence according to SEQ ID NO: 227;
(d) a VH comprising an amino acid sequence corresponding to SEQ ID NO: 231 and a VL comprising an amino acid sequence according to SEQ ID NO: 235; or
(c) a VH comprising an amino acid sequence corresponding to SEQ ID NO: 239 and a VL comprising an amino acid sequence according to SEQ ID NO: 243.

In certain embodiments, the CD3 paratope comprises VH and VL that are about 90%, about 91%, about 92%, about 93%, about 94%, about 95%, about 96%, about 97%, about 98% or about 99% identical to:
(a) a VH comprising an amino acid sequence corresponding to SEQ ID NO: 2 and a VL comprising an amino acid sequence according to SEQ ID NO: 1;
(b) a VH comprising an amino acid sequence corresponding to SEQ ID NO: 206 and a VL comprising an amino acid sequence according to SEQ ID NO: 210;
(c) a VH comprising an amino acid sequence corresponding to SEQ ID NO: 215 and a VL comprising an amino acid sequence according to SEQ ID NO: 219;
(d) a VH comprising an amino acid sequence corresponding to SEQ ID NO: 223 and a VL comprising an amino acid sequence according to SEQ ID NO: 227;
(c) a VH comprising an amino acid sequence corresponding to SEQ ID NO: 231 and a VL comprising an amino acid sequence according to SEQ ID NO: 235; or
(f) a VH comprising an amino acid sequence corresponding to SEQ ID NO: 239 and a VL comprising an amino acid sequence according to SEQ ID NO: 243.

In certain embodiments, the anti-CD3 paratope comprises a VH comprising 3 heavy chain CDRs HCDR1, HCDR2 and HCDR3 comprising amino acid sequences corresponding to SEQ ID NOS: 207, 208 and 209, and a VL comprising 3 light chain CDRs LCDR1, LCDR2 and LCDR3 comprising amino acid sequences corresponding to SEQ ID NOS: 211, 212 and 214. In certain embodiments, the anti-CD3 paratope comprises a VH comprising 3 heavy chain CDRs HCDR1, HCDR2 and HCDR3 comprising amino acid sequences corresponding to SEQ ID NOS: 224, 225 and 226, and a VL comprising 3 light chain CDRS LCDR1, LCDR2 and LCDR3 comprising amino acid sequences corresponding to SEQ ID NOS: 228, 229 and 230. In certain embodiments, the anti-CD3 paratope comprises a VH comprising 3 heavy chain CDRs HCDR1, HCDR2 and HCDR3 comprising amino acid sequences corresponding to SEQ ID NOS: 232, 233 and 234, and a VL comprising 3 light chain CDRS LCDR1, LCDR2 and LCDR3 comprising amino acid sequences corresponding to SEQ ID NOS: 236, 237 and 238. In certain embodiments, the anti-CD3 paratope comprises a VH comprising 3 heavy chain CDRs HCDR1, HCDR2 and HCDR3 comprising amino acid sequences corresponding to SEQ ID NOS: 240, 241 and 242, and a VL comprising 3 light chain CDRS LCDR1, LCDR2 and LCDR3 comprising amino acid sequences corresponding to SEQ ID NOS: 244, 245 and 246.

In some embodiments, the anti-CD3 binding domain comprises a VH domain comprising a HCDR1 sequence selected from the group consisting of RSTMH (SEQ ID NO: 207), YYGMS (SEQ ID NO: 303), KYAMN (SEQ ID NO: 224) and TYAMN (SEQ ID NO: 232), a HCDR2 sequence selected from the group consisting of YINPSSAYTNYNQKFKD (SEQ ID NO: 208), SITSSGGRIYYPDSVKG (SEQ ID NO: 301), SITRSGGRIYYPDSVKG (SEQ ID NO: 217), RIRSKYNNYATYYADSVKD (SEQ ID NO: 225) and RIRSKYNNYATYYADSVKG (SEQ ID NO: 233), and a HCDR3 sequence selected from the group consisting of PQVHYDYNGFPY (SEQ ID NO: 209), DGRDGWVAY (SEQ ID NO: 275), HGNFGNSYISYWAY (SEQ ID NO: 226) and HGNFGNSYVSWFAY (SEQ ID NO: 234), and a VL domain comprising an LCDR1 sequence selected from the group consisting of SASSSVSYMN (SEQ ID NO: 211), KRNTGNIGSNYVN (SEQ ID NO: 287), TGNTGNIGSNYVN (SEQ ID NO: 220), GSSTGAVTSGNYPN (SEQ ID NO: 228) and GSSTGAVTTSNYAN (SEQ ID NO: 236), an LCDR2 sequence selected from the group consisting of DSSKLAS (SEQ ID NO: 212), RNDKRPD (SEQ ID NO: 298), RDDKRPS (SEQ ID NO: 221), GTKFLAP (SEQ ID NO: 229), RSYQRPS (SEQ ID NO: 199) and GTNKRAP (SEQ ID NO: 237), and an LCDR3 sequence selected from the group consisting of QQWSRNPPT (SEQ ID NO: 214), QSYSSGFI (SEQ ID NO: 295), VLWYSNRWV (SEQ ID NO: 230), ATWDDSLDGWV (SEQ ID NO: 200) and ALWYSNLWV (SEQ ID NO: 238).

### VIII. Sequence Homology

Certain embodiments of the present disclosure relate to an isolated polynucleotide or a set of polynucleotides encoding a fusion protein described herein. A polynucleotide in this context can encode all or part of a fusion protein.

The terms “nucleic acid,” “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers.

A polynucleotide that “encodes” a given polypeptide is a polynucleotide that is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A transcription termination sequence can be located 3′ to the coding sequence.

In certain embodiments, the present disclosure relates to polynucleotide and polypeptide sequences that are identical or substantially identical to a polynucleotide encoding at least a portion of a fusion protein described herein, e.g., a first or second polypeptide of a biologically functional protein. The term “identical” in the context of two or more polynucleotide or polypeptide sequences, refers to two or more sequences or subsequences that are the same. Sequences are “substantially identical” if they have a percentage of amino acid residues or nucleotides that are the same (for example, about 80%, about 85%, about 90%, or about 95% identity over a specified region) when compared and aligned for maximum correspondence over a comparison window or over a designated region as measured using one of the commonly used sequence comparison algorithms as known to persons of ordinary skill in the art or by manual alignment and visual inspection. This definition also refers to the complement of a test polynucleotide sequence. The identity can exist over a region that is at least about 50 amino acids or nucleotides in length, or over a region that is 75-100 amino acids or nucleotides in length, or, where not specified, across the entire sequence of a polypeptide or polynucleotide. For sequence comparison, typically test sequences are compared to a designated reference sequence. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

“Comparison window,” as used herein refers to a segment of a sequence comprising contiguous amino acid or nucleotide positions which can be from 20 to 1000 contiguous amino acid or nucleotide positions, for example from about 50 to about 600 or from about 100 to about 300 or from about 150 to about 200 contiguous amino acid or nucleotide positions over which a test sequence can be compared to a reference sequence of the same number of contiguous positions after the two sequences are optimally aligned. Longer segments up to and including the full-length sequence may also be used as a comparison window in certain embodiments. Methods of alignment of sequences for comparison are known to those of ordinary skill in the art. Optimal alignment of sequences for comparison can be conducted, for example, by the local homology algorithm of Smith & Waterman, 1970, Adv. Appl. Math., 2: 482c; by the homology alignment algorithm of Needleman & Wunsch, 1970, J. Mol. Biol., 48:443; by the search for similarity method of Pearson & Lipman, 1988, Proc. Natl. Acad. Sci. USA, 85:2444, or by computerized implementations of these algorithms (for example, GAP, BESTFIT, FASTA or TFASTA in the Wisconsin Genetics Software Package, Genetics Computer Group, Madison, WI), or by manual alignment and visual inspection (see, for example, Ausubel et al., Current Protocols in Molecular Biology, (1995 supplement), Cold Spring Harbor Laboratory Press). Examples of available algorithms suitable for determining percent sequence identity are the BLAST and BLAST 2.0 algorithms, which are described in Altschul et al., 1997, Nuc. Acids Res., 25:3389-3402, and Altschul et al., 1990, J. Mol. Biol., 215:403-410, respectively. Software for performing BLAST analyses is publicly available through the website for the National Center for Biotechnology Information (NCBI). Certain embodiments described herein relate to variant sequences that comprise one or more amino acid substitutions. In some embodiments, the amino acid substitutions are conservative substitutions. In general, a “conservative substitution” is considered to be a substitution of one amino acid with another amino acid having similar physical, chemical and/or structural properties. Common conservative substitutions are listed under Column 1 of Table 4.

TABLE 4
Conservative Amino Acid Substitutions

| Original | | |
|---|---|---|
| Amino | | |
| Acid | Column 1 | Column 2 |
| Ala (A) | Gly, Ile, Leu, Met, | Cys, Gly, Ile, Leu, Met, Norleucine, |
| | Norleucine, Val | Phe, Trp, Tyr, Val |
| Arg (R) | His, Lys | His, Lys |
| Asn (N) | Cys, Gln, Ser, Thr | Asp, Cys, Gln, Glu, Ser, Thr |
| Asp (D) | Glu | Asn, Cys, Gln, Glu, Ser, Thr |
| Cys (C) | Asn, Gln, Ser, Thr | Asn, Asp, Gln, Glu, Ser, Thr |
| Gln (Q) | Asn, Cys, Ser, Thr | Asn, Asp, Cys, Glu, Ser, Thr |
| Glu (E) | Asp | Asp, Asn, Cys, Gln, Ser, Thr |
| Gly (G) | Pro | Ala, Ile, Leu, Met, Norleucine, Pro, |
| | | Val |
| His (H) | Arg, Lys | Arg, Lys, Phe, Trp, Tyr |
| Ile (I) | Ala, Gly, Leu, Met, | Ala, Cys, Gly, Leu, Met, Norleucine, |
| | Norleucine, Val | Phe, Trp, Tyr, Val |
| Leu (L) | Ala, Gly, Ile, Met, | Ala, Cys, Gly, Ile, Met, Norleucine, |
| | Norleucine, Val | Phe, Trp, Tyr, Val |
| Lys (K) | Arg, His | Arg, His |
| Met (M) | Ala, Gly, Ile, Leu, | Ala, Cys, Gly, Ile, Leu, Norleucine, |
| | Norleucine, Val | Phe, Trp, Tyr, Val |
| Phe (F) | Tyr, Trp | Ala, Cys, Gly, His, Ile, Leu, Met, |
| | | Norleucine, Trp, Tyr, Val |
| Pro (P) | Gly | Gly |
| Ser (S) | Asn, Cys, Gln, Thr | Asp, Asn, Cys, Gln, Glu, Thr |
| Thr (T) | Asn, Cys, Gln, Ser | Asp, Asn, Cys, Gln, Glu, Ser |
| Trp (W) | Phe, Tyr | Ala, Cys, Gly, His, Ile, Leu, Met, |
| | | Norleucine, Phe, Tyr, Val |
| Tyr (Y) | Phe, Trp | Ala, Cys, Gly, His, Ile, Leu, Met, |
| | | Norleucine, Phe, Trp, Val |
| Val (V) | Ala, Gly, Ile, Leu, Met, | Ala, Cys, Gly, Ile, Leu, Met, |
| | Norleucine | Norleucine, Phe, Trp, Tyr |

One skilled in the art will appreciate that the main factors in determining what constitutes a conservative substitution are usually the size of the amino acid side chain and its physical/chemical properties, but that certain environments allow for substitution of a given amino acid with a broader range of amino acids than those listed in Column 1. These additional amino acids tend to either have similar properties to the amino acid being substituted but to vary more widely in size or be of similar size but vary more widely in physical/chemical properties. This broader range of conservative substitutions is listed under Column 2 of Table 4. The skilled person could readily ascertain the most appropriate group of substituents to select from in view of the particular protein environment in which the amino acid substitution is being made.

### IX. Preparation of Fusion Proteins

The fusion proteins described herein can be produced using standard recombinant methods known in the art (see, for example, U.S. Pat. No. 4,816,567 and “Antibodies: A Laboratory Manual,” 2^{nd} Edition, Ed. Greenfield, Cold Spring Harbor Laboratory Press, New York, 2014).

### Vectors Encoding Fusion Proteins

For recombinant production of a fusion protein described herein, a polynucleotide or set of polynucleotides encoding the fusion protein is generated and inserted into one or more vectors for further cloning and/or expression in a host cell. Polynucleotide(s) encoding the fusion protein can be produced by standard methods known in the art (see, for example, Ausubel et al., Current Protocols in Molecular Biology, John Wiley & Sons, New York, 1994 & update, and “Antibodies: A Laboratory Manual,” 2^{nd} Edition, Ed. Greenfield, Cold Spring Harbor Laboratory Press, New York, 2014). As would be appreciated by one of skill in the art, the number of polynucleotides required for expression of the fusion protein will be dependent on the format of the fusion protein, including whether or not the fusion protein comprises an antibody and the number of polypeptides within the fusion protein. For example, when a fusion protein comprises two polypeptide chains, two polynucleotides each encoding one polypeptide chain may be required. Similarly, in certain embodiments, when the fusion protein comprises a biologically functional protein in a mAb format, two polynucleotides each encoding one polypeptide chain may be required. When multiple polynucleotides are required, they can be incorporated into one vector or into more than one vector.

Generally, for expression, the polynucleotide or set of polynucleotides is incorporated into an expression vector together with one or more regulatory elements, such as transcriptional elements, which are required for efficient transcription of the polynucleotide. Examples of such regulatory elements include, but are not limited to, promoters, enhancers, terminators, and polyadenylation signals. One skilled in the art will appreciate that the choice of regulatory elements is dependent on the host cell selected for expression of the polypeptides of the fusion protein and that such regulatory elements can be derived from a variety of sources, including bacterial, fungal, viral, mammalian or insect genes. The expression vector can optionally further contain heterologous nucleic acid sequences that facilitate expression or purification of the expressed protein. Examples include, but are not limited to, signal peptides and affinity tags such as metal-affinity tags, histidine tags, avidin/streptavidin encoding sequences, glutathione-S-transferase (GST) encoding sequences and biotin encoding sequences. The expression vector can be an extrachromosomal vector or an integrating vector.

Certain embodiments of the present disclosure relate to vectors (such as expression vectors) comprising one or more polynucleotides encoding at least a portion of a fusion protein described herein. The polynucleotide(s) can be comprised by a single vector or by more than one vector. In some embodiments, the polynucleotides are comprised by a multicistronic vector.

Expression vectors to be used to express polynucleotides include, but are not limited to, pTT5 and pUC15, Cells comprising vectors encoding fusion proteins.

Suitable host cells for cloning or expression of the fusion protein polypeptides include various prokaryotic or eukaryotic cells as known in the art. Eukaryotic host cells include, for example, mammalian cells, plant cells, insect cells and yeast cells (such as Saccharomyces or Pichia cells). Prokaryotic host cells include, for example, E. coli, A. salmonicida or B. subtilis cells.

In certain embodiments, the fusion proteins are produced in bacteria, in particular when glycosylation and Fc effector function are not needed, as described for example in U.S. Pat. Nos. 5,648,237, 5,789,199, and 5,840,523, and in Charlton, Methods in Molecular Biology, Vol. 248, pp. 245-254, B. K. C. Lo, ed., Humana Press, Totowa, N.J., 2003.

Eukaryotic microbes such as filamentous fungi or yeast are suitable expression host cells in certain embodiments, in particular fungi and yeast strains whose glycosylation pathways have been “humanized” resulting in the production of an antibody with a partially or fully human glycosylation pattern (see, for example, Gerngross, 2004, Nat. Biotech. 22:1409-1414, and Li et al., 2006, Nat. Biotech. 24:210-215).

Suitable host cells for the expression of glycosylated fusion proteins are usually eukaryotic cells. For example, U.S. Pat. Nos. 5,959,177, 6,040,498, 6,420,548, 7,125,978 and 6,417,429 describe PLANTIBODIES™ technology for producing antibodies in transgenic plants. Mammalian cell lines adapted to grow in suspension are particularly useful for expression of fusion proteins. Examples include, but are not limited to, monkey kidney CV1 line transformed by SV40 (COS-7), human embryonic kidney (HEK) line 293 or 293 cells (see, for example, Graham et al., 1977, J. Gen Virol., 36:59), baby hamster kidney cells (BHK), mouse sertoli TM4 cells (see, for example, Mather, 1980, Biol Reprod, 23:243-251); monkey kidney cells (CV1), African green monkey kidney cells (VERO-76), human cervical carcinoma (HeLa) cells, canine kidney cells (MDCK), buffalo rat liver cells (BRL 3A), human lung cells (W138), human liver cells (Hep G2), mouse mammary tumour (MMT 060562), TRI cells (see, for example, Mather et al., 1982, Annals N.Y. Acad Sci, 383:44-68), MRC 5 cells, FS4 cells, Chinese hamster ovary (CHO) cells (including DHFR⁻ CHO cells, see Urlaub et al., 1980, Proc Natl Acad Sci USA, 77:4216), and myeloma cell lines (such as Y0, NS0 and Sp2/0). Exemplary mammalian host cell lines suitable for production of antibodies are reviewed in Yazaki & Wu, Methods in Molecular Biology, Vol. 248, pp. 255-268 (B. K. C. Lo, ed., Humana Press, Totowa, N.J., 2003).

In certain embodiments, the host cell is a transient or stable higher eukaryotic cell line, such as a mammalian cell line. In some embodiments, the host cell is a mammalian HEK293T, CHO, HeLa, NS0 or COS cell. In some embodiments, the host cell is a stable cell line that allows for mature glycosylation of the fusion protein.

The host cells comprising the expression vector(s) encoding the fusion protein can be cultured using routine methods to produce the fusion protein. Alternatively, in some embodiments, host cells comprising the expression vector(s) encoding the fusion protein can be used therapeutically or prophylactically to deliver the fusion protein to a subject, or polynucleotides or expression vectors can be administered to a cell from a subject ex vivo and the cell then returned to the body of the subject.

In some embodiments, a host cell comprises (for example, has been transformed with) a vector comprising a polynucleotide that encodes the VL of a binding domain described herein and the VH of the binding domain. In some embodiments, a host cell comprises a first vector comprising a polynucleotide that encodes the VL of a binding domain described herein and a second vector comprising a polynucleotide that encodes the corresponding VH of the binding domain. In some embodiments, the host cell is eukaryotic, for example, a Chinese Hamster Ovary (CHO) cell, a human embryonic kidney (HEK) cell or a lymphoid cell (e.g., Y0, NS0, Sp20 cell).

In certain embodiments, the host cell is Expi293™ (Thermo Fisher, Waltham, MA). In certain embodiments, the host cell is CHO-S cells (National Research Council Canada) or HEK293 cells.

Certain embodiments of the present disclosure relate to a method of making a fusion protein comprising culturing a host cell into which one or more polynucleotides encoding the fusion protein, or one or more expression vectors encoding the fusion protein, have been introduced, under conditions suitable for expression of the fusion protein, and optionally recovering the fusion protein from the host cell (or from host cell culture medium)

Cell culture media that can be used include, but are not limited to, DMEM (Thermo Fisher, Waltham, MA), Opti-MEM™ (Thermo Fisher, Waltham, MA), Opti-MEM™ I Reduced Serum Medium (Thermo Fisher, Waltham, MA), RPMI-1640 medium, Expi293™ Expression Medium (Thermo Fisher, Waltham, MA), and FreeStyle CHO expression medium (Thermo Fisher Scientific, Waltham, MA).

The cell culture medium can be supplemented with serum, e.g., fetal bovine serum (FBS), amino acids, e.g., L-glutamine, antibiotics, e.g., penicillin, and streptomycin, and/or antimycotics, e.g., amphotericin, or any other supplements routinely used in the to support cell culture.

### Purification of Fusion Proteins

Typically, the fusion proteins are purified after expression. Proteins can be isolated or purified in a variety of ways known to those skilled in the art (see, for example, Protein Purification: Principles and Practice, 3^{rd} Ed., Scopes, Springer-Verlag, NY, 1994). Standard purification methods include chromatographic techniques, including ion exchange, hydrophobic interaction, affinity, sizing or gel filtration, and reverse-phase, carried out at atmospheric pressure or at high pressure using systems such as FPLC and HPLC. Additional purification methods include electrophoretic, immunological, precipitation, dialysis and chromatofocusing techniques. Ultrafiltration and diafiltration techniques, in conjunction with protein concentration, are also useful. As is well known in the art, a variety of natural proteins bind Fc and antibodies, and these proteins are used for purification of certain antibodies. For example, the bacterial proteins A and G bind to the Fc region. Likewise, the bacterial protein L binds to the Fab region of some antibodies. Purification can also be enabled by a particular fusion partner. For example, antibodies can be purified using glutathione resin if a GST fusion is employed, Ni⁺² affinity chromatography if a His-tag is employed or immobilized anti-flag antibody if a flag-tag is used. The degree of purification necessary will vary depending on the use of the antibodies. In some instances, no purification can be necessary.

In certain embodiments, fusion proteins are substantially pure. The term “substantially pure” (or “substantially purified”) when used in reference to a fusion protein described herein, means that the fusion protein is substantially or essentially free of components that normally accompany or interact with the protein as found in its naturally occurring environment, such as a native cell, or a host cell in the case of recombinantly produced fusion protein. In certain embodiments, a fusion protein that is substantially pure is a protein preparation having less than about 30%, less than about 25%, less than about 20%, less than about 15%, less than about 10%, or less than about 5% (by dry weight) of contaminating protein.

Assessment of protein purification and/or homogeneity can be performed by any method known in the art, including, but not limited to, non-reducing/reducing CE-SDS, non-reducing/reducing SDS-PAGE, Ultra-high performance liquid chromatography-size exclusion chromatography (UPLC-SEC), High Performance Liquid Chromoatography (HPLC), mass spectrometry, multi angle light scattering (MALS), dynamic light scattering (DLS).

### Post-Translational Modifications

In certain embodiments, the fusion proteins described herein comprise one or more post-translational modifications. Such post-translational modifications can occur in vivo, or they be conducted in vitro after isolation of the fusion protein from the host cell.

Post-translational modifications include various modifications as are known in the art (scc, for example, Proteins—Structure and Molecular Properties, 2nd Ed., T. E. Creighton, W. H. Freeman and Company, New York, 1993; Post-Translational Covalent Modification of Proteins, B. C. Johnson, Ed., Academic Press, New York, pgs. 1-12, 1983; Seifter et al., 1990, Meth. Enzymol., 182:626-646, and Rattan et al., 1992, Ann. N.Y. Acad. Sci., 663:48-62). In those embodiments in which the fusion proteins comprise one or more post-translational modifications, the fusion proteins can comprise the same type of modification at one or several sites, or it can comprise different modifications at different sites.

Examples of post-translational modifications include glycosylation, acetylation, phosphorylation, amidation, derivatization by known protecting/blocking groups, formylation, oxidation, reduction, proteolytic cleavage or specific chemical cleavage by cyanogen bromide, trypsin, chymotrypsin, papain, V8 protease or NaBH₄.

Other examples of post-translational modifications include, for example, addition or removal of N-linked or O-linked carbohydrate chains, chemical modifications of N-linked or O-linked carbohydrate chains, processing of N-terminal or C-terminal ends, attachment of chemical moieties to the amino acid backbone, and addition or deletion of an N-terminal methionine residue resulting from prokaryotic host cell expression. Post-translational modifications can also include modification with a detectable label, such as an enzymatic, fluorescent, isotopic or affinity label to allow for detection and isolation of the protein. Examples of suitable enzyme labels include, but are not limited to, horseradish peroxidase, alkaline phosphatase, beta-galactosidase and acetylcholinesterase. Examples of suitable prosthetic group complexes include, but are not limited to, streptavidin/biotin and avidin/biotin. Examples of suitable fluorescent materials include, but are not limited to, umbelliferone, fluorescein, fluorescein isothiocyanate, rhodamine, dichlorotriazinylamine fluorescein, dansyl chloride and phycoerythrin. An example of a luminescent material is luminol, examples of bioluminescent materials include luciferase, luciferin and acquorin, and examples of suitable radioactive materials include iodine, carbon, sulfur, tritium, indium, technetium, thallium, gallium, palladium, molybdenum, xenon and fluorine.

Additional examples of post-translational modifications include acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, gamma-carboxylation, GPI anchor formation, hydroxylation, iodination, methylation, myristylation, pegylation, prenylation, racemization, selenoylation, sulfation, transfer-RNA mediated addition of amino acids to proteins such as arginylation, and ubiquitination.

### X. Methods of Treatment

In certain aspects, the present disclosure includes methods for the treatment of a disease or condition comprising administration of a fusion protein described herein to a subject in need thereof. In certain embodiments, the subject is a mammal. In certain embodiments, the subject is human.

In certain embodiments, the methods disclosed herein are for the treatment of cancer. Cancers can include, but are not limited to, hematologic neoplasms (including leukemias, myelomas and lymphomas), carcinomas (including adenocarcinomas and squamous cell carcinomas), melanomas and sarcomas. Carcinomas and sarcomas are also frequently referred to as “solid tumors”. In certain embodiments, the cancer is a solid tumor. In certain embodiments, the cancer is leukemia. In certain embodiments, the cancer is lymphoma.

The fusion protein can exert either a cytotoxic or cytostatic effect and can result in one or more of a reduction in the size of a tumor, the slowing or prevention of an increase in the size of a tumor, an increase in the disease-free survival time between the disappearance or removal of a tumor and its reappearance, prevention of an initial or subsequent occurrence of a tumor (for example, metastasis), an increase in the time to progression, reduction of one or more adverse symptom associated with a tumor, or an increase in the overall survival time of a subject having a tumor.

In certain embodiments, the methods disclosed herein are for the treatment of an immunodeficiency disorder or disease.

In certain embodiments, the methods disclosed herein are for the treatment of autoimmune diseases or conditions.

The methods described herein comprise administering a pharmaceutical composition comprising a fusion protein described herein to a subject in need thereof. The fusion protein can be administered to a subject by an appropriate route of administration. As will be appreciated by the person of skill in the art, the route and/or mode of administration will vary depending upon the desired results. Typically, immunotherapeutic antibodies are administered by systemic administration or local administration. Local administration can be at the site of a tumor or into a tumor draining lymph node. Generally, the fusion proteins will be administered by parenteral administration, for example, by intravenous, intramuscular, intradermal, intraperitoneal, subcutaneous or spinal administration, such as by injection or infusion.

Treatment is achieved by administration of a “therapeutically effective amount” of the fusion protein. A “therapeutically effective amount” refers to an amount that is effective, at dosages and for periods of time necessary, to achieve a desired therapeutic result. A therapeutically effective amount can vary according to factors such as the disease state, age, sex, and weight of the subject. A therapeutically effective amount is also one in which any toxic or detrimental effects of the fusion protein are outweighed by the therapeutically beneficial effects. “Sufficient amount” means an amount sufficient to produce a desired effect, e.g., an amount sufficient to modulate an immune response to a target cell or tissue, e.g., by immunomodulatory ligand-receptor binding to an immune cell.

A suitable dosage of the pharmaceutical composition comprising the fusion protein can be determined by a skilled medical practitioner. The selected dosage level will depend upon a variety of pharmacokinetic factors including the activity of the particular fusion protein employed, the route of administration, the time of administration, the rate of excretion of the polypeptide, the duration of the treatment, other drugs, compounds and/or materials used in combination with the fusion protein, e.g., anti-cancer agents, the age, sex, weight, condition, general health and prior medical history of the subject being treated, and like factors well known in the medical arts.

### Methods of Modulating an Immune Cell or an Immune Response

In certain embodiments, the fusion proteins described herein are administered to a subject in need thereof, for example a subject having cancer, in order to modulate the immune system of the subject. Thus, in certain embodiments, the fusion proteins described herein downregulate an immune response or upregulate an immune response.

In accordance with this embodiment, administration of a sufficient amount of fusion protein to the subject can effect one or more of the following to activate or upregulate an immune response: modulation of an immune checkpoint, modulation of T-cell receptor signaling, modulation of T-cell activation, modulation of pro-inflammatory cytokines, modulation of interferon-γ production by T cells, modulation of T-cell suppression, modulation of M2-type tumor associated macrophages (TAM) or myeloid-derived suppressor cell (MDSC) survival and/or differentiation, and/or modulation of cytotoxic or cytostatic effects on cells.

In certain embodiments, provided herein are methods of modulating an immune response, comprising inhibition of an immune checkpoint, stimulation of an immune checkpoint, immune cell activation, stimulation of T-cell receptor signaling, and stimulation of antibody-dependent cellular cytotoxicity (ADCC), T cell-dependent cytotoxicity (TDCC)), Cell-dependent cytotoxicity (CDC), or antibody-dependent cellular phagocytosis (ADCP).

In certain embodiments, the fusion protein is capable of agonizing a target leukocyte costimulatory receptor. Functional effects of leukocyte costimulatory receptor agonism include activation of T effector cells. Activation of T effector cells can result in increased production of one or more cytokines by the T cells, such as interferon gamma (IFN-γ), interleukin-2 (IL-2), interleukin-12 (IL-12), interleukin-17 (IL-17), interleukin-21 (IL-21), granulocyte-macrophage colony-stimulating factor (GM-CSF), tumor necrosis factor-α (TNF-α), macrophage inflammatory protein 1β (MIP-1β) and/or C—X—C motif ligand 13 (CXCL13). Increased production of IL-21 and CXCL13 by T effector cells may, for example, support the differentiation and activation of inflammatory myeloid cells in the TME, recruit anti-tumor lymphoid cells such as B and NKT cells and/or support the formation of tertiary lymphoid structures.

In certain embodiments, the fusion protein activates T effector cells. In some embodiments, the fusion protein increases production of GM-CSF, TNF-α, MIP-1β, IL-17, IL-12, IL-21 and/or C—X—C motif ligand 13 (CXCL13) by T effector cells.

Certain embodiments of the present disclosure relate to methods of using the fusion proteins to modulate leukocyte costimulatory receptor agonism in vivo, for example, in order to treat cancer.

In certain embodiments, the methods relate to inhibition or downregulation of an immune cell or immune response, e.g., for treating an autoimmune disease or disorder. Thus, in certain embodiments, the fusion protein is administered in a sufficient amount to modulate an immune cell. In certain embodiments, the downregulation of an immune response is by modulation of an immune checkpoint, modulation of T-cell receptor signaling, modulation of T cell activation, modulation of pro-inflammatory cytokines, modulation of interferon-γ production by T cells, modulation of T cell suppression, modulation of M2-type tumor associated macrophages (TAM) or myeloid-derived suppressor cell (MDSC) survival and/or differentiation, and/or modulation of cytotoxic or cytostatic effects on cells.

### Methods to Modify ADCC of a Target Cell

In certain embodiments, the fusion proteins described herein induce antibody dependent cell-mediated cytotoxicity (ADCC), which in turn results in increased lysis of the target cell. In certain embodiments, the fusion protein comprises an Fc region with increased binding affinity of the Fc for FcγRIIIa (an activating receptor) resulting in increased antibody dependent cell-mediated cytotoxicity (ADCC) and increased lysis of the target cell. In certain embodiments, the Fc region is with modified CH2 domains comprising amino acid modifications that result in increased binding affinity of the Fc for FcγRIIla (an activating receptor) resulting in increased antibody dependent cell-mediated cytotoxicity (ADCC).

In certain embodiments, fusion proteins described herein reduce antibody dependent cell-mediated cytotoxicity (ADCC). In certain indications, a decrease in, or elimination of, ADCC and complement-mediated cytotoxicity (CDC) is desirable. In certain embodiments, fusion proteins comprise and Fc region with modified CH2 domains comprising amino acid modifications that result in increased binding to FcγRIIb or amino acid modifications that decrease or eliminate binding of the Fc region to all of the Fcγ receptors (“knock-out” variants) can be useful. In certain embodiments, the fusion protein comprises an Fc region with decreased binding to FcγRIIb (an inhibitory receptor).

In some embodiments, described herein is a method of treating a disease in a subject in need thereof, the method comprising administering to the subject a trispecific fusion protein, the trispecific fusion protein comprising: (i) a first binding domain capable of binding an antigen on the surface of a cytotoxic effector cell; (ii) a second binding domain capable of binding a tumor-associated antigen (TAA) on the surface of a first tumor cell; (iii) a third binding domain capable of binding PD-L1 on the surface of a second tumor cell; and (iv) a scaffold, wherein the first binding domain, the second binding domain and the third binding domain are operably linked to the scaffold.

In yet other embodiments, described herein is a method of killing cancer cells in a subject in need thereof, the method comprising administering to the subject a trispecific fusion protein, the trispecific fusion protein comprising: (i) a first binding domain capable of binding an antigen on the surface of a cytotoxic effector cell; (ii) a second binding domain capable of binding a tumor-associated antigen (TAA) on the surface of a first tumor cell; (iii) a third binding domain capable of binding PD-L1 on the surface of a second tumor cell; and (iv) a scaffold, wherein the first binding domain, the second binding domain and the third binding domain are operably linked to the scaffold.

In various embodiments, such trispecific fusion proteins used in the methods described herein can be either trivalent or tetravalent and can have a format and properties, as further described herein.

In various embodiments, an anti-PD-L1/anti-CD3/anti-TAA trispecific fusion protein of the present disclosure is capable of higher PD-1:PD-L1 checkpoint blockade in a cell population comprising T cells expressing CD3 and tumor cells expressing the TAA and PD-L1 when compared to (i) a format-matched anti-CD3/anti-TAA bispecific antibody, and/or (ii) a format-matched anti-CD3/anti-TAA bispecific antibody in combination with an anti-PD-L1 agent, e.g., an anti-PD-L1 antibody. In some embodiments, such increase in checkpoint blockade, as measured using, e.g., a reporter gene assay (RGA), over a combination treatment is about 1.2-fold, 1.5-fold, 2-fold, 3-fold, or higher. In some embodiments, such increase in checkpoint blockade over a combination treatment is from about 1.1-fold to about 1.5-fold higher, from about 1.2-fold to about 1.7-fold higher, or from about 1.2-fold to about 2-fold higher.

### XI. Pharmaceutical Compositions

The fusion proteins according to the present disclosure can be formulated in pharmaceutical compositions. These compositions can comprise, in addition to one or more of the fusion proteins, a pharmaceutically acceptable excipient, carrier, buffer, stabiliser or other materials well known to those skilled in the art. Such materials should be non-toxic and should not interfere with the efficacy of the active ingredient. The precise nature of the carrier or other material can depend on the route of administration, e.g., oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

For fusion proteins according to the present disclosure that are to be given to an individual, administration is preferably in a “therapeutically effective amount” that is sufficient to show benefit to the individual. A “prophylactically effective amount” can also be administered, when sufficient to show benefit to the individual. The actual amount administered, and rate and time-course of administration, will depend on the nature and severity of protein aggregation disease being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

In some embodiments, described herein is a pharmaceutical composition comprising a trispecific fusion protein, the trispecific fusion protein comprising: (i) a first binding domain capable of binding an antigen on the surface of a cytotoxic effector cell; (ii) a second binding domain capable of binding a tumor-associated antigen (TAA) on the surface of a first tumor cell; (iii) a third binding domain capable of binding PD-L1 on the surface of a second tumor cell; and (iv) a scaffold, wherein the first binding domain, the second binding domain and the third binding domain are operably linked to the scaffold.

In various embodiments, such trispecific fusion proteins used in the pharmaceutical compositions described herein can be either trivalent or tetravalent and can have a format and properties, as further described herein.

### XII. Kits

The present disclosure also provides for kits comprising one or more of the trispecific fusion proteins and/or pharmaceutical compositions described herein and instructions for use. Thus, in certain embodiments, described herein are kits comprising vectors for expressing a fusion protein described herein and instructions for use. In certain embodiments, described herein are kits comprising host cells comprising a vector for expressing a fusion protein and instructions for use. In certain embodiments, are kits comprising a purified fusion protein and instructions for use. The purified fusion protein can be lyophilized or provided in a dry form, such as a powder or granules, and the kit can additionally contain a suitable solvent for reconstitution of the lyophilized or dried component(s).

The kit typically will comprise a container and a label and/or package insert on or associated with the container. The label or package insert contains instructions customarily included in commercial packages of therapeutic products, providing information or instructions about the indications, usage, dosage, administration, contraindications and/or warnings concerning the use of such therapeutic products. The label or package insert can further include a notice in the form prescribed by a governmental agency regulating the manufacture, use or sale of pharmaceuticals or biological products, which notice reflects approval by the agency of manufacture, for use or sale for human or animal administration. The container holds a composition comprising the fusion protein. In some embodiments, the container can have a sterile access port. For example, the container can be an intravenous solution bag or a vial having a stopper that can be pierced by a hypodermic injection needle.

In addition to the container containing the composition comprising the fusion protein, the kit can comprise one or more additional containers comprising other components of the kit. For example, a pharmaceutically-acceptable buffer (such as bacteriostatic water for injection) (BWFI), phosphate-buffered saline, Ringer's solution or dextrose solution), other buffers or diluents.

Suitable containers include, for example, bottles, vials, syringes, intravenous solution bags, and the like. The containers can be formed from a variety of materials such as glass or plastic. If appropriate, one or more components of the kit can be lyophilized or provided in a dry form, such as a powder or granules, and the kit can additionally contain a suitable solvent for reconstitution of the lyophilized or dried component(s).

The kit can further include other materials desirable from a commercial or user standpoint, such as filters, needles, and syringes.

The following examples are offered for illustrative purposes only and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. Sec, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### XIII. Certain Embodiments of the Present Disclosure

In various embodiments, the present disclosure relates to any one of the embodiments 1-218, and combinations thereof.

Embodiment 1. A trispecific fusion protein comprising:
(i) a first binding domain capable of binding CD3 on the surface of a cytotoxic effector cell;
(ii) a second binding domain capable of binding a tumor-associated antigen (TAA) on the surface of a tumor cell;
(iii) a third binding domain capable of binding PD-L1 on the surface of a tumor cell; and
(iv) a scaffold,
wherein the first binding domain, the second binding domain and the third binding domain are operably linked to the scaffold.

Embodiment 2. The trispecific fusion protein of embodiment 1, wherein the scaffold comprises a dimeric Fc domain comprising a first Fc polypeptide and a second Fc polypeptide.

Embodiment 3. The trispecific fusion protein of embodiment 2, wherein the dimeric Fc domain is a heterodimeric Fc domain, and wherein the amino acid sequence of the first Fc polypeptide differs in at least one amino acid residue from the amino acid sequence of the second Fc polypeptide.

Embodiment 4. The trispecific fusion protein of any one of embodiments 2-3, wherein the first binding domain is linked to the N-terminus of the first Fc polypeptide and the second binding domain is linked to the N-terminus of the second binding domain.

Embodiment 5. The trispecific fusion protein of any one of embodiments 1-4, wherein the first binding domain and the second binding domain are each independently a Fab or an scFv. Embodiment 6. The trispecific fusion protein of embodiment 5, wherein:
a) the first binding domain is a Fab and the second binding domain is an scFv; or
b) the first binding domain is an scFv and the second binding domain is a Fab; or
c) the first binding domain is a Fab and the second binding domain is a Fab; or
d) the first binding domain is an scFv and the second binding domain is an scFv.

Embodiment 7. The trispecific fusion protein of any one of embodiments 2-6, wherein the first binding domain is linked to the first Fc polypeptide via a first linker^{Fc}.

Embodiment 8. The trispecific fusion protein of any one of embodiments 2-7, wherein the second binding domain is linked to the second Fc polypeptide via a second linker^{Fc}.

Embodiment 9. The trispecific fusion protein of any one of embodiments 7-8, wherein the first linker^{Fc}, the second linker^{Fc}, or both, comprise or consist of an IgG hinge region, or a portion or variant thereof.

Embodiment 10. The trispecific fusion protein of any one of embodiments 7-9, wherein the first linker^{Fc}, the second linker^{Fc}, or both, comprise or consist of an amino acid sequence having at least 80%, 90%, or 95% sequence identity to the amino acid sequence set forth in SEQ ID NO: 50 or a fragment thereof.

Embodiment 11. The trispecific fusion protein of any one of embodiments 2-10, wherein the first binding domain is a Fab and is linked to the N-terminus of the first Fc polypeptide via the C-terminus of the Fab.

Embodiment 12. The trispecific fusion protein of any one of embodiments 2-11, wherein the second binding domain is an scFv comprising, from N- to C-terminus, a VH domain linked to a VL domain or a VL domain linked to a VH domain and is linked via its C-terminus to the N-terminus of the second Fc polypeptide.

Embodiment 13. The trispecific fusion protein of any one of embodiments 2-5, wherein the fusion protein comprises a first immunoglobulin G heavy chain, a second immunoglobulin G heavy chain and an immunoglobulin light chain, wherein the first heavy chain comprises, from N- to C-terminus, a Fab VH and CH1 domains linked to the first Fc polypeptide, the second heavy chain comprises, from N- to C-terminus, an scFv VH and VL or VL and VH domains linked to the second Fc polypeptide, and the light chain comprises, from N- to C-terminus, the Fab VL and CL domains.

Embodiment 14. The trispecific fusion protein of any one of embodiments 1-13, wherein the third binding domain is linked to (i) the first binding domain, (ii) the second binding domain, or (iii) the scaffold.

Embodiment 15. The trispecific fusion protein of embodiment 14, wherein the third binding domain is linked to (i) the N-terminus of the Fab VH domain, (ii) the N-terminus of the Fab VL domain, (iii) the C-terminus of the CL domain, (iv) the N-terminus of the scFv VH or VL domain, (v) the C-terminus of the first Fc polypeptide, or (vi) the C-terminus of the second Fc polypeptide.

Embodiment 16. The trispecific fusion protein of any one of embodiments 1-15, wherein the third binding domain comprises a PD-1 polypeptide.

Embodiment 17. The trispecific fusion protein of any one of embodiments 1-16, wherein the third binding domain consists of a PD-1 polypeptide.

Embodiment 18. The trispecific fusion protein of any one of embodiments 16-17, wherein the PD-1 polypeptide is a wildtype PD-1 polypeptide comprising or consisting of the amino acid sequence set forth in SEQ ID NO: 7, or a portion of fragment thereof.

Embodiment 19. The trispecific fusion protein of any one of embodiments 16-17, wherein the PD-1 polypeptide comprises one or more amino acid modifications compared to a corresponding wildtype PD-1 polypeptide that increase or decrease the binding affinity of the PD-1 polypeptide to PD-L1 when compared to the binding affinity of the corresponding wildtype PD-1 polypeptide to PD-L1.

Embodiment 20. The trispecific fusion protein of embodiment 19, wherein the one or more amino acid modifications comprise one or more amino acid substitutions.

Embodiment 21. The trispecific fusion protein of embodiment 20, wherein the PD-1 polypeptide has a binding affinity for PD-L1 of from about 100 μM to about 10 pM, from about 10 μM to about 150 pM, from about 100 nM to about 150 pM, or from about 5 nM to about 90 nM.

Embodiment 22. The trispecific fusion protein of any one of embodiments 16-21, wherein the PD-1 polypeptide comprises or consists of an amino acid sequence having at least about 80%, 90%, 95%, 99%, or 100% sequence identity to the amino acid sequence set forth in SEQ ID NO: 9.

Embodiment 23. The trispecific fusion protein of any one of embodiments 1-22, wherein the anti-CD3 binding domain comprises a VH domain comprising a HCDR1 sequence selected from the group consisting of RSTMH (SEQ ID NO: 207), YYGMS (SEQ ID NO: 303), KYAMN (SEQ ID NO: 224) and TYAMN (SEQ ID NO: 232), a HCDR2 sequence selected from the group consisting of YINPSSAYTNYNQKFKD (SEQ ID NO: 208), SITSSGGRIYYPDSVKG (SEQ ID NO: 301), SITRSGGRIYYPDSVKG (SEQ ID NO: 217), RIRSKYNNYATYYADSVKD (SEQ ID NO: 225) and RIRSKYNNYATYYADSVKG (SEQ ID NO: 233), and a HCDR3 sequence selected from the group consisting of PQVHYDYNGFPY (SEQ ID NO: 209), DGRDGWVAY (SEQ ID NO: 275), HGNFGNSYISYWAY (SEQ ID NO: 226) and HGNFGNSYVSWFAY (SEQ ID NO: 234), and a VL domain comprising an LCDR1 sequence selected from the group consisting of SASSSVSYMN (SEQ ID NO: 211), KRNTGNIGSNYVN (SEQ ID NO: 287), TGNTGNIGSNYVN (SEQ ID NO: 220), GSSTGAVTSGNYPN (SEQ ID NO: 228) and GSSTGAVTTSNYAN (SEQ ID NO: 236), an LCDR2 sequence selected from the group consisting of DSSKLAS (SEQ ID NO: 212), RNDKRPD (SEQ ID NO: 298), RDDKRPS (SEQ ID NO: 221), GTKFLAP (SEQ ID NO: 229), RSYQRPS (SEQ ID NO: 199) and GTNKRAP (SEQ ID NO: 237), and an LCDR3 sequence selected from the group consisting of QQWSRNPPT (SEQ ID NO: 214), QSYSSGFI (SEQ ID NO: 295), VLWYSNRWV (SEQ ID NO: 230), ATWDDSLDGWV (SEQ ID NO: 200) and ALWYSNLWV (SEQ ID NO: 238).

Embodiment 24. The trispecific fusion protein of any one of embodiments 1-23, wherein the anti-CD3 binding domain comprises a VH domain comprising the HCDR sequences YYGMS (SEQ ID NO: 303), SITSSGGRIYYPDSVKG (SEQ ID NO: 301) and DGRDGWVAY (SEQ ID NO: 275), and a VL domain comprising the LCDR sequences KRNTGNIGSNYVN (SEQ ID NO: 287), RNDKRPD (SEQ ID NO: 298) and QSYSSGFI (SEQ ID NO: 295).

Embodiment 25. The trispecific fusion protein of any one of embodiments 1-23, wherein the anti-CD3 binding domain comprises a VH domain comprising the HCDR sequences YYGMS (SEQ ID NO: 302), SITRSGGRIYYPDSVKG (SEQ ID NO: 217) and DGRDGWVAY (SEQ ID NO: 275), and a VL domain comprising the LCDR sequences TGNTGNIGSNYVN (SEQ ID NO: 220), RDDKRPS (SEQ ID NO: 221) and QSYSSGFI (SEQ ID NO: 295).

Embodiment 26. The trispecific fusion protein of any one of embodiments 1-23, wherein the anti-CD3 binding domain comprises a VH domain comprising the HCDR sequences KYAMN (SEQ ID NO: 224), RIRSKYNNYATYYADSVKD (SEQ ID NO: 225) and HGNFGNSYISYWAY (SEQ ID NO: 226), and a VL domain comprising the LCDR sequences GSSTGAVTSGNYPN (SEQ ID NO: 228), GTKFLAP (SEQ ID NO: 229) and VLWYSNRWV (SEQ ID NO: 230).

Embodiment 27. The trispecific fusion protein of any one of embodiments 1-23, wherein the anti-CD3 binding domain comprises a VH domain comprising the HCDR sequences TYAMN (SEQ ID NO: 232), RIRSKYNNYATYYADSVKG (SEQ ID NO: 233) and HGNFGNSYVSWFAY (SEQ ID NO: 234), and a VL domain comprising the LCDR sequences GSSTGAVTTSNYAN (SEQ ID NO: 236), GTNKRAP (SEQ ID NO: 237) and ALWYSNLWV (SEQ ID NO: 238).

Embodiment 28. The trispecific fusion protein of any one of embodiments 1-23, wherein the anti-CD3 binding domain comprises a VH domain comprising the HCDR sequences RSTMH (SEQ ID NO: 207), YINPSSAYTNYNQKFKD (SEQ ID NO: 208) and PQVHYDYNGFPY (SEQ ID NO: 209), and a VL domain comprising the LCDR sequences SASSSVSYMN (SEQ ID NO: 211), DSSKLAS (SEQ ID NO: 212) and QQWSRNPPT (SEQ ID NO: 214).

Embodiment 29. The trispecific fusion protein of any one of embodiments 1-23, wherein the anti-CD3 binding domain comprises a VH domain comprising the HCDR sequences RSTMH (SEQ ID NO: 207), YINPSSAYTNYNQKFKD (SEQ ID NO: 208) and PQVHYDYNGFPY (SEQ ID NO: 209), and a VL domain comprising the LCDR sequences RSYQRPS (SEQ ID NO: 199) and ATWDDSLDGWV (SEQ ID NO: 200).

Embodiment 30. The trispecific fusion protein of any one of embodiments 1-23, wherein the anti-CD3 binding domain comprises a VH domain comprising an amino acid sequence having at least about 90%, 95%, 97%, 99%, or 100% sequence identity to the sequence set forth in any one of SEQ ID NOs: 2, 215, 223, and 231, and a VL domain comprising an amino acid sequence having at least about 90%, 95%, 97%, 99%, or 100% sequence identity to the sequence set forth in any one of SEQ ID NOs: 1, 219, 227, and 235.

Embodiment 31. The trispecific fusion protein of any one of embodiments 1-30, wherein the TAA is not HER2.

Embodiment 32. The trispecific fusion protein of any one of embodiments 1-31, wherein the TAA is Cldn18.2.

Embodiment 33. The trispecific fusion protein of embodiment 32, wherein the anti-Cldn18.2 VH sequence of the second binding comprises a HCDR1 sequence comprising SNPMI (SEQ ID NO: 310), a HCDR2 sequence comprising IIDTDGSTYYADWAKG (SEQ ID NO: 311) and a HCDR3 sequence comprising RLHGSSNGYYDDL (SEQ ID NO: 312), and the anti-Cldn18.2 VL sequence of the second binding comprises an LCDR1 sequence comprising QASQSIYSYLS (SEQ ID NO: 313), an LCDR2 sequence comprising KASTLAS (SEQ ID NO: 314) and an LCDR3 sequence comprising QQGYTVTNVDKNT (SEQ ID NO: 315).

Embodiment 34. The trispecific fusion protein of any one of embodiments 32-33, wherein the anti-Cldn18.2 VH sequence of the second binding domain comprises an amino acid sequence having at least about 90%, 95%, 97%, 99%, or 100% sequence identity to the sequence set forth in SEQ ID NO: 316, and the anti-Cldn18.2 VL sequence of the second binding comprises an amino acid sequence having at least about 90%, 95%, 97%, 99%, or 100% sequence identity to the sequence set forth in SEQ ID NO: 317.

Embodiment 35. The trispecific fusion protein of any one of embodiments 2-34, wherein the first Fc polypeptide and the second Fc polypeptide each comprise a CH2 domain comprising an amino acid sequence having at least about 90%, 95%, 97%, 99%, or 100% sequence identity to the sequence set forth in SEQ ID NO: 6.

Embodiment 36. The trispecific fusion protein of any one of embodiments 2-35, wherein one of the first Fc polypeptide or the second Fc polypeptide comprises a CH3 domain comprising an amino acid sequence having at least about 90%, 95%, 97%, 99%, or 100% sequence identity to the sequence set forth in SEQ ID NO: 4, and the other Fc polypeptide comprises a CH3 domain comprising an amino acid sequence having at least about 90%, 95%, 97%, 99%, or 100% sequence identity to the sequence set forth in SEQ ID NO: 5.

Embodiment 37. The trispecific fusion protein of embodiment 1, wherein the trispecific fusion protein is not v31929.

Embodiment 38. The trispecific fusion protein of any one of embodiments 1-30, wherein the TAA is HER2 and the trispecific fusion protein comprises (i) a first heavy chain having at least 90%, 95%, 97%, 99% or 100% sequence identity to the clone sequence set forth in 22080 or 23734, (ii) a second heavy chain having at least 90%, 95%, 97%, 99% or 100% sequence identity to the clone sequence set forth in 21490, and (iii) a light chain having at least 90%, 95%, 97%, 99% or 100% sequence identity to the clone sequence set forth in 12985.

Embodiment 39. The trispecific fusion protein of embodiment 38, wherein the trispecific fusion protein comprises (i) a first heavy chain having at least 90%, 95%, 97%, 99% or 100% sequence identity to the clone sequence set forth in 22080, (ii) a second heavy chain having at least 90%, 95%, 97%, 99% or 100% sequence identity to the clone sequence set forth in 21490, and (iii) a light chain having at least 90%, 95%, 97%, 99% or 100% sequence identity to the clone sequence set forth in 12985.

Embodiment 40. The trispecific fusion protein of embodiment 38, wherein the trispecific fusion protein comprises (i) a first heavy chain having at least 90%, 95%, 97%, 99% or 100% sequence identity to the clone sequence set forth in 23734, (ii) a second heavy chain having at least 90%, 95%, 97%, 99% or 100% sequence identity to the clone sequence set forth in 21490, and (iii) a light chain having at least 90%, 95%, 97%, 99% or 100% sequence identity to the clone sequence set forth in 12985.

Embodiment 41. The trispecific fusion protein of any one of embodiments 1-31, wherein the TAA is MSLN and the trispecific fusion protein comprises (i) a first heavy chain having at least 90%, 95%, 97%, 99% or 100% sequence identity to the clone sequence set forth in 29207, 29208, 29276, 29238, 29282, 22080 or 23734, (ii) a second heavy chain having at least 90%, 95%, 97%, 99% or 100% sequence identity to the clone sequence set forth in 23867, 23270, 29275 or 25095 and (iii) a light chain having at least 90%, 95%, 97%, 99% or 100% sequence identity to the clone sequence set forth in 16412, 23570 or 12985.

Embodiment 42. The trispecific fusion protein of embodiment 41, wherein the trispecific fusion protein comprises (i) a first heavy chain having at least 90%, 95%, 97%, 99% or 100% sequence identity to the clone sequence set forth in 29207, (ii) a second heavy chain having at least 90%, 95%, 97%, 99% or 100% sequence identity to the clone sequence set forth in 29275, and (iii) a light chain having at least 90%, 95%, 97%, 99% or 100% sequence identity to the clone sequence set forth in 16412.

Embodiment 43. The trispecific fusion protein of embodiment 41, wherein the trispecific fusion protein comprises (i) a first heavy chain having at least 90%, 95%, 97%, 99% or 100% sequence identity to the clone sequence set forth in 29208, (ii) a second heavy chain having at least 90%, 95%, 97%, 99% or 100% sequence identity to the clone sequence set forth in v29275, and (iii) a light chain having at least 90%, 95%, 97%, 99% or 100% sequence identity to the clone sequence set forth in 16412.

Embodiment 44. The trispecific fusion protein of embodiment 41, wherein the trispecific fusion protein comprises (i) a first heavy chain having at least 90%, 95%, 97%, 99% or 100% sequence identity to the clone sequence set forth in 29276, (ii) a second heavy chain having at least 90%, 95%, 97%, 99% or 100% sequence identity to the clone sequence set forth in 25095, and (iii) a light chain having at least 90%, 95%, 97%, 99% or 100% sequence identity to the clone sequence set forth in 23570.

Embodiment 45. The trispecific fusion protein of embodiment 41, wherein the trispecific fusion protein comprises (i) a first heavy chain having at least 90%, 95%, 97%, 99% or 100% sequence identity to the clone sequence set forth in 29238, (ii) a second heavy chain having at least 90%, 95%, 97%, 99% or 100% sequence identity to the clone sequence set forth in 29275, and (iii) a light chain having at least 90%, 95%, 97%, 99% or 100% sequence identity to the clone sequence set forth in 16412.

Embodiment 46. The trispecific fusion protein of any one of embodiments 1-3, wherein the trispecific fusion protein comprises (i) a first heavy chain having at least 90%, 95%, 97%, 99% or 100% sequence identity to the clone sequence set forth in 29282, (ii) a second heavy chain having at least 90%, 95%, 97%, 99% or 100% sequence identity to the clone sequence set forth in 23270, and (iii) a light chain having at least 90%, 95%, 97%, 99% or 100% sequence identity to the clone sequence set forth in 16412.

Embodiment 47. The trispecific fusion protein of embodiment 41, wherein the trispecific fusion protein comprises (i) a first heavy chain having at least 90%, 95%, 97%, 99% or 100% sequence identity to the clone sequence set forth in 22080, (ii) a second heavy chain having at least 90%, 95%, 97%, 99% or 100% sequence identity to the clone sequence set forth in 23867, and (iii) a light chain having at least 90%, 95%, 97%, 99% or 100% sequence identity to the clone sequence set forth in 12985.

Embodiment 48. The trispecific fusion protein of embodiment 41, wherein the trispecific fusion protein comprises (i) a first heavy chain having at least 90%, 95%, 97%, 99% or 100% sequence identity to the clone sequence set forth in 23734, (ii) a second heavy chain having at least 90%, 95%, 97%, 99% or 100% sequence identity to the clone sequence set forth in 23867, and (iii) a light chain having at least 90%, 95%, 97%, 99% or 100% sequence identity to the clone sequence set forth in 12985.

Embodiment 49. The trispecific fusion protein of any one of embodiments 1-36, wherein the TAA is Cldn18.2 and the trispecific fusion protein comprises (i) a first heavy chain having at least 90%, 95%, 97%, 99% or 100% sequence identity to the clone sequence set forth in 29241, 29238, 29208 or 29211, (ii) a second heavy chain having at least 90%, 95%, 97%, 99% or 100% sequence identity to the clone sequence set forth in 29264, 29261, 29267 or 28373 and (iii) a light chain having at least 90%, 95%, 97%, 99% or 100% sequence identity to the clone sequence set forth in 16412.

Embodiment 50. The trispecific fusion protein of embodiment 49, wherein the trispecific fusion protein comprises (i) a first heavy chain having at least 90%, 95%, 97%, 99% or 100% sequence identity to the clone sequence set forth in 29241, (ii) a second heavy chain having at least 90%, 95%, 97%, 99% or 100% sequence identity to the clone sequence set forth in 28373, and (iii) a light chain having at least 90%, 95%, 97%, 99% or 100% sequence identity to the clone sequence set forth in 16412.

Embodiment 51. The trispecific fusion protein of embodiment 49, wherein the trispecific fusion protein comprises (i) a first heavy chain having at least 90%, 95%, 97%, 99% or 100% sequence identity to the clone sequence set forth in 29238, (ii) a second heavy chain having at least 90%, 95%, 97%, 99% or 100% sequence identity to the clone sequence set forth in 28373, and (iii) a light chain having at least 90%, 95%, 97%, 99% or 100% sequence identity to the clone sequence set forth in 16412.

Embodiment 52. The trispecific fusion protein of embodiment 49, wherein the trispecific fusion protein comprises (i) a first heavy chain having at least 90%, 95%, 97%, 99% or 100% sequence identity to the clone sequence set forth in 29208, (ii) a second heavy chain having at least 90%, 95%, 97%, 99% or 100% sequence identity to the clone sequence set forth in 28373, and (iii) a light chain having at least 90%, 95%, 97%, 99% or 100% sequence identity to the clone sequence set forth in 16412.

Embodiment 53. The trispecific fusion protein of embodiment 49, wherein the trispecific fusion protein comprises (i) a first heavy chain having at least 90%, 95%, 97%, 99% or 100% sequence identity to the clone sequence set forth in 29211, (ii) a second heavy chain having at least 90%, 95%, 97%, 99% or 100% sequence identity to the clone sequence set forth in 28373, and (iii) a light chain having at least 90%, 95%, 97%, 99% or 100% sequence identity to the clone sequence set forth in 16412.

Embodiment 54. The trispecific fusion protein of any one of embodiments 1-36, wherein the trispecific fusion protein comprises a fourth binding domain, and wherein the fourth binding domain is linked either directly or via a linker to the first binding domain, the second binding domain, the third binding domain or the scaffold.

Embodiment 55. The trispecific fusion protein of embodiment 54, wherein the fourth binding domain is capable of binding the TAA.

Embodiment 56. The trispecific fusion protein of embodiment 55, wherein both the second binding domain and the fourth binding domain that are capable of binding the TAA are scFv domains.

Embodiment 57. The trispecific fusion protein of embodiment 56, wherein the second binding domain and the fourth binding domain that are capable of binding the TAA each comprise or consist of the same anti-TAA VH and VL sequences.

Embodiment 58. The trispecific fusion protein of any one of embodiments 55-57, wherein the TAA is MSLN and the trispecific fusion protein comprises (i) a first heavy chain having at least 90%, 95%, 97%, 99% or 100% sequence identity to the clone sequence set forth in 29257 or 29283, (ii) a second heavy chain having at least 90%, 95%, 97%, 99% or 100% sequence identity to the clone sequence set forth in 23867, 29258, 29264, 23867, 29263, 29267 or 29261 and (iii) a light chain having at least 90%, 95%, 97%, 99% or 100% sequence identity to the clone sequence set forth in 16412, 29226 or 29220.

Embodiment 59. The trispecific fusion protein of embodiment 58, wherein the trispecific fusion protein comprises (i) a first heavy chain having at least 90%, 95%, 97%, 99% or 100% sequence identity to the clone sequence set forth in 29257, (ii) a second heavy chain having at least 90%, 95%, 97%, 99% or 100% sequence identity to the clone sequence set forth in 23867, and (iii) a light chain having at least 90%, 95%, 97%, 99% or 100% sequence identity to the clone sequence set forth in 29220.

Embodiment 60. The trispecific fusion protein of embodiment 58, wherein the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 29258, and (iii) a light chain having at least 90%, 95%, 97%, 99% or 100% sequence identity to the clone sequence set forth in 16412.

Embodiment 61. The trispecific fusion protein of embodiment 58, wherein the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 29264, and (iii) a light chain having at least 90%, 95%, 97%, 99% or 100% sequence identity to the clone sequence set forth in 16412.

Embodiment 62. The trispecific fusion protein of embodiment 58, wherein the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 23867, and (iii) a light chain having at least 90%, 95%, 97%, 99% or 100% sequence identity to the clone sequence set forth in 29220.

Embodiment 63. The trispecific fusion protein of embodiment 58, wherein the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 23867, and (iii) a light chain having at least 90%, 95%, 97%, 99% or 100% sequence identity to the clone sequence set forth in 29226.

Embodiment 64. The trispecific fusion protein of embodiment 58, wherein the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 29263, and (iii) a light chain having at least 90%, 95%, 97%, 99% or 100% sequence identity to the clone sequence set forth in 16412.

Embodiment 65. The trispecific fusion protein of embodiment 58, wherein the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 29264, and (iii) a light chain having at least 90%, 95%, 97%, 99% or 100% sequence identity to the clone sequence set forth in 16412.

Embodiment 66. The trispecific fusion protein of embodiment 58, wherein the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 29261, and (iii) a light chain having at least 90%, 95%, 97%, 99% or 100% sequence identity to the clone sequence set forth in 16412.

Embodiment 67. The trispecific fusion protein of embodiment 58, wherein the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 29267, and (iii) a light chain having at least 90%, 95%, 97%, 99% or 100% sequence identity to the clone sequence set forth in 16412.

Embodiment 68. The trispecific fusion protein of any one of embodiments 55-57, wherein the TAA is Cldn18.2 and the trispecific fusion protein comprises (i) a first heavy chain having at least 90%, 95%, 97%, 99% or 100% sequence identity to the clone sequence set forth in 29244, (ii) a second heavy chain having at least 90%, 95%, 97%, 99% or 100% sequence identity to the clone sequence set forth in 29245, 29248, 29251 or 29254 and (iii) a light chain having at least 90%, 95%, 97%, 99% or 100% sequence identity to the clone sequence set forth in 16412.

Embodiment 69. The trispecific fusion protein of embodiment 68, wherein the trispecific fusion protein comprises (i) a first heavy chain having at least 90%, 95%, 97%, 99% or 100% sequence identity to the clone sequence set forth in 29244, (ii) a second heavy chain having at least 90%, 95%, 97%, 99% or 100% sequence identity to the clone sequence set forth in 29245, and (iii) a light chain having at least 90%, 95%, 97%, 99% or 100% sequence identity to the clone sequence set forth in 16412.

Embodiment 70. The trispecific fusion protein of embodiment 68, wherein the trispecific fusion protein comprises (i) a first heavy chain having at least 90%, 95%, 97%, 99% or 100% sequence identity to the clone sequence set forth in 29244, (ii) a second heavy chain having at least 90%, 95%, 97%, 99% or 100% sequence identity to the clone sequence set forth in 29248, and (iii) a light chain having at least 90%, 95%, 97%, 99% or 100% sequence identity to the clone sequence set forth in 16412.

Embodiment 71. The trispecific fusion protein of embodiment 68, wherein the trispecific fusion protein comprises (i) a first heavy chain having at least 90%, 95%, 97%, 99% or 100% sequence identity to the clone sequence set forth in 29244, (ii) a second heavy chain having at least 90%, 95%, 97%, 99% or 100% sequence identity to the clone sequence set forth in 29251, and (iii) a light chain having at least 90%, 95%, 97%, 99% or 100% sequence identity to the clone sequence set forth in v16412.

Embodiment 72. The trispecific fusion protein of embodiment 68, wherein the trispecific fusion protein comprises (i) a first heavy chain having at least 90%, 95%, 97%, 99% or 100% sequence identity to the clone sequence set forth in 29244, (ii) a second heavy chain having at least 90%, 95%, 97%, 99% or 100% sequence identity to the clone sequence set forth in 29254, and (iii) a light chain having at least 90%, 95%, 97%, 99% or 100% sequence identity to the clone sequence set forth in 16412.

Embodiment 73. The trispecific fusion protein of any one of embodiments 1-72, wherein upon binding to CD3 on the cytotoxic effector cell, the TAA on a tumor cell and PD-L1 on a tumor cell, the trispecific fusion protein forms a TCR-independent immune synapse capable of inducing effector cell mediated cytotoxicity against the tumor cell.

Embodiment 74. The trispecific fusion protein of any one of embodiments 1-73, wherein the trispecific fusion protein binds the TAA and PD-L1 on the same tumor cell.

Embodiment 75. The trispecific fusion protein of any one of embodiments 1-73, wherein the trispecific fusion protein binds the TAA and PD-L1 on different tumor cells.

Embodiment 76. A tetravalent and trispecific fusion protein comprising:
(i) a first binding domain capable of binding CD3 on the surface of a cytotoxic effector cell;
(ii) a second binding domain and a third binding domain capable of binding a tumor-associated antigen (TAA) on the surface of a tumor cell;
(iii) a fourth binding domain capable of binding PD-L1 on the surface of a tumor cell; and
(iv) a scaffold,
wherein the first binding domain, the second binding domain, the third binding domain and the fourth binding domain are operably linked to the scaffold.

Embodiment 77. A pharmaceutical composition comprising the trispecific fusion protein of any one of embodiments 1-76, and a pharmaceutically acceptable carrier, excipient, diluent, or combination thereof.

Embodiment 78. A nucleic acid molecule or a set of nucleic acid molecules encoding the trispecific fusion protein of any one of embodiments 1-76.

Embodiment 79. A vector or a set of vectors comprising the nucleic acid molecule or the set of nucleic acid molecules of embodiment 78.

Embodiment 80. A cell comprising the nucleic acid molecule or the set of nucleic acid molecules of embodiment 78, or the vector or the set of vectors of embodiment 79.

Embodiment 81. A method of producing a trispecific fusion protein of any one of embodiments 1-76, the method comprising:
(a) obtaining a host cell culture comprising at least one host cell comprising one or more nucleic acid molecules encoding the trispecific fusion protein; and
(b) recovering the trispecific fusion protein from the host cell culture.

Embodiment 82. The method of embodiment 81, further comprising, subsequent to step (b), purifying the trispecific fusion protein.

Embodiment 83. A method of eliciting an anti-tumor immune response in a cell population comprising cytotoxic effector cells and tumor cells, the method comprising contacting the cell population with an effective amount of the trispecific fusion protein of any one of embodiments 1-76, wherein the cytotoxic effector cells express CD3, and the tumor cells express the TAA and PD-L1.

Embodiment 84. A method of inhibiting the proliferation of tumor cells expressing the TAA and PD-L1, the method comprising contacting a cell population comprising the tumor cells and cytotoxic effector cells with an effective amount of the trispecific fusion protein of any one of embodiments 1-76, wherein the cytotoxic effector cells express CD3.

Embodiment 85. A method of killing tumor cells expressing the TAA and PD-L1, the method comprising contacting a cell population comprising the tumor cells and cytotoxic effector cells with an effective amount of the trispecific fusion protein of any one of embodiments 1-76, wherein the cytotoxic effector cells express CD3.

Embodiment 86. The method of any one of embodiments 83-85, wherein the cytotoxic effector cells comprise T cells.

Embodiment 87. The method of any one of embodiments 83-86, wherein the TAA and PD-L1 are located on the same tumor cell.

Embodiment 88. The method of any one of embodiments 83-86, wherein the TAA and PD-L1 are located on different tumor cells.

Embodiment 89. The method of any one of embodiments 83-88, wherein the binding of CD3, the TAA and PD-L1 by the trispecific fusion protein forms a TCR-independent artificial immune synapse between the immune cell and the tumor cell(s), thereby eliciting a cytotoxic immune response of the immune cell against the tumor cell(s).

Embodiment 90. The method of any one of embodiments 83-89, wherein the cell population is located in a living subject.

Embodiment 91. A method for treating a cancer in a subject in need thereof, the method comprising administering to the subject a trispecific fusion protein of any one of embodiments 1-76.

Embodiment 92. The method of embodiment 91, wherein the trispecific fusion protein elicits a cytotoxic immune response against the cancer in the subject, thereby treating the cancer in the subject.

Embodiment 93. The method of any one of embodiments 90-92, wherein the subject is a rodent, a non-human primate, or a human.

Embodiment 94. A trispecific fusion protein of any one of embodiments 1-76 for use in the treatment of cancer.

Embodiment 95. Use of a trispecific fusion protein of any one of embodiments 1-76, in the manufacture of a medicament for the treatment of cancer.

Embodiment 96. A trispecific fusion protein comprising:
(i) a first binding domain capable of binding CD3 on the surface of a cytotoxic effector cell;
(ii) a second binding domain capable of binding a TAA on the surface of a tumor cell;
(iii) a third binding domain capable of binding PD-L1 on the surface of a tumor cell; and
(iv) a scaffold,
wherein the first binding domain, the second binding domain and the third binding domain are operably linked to the scaffold, and
wherein the trispecific fusion protein is not v31929.

Embodiment 97. A trispecific fusion protein comprising:
(i) a first binding domain capable of binding CD3 on the surface of a cytotoxic effector cell;
(ii) a second binding domain capable of binding a TAA on the surface of a tumor cell;
(iii) a third binding domain capable of binding PD-L1 on the surface of a tumor cell; and
(iv) a scaffold,
wherein the first binding domain, the second binding domain and the third binding domain are operably linked to the scaffold, and
wherein the TAA is not HER2.

Embodiment 97. The trispecific fusion protein of any one of embodiments 96-97, wherein the third binding domain is linked to (i) the first binding domain, (ii) the second binding domain, or (iii) the scaffold.

Embodiment 98. A trispecific fusion protein comprising:
(i) a first binding domain capable of binding CD3 on the surface of a cytotoxic effector cell;
(ii) a second binding domain capable of binding a tumor-associated antigen (TAA) on the surface of a tumor cell;
(iii) a third binding domain capable of binding PD-L1 on the surface of a tumor cell; and
(iv) a scaffold,
wherein the first binding domain, the second binding domain and the third binding domain are operably linked to the scaffold, and
wherein the third binding domain is linked to the second binding domain.

Embodiment 99. A trispecific fusion protein comprising:
(i) a first binding domain capable of binding CD3 on the surface of a cytotoxic effector cell;
(ii) a second binding domain capable of binding a tumor-associated antigen (TAA) on the surface of a tumor cell;
(iii) a third binding domain capable of binding PD-L1 on the surface of a tumor cell; and
(iv) a scaffold,
wherein the first binding domain, the second binding domain and the third binding domain are operably linked to the scaffold, and
wherein the third binding domain is linked to the scaffold.

Embodiment 100. A trispecific fusion protein comprising:
(i) a first binding domain capable of binding CD3 on the surface of a cytotoxic effector cell;
(ii) a second binding domain capable of binding a tumor-associated antigen (TAA) on the surface of a first tumor cell;
(iii) a third binding domain capable of binding PD-L1 on the surface of a second tumor cell; and
(iv) a scaffold,
wherein the first binding domain, the second binding domain and the third binding domain are operably linked to the scaffold, and
wherein the third binding domain is linked to (i) the second binding domain or (ii) the scaffold.

Embodiment 101. The trispecific fusion protein of any one of embodiments 96-100, wherein:
a) the first binding domain is a Fab and the second binding domain is an scFv; or
b) the first binding domain is an scFv and the second binding domain is a Fab; or
c) the first binding domain is a Fab and the second binding domain is a Fab; or
d) the first binding domain is an scFv and the second binding domain is an scFv.

Embodiment 102. A trispecific fusion protein comprising:
(i) a first binding domain capable of binding CD3 on the surface of a cytotoxic effector cell, wherein the first binding domain is a Fab domain;
(ii) a second binding domain capable of binding a tumor-associated antigen (TAA) on the surface of a tumor cell;
(iii) a third binding domain capable of binding PD-L1 on the surface of a tumor cell; and
(iv) a scaffold,
wherein the first binding domain, the second binding domain and the third binding domain are operably linked to the scaffold, and
wherein the third binding domain is linked to (i) the N- or C-terminus of the Fab light chain, (ii) second binding domain, or (iii) the scaffold.

Embodiment 103. The trispecific fusion protein of any one of embodiments 96-102, wherein the scaffold comprises or consists of a dimeric Fc domain comprising a first Fc polypeptide and a second Fc polypeptide.

Embodiment 104. The trispecific fusion protein of embodiment 103, wherein the dimeric Fc domain is a heterodimeric Fc domain, and wherein the amino acid sequence of the first Fc polypeptide differs in at least one amino acid residue from the amino acid sequence of the second Fc polypeptide.

Embodiment 105. The trispecific fusion protein of any one of embodiments 103-104, wherein the first binding domain is linked to the first Fc polypeptide via a first linker^{Fc}.

Embodiment 106. The trispecific fusion protein of any one of embodiments 103-105, wherein the second binding domain is linked to the second Fc polypeptide via a second linker^{Fc}.

Embodiment 107. The trispecific fusion protein of any one of embodiments 105-106, wherein the first linker^{Fc}, the second linker^{Fc}, or both, comprise or consist of an IgG hinge region, or a portion or variant thereof.

Embodiment 108. The trispecific fusion protein of any one of embodiments 105-107, wherein the first linker^{Fc}, the second linker^{Fc}, or both, comprise or consist of an amino acid sequence having at least 80%, 90%, or 95% sequence identity to the amino acid sequence set forth in SEQ ID NO: 50 or a fragment thereof.

Embodiment 109. The trispecific fusion protein of any one of embodiments 96-108, wherein the first binding domain is a Fab domain and is linked to the N-terminus of the first Fc polypeptide via the C-terminus of the Fab heavy chain.

Embodiment 110. The trispecific fusion protein of any one of embodiments 96-109, wherein the second binding domain is an scFv domain and is linked via its C-terminus to the N-terminus of the second Fc polypeptide.

Embodiment 111. The trispecific fusion protein of any one of embodiments 96-110, wherein the trispecific fusion protein comprises or consists of three polypeptide chains comprising two immunoglobulin G heavy chains and one immunoglobulin light chain, wherein the first heavy chain comprises, from N- to C-terminus, the Fab VH and CH1 domains linked to the CH2 and CH3 domains of the first Fc polypeptide, the second heavy chains comprises, from N- to C-terminus, the scFv VH and VL or VL and VH domains, linked to the CH2 and CH3 domains of the second Fc polypeptide, and the light chain comprises, from N- to C-terminus, the Fab VL and CL domains, wherein the light chain is capable of forming a Fab domain with the Fab VH and CH1 domains of the first heavy chain.

Embodiment 112. The trispecific fusion protein of any one of embodiments 96-111, wherein the third binding domain comprises a PD-1 polypeptide.

Embodiment 113. The trispecific fusion protein of any one of embodiments 96-112, wherein the third binding domain consists of a PD-1 polypeptide.

Embodiment 114. The trispecific fusion protein of any one of embodiments 112-113, wherein the PD-1 polypeptide is a wildtype PD-1 polypeptide comprising or consisting of the amino acid sequence set forth in SEQ ID NO: 7, or a portion of fragment thereof.

Embodiment 115. The trispecific fusion protein of any one of embodiments 112-113, wherein the PD-1 polypeptide comprises one or more amino acid modifications compared to a corresponding wildtype PD-1 polypeptide (e.g., SEQ ID NO: 7) that increase or decrease the binding affinity of the PD-1 polypeptide to PD-L1 when compared to the binding affinity of the corresponding wildtype PD-1 polypeptide to PD-L1.

Embodiment 116. The trispecific fusion protein of embodiment 115, wherein the one or more amino acid modifications comprise one or more amino acid substitutions.

Embodiment 117. The trispecific fusion protein of embodiment 116, wherein the PD-1 polypeptide has a binding affinity for PD-L1 of from about 100 μM to about 10 pM, from about 10 μM to about 150 pM, from about 100 nM and 150 pM, or from about 5 nM to about 90 nM.

Embodiment 118. The trispecific fusion protein of any one of embodiments 112-117, wherein the PD-1 polypeptide comprises or consists of an amino acid sequence having at least about 80%, 90%, 95%, 99%, or 100% sequence identity to the amino acid sequence set forth in SEQ ID NO: 9.

Embodiment 119. The trispecific fusion protein of any one of embodiments 96-118, wherein the anti-CD3 binding domain comprises a VH domain comprising a HCDR1 sequence selected from the group consisting of RSTMH (SEQ ID NO: 207), YYGMS (SEQ ID NO: 303), KYAMN (SEQ ID NO: 224) and TYAMN (SEQ ID NO: 232), a HCDR2 sequence selected from the group consisting of YINPSSAYTNYNQKFKD (SEQ ID NO: 208), SITSSGGRIYYPDSVKG (SEQ ID NO: 301), SITRSGGRIYYPDSVKG (SEQ ID NO: 217), RIRSKYNNYATYYADSVKD (SEQ ID NO: 225) and RIRSKYNNYATYYADSVKG (SEQ ID NO: 233), and a HCDR3 sequence selected from the group consisting of PQVHYDYNGFPY (SEQ ID NO: 209), DGRDGWVAY (SEQ ID NO: 275), HGNFGNSYISYWAY (SEQ ID NO: 226) and HGNFGNSYVSWFAY (SEQ ID NO: 234), and a VL domain comprising an LCDR1 sequence selected from the group consisting of SASSSVSYMN (SEQ ID NO: 211), KRNTGNIGSNYVN (SEQ ID NO: 287), TGNTGNIGSNYVN (SEQ ID NO: 220), GSSTGAVTSGNYPN (SEQ ID NO: 228) and GSSTGAVTTSNYAN (SEQ ID NO: 236), an LCDR2 sequence selected from the group consisting of DSSKLAS (SEQ ID NO: 212), RNDKRPD (SEQ ID NO: 298), RDDKRPS (SEQ ID NO: 221), GTKFLAP (SEQ ID NO: 229), RSYQRPS (SEQ ID NO: 199) and GTNKRAP (SEQ ID NO: 237), and an LCDR3 sequence selected from the group consisting of QQWSRNPPT (SEQ ID NO: 214), QSYSSGFI (SEQ ID NO: 295), VLWYSNRWV (SEQ ID NO: 230), ATWDDSLDGWV (SEQ ID NO: 200) and ALWYSNLWV (SEQ ID NO: 238).

Embodiment 120. The trispecific fusion protein of any one of embodiments 96-119, wherein the anti-CD3 binding domain comprises a VH domain comprising the HCDR sequences YYGMS (SEQ ID NO: 303), SITSSGGRIYYPDSVKG (SEQ ID NO: 301) and DGRDGWVAY (SEQ ID NO: 275), and a VL domain comprising the LCDR sequences KRNTGNIGSNYVN (SEQ ID NO: 287), RNDKRPD (SEQ ID NO: 298) and QSYSSGFI (SEQ ID NO: 295).

Embodiment 121. The trispecific fusion protein of any one of embodiments 96-119, wherein the anti-CD3 binding domain comprises a VH domain comprising the HCDR sequences YYGMS (SEQ ID NO: 302), SITRSGGRIYYPDSVKG (SEQ ID NO: 217) and DGRDGWVAY (SEQ ID NO: 275), and a VL domain comprising the LCDR sequences TGNTGNIGSNYVN (SEQ ID NO: 220), RDDKRPS (SEQ ID NO: 221) and QSYSSGFI (SEQ ID NO: 295).

Embodiment 122. The trispecific fusion protein of any one of embodiments 96-119, wherein the anti-CD3 binding domain comprises a VH domain comprising the HCDR sequences KYAMN (SEQ ID NO: 224), RIRSKYNNYATYYADSVKD (SEQ ID NO: 225) and HGNFGNSYISYWAY (SEQ ID NO: 226), and a VL domain comprising the LCDR sequences GSSTGAVTSGNYPN (SEQ ID NO: 228), GTKFLAP (SEQ ID NO: 229) and VLWYSNRWV (SEQ ID NO: 230).

Embodiment 123. The trispecific fusion protein of any one of embodiments 96-119, wherein the anti-CD3 binding domain comprises a VH domain comprising the HCDR sequences TYAMN (SEQ ID NO: 232), RIRSKYNNYATYYADSVKG (SEQ ID NO: 233) and HGNFGNSYVSWFAY (SEQ ID NO: 234), and a VL domain comprising the LCDR sequences GSSTGAVTTSNYAN (SEQ ID NO: 236), GTNKRAP (SEQ ID NO: 237) and ALWYSNLWV (SEQ ID NO: 238).

Embodiment 124. The trispecific fusion protein of any one of embodiments 96-119, wherein the anti-CD3 binding domain comprises a VH domain comprising the HCDR sequences RSTMH (SEQ ID NO: 207), YINPSSAYTNYNQKFKD (SEQ ID NO: 208) and PQVHYDYNGFPY (SEQ ID NO: 209), and a VL domain comprising the LCDR sequences SASSSVSYMN (SEQ ID NO: 211), DSSKLAS (SEQ ID NO: 212) and QQWSRNPPT (SEQ ID NO: 214).

Embodiment 125. The trispecific fusion protein of any one of embodiments 96-119, wherein the anti-CD3 binding domain comprises a VH domain comprising the HCDR sequences RSTMH (SEQ ID NO: 207), YINPSSAYTNYNQKFKD (SEQ ID NO: 208) and PQVHYDYNGFPY (SEQ ID NO: 209), and a VL domain comprising the LCDR sequences RSYQRPS (SEQ ID NO: 199) and ATWDDSLDGWV (SEQ ID NO: 200).

Embodiment 126. The trispecific fusion protein of any one of embodiments 96-119, wherein the anti-CD3 binding domain comprises a VH domain comprising an amino acid sequence having at least about 90%, 95%, 97%, 99%, or 100% sequence identity to the sequence set forth in any one of SEQ ID NOs: 2, 215, 223, and 231, and a VL domain comprising an amino acid sequence having at least about 90%, 95%, 97%, 99%, or 100% sequence identity to the sequence set forth in any one of SEQ ID NOs: 1, 219, 227, and 235.

Embodiment 127. The trispecific fusion protein of any one of embodiments 96-126, wherein the TAA is not HER2.

Embodiment 128. The trispecific fusion protein of any one of embodiments 96-127, wherein the TAA is Cldn18.2.

Embodiment 129. The trispecific fusion protein of any one of embodiments 96 or 98-127, wherein the TAA is HER2 and the anti-HER2 VH sequence of the second binding domain comprises a HCDR1 sequence comprising DTYIH (SEQ ID NO: 121), a HCDR2 sequence comprising RIYPTNGYTRYADSVKG (SEQ ID NO: 122), and a HCDR3 sequence comprising WGGDGFYAMDY (SEQ ID NO: 123), and the anti-HER2 VL sequence of the second binding domain comprises an LCDR1 sequence comprising RASQDVNTAVA (SEQ ID NO: 125), an LCDR2 sequence comprising SASFLYS (SEQ ID NO: 126), and an LCDR3 sequence comprising QQHYTTPPT (SEQ ID NO: 127).

Embodiment 130. The trispecific fusion protein of embodiment 129, wherein the anti-HER2 binding domain comprises a VH domain comprising an amino acid sequence having at least about 90%, 95%, 97%, 99%, or 100% sequence identity to the sequence set forth in SEQ ID NO: 120, and 231, and a VL domain comprising an amino acid sequence having at least about 90%, 95%, 97%, 99%, or 100% sequence identity to the sequence set forth in SEQ ID NO: 124.

Embodiment 131. The trispecific fusion protein of any one of embodiments 96-127, wherein the TAA is MSLN and the anti-MSLN VH sequence of the second binding domain comprises a HCDR1 sequence comprising GYTMN (SEQ ID NO: 286), a HCDR2 sequence comprising LITPYNGASSYNQKFRG (SEQ ID NO: 288) and a HCDR3 sequence comprising GGYDGRGFDY (SEQ ID NO: 285), and the anti-MSLN VL sequence of the second binding domain comprises an LCDR1 sequence comprising SASSSVSYMH (SEQ ID NO: 300), an LCDR2 sequence comprising DTSKLAS (SEQ ID NO: 279) and an LCDR3 sequence comprising QQWSGYPLT (SEQ ID NO: 294).

Embodiment 132. The trispecific fusion protein of embodiment 131, wherein the anti-MSLN VH sequence of the second binding comprises an amino acid sequence having at least about 90%, 95%, 97%, 99%, or 100% sequence identity to the sequence set forth in SEQ ID NO: 297, and the anti-MSLN VL sequence of the second binding comprises an amino acid sequence having at least about 90%, 95%, 97%, 99%, or 100% sequence identity to the sequence set forth in SEQ ID NOs: 276 or 277.

Embodiment 133. The trispecific fusion protein of embodiment 128, wherein the TAA is Cldn18.2 and the anti-Cldn18.2 VH sequence of the second binding domain comprises a HCDR1 sequence comprising SNPMI (SEQ ID NO: 310), a HCDR2 sequence comprising IIDTDGSTYYADWAKG (SEQ ID NO: 311) and a HCDR3 sequence comprising RLHGSSNGYYDDL (SEQ ID NO: 312), and the anti-Cldn18.2 VL sequence of the second binding domain comprises an LCDR1 sequence comprising QASQSIYSYLS (SEQ ID NO: 313), an LCDR2 sequence comprising KASTLAS (SEQ ID NO: 314) and an LCDR3 sequence comprising QQGYTVTNVDKNT (SEQ ID NO: 315).

Embodiment 134. The trispecific fusion protein of embodiment 133, wherein the anti-Cldn18.2 VH sequence of the second binding domain comprises an amino acid sequence having at least about 90%, 95%, 97%, 99%, or 100% sequence identity to the sequence set forth in SEQ ID NO: 316, and the anti-Cldn18.2 VL sequence of the second binding domain comprises an amino acid sequence having at least about 90%, 95%, 97%, 99%, or 100% sequence identity to the sequence set forth in SEQ ID NO: 317.

Embodiment 135. The trispecific fusion protein of any one of embodiments 103-134, wherein the first Fc polypeptide and the second Fc polypeptide each comprise a CH2 domain comprising an amino acid sequence having at least about 90%, 95%, 97%, 99%, or 100% sequence identity to the sequence set forth in SEQ ID NO: 6.

Embodiment 136. The trispecific fusion protein of any one of embodiments 103-135, wherein one of the first Fc polypeptide or the second Fc polypeptide comprises a CH3 domain comprising an amino acid sequence having at least about 90%, 95%, 97%, 99%, or 100% sequence identity to the sequence set forth in SEQ ID NO: 4, and the other Fc polypeptide comprises a CH3 domain comprising an amino acid sequence having at least about 90%, 95%, 97%, 99%, or 100% sequence identity to the sequence set forth in SEQ ID NO: 5.

Embodiment 137. The trispecific fusion protein of any one of embodiments 96 or 100-103, wherein the TAA is HER2 and wherein the trispecific fusion protein comprises (i) a first heavy chain having at least 90%, 95%, 97%, 99% or 100% sequence identity to the clone sequence set forth in 23734, (ii) a second heavy chain having at least 90%, 95%, 97%, 99% or 100% sequence identity to the clone sequence set forth in 21490, and (iii) a light chain having at least 90%, 95%, 97%, 99% or 100% sequence identity to the clone sequence set forth in 12985.

Embodiment 138. The trispecific fusion protein of any one of embodiments 96-97 or 100-103, wherein the TAA is MSLN and the trispecific fusion protein comprises (i) a first heavy chain having at least 90%, 95%, 97%, 99% or 100% sequence identity to the clone sequence set forth in 29207, 29208, 29276, 29238, 29282, 22080 or 23734, (ii) a second heavy chain having at least 90%, 95%, 97%, 99% or 100% sequence identity to the clone sequence set forth in 23867, 23270, 29275 or 25095 and (iii) a light chain having at least 90%, 95%, 97%, 99% or 100% sequence identity to the clone sequence set forth in 16412, 23570 or 12985.

Embodiment 139. The trispecific fusion protein of any one of embodiments 96-100, wherein the trispecific fusion protein comprises (i) a first heavy chain having at least 90%, 95%, 97%, 99% or 100% sequence identity to the clone sequence set forth in 29207, (ii) a second heavy chain having at least 90%, 95%, 97%, 99% or 100% sequence identity to the clone sequence set forth in 29275, and (iii) a light chain having at least 90%, 95%, 97%, 99% or 100% sequence identity to the clone sequence set forth in 16412.

Embodiment 140. The trispecific fusion protein of any one of embodiments 96-100, wherein the trispecific fusion protein comprises (i) a first heavy chain having at least 90%, 95%, 97%, 99% or 100% sequence identity to the clone sequence set forth in 29208, (ii) a second heavy chain having at least 90%, 95%, 97%, 99% or 100% sequence identity to the clone sequence set forth in 29275, and (iii) a light chain having at least 90%, 95%, 97%, 99% or 100% sequence identity to the clone sequence set forth in 16412.

Embodiment 141. The trispecific fusion protein of any one of embodiments 96-100, wherein the trispecific fusion protein comprises (i) a first heavy chain having at least 90%, 95%, 97%, 99% or 100% sequence identity to the clone sequence set forth in 29276, (ii) a second heavy chain having at least 90%, 95%, 97%, 99% or 100% sequence identity to the clone sequence set forth in 25095, and (iii) a light chain having at least 90%, 95%, 97%, 99% or 100% sequence identity to the clone sequence set forth in 23570.

Embodiment 142. The trispecific fusion protein of any one of embodiments 96-100 or 102-103, wherein the trispecific fusion protein comprises (i) a first heavy chain having at least 90%, 95%, 97%, 99% or 100% sequence identity to the clone sequence set forth in 29238, (ii) a second heavy chain having at least 90%, 95%, 97%, 99% or 100% sequence identity to the clone sequence set forth in 29275, and (iii) a light chain having at least 90%, 95%, 97%, 99% or 100% sequence identity to the clone sequence set forth in 16412.

Embodiment 143. The trispecific fusion protein of any one of embodiments 96-97, wherein the trispecific fusion protein comprises (i) a first heavy chain having at least 90%, 95%, 97%, 99% or 100% sequence identity to the clone sequence set forth in 29282, (ii) a second heavy chain having at least 90%, 95%, 97%, 99% or 100% sequence identity to the clone sequence set forth in 23270, and (iii) a light chain having at least 90%, 95%, 97%, 99% or 100% sequence identity to the clone sequence set forth in 16412.

Embodiment 144. The trispecific fusion protein of any one of embodiments 96-100, wherein the trispecific fusion protein comprises (i) a first heavy chain having at least 90%, 95%, 97%, 99% or 100% sequence identity to the clone sequence set forth in 22080, (ii) a second heavy chain having at least 90%, 95%, 97%, 99% or 100% sequence identity to the clone sequence set forth in 23867, and (iii) a light chain having at least 90%, 95%, 97%, 99% or 100% sequence identity to the clone sequence set forth in 12985.

Embodiment 145. The trispecific fusion protein of any one of embodiments 96-100, wherein the trispecific fusion protein comprises (i) a first heavy chain having at least 90%, 95%, 97%, 99% or 100% sequence identity to the clone sequence set forth in 23734, (ii) a second heavy chain having at least 90%, 95%, 97%, 99% or 100% sequence identity to the clone sequence set forth in 23867, and (iii) a light chain having at least 90%, 95%, 97%, 99% or 100% sequence identity to the clone sequence set forth in 12985.

Embodiment 146. The trispecific fusion protein of any one of embodiments 96-103, wherein the TAA is Cldn18.2 and the trispecific fusion protein comprises (i) a first heavy chain having at least 90%, 95%, 97%, 99% or 100% sequence identity to the clone sequence set forth in 29241, 29238, 29208 or 29211, (ii) a second heavy chain having at least 90%, 95%, 97%, 99% or 100% sequence identity to the clone sequence set forth in 29264, 29261, 29267 or 28373, and (iii) a light chain having at least 90%, 95%, 97%, 99% or 100% sequence identity to the clone sequence set forth in 16412.

Embodiment 147. The trispecific fusion protein of any one of embodiments 96-100 or 103, wherein the trispecific fusion protein comprises (i) a first heavy chain having at least 90%, 95%, 97%, 99% or 100% sequence identity to the clone sequence set forth in 29241, (ii) a second heavy chain having at least 90%, 95%, 97%, 99% or 100% sequence identity to the clone sequence set forth in 28373, and (iii) a light chain having at least 90%, 95%, 97%, 99% or 100% sequence identity to the clone sequence set forth in 16412.

Embodiment 148. The trispecific fusion protein of any one of embodiments 96-100 or 103, wherein the trispecific fusion protein comprises (i) a first heavy chain having at least 90%, 95%, 97%, 99% or 100% sequence identity to the clone sequence set forth in 29238, (ii) a second heavy chain having at least 90%, 95%, 97%, 99% or 100% sequence identity to the clone sequence set forth in 28373, and (iii) a light chain having at least 90%, 95%, 97%, 99% or 100% sequence identity to the clone sequence set forth in 16412.

Embodiment 149. The trispecific fusion protein of any one of embodiments 96-100, wherein the trispecific fusion protein comprises (i) a first heavy chain having at least 90%, 95%, 97%, 99% or 100% sequence identity to the clone sequence set forth in 29208, (ii) a second heavy chain having at least 90%, 95%, 97%, 99% or 100% sequence identity to the clone sequence set forth in 28373, and (iii) a light chain having at least 90%, 95%, 97%, 99% or 100% sequence identity to the clone sequence set forth in 16412.

Embodiment 150. The trispecific fusion protein of any one of embodiments 96-100, wherein the trispecific fusion protein comprises (i) a first heavy chain having at least 90%, 95%, 97%, 99% or 100% sequence identity to the clone sequence set forth in 29211, (ii) a second heavy chain having at least 90%, 95%, 97%, 99% or 100% sequence identity to the clone sequence set forth in 28373, and (iii) a light chain having at least 90%, 95%, 97%, 99% or 100% sequence identity to the clone sequence set forth in 16412.

Embodiment 151. A tetravalent and trispecific fusion protein comprising:
(i) a first binding domain capable of binding CD3 on the surface of a cytotoxic effector cell;
(ii) a second binding domain and a third binding domain capable of binding a tumor-associated antigen (TAA) on the surface of a tumor cell;
(iii) a fourth binding domain capable of binding PD-L1 on the surface of a tumor cell; and
(iv) a scaffold,
wherein the first binding domain, the second binding domain, the third binding domain and the fourth binding domain are operably linked to the scaffold.

Embodiment 152. The tetravalent and trispecific fusion protein of embodiment 151, wherein the scaffold comprises or consists of a dimeric Fc domain comprising a first Fc polypeptide and a second Fc polypeptide.

Embodiment 153. The tetravalent and trispecific fusion protein of embodiment 152, wherein the dimeric Fc domain is a heterodimeric Fc domain, wherein the amino acid sequence of the first Fc polypeptide differs in at least one amino acid residue from the amino acid sequence of the second Fc polypeptide.

Embodiment 154. The tetravalent and trispecific fusion protein of any one of embodiments 151-153, wherein:
a) the first binding domain is a Fab and the second and third binding domains are both scFv domains; or
b) the first binding domain is an scFv and the second and third binding domains are both Fab domains; or
c) the first binding domain, the second binding domain and the third binding domain are Fab domains; or
d) the first binding domain, the second binding domain and the third binding domain are scFv domains.

Embodiment 155. The tetravalent and trispecific fusion protein of any one of embodiments 151-154, wherein (i) the first binding domain is a Fab domain, (ii) the second binding domain is a first scFv domain, and (iii) the third binding domain is a second scFv domain.

Embodiment 156. The tetravalent and trispecific fusion protein of any one of embodiments 151-155, wherein the first binding domain is linked to the N-terminus of the first Fc polypeptide, and the second binding domain is linked to the N-terminus of the second Fc polypeptide.

Embodiment 157. The tetravalent and trispecific fusion protein of any one of embodiments 151-156, wherein the third binding domain is linked to (i) the first binding domain, (ii) the second binding domain, or (iii) the scaffold.

Embodiment 158. The tetravalent and trispecific fusion protein of any one of embodiments 151-157, wherein:
(a) the first binding domain is a Fab domain comprising a Fab heavy chain and a Fab light chain, wherein the C-terminus of the Fab heavy chain is linked to the N-terminus of the first Fc polypeptide;
(b) the second binding domain is a first scFv domain comprising the structure, from N- to C-terminus, VH-Linker-VL or VL-Linker-VH, wherein the C-terminus of the first scFv domain is linked to the N-terminus of the second Fc polypeptide;
(c) the third binding domain is a second scFv domain comprising the structure, from N- to C-terminus, VH-Linker-VL or VL-Linker-VH, wherein the C-terminus of the second scFv domain is linked to (i) the N-terminus of the Fab heavy or light chain, or (ii) the C-terminus of the first or second Fc polypeptide.

Embodiment 159. The tetravalent and trispecific fusion protein of any one of embodiments 151-158, wherein the second binding domain and the third binding domain are capable of binding the same TAA.

Embodiment 160. The tetravalent and trispecific fusion protein of embodiment 159, wherein the second binding domain and the third binding domain are capable of binding the same epitope on the TAA.

Embodiment 161. The tetravalent and trispecific fusion protein of any one of embodiments 151-160, wherein the second binding domain and the third binding domain comprise the same anti-TAA VH and VL sequences.

Embodiment 162. The tetravalent and trispecific fusion protein of any one of embodiments 151-161, wherein the fourth binding domain is linked to (i) the first binding domain, (ii) the second binding domain, (iii) the third binding domain, or (iv) the scaffold.

Embodiment 163. The tetravalent and trispecific fusion protein of any one of embodiments 151-155, wherein the TAA is MSLN and the trispecific fusion protein comprises (i) a first heavy chain having at least 90%, 95%, 97%, 99% or 100% sequence identity to the clone sequence set forth in 29257 or 29283, (ii) a second heavy chain having at least 90%, 95%, 97%, 99% or 100% sequence identity to the clone sequence set forth in 23867, 29258, 29264, 23867, 29263, 29267 or 29261 and (iii) a light chain having at least 90%, 95%, 97%, 99% or 100% sequence identity to the clone sequence set forth in 16412, 29226 or 29220.

Embodiment 164. The tetravalent and trispecific fusion protein of embodiment 163, wherein the trispecific fusion protein comprises (i) a first heavy chain having at least 90%, 95%, 97%, 99% or 100% sequence identity to the clone sequence set forth in 29257, (ii) a second heavy chain having at least 90%, 95%, 97%, 99% or 100% sequence identity to the clone sequence set forth in 23867, and (iii) a light chain having at least 90%, 95%, 97%, 99% or 100% sequence identity to the clone sequence set forth in 29220.

Embodiment 165. The tetravalent and trispecific fusion protein of embodiment 163, wherein the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 29258, and (iii) a light chain having at least 90%, 95%, 97%, 99% or 100% sequence identity to the clone sequence set forth in 16412.

Embodiment 166. The tetravalent and trispecific fusion protein of embodiment 163, wherein the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 29264, and (iii) a light chain having at least 90%, 95%, 97%, 99% or 100% sequence identity to the clone sequence set forth in 16412.

Embodiment 167. The tetravalent and trispecific fusion protein of embodiment 163, wherein the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 23867, and (iii) a light chain having at least 90%, 95%, 97%, 99% or 100% sequence identity to the clone sequence set forth in 29220.

Embodiment 168. The tetravalent and trispecific fusion protein of embodiment 163, wherein the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 23867, and (iii) a light chain having at least 90%, 95%, 97%, 99% or 100% sequence identity to the clone sequence set forth in 29226.

Embodiment 169. The tetravalent and trispecific fusion protein of embodiment 163, wherein the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 29263, and (iii) a light chain having at least 90%, 95%, 97%, 99% or 100% sequence identity to the clone sequence set forth in 16412.

Embodiment 170. The tetravalent and trispecific fusion protein of embodiment 163, wherein the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 29264, and (iii) a light chain having at least 90%, 95%, 97%, 99% or 100% sequence identity to the clone sequence set forth in 16412.

Embodiment 171. The tetravalent and trispecific fusion protein of embodiment 163, wherein the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 29261, and (iii) a light chain having at least 90%, 95%, 97%, 99% or 100% sequence identity to the clone sequence set forth in 16412.

Embodiment 172. The tetravalent and trispecific fusion protein of embodiment 163, wherein the trispecific fusion protein comprises (i) a first heavy chain having at least 90%, 95%, 97%, 99% or 100% sequence identity to the clone sequence set forth in 29283, (ii) a second heavy chain having at least 90%, 95%, 97%, 99% or 100% sequence identity to the clone sequence set forth in 29267, and (iii) a light chain having at least 90%, 95%, 97%, 99% or 100% sequence identity to the clone sequence set forth in 16412.

Embodiment 173. The tetravalent and trispecific fusion protein of any one of embodiments 151-155, wherein the TAA is Cldn18.2 and the trispecific fusion protein comprises (i) a first heavy chain having at least 90%, 95%, 97%, 99% or 100% sequence identity to the clone sequence set forth in 29244, (ii) a second heavy chain having at least 90%, 95%, 97%, 99% or 100% sequence identity to the clone sequence set forth in 29245, 29248, 29251 or 29254 and (iii) a light chain having at least 90%, 95%, 97%, 99% or 100% sequence identity to the clone sequence set forth in 16412.

Embodiment 174. The tetravalent and trispecific fusion protein of embodiment 173, wherein the trispecific fusion protein comprises (i) a first heavy chain having at least 90%, 95%, 97%, 99% or 100% sequence identity to the clone sequence set forth in 29244, (ii) a second heavy chain having at least 90%, 95%, 97%, 99% or 100% sequence identity to the clone sequence set forth in 29245, and (iii) a light chain having at least 90%, 95%, 97%, 99% or 100% sequence identity to the clone sequence set forth in 16412.

Embodiment 175. The tetravalent and trispecific fusion protein of embodiment 173, wherein the trispecific fusion protein comprises (i) a first heavy chain having at least 90%, 95%, 97%, 99% or 100% sequence identity to the clone sequence set forth in 29244, (ii) a second heavy chain having at least 90%, 95%, 97%, 99% or 100% sequence identity to the clone sequence set forth in 29248, and (iii) a light chain having at least 90%, 95%, 97%, 99% or 100% sequence identity to the clone sequence set forth in 16412.

Embodiment 176. The tetravalent and trispecific fusion protein of embodiment 173, wherein the trispecific fusion protein comprises (i) a first heavy chain having at least 90%, 95%, 97%, 99% or 100% sequence identity to the clone sequence set forth in 29244, (ii) a second heavy chain having at least 90%, 95%, 97%, 99% or 100% sequence identity to the clone sequence set forth in 29251, and (iii) a light chain having at least 90%, 95%, 97%, 99% or 100% sequence identity to the clone sequence set forth in v16412.

Embodiment 177. The tetravalent and trispecific fusion protein of embodiment 173, wherein the trispecific fusion protein comprises (i) a first heavy chain having at least 90%, 95%, 97%, 99% or 100% sequence identity to the clone sequence set forth in 29244, (ii) a second heavy chain having at least 90%, 95%, 97%, 99% or 100% sequence identity to the clone sequence set forth in 29254, and (iii) a light chain having at least 90%, 95%, 97%, 99% or 100% sequence identity to the clone sequence set forth in 16412.

Embodiment 178. The trispecific fusion protein of any one of embodiments 96-150 or the tetravalent and trispecific fusion protein of any one of embodiments 151-177, wherein upon binding to CD3 on the cytotoxic effector cell, the TAA on a tumor cell and PD-L1 on a tumor cell, the trispecific fusion protein forms a TCR-independent immune synapse capable of inducing effector cell mediated cytotoxicity against the tumor cell.

Embodiment 179. The trispecific fusion protein of any one of embodiments 96-150 or the tetravalent and trispecific fusion protein of any one of embodiments 151-177, wherein the trispecific fusion protein binds the TAA and PD-L1 on the same tumor cell.

Embodiment 180. The trispecific fusion protein of any one of embodiments 96-150 or the tetravalent and trispecific fusion protein of any one of embodiments 151-177, wherein the trispecific fusion protein binds the TAA and PD-L1 on different tumor cells.

Embodiment 181. A pharmaceutical composition comprising trispecific fusion protein of any one of embodiments 96-150 or the tetravalent and trispecific fusion protein of any one of embodiments 151-177, and a pharmaceutically acceptable carrier, excipient, diluent, or combination thereof.

Embodiment 182. A nucleic acid molecule or a set of nucleic acid molecules encoding the trispecific fusion protein of any one of embodiments 96-150 or the tetravalent and trispecific fusion protein of any one of embodiments 151-177.

Embodiment 183. A vector or a set of vectors comprising the nucleic acid molecule or the set of nucleic acid molecules of embodiment 182.

Embodiment 184. A cell comprising the nucleic acid molecule or the set of nucleic acid molecules of embodiment 182, or the vector or the set of vectors of embodiment 183.

Embodiment 185. A method of producing the trispecific fusion protein of any one of embodiments 96-150 or the tetravalent and trispecific fusion protein of any one of embodiments 151-177, the method comprising:
(a) obtaining a host cell culture comprising at least one host cell comprising one or more nucleic acid molecules encoding the trispecific fusion protein; and
(b) recovering the trispecific fusion protein from the host cell culture.

Embodiment 186. The method of embodiment 185, further comprising, subsequent to step (b), purifying the trispecific fusion protein.

Embodiment 187. A method of eliciting an anti-tumor immune response in a cell population comprising cytotoxic effector cells and tumor cells, the method comprising contacting the cell population with an effective amount of the trispecific fusion protein of any one of embodiments 96-150 or the tetravalent and trispecific fusion protein of any one of embodiments 151-177, wherein the cytotoxic effector cells express CD3, and the tumor cells express the TAA and PD-L1.

Embodiment 188. A method of inhibiting the proliferation of tumor cells expressing the TAA and PD-L1, the method comprising contacting a cell population comprising the tumor cells and cytotoxic effector cells with an effective amount of the trispecific fusion protein of any one of embodiments 96-150 or the tetravalent and trispecific fusion protein of any one of embodiments 151-177, wherein the cytotoxic effector cells express CD3.

Embodiment 189. A method of killing tumor cells expressing the TAA and PD-L1, the method comprising contacting a cell population comprising the tumor cells and cytotoxic effector cells with an effective amount of the trispecific fusion protein of any one of embodiments 96-150 or the tetravalent and trispecific fusion protein of any one of embodiments 151-177, wherein the cytotoxic effector cells express CD3.

Embodiment 190. The method of any one of embodiments 187-189, wherein the cytotoxic effector cells comprise T cells.

Embodiment 191. The method of any one of embodiments 187-190, wherein the TAA and PD-L1 are located on the same tumor cell.

Embodiment 192. The method of any one of embodiments 187-191, wherein the TAA and PD-L1 are located on different tumor cells.

Embodiment 193. The method of any one of embodiments 187-192, wherein the binding of CD3, the TAA and PD-L1 forms a TCR-independent artificial immune synapse between the immune cell and the tumor cell, thereby eliciting a cytotoxic immune response of the immune cell against the tumor cell.

Embodiment 194. The method of any one of embodiments 187-193, wherein the cell population is located in a living subject.

Embodiment 195. A method for treating a cancer in a subject in need thereof, the method comprising administering to the subject the trispecific fusion protein of any one of embodiments 96-150 or the tetravalent and trispecific fusion protein of any one of embodiments 151-177.

Embodiment 196. The method of embodiment 195, wherein the trispecific fusion protein elicits a cytotoxic immune response against the cancer in the subject, thereby treating the cancer in the subject.

Embodiment 197. The method of any one of embodiments 194-196, wherein the subject is a rodent, a non-human primate, or a human.

Embodiment 198. A trispecific fusion protein of any one of embodiments 96-150 or the tetravalent and trispecific fusion protein of any one of embodiments 151-177 for use in the treatment of cancer.

Embodiment 199. Use of a trispecific fusion protein of any one of embodiments 96-150 or the tetravalent and trispecific fusion protein of any one of embodiments 151-177, in the manufacture of a medicament for the treatment of cancer.

Embodiment 200. A pharmaceutical composition comprising a trispecific fusion protein, the trispecific fusion protein comprising:
(i) a first binding domain capable of binding an antigen on the surface of a cytotoxic effector cell;
(ii) a second binding domain capable of binding a tumor-associated antigen (TAA) on the surface of a first tumor cell;
(iii) a third binding domain capable of binding PD-L1 on the surface of a second tumor cell; and
(iv) a scaffold,
wherein the first binding domain, the second binding domain and the third binding domain are operably linked to the scaffold.

Embodiment 201. A method of treating a disease in a subject in need thereof, the method comprising administering to the subject a trispecific fusion protein, the trispecific fusion protein comprising:
(i) a first binding domain capable of binding an antigen on the surface of a cytotoxic effector cell;
(ii) a second binding domain capable of binding a tumor-associated antigen (TAA) on the surface of a first tumor cell;
(iii) a third binding domain capable of binding PD-L1 on the surface of a second tumor cell; and
(iv) a scaffold,
wherein the first binding domain, the second binding domain and the third binding domain are operably linked to the scaffold.

Embodiment 202. A method of killing cancer cells in a subject in need thereof, the method comprising administering to the subject a trispecific fusion protein, the trispecific fusion protein comprising:
(i) a first binding domain capable of binding an antigen on the surface of a cytotoxic effector cell;
(ii) a second binding domain capable of binding a tumor-associated antigen (TAA) on the surface of a first tumor cell;
(iii) a third binding domain capable of binding PD-L1 on the surface of a second tumor cell; and
(iv) a scaffold,
wherein the first binding domain, the second binding domain and the third binding domain are operably linked to the scaffold.

Embodiment 203. A trispecific fusion protein comprising
a) an anti-CD3 binding domain comprising a VH region and a VL region that specifically binds a CD3 (Cluster of Differentiation 3) antigen on the surface of a T cell;
b) one or two anti-TAA binding domains comprising a VH region and a VL region that specifically binds a TAA (tumor associated antigen) on the surface of a tumor cell;
c) a PD-1 polypeptide capable of binding to a PD-L1 receptor on the surface of a tumor cell;
wherein the anti-CD3 binding domain, the anti-TAA binding domain and the PD-1 polypeptide are operably linked to a scaffold.

Embodiment 204. The fusion protein according to embodiment 203, wherein the scaffold is an antibody comprising a heterodimeric Fc region.

Embodiment 205. The fusion protein according to embodiments 203 or 204, wherein the anti-CD3 binding domain is a Fab, the anti-TAA binding domain is an scFv, and the PD-1 polypeptide is fused to the N-terminus of the VH of the anti-CD3 binding domain Fab via a peptidic linker.

Embodiment 206. The fusion protein according to embodiments 203 or 204, wherein the anti-CD3 binding domain is a Fab, the anti-TAA binding domain is an scFv, and the PD-1 polypeptide is fused to the N-terminus of the VL of the anti-CD3 binding domain Fab via a peptidic linker.

Embodiment 207. The fusion protein according to any of the preceding embodiments, wherein the PD-1 polypeptide is a wildtype PD-1.

Embodiment 208. The fusion protein according to embodiments 203-206, wherein the PD-1 polypeptide comprises one or more mutations that increase or decrease its binding affinity for PD-L1.

Embodiment 209. The fusion protein according to embodiments 203-206, wherein the PD-1 polypeptide comprises one or more mutations that increase its binding affinity for PD-L1.

Embodiment 210. The fusion protein according to embodiments 203-206, wherein the PD-1 polypeptide has an affinity for PD-L1 of between about 100 μM and 10 pM, or between about 10 μM and about 150 pM, or between about 100 nM and 150 pM.

Embodiment 211. The fusion protein according to embodiments 203-205, wherein the PD-1 polypeptide comprises the amino acid sequence of SEQ ID NO: 9.

Embodiment 212. The fusion protein according to any of the preceding embodiments, wherein the TAA is selected from mesothelin, Claudin18.2, GPC3, DLL3, PSMA, MUC17, LIV1, ROR1 and EGFRvIII.

Embodiment 213. The fusion protein according to any of the preceding embodiments, wherein the anti-CD3 antibody domain VH and VL comprise an amino acid sequence selected from SEQ ID NOS: 2 and 1; SEQ ID NOS: 215 and 219; SEQ ID NOS: 223 and 227; or SEQ ID NOS: 231 and 235 respectively.

Embodiment 214. The fusion protein according to any one of embodiments 205 to 213, wherein the peptidic linker is selected from SEQ ID NOS: 15, 16, 17, 18, 19 and 20.

Embodiment 215. The fusion protein according to any of the preceding embodiments, wherein the trispecific fusion protein reduces or inhibits the binding of PD-1 on a T cell to PD-L1 on a cancer cell.

Embodiment 216. A method of treating cancer in a subject in need thereof comprising administering the fusion protein of any of the preceding embodiments to the subject.

Embodiment 217. A method of overcoming or preventing the exhaustion of a T cell comprising exposing the T cell to the fusion protein according to any of the preceding embodiments.

Embodiment 218. A trispecific fusion protein comprising
a) an anti-CD3 binding domain comprising a VH region and a VL region that specifically binds a CD3 (Cluster of Differentiation 3) antigen on the surface of a T cell;
b) one or two anti-TAA binding domains comprising a VH region and a VL region that specifically binds a TAA (tumor associated antigen) on the surface of a tumor cell;
c) a PD-1 polypeptide capable of binding to a PD-L1 receptor on the surface of a tumor cell;
wherein the fusion protein has a format according to any one of FIGS. 3(A)-3(LL).

### EXAMPLES

The following examples are offered for illustrative purposes only and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. Scc, e.g., T. E. Creighton, Proteins: Structures and Molecular Properties (W.H. Freeman and Company, 1993); A. L. Lchninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carcy and Sundberg Advanced Organic Chemistry 3^{rd} Ed. (Plenum Press) Vols A and B (1992).

### Example 1: Design of Exemplary PD-1/Anti-CD3/Anti-Her2 and PD-1/Anti-CD3/Anti-MSLN and PD-1/Anti-CD3/Anti-CLDN18.2 T Cell-Engager Fusion Protein Variants and Controls

An anti-CD3 Fab x anti-Her2 scFv Fc was appended with an extracellular PD-1 IgV polypeptide on the anti-CD3 Fab by linking PD-1 to the N-terminus of the light chain of the Fab. Multiple PD-1/anti-CD3/anti-MSLN antibody geometries were designed by linking PD-1 to the N- or C-terminus of light chain, N-terminus of Fab heavy chain or scFv, or C-terminus of Fc. All formats contained a single PD-1 domain, a single anti-CD3 Fab or scFv, and either one or two anti-MSLN Fab and/or scFv with Fc.

The PD-1/anti-CD3/anti-Her2 fusion proteins were in a modified bispecific Fab x scFv Fc format with a half-antibody comprising the anti-CD3 heavy and light chain that forms a heterodimer with an anti-Her2 scFv fused to an Fc. The anti-CD3 paratope was described in US20150232557A1 (VL, SEQ ID NO: 1, VH, SEQ ID NO: 2). The anti-Her2 paratope was in an scFv format that is based on trastuzumab VL and VH (Carter, P. et al. Humanization of an anti-p185HER2 antibody for human cancer therapy. Proc Natl Acad Sci USA 89, 4285-4289, doi: 10.1073/pnas.89.10.4285 (1992)) connected by a glycine serine linker as described in U.S. Pat. No. 10,000,576B1 (SEQ ID NO: 3). The PD-1/anti-CD3/anti-MSLN fusion proteins were in modified bispecific formats including Fab x scFv Fc, Fab with N-terminal scFv Fc, scFv x Fab with N- or C-terminal scFv Fc. The anti-CD3 paratope was either C3E6 (see, e.g., U.S. Pat. Publ. No. 2019/0359712) was in either Fab or scFv format or the anti-CD3 paratope described in US20150232557A1 (VL SEQ ID NO: 1, VH SEQ ID NO: 2) in scFv format. The anti-MSLN paratope scFv and Fab domain sequences were generated from the VH and VL sequences of the mouse anti-MSLN SS antibody (Chowdhury et al., 1998, PNAS, 95:669-674) with additional humanizing mutations included (see, e.g., U.S. Pat. No. 9,388,222). The PD-1/anti-CD3/anti-Cldn18.2 fusion proteins were in modified bispecific formats including Fab x scFv Fc, Fab with N-terminal scFv Fc, scFv x Fab with N- or C-terminal scFv Fc. The anti-CD3 paratope was cither C3E6 (see, e.g., U.S. Pat. Publ. No. 2019/0359712) was in either Fab or scFv format or the anti-CD3 paratope described in US20150232557A1 (VL SEQ ID NO: 1, VH SEQ ID NO: 2) in scFv format. The anti-Cldn18.2 paratope scFv and Fab domain sequences were generated as described in, e.g., co-pending U.S. Prov. Appl. No. 63/417,542. To allow for selective heterodimeric pairing, mutations were introduced in the anti-CD3 CH3 as well as the anti-Her2 scFv-Fc CH3 chain as described previously (Von Kreudenstein, T. S. et al. Improving biophysical properties of a bispecific antibody scaffold to aid developability: quality by molecular design. MAbs 5, 646-654, doi: 10.4161/mabs.25632 (2013); (A chain CH3 domain, SEQ ID NO: 4, B chain CH3 domain SEQ ID NO: 5). Mutations (L234A_L235A_D265S as compared to a wild-type human IgG1 CH2) were also introduced in both CH2 domains to reduce binding to the Fc gamma receptors (SEQ ID NO: 6). Furthermore, polypeptides based on the modified protein sequences of the IgV domains of human PD-1 (SEQ ID NO: 7) (West, S. M. & Deng, X. A. Considering B7-CD28 as a family through sequence and structure. Exp Biol Med (Maywood), 1535370219855970, doi: 10.1177/1535370219855970 (2019) were fused to the N-termini of heavy chain (VH-CH1-hinge-CH2-CH3) of the anti-CD3 variable domain, respectively, using linkers that were comprised of a variable number of repeats of sequences predicted to be flexible (GGGGSG) or form helical turns ((EAAAK)n, Chen, X., Zaro, J. L. & Shen, W. C. Fusion protein linkers: property, design and functionality. Adv Drug Deliv Rev 65, 1357-1369, doi: 10.1016/j.addr.2012.09.039 (2013)). In v31929, the high affinity (HAC) PD-1 sequence used contained mutations known to increase the affinity of the PD-1:PD-L1 complex as described before (Maute, R. L. et al. Engineering high-affinity PD-1 variants for optimized immunotherapy and immuno-PET imaging. Proc Natl Acad Sci USA 112, E6506-6514, doi: 10.1073/pnas. 1519623112 (2015); SEQ ID NO: 9; Liang, Z. et al. High-affinity human PD-L1 variants attenuate the suppression of T cell activation. Oncotarget 8, 88360-88375, doi:10.18632/oncotarget.21729 (2017); SEQ ID NO: 10, high affinity PD-1). Moderate affinity human PD-1 sequence was obtained by introducing mutation A132L as described before (Lazar-Molnar, E. et al. Structure-guided development of a high-affinity human Programmed Cell Death-1: Implications for tumor immunotherapy. EBioMedicine, 17 (2017) 30-44, doi: 10.1016/j.cbiom.2017.02.004 (2017) or the set of mutations: V39R, N41V, Y43H, M45E, S48G, N49Y, Q50E, K53T, A56S, Q63T, G65L, Q66P, L97V, S102A, L103F, A104H, K106V, A107I (referred throughout as G1 4-1) as described before (Maute, R. L. et al. Engineering high-affinity PD-1 variants for optimized immunotherapy and immuno-PET imaging. Proc Natl Acad Sci USA 112, E6506-6514, doi: 10.1073/pnas. 1519623112 (2015); SEQ ID NO: XX) and used to generate PD-1 variants with increased affinity for PD-L1 relative to wildtype PD-1 sequence. In v32497 the known mutations K78A,C93S,I126A (Lazar-Molnar et. Al. Structure-guided development of a high-affinity Programmed Cell Death-1: Implications for tumor immunotherapy. EbioMedicine 17 (2017) 30-44. http://dx.doi.org/10.1016/j.cbiom.2017.02.004) were introduced into the sequence of v31929 to attenuate and knockout (KO) the binding of the PD-1 to its cognate ligand, PD-L1. The mutations K78A, C93S, I126A are also used in PD-1 of select PD-1/anti-CD3/anti-MSLN and PD-1/anti-CD3/anti-Cldn18.2 variants to attenuate and knockout (KO) the binding of the PD-1 to its cognate ligand, PD-L1. A schematic of the construct design for a PD-1/anti-CD3/anti-HER2 is shown in FIG. 2, with the Fab x scFv Fc molecules that contain an anti-CD3 Fab and an anti-HER2 scFv and a PD-1 IgV domain fused to the CD3 binding domain VH chain via a peptidic linker. Schematics and further details of the exemplary PD-1/anti-CD3/anti-Her2 and PD-1/anti-CD3/anti-MSLN and PD-1/anti-CD3/anti-Cldn18.2 variants are shown in Table X1. Schematic drawings showing various non-limiting examples of alternative formats for PD-1/anti-CD3/anti-TAA antibody constructs are provided in FIG. 3A-3LL.

Using SPR with PD-L1 coupled and PD-1 variants flowed, the KD of (a) a wild-type PD-1, (b) a high affinity PD-1 used in v31929, and (c) an attenuated PD-1 used in v32497 were determined to be (a) 236 nM, (b) 1.8 nM and (c) greater than 5 μM respectively.

In some experiments, the anti-PD-L1 antibody atezolizumab and/or the anti-PD-1 antibody nivolumab were used as controls, and palivizumab was used as an isotype control. The VH and VL sequences of these control antibodies are shown in Table AA.

TABLE X1
Composition and Clone Sequences of certain Trispecific Fusion Protein Variants*

| Variant No | Schematic | Description | Clone H1 | Clone L1 | Clone H2 |
|---|---|---|---|---|---|
| v31929 | [symbol] | CD3 x Her2 Fab x scFv Fc, high affinity (HAC) PD-1 attached to CD3 HC | 22080 | 12985 | 21490 |
| v32497 | [symbol] | CD3 x Her2 Fab x scFv Fc, attenuated (KO) PD-1 attached to CD3 HC | 23734 | 12985 | 21490 |
| v38400 | [symbol] | CD3 x Her2 Fab x scFv Fc, high affinity (HAC) PD-1 attached to CD3 HC | 29207 | 16412 | 21490 |
| v38401 | [symbol] | CD3 x Her2 Fab x scFv Fc, attenuated (KO) PD-1 attached to CD3 HC | 29206 | 16412 | 21490 |
| v38403 | [symbol] | CD3 x Her2 Fab x scFv Fc, wildtype (WT) PD-1 attached to CD3 HC | 29204 | 16412 | 21490 |
| v38404 | [symbol] | CD3 x Her2 Fab x scFv Fc, moderate affinity (A132L) PD-1 attached to CD3 HC | 29203 | 16412 | 21490 |
| v38405 | [symbol] | CD3 x Her2 Fab x scFv Fc, moderate affinity (G1 4-1) PD-1 attached to CD3 HC | 29202 | 16412 | 21490 |
| v30421 | [symbol] | CD3 Fab x Her2 scFv Fc | 12989 | 12985 | 21490 |
| v31927 | [symbol] | CD3 x Her2 Fab x scFv Fc, wildtype (wt) PD-1 attached to CD3 HC | 22082 | 12985 | 21490 |
| v31928 | [symbol] | CD3 x Her2 Fab x scFv Fc, moderate affinity (A132L) PD-1 attached to CD3 HC | 22081 | 12985 | 21490 |
| v38406 | [symbol] | CD3 x Her2 Fab x scFv Fc, modulated affinity PD-1 attached to CD3 HC | 29201 | 12985 | 21490 |
| v38407 | [symbol] | CD3 x Her2 Fab x scFv Fc, moderate affinity (G1 4-1) PD-1 attached to CD3 HC | 29200 | 12985 | 21490 |
| v33449 | Monospecific | atezolizumab | | | |
| | anti-PD-L1 | | | | |
| | control | | | | |
| v19078 | Monospecific | nivolumab | | | |
| | anti-PD-1 | | | | |
| | control | | | | |
| v22277 | Monospecific | palivizumab | | | |
| | isotype control | | | | |
| v38520 | [symbol] | CD3 Fab x MSLN scFv Fc, high affinity (HAC) PD-1 attached to CD3 HC (N-term) with linker EAAAKEAAAK | 29207 | 16412 | 29275 |
| v38440 | [symbol] | CD3 Fab x MSLN scFv Fc, high affinity (HAC) PD-1 attached to CD3 HC (N-term) with linker sequence GGGGSG | 29208 | 16412 | 29275 |
| v38441 | [symbol] | MSLN Fab x CD3 scFv Fc, high affinity (HAC) PD-1 attached to MSLN HC (N-term) | 29276 | 23570 | 25095 |
| v38442 | [symbol] | CD3 Fab x MSLN scFv Fc, high affinity (HAC) PD-1 attached to CD3 HC (C-term) | 29238 | 16412 | 29275 |
| v38443 | [symbol] | CD3 Fab with MSLN scFv attached to CD3 HC (N-term) x high affinity (HAC) PD-1 attached to Fc hinge | 29282 | 16412 | 23270 |
| v38448 | [symbol] | CD3 Fab with C- term MSLN scFv x MSLN scFv Fc with high affinity (HAC) PD-1 attached to CD3 LC (N-term) | 29257 | 29220 | 23867 |
| v38449 | [symbol] | CD3 Fab with MSLN scFv attached to CD3 HC (N-term) x MLSN scFv Fc with high affinity (HAC) PD-1 attached (N-term) | 29283 | 16412 | 29258 |
| v38450 | [symbol] | CD3 Fab with MSLN scFv attached to CD3 HC (N-term) x MLSN scFv Fc with high affinity (HAC) PD-1 attached (C-term) | 29283 | 16412 | 29264 |
| v38451 | [symbol] | CD3 Fab with MSLN scFv attached to CD3 HC (N-term) x MLSN scFv Fc with high affinity (HAC) PD-1 attached to CD3 Fab LC (N-term) | 29283 | 29220 | 23867 |
| v38452 | [symbol] | CD3 Fab with MSLN scFv attached to CD3 HC (N-term) x MLSN scFv Fc with high affinity (HAC) PD-1 attached to CD3 Fab LC (C-term) | 29283 | 29226 | 23867 |
| v38454 | [symbol] | CD3 Fab with MSLN scFv attached to CD3 HC (N-term) x MLSN scFv Fc with attenuated (KO) PD-1 attached (N-term) | 29283 | 16412 | 29263 |
| v38455 | [symbol] | CD3 Fab x (MSLN scFv)² Fc, attenuated (KO) PD-1 attached to HC2 (C-term) | 29283 | 16412 | 29269 |
| v38463 | [symbol] | CD3 Fab x MSLN scFv Fc | 29283 | 16412 | 23867 |
| v38344 | [symbol] | CD3 Fab x MSLN scFv Fc, high affinity (HAC) PD-1 attached to CD3 HC (N-term) | 22080 | 12985 | 23867 |
| v38345 | [symbol] | CD3 Fab x MSLN scFv Fc, attenuated PD-1 attached to CD3 HC (N-term) | 23734 | 12985 | 23867 |
| v38910 | [symbol] | CD3 Fab with MSLN scFv attached to CD3 HC (N-term) x high affinity (HAC) PD-1 attached to Fc hinge | 29283 | 16412 | 23270 |
| v38911 | [symbol] | CD3 Fab with MSLN scFv attached to CD3 HC (N-term) x moderate affinity (G1 4-1) PD-1 attached to Fc hinge | 29283 | 16412 | 29232 |
| v38913 | [symbol] | CD3 Fab with MSLN scFv attached to CD3 HC (N-term) x moderate affinity (A132L) PD-1 attached to Fc hinge | 29283 | 16412 | 29234 |
| v38914 | [symbol] | CD3 Fab with MSLN scFv attached to CD3 HC (N-term) x wildtype (wt) PD-1 attached to Fc hinge | 29283 | 16412 | 29235 |
| v38915 | [symbol] | CD3 Fab with MSLN scFv attached to CD3 HC (N-term) x MLSN scFv Fc with moderate affinity (G1 4-1) PD-1 attached (N-term) | 29283 | 16412 | 29259 |
| v38919 | [symbol] | CD3 Fab x (MSLN scFv)² Fc, moderate affinity (G1 4-1) PD-1 attached to HC2 (C-term) | 29283 | 16412 | 29265 |
| v38921 | [symbol] | CD3 Fab x (MSLN scFv)² Fc, moderate affinity (A132L) PD-1 attached to HC2 (C-term) | 29283 | 16412 | 29267 |
| v38922 | [symbol] | CD3 Fab x (MSLN scFv)² Fc, wildtype (wt) PD-1 attached to HC2 (C-term) | 29283 | 16412 | 29268 |
| v18490 | Monospecific | RG7787 | | | |
| | anti-MSLN | | | | |
| | antibody | | | | |
| | control | | | | |
| v38408 | [symbol] | CD3 Fab x Cldn18.2 scFv Fc, high affinity (HAC) PD-1 attached to HC1 (N-term) with GGGGSG linker sequence | 29208 | 16412 | 28373 |
| v38409 | [symbol] | Cldn18.2 Fab x CD3 scFv Fc, high affinity (HAC) PD-1 attached to HC2 (N-term) | 29214 | 28196 | 25095 |
| v38410 | [symbol] | CD3 Fab x Cldn18.2 scFv Fc, high-affinity (HAC) PD-1 attached to HC1 (C-term) | 29238 | 16412 | 28373 |
| v38411 | [symbol] | CD3 Fab with Cldn18.2 scFv attached to CD3 HC1 (N-term) x high affinity (HAC) PD-1 attached to Fc hinge | 29237 | 16412 | 23270 |
| v38412 | [symbol] | CD3 Fab with C- term Cldn18.2 scFv x Cldn18.2 scFv Fc with high affinity (HAC) PD-1 attached to CD3 LC (N-term) | 29244 | 29220 | 28373 |
| v38413 | [symbol] | CD3 Fab with Cldn18.2 scFv attached to CD3 HC1 (N-term) x Cldn18.2 scFv Fc with high affinity (HAC) PD-1 attached (N-term) | 29237 | 16412 | 29245 |
| v38414 | [symbol] | CD3 Fab with Cldn18.2 scFv attached to CD3 HC1 (N-term) x Cldn18.2 scFv Fc with high affinity (HAC) PD-1 attached (C-term) | 29237 | 16412 | 29251 |
| v38415 | [symbol] | CD3 Fab with Cldn18.2 scFv attached to CD3 HC1 (N-term) x CLDN18.2 scFv Fc with high affinity (HAC) PD-1 attached to CD3 Fab LC (N- term) | 29237 | 29220 | 28273 |
| v38416 | [symbol] | CD3 Fab with Cldn18.2 scFv attached to CD3 HC1 (N-term) x MLSN scFv Fc with high affinity (HAC) PD-1 attached to CD3 Fab LC (C-term) | 29237 | 29226 | 28273 |
| v38417 | [symbol] | CD3 Fab x Cldn18.2 scFv Fc | 25094 | 16412 | 28373 |
| v38422 | [symbol] | CD3 Fab x Cldn18.2 scFv Fc, attenuated (KO) PD-1 attached to HC1 (N-term) with GGGGSG linker sequence | 29213 | 16412 | 28373 |
| v38423 | [symbol] | Cldn18.2 Fab x CD3 scFv Fc, attenuated (KO) PD-1 attached to HC2 (N-term) | 29219 | 28196 | 25095 |
| v38424 | [symbol] | CD3 Fab x Cldn18.2 scFv Fc, attenuated (KO) PD-1 attached to HC1 (C-term) | 29243 | 16412 | 28373 |
| v38426 | [symbol] | CD3 Fab with C- term Cldn18.2 scFv x Cldn18.2 scFv Fc with attenuated (KO) PD-1 attached to CD3 LC (N- term) | 29244 | 29225 | 28373 |
| v38522 | [symbol] | CD3 Fab x Cldn18.2 scFv Fc, high affinity (HAC) PD-1 attached to HC1 (N-term) with (EAAAK)₂ linker sequence | 29207 | 16412 | 28373 |
| v38523 | [symbol] | CD3 Fab x Cldn18.2 scFv Fc, attenuated (KO) PD-1 attached to HC1 (N-term) with GGGGSG linker sequence | 29206 | 16412 | 28373 |
| v38729 | [symbol] | CD3 Fab x Cldn18.2 scFv Fc, moderate affinity (G1 4-1) PD-1 attached to HC1 (N-term) | 29209 | 16412 | 28373 |
| v38731 | [symbol] | CD3 Fab x Cldn18.2 scFv Fc, moderate affinity (A132L) PD-1 attached to HC1 (N-term) | 29211 | 16412 | 28373 |
| v38732 | [symbol] | CD3 Fab x Cldn18.2 scFv Fc, wildtype (WT) PD-1 attached to HC1 (N-term) | 29212 | 16412 | 28373 |
| v38733 | [symbol] | Cldn18.2 Fab x CD3 scFv Fc, moderate affinity (G1 4-1) PD-1 attached to HC2 (N-term) | 29215 | 28423 | 25095 |
| v38735 | [symbol] | Cldn18.2 Fab x CD3 scFv Fc, moderate affinity (A132L) PD-1 attached to HC2 (N-term) | 29217 | 28423 | 25095 |
| v38736 | [symbol] | Cldn18.2 Fab x CD3 scFv Fc, wildtype (WT) PD-1 attached to HC2 (N-term) | 29218 | 28423 | 25095 |
| v38737 | [symbol] | CD3 Fab x Cldn18.2 scFv Fc, moderate affinity (G1 4-1) PD-1 attached to HC1 (C-term) | 29239 | 16412 | 28373 |
| v38739 | [symbol] | CD3 Fab x Cldn18.2 scFv Fc, moderate affinity (A132L) PD-1 attached to HC1 (C-term) | 29241 | 16412 | 28373 |
| v38740 | [symbol] | CD3 Fab x Cldn18.2 scFv Fc, wildtype (WT) PD-1 attached to HC1 (C-term) | 29242 | 16412 | 28373 |
| v38741 | [symbol] | CD3 Fab with C-term Cldn18.2 scFv x Cldn18.2 scFv Fc with moderate affinity (G1 4-1) PD-1 attached to CD3 LC (N-term) | 29244 | 29221 | 28373 |
| v38743 | [symbol] | CD3 Fab with C-term Cldn18.2 scFv x Cldn18.2 scFv Fc with moderate affinity (A132L) PD-1 attached to CD3 LC (N-term) | 29244 | 29223 | 28373 |
| v38744 | [symbol] | CD3 Fab with C-term Cldn18.2 scFv x Cldn18.2 scFv Fc with wildtype (WT) PD-1 attached to CD3 LC (N-term) | 29244 | 29224 | 28373 |
| v38917 | [symbol] | CD3 Fab with MSLN scFv attached to CD3 HC (N-term) x MLSN scFv Fc with moderate affinity (A132L) PD-1 attached (N-term) | 29283 | 16412 | 29261 |
| v38918 | [symbol] | CD3 Fab with MSLN scFv attached to CD3 HC (N-term) x MLSN scFv Fc with wildtype (WT) PD-1 attached (N-term) | 29283 | 16412 | 29262 |
| v38999 | [symbol] | CD3 Fab x (Cldn18.2 scFv)² Fc, high affinity (HAC)-PD-1 attached to HC2 (N-term) | 29244 | 16412 | 29245 |
| v39000 | [symbol] | CD3 Fab x (Cldn18.2 scFv)² Fc, moderate affinity (A132L) PD-1 attached to HC2 (N-term) | 29244 | 16412 | 29248 |
| v39001 | [symbol] | CD3 Fab x (Cldn18.2 scFv)² Fc, attenuated (KO) PD-1 attached to HC2 (N-term) | 29244 | 16412 | 29250 |
| v39003 | [symbol] | CD3 Fab x (Cldn18.2 scFv)² Fc, high affinity (HAC) PD-1 attached to HC2 (C-term) | 29244 | 16412 | 29251 |
| v39004 | [symbol] | CD3 Fab x (Cldn18.2 scFv)² Fc, moderate affinity (A132L) PD-1 attached to HC2 (C-term) | 29244 | 16412 | 29254 |
| v39005 | [symbol] | CD3 Fab x (Cldn18.2 scFv)² Fc, attenuated (KO) PD-1 attached to HC2 (C-term) | 29244 | 16412 | 29256 |
| v39007 | [symbol] | CD3 Fab x (Cldn18.2 scFv)² Fc, high affinity (HAC) PD-1 attached to LC (C-term) | 29244 | 29226 | 28373 |
| v39009 | [symbol] | CD3 Fab x (Cldn18.2 scFv)² Fc, attenuated (KO) PD-1 attached to LC (C-term) | 29244 | 29231 | 28373 |
| v35923 | | AMG-910 | | | |

*The PD-1 IgV domain attached to the heavy chain is indicated with a striped pattern in the cartoons.

### Example 2: Production and Testing of Fusion Protein Variants

Sequences of PD-1/anti-CD3/Her2 variants, PD-1/anti-CD3/anti-MSLN variants and PD-1/anti-CD3/anti-Cldn18.2 variants were ported into expression vectors and expressed and purified as follows.

### Methods

Heavy chain vector inserts comprising a signal peptide (Barash et al., 2002, Biochem and Biophys Res. Comm., 294:835-842, SEQ ID 27) and the heavy chain clone terminating at G446 (EU numbering) of CH3 were ligated into a pTT5 vector to produce heavy chain expression vectors. Light chain vector inserts comprising the same signal peptide and the light chain clone were ligated into a pTT5 vector to produce light chain expression vectors. The resulting heavy and light chain expression vectors were sequenced to confirm correct reading frame and sequence of the coding DNA.

Heavy and light chains of the modified PD-1/anti-CD3/anti-Her2 variants, PD-1/anti-CD3/anti-MSLN variants or PD-1/anti-CD3/anti-Cldn18.2 variants were co-expressed in 25 mL cultures of Expi293F™ cells (Thermo Fisher, Waltham, MA). Expi293™ cells were cultured at 37° C. in Expi293™ Expression Medium (Thermo Fisher, Waltham, MA) on an orbital shaker rotating at 125 rpm in a humidified atmosphere of 8% CO₂. A volume of 25 mL with a total cell count of 7.5×10⁷ cells was transfected with a total of 25 μg DNA. Prior to transfection the DNA was diluted in 1.5 mL Opti-MEM™ I Reduced Serum Medium (Thermo Fisher, Waltham, MA). In a volume of 1.42 mL Opti-MEM™ I Reduced Serum Medium, 80 μL of ExpiFectamine™ 293 reagent (Thermo Fisher, Waltham, MA) were diluted and, after incubation for five minutes, combined with the DNA transfection mix to a total volume of 3 mL. After 10 to 20 minutes the DNA-ExpiFectamine™293 reagent mixture was added to the cell culture. After incubation at 37° C. for 18-22 hours, 150 μL of ExpiFectamine™ 293 Enhancer 1 and 1.5 mL of ExpiFectamine™ 293 Enhancer 2 (Thermo Fisher, Waltham, MA) were added to each culture. Cells were incubated for five to seven days, and supernatants were harvested for protein purification.

Clarified supernatant samples were applied to 1 mL of slurry containing 50% mAb Select SuRe resin (GE Healthcare, Chicago, IL) in batch mode. Columns were equilibrated in PBS. After loading, columns were washed with PBS and protein eluted with 100 mM sodium citrate buffer pH 3.5. The eluted samples were pH adjusted by adding 10% (v/v) 1 M Tris pH 9 to yield a final pH of 6-7. After concentration, all of the material was injected into an AKTA Pure FPLC System (GE Life Sciences) and run on a Superdex 200 Increase 10/300 GL (GE Life Sciences) column pre-equilibrated with PBS pH 7.4. The protein was eluted from the column at a rate of 0.75 mL/min and collected in 0.5 mL fractions. Peak fractions were pooled and concentrated using Vivaspin 20, 30 kDa MWCO polyethersulfone concentrators (MilliporeSigma Burlington MA, USA). After sterile filtering through 0.2 μm PALL Acrodisc™ Syringe Filters with Supor™ Membrane, proteins were quantitated based on A280 nm (Nanodrop), frozen and stored at −80° C. until further use. Preparative SEC was used in order to obtain samples of high purity. Yields after preparative SEC ranged from 0.5-5 mg per variant.

Following purification, purity of samples was assessed by non-reducing and reducing High Throughput Protein Express assay using CE-SDS LabChip® GXII (Perkin Elmer, Waltham, MA). Procedures were carried out according to HT Protein Express LabChip® User Guide version 2 with the following modifications. mAb samples, at either 2 μL or 5 μL (concentration range 5-2000 ng/ul), were added to separate wells in 96 well plates (BioRad, Hercules, CA) along with 7 μL of HT Protein Express Sample Buffer (Perkin Elmer #760328). The reducing buffer is prepared by adding 3.5 μl of DTT (IM) to 100 μl of HT Protein Express Sample Buffer. mAb samples were then denatured at 90° C. for 5 mins and 35 μl of water is added to each sample well. The LabChip® instrument was operated using the HT Protein Express Chip (Perkin Elmer #760499) and the HT Protein Express 200 assay setting (14 kDa-200 kDa).

UPLC-SEC was performed on an Agilent Technologies 1260 Infinity LC system using an Agilent Technologies AdvanceBio SEC 300A column at 25° C. Before injection, samples were centrifuged at 10000 g for 5 minutes, and 5 μl was injected into the column. Samples were run for 7 min at a flow rate of 1 mL/min in PBS, pH 7.4 and elution was monitored by UV absorbance at 190-400 nm. Chromatograms were extracted at 280 nm. Peak integration was performed using the OpenLAB CDS ChemStation software.

UPLC-SEC traces of samples after preparative SEC purification of the variants showed highly homogencous samples that contained 89%-94% of correct species. Analysis of non-reducing CE-SDS showed a single predominant species and only bands corresponding to the intact chains of all variants were found in the reducing CE-SDS run.

### Example 3: In Vivo Efficacy Study of a Trispecific PD-1/Anti-HER2/Anti-CD3 Fusion Protein Compared to a Structurally Similar Antibody Construct Having an Attenuated PD-1 with and without an Anti-PD-L1 Antibody

In order to compare the anti-tumor activity of PD-1/anti-HER2/anti-CD3 antibody (referred to as the “Trispecific”) with an PD-1 (attenuated)/anti-HER2/anti-CD3 (referred to as the “Bispecific”), an in vivo study was carried out. First, it was important to establish effective dose ranges of PD-1 (attenuated)/anti-HER2/anti-CD3 control and an anti-PD-1/anti-HER2/a-CD3 in a humanized CDX model. The efficacy of the trispecific fusion protein was also compared with that of the Bispecific combined with Atczolizumab, an anti-PD-L1 antibody.

The first step was to identify suitable donors. Five PBMC donors were screened using IncuCyte™. Labeled JIMT-1 target cells that express HER2 were seeded 24 hours prior to the addition of donor PBMC at E:T ratios of 3:1 and 10:1. Test articles were added concurrently with PBMCs. Cells were imaged every 3 hours. v31929 (Trispecific) @ 10 pM, 1 pM, 0.1 pM and v32497 (Bispecific) @ 10 pM, 1 pM, 0.1 pM were screened. Donors that displayed the largest differential between matched dose of test articles and that showed demonstrated activity by v31929 were selected for the in vivo study.

For the in vivo efficacy study, NOG (NOD/Shi-scid/IL-2Rg^{null}) immunodeficient mice were orthotopically implanted with JIMT-1 tumor cells in the mammary fat pad. Once tumors were palpable around one week after implant, 10×10⁶ human PBMCs were administered intravenously and tumor measurements along with body weights were taken twice a week. Once tumors reached an average of 120 mm³ in size on day 12 post tumor implant, mice were randomized into five study groups and test article dosing was initiated. Mice received intravenous injections of either the Trispecific (v31929) or Bispecific (v32497) test articles once a week for four weeks at a dose of either 0.5 or 1.0 mg/kg. One group of mice receiving 1 mg/kg of the Bispecific antibody also received 5 mg/kg Atezolizumab twice a week for four weeks by intraperitoneal injections to achieve saturation of PD1 checkpoint blockade. General health and welfare of animals was monitored daily, and study concluded 65 days after tumor implant or when the tumor burden of an individual animal reached >2000 mm³.

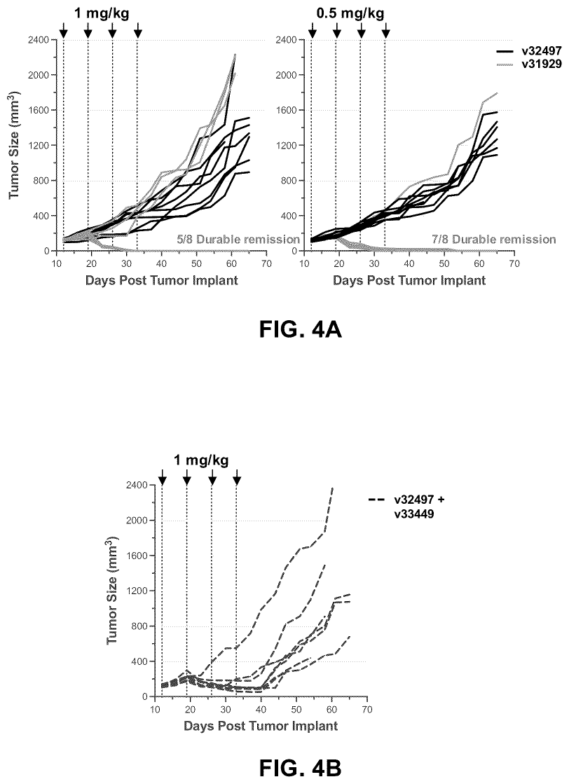

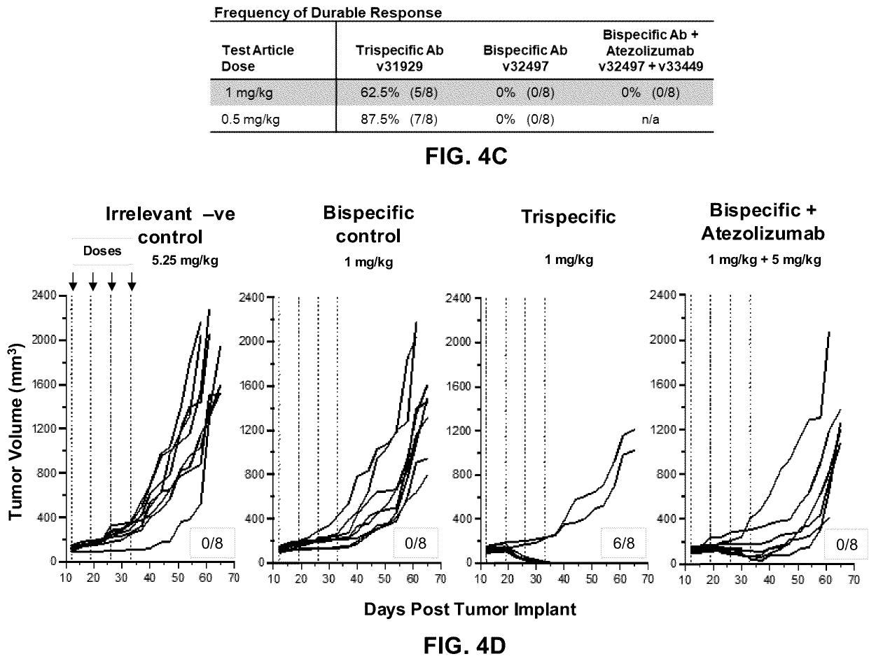

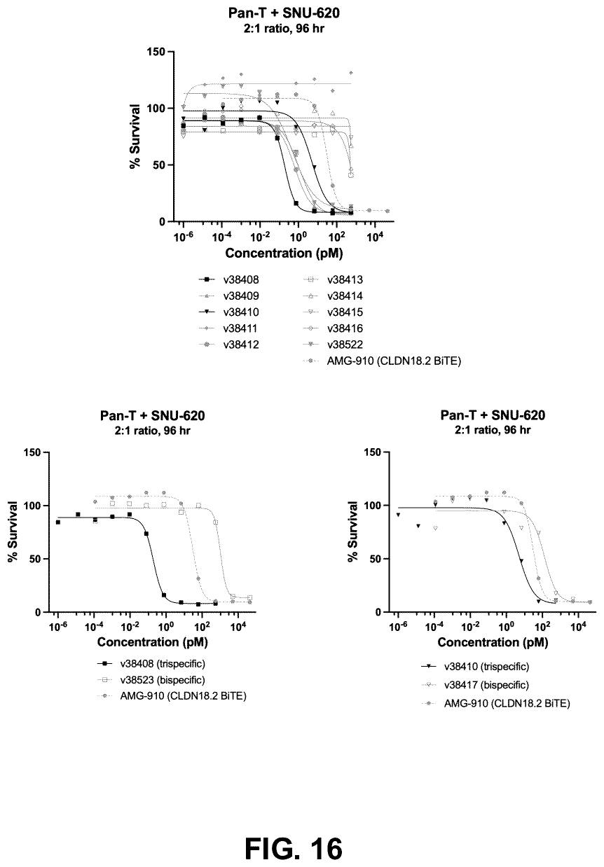

FIGS. 4A-4D and FIG. 16 show the tumor burden of mice that received either the Trispecific (v31929) or the Bispecific (v32497) antibody. None of the animals treated with the Bispecific v32497 Ab at either 0.5 mg/kg or 1.0 mg/kg) maintained durable remission during the study term. In contrast, the majority (7/8 that received 0.5 mg/kg and 5/8 that received 1.0 mg/kg) of animals treated with the Trispecific v31929 antibody exhibited anti-tumor activity and achieved durable remission for the duration of study (FIGS. 4A, 4C, 4D). None of the animals that received 1.0 mg/kg of the Bispecific Ab in combination with the anti-PD-L1 antibody (Atezolizumab) achieved durable remission (FIG. 4B). This demonstrated the superior efficacy of the Trispecific (v31929) over either the Bispecific (v32497) or the combination of Bispecific with Atezolizumab. These findings are suggestive of two observations. Firstly, it appears that the checkpoint inhibitor (PD-1) domain of the Trispecific T cell engager contributed to or enhanced its anti-tumor efficacy. Secondly, treatment with a checkpoint inhibitor and a T cell engager on a single construct may be superior to treatment with a checkpoint inhibitor and a T cell engager on separate constructs.

### Example 4: Ability of Trispecific PD-1/Anti-CD3/Anti-HER2 to Overcome T Cell Exhaustion

T cell dysfunction in the form of T cell exhaustion arises during many chronic infections and cancer. Loss of effector function, sustained expression of inhibitory receptors, and a transcriptional state distinct from that of functional effector or memory T cells are hallmarks of this state. T cell exhaustion prevents optimal control of infection and inhibits anti-tumour immunity. (Ref: https://pubmed.ncbi.nlm.nih.gov/21739672/).

Protocols for modelling exhausted T cells in vitro have been described (Ref: https://www.cell.com/iscience/pdf/S2589-0042 (18) 30018-X.pdf, https://link.springer.com/protocol/10.1007% 2F978-1-0716-0171-6_6). These models were used to investigate whether trispecific PD-1/anti-CD3/anti-HER2 antibody constructs can restore effector function in exhausted T cells as described below.

### T Cell Exhaustion Method

Primary CD3+ T cells (Stemcell Technologies, Vancouver BC) were thawed in pre-warmed media (AIM-V [Life Technologies, California, USA]+10% FBS), washed and resuspended at 1×10\hat{ }6 cells/mL in media and added to a T-75 flask (VWR, Radnor, PA). The same number of T-Activator CD3/CD28 Dynabeads, (Life Technologies, California USA) were washed with wash buffer (PBS [Thermo Fisher Scientific, Waltham, Massachusetts, USA]+0.1% BSA [Sigma-Aldrich, Missouri, USA]+2 mM EDTA [Life Technologies, California USA]), resuspended in 100 μL of media and added to the T cell culture at a 1:1 bead-to-T cell ratio. On day 2, the T cells in the flask were pipetted up and down to dislodge rosettes and create a single-cell suspension. The T cells were then pelleted, supernatant was collected, and additional media was added to maintain the cell suspension at 1×10\hat{ }6 cells/mL. Additional T-Activator CD3/CD28 Dynabeads were washed in wash buffer and added to the culture to maintain the 1:1 bead-to-T cell ratio. The process was repeated every 2-3 days for a total of 4 restimulations over 8-9 days.

### Exhaustion Marker Determination and Cytokine Analysis

In order to confirm that the T cells that underwent the exhaustion protocol above are in an “exhausted” state, a phenotypic and functional analysis was performed as follows. During the exhaustion, T cells were sampled at days 0 and stimulation days 2-8, and supernatants were collected for cytokine analysis. A sample of T cells was left unstimulated as a “naïve” control. The cells were then analysed for upregulation of exhaustion markers PD-1, LAG-3, and TIM-3. Cells were washed twice with FACS buffer (PBS [Thermo Fisher Scientific, Waltham, Massachusetts, USA]+2% FBS [Thermo Fisher Scientific, Waltham, Massachusetts, USA] and resuspended in a master mix containing diluted BB700 Mouse Anti-Human CD3 Clone SP34-2 (BD Biosciences, Franklin Lakes, New Jersey, USA), BV421 Mouse Anti-Human CD279 (PD-1) Clone MIH4, (BD Biosciences, Franklin Lakes, New Jersey, USA), BV711 Mouse Anti-Human TIM-3 (CD366) Clone 7D3 (BD Biosciences, Franklin Lakes, New Jersey, USA), PE Mouse Anti-Human LAG-3 (CD223) Clone T47-530 (BD Biosciences, Franklin Lakes, New Jersey, USA), and Fixable Viability Dye eFluor 520, (Thermo Fisher Scientific, Waltham, Massachusetts, USA) in PBS. The samples were incubated for 20 minutes at room temperature in the dark, washed twice with FACS buffer, and resuspended in 100 μL of FACS buffer for flow cytometry analysis on a FACS Celesta [BD Biosciences, Franklin Lakes, New Jersey, USA]. For analysis, % of cells expressing exhaustion markers was determined by gating marker-positive cells based on FMO controls out of the live/CD3+ gate.

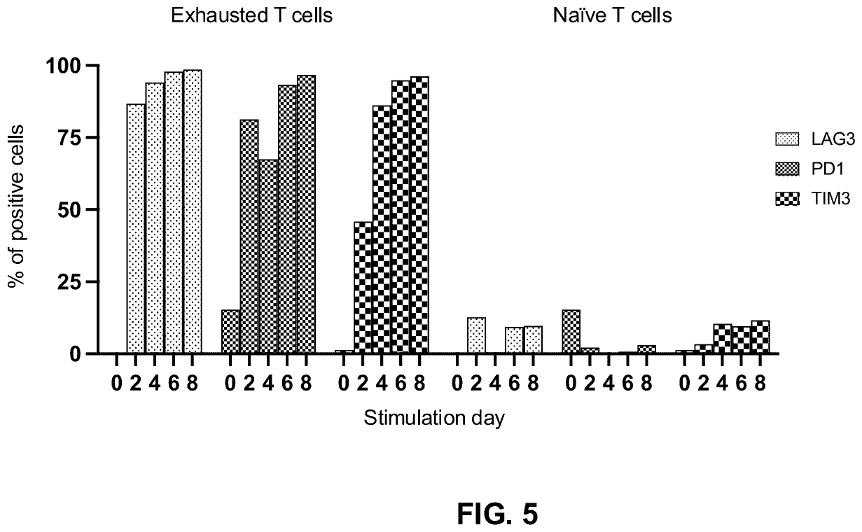

Upregulation of LAG3, PD1 and TIM3, known markers of exhaustion, was observed in cells that underwent the exhaustion protocol but not the naïve cells (FIG. 5). The supernatants were tested for the presence of inflammatory cytokines IFNg, IL-2, and TNFa by Meso Scale as stipulated by the vendor (MSD, Rockville, Maryland, USA). The plots show that inflammatory cytokines peak after the first stimulation and drop sharply with subsequent re-stimulations

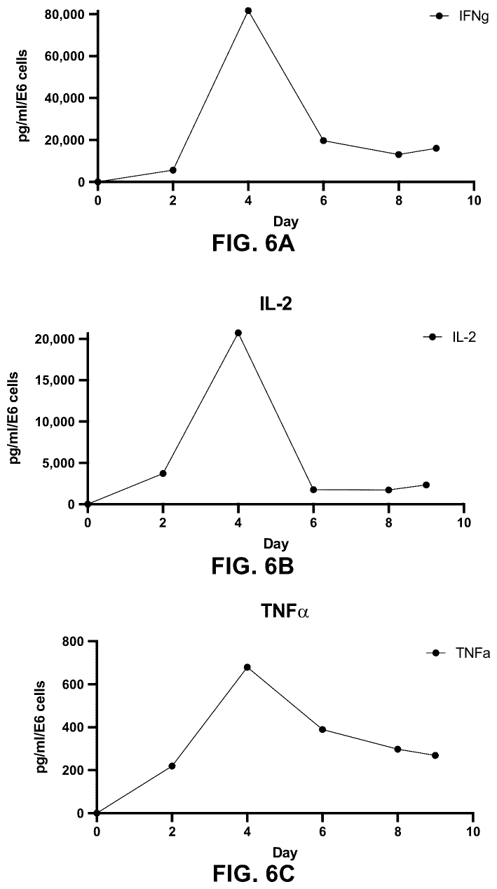

(FIGS. 6A-C). Taken together, this confirmed that the T cells that underwent the exhaustion protocol are phenotypically and functionally in a state of exhaustion and were deemed appropriate to use for the following studies.

The activity of the trispecific variant v31929 on T cells in an exhausted state was evaluated by measuring proliferation, T cell dependent cytotoxicity, and cytokine secretion.

### Proliferation

Variants were added to a 6-well cell culture treated plate (Corning, New York, USA) at 1, 10 and 100 pM in 500 μL of media. Exhausted T cells were labelled with 5 UM of Cell Proliferation dye eFluor 670 (Thermo Fisher Scientific, Waltham, Massachusetts, USA) according to manufacturer's instructions. They were mixed with JIMT-1 cells at a 5:1 ratio and added to the plate in a total of 1 mL at 1E6/ml concentration. After 3 days of incubation, supernatants were collected and frozen down. After 5 days of incubation, cells were collected and quantified as follows. The T cells washed twice with FACS buffer and resuspended in a solution containing Fixable Viability Dye eFluor 520 at 1/500 dilution in PBS. The cells were incubated for 15 mins at room temperature in the dark. The cells were washed twice with FACS buffer and resuspended in 100 μL of FACS buffer for flow cytometry analysis on a BD FACS Celesta. Proliferation was determined by quantifying total numbers of eFluor 670-low T cells.

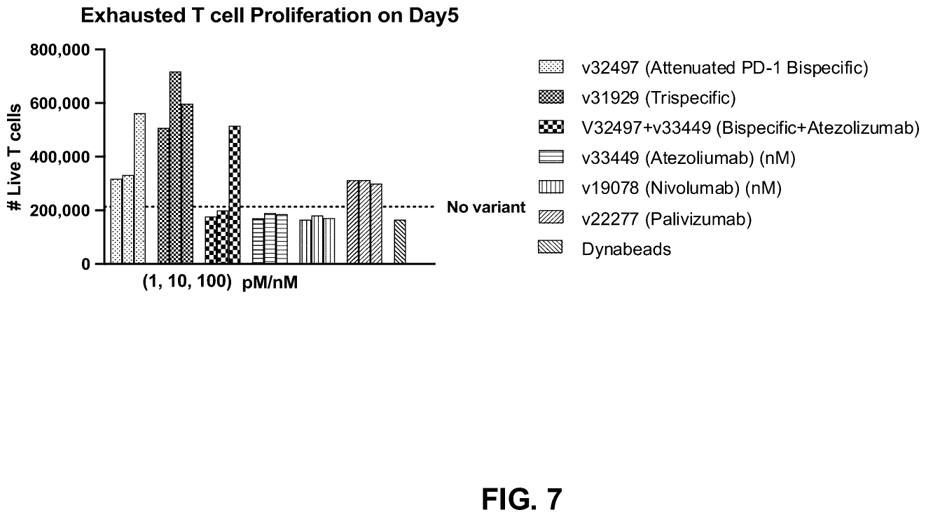

As shown in FIG. 7, the Trispecific (v31929) induced proliferation of exhausted T cells at ˜100× lower concentration than the Bispecific (v32497) or the Bispecific in combination with Atezolizumab (v32497+v33449), while single checkpoint inhibitors (Atezolizumab and Nivolumab), isotype control (Palivizumab) and Dynabeads (activation control) were no different than the no-variant control. This demonstrated that the Trispecific had the ability to rescue proliferation in exhausted T cells.

### Cytokine Analysis

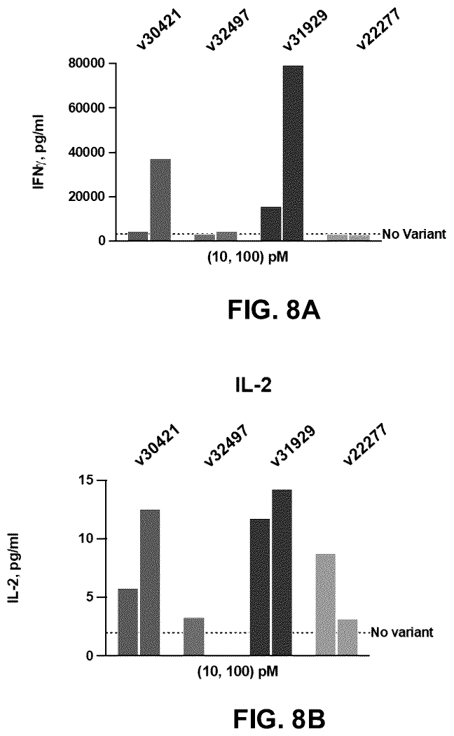

Supernatants from day 3 of the above cultures were thawed, and IFN gamma and IL-2 were quantified by Meso Scale as stipulated by the vendor. The results are shown in FIGS. 8A and 8B. The Trispecific (v31929) induced higher levels of IFN gamma as compared to the Bispecific and elicited higher IL-2 secretion at the lower concentration.

### TDCC Analysis

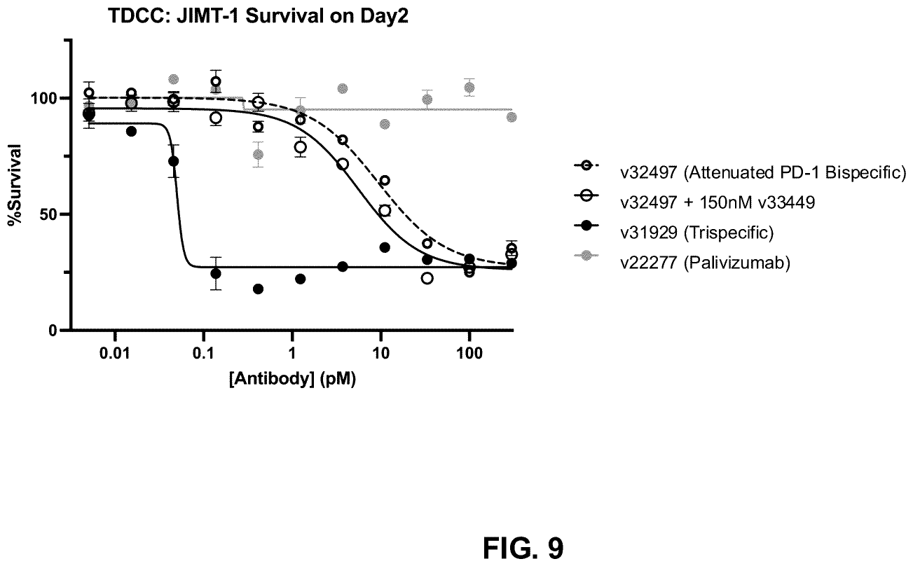

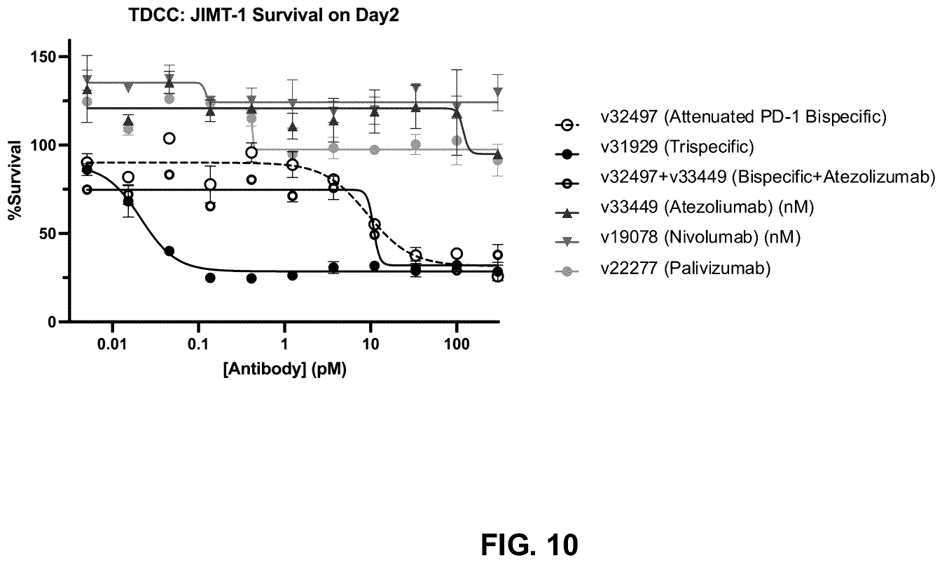

A coculture assay was carried up as using exhausted T cells as effector cells. JIMT-1 cells were thawed and cultured in growth medium (DMEM medium [Thermo Fisher Scientific, Waltham, Massachusetts, USA]) supplemented with 10% Fetal Bovine Serum (Thermo Fisher Scientific, Waltham, Massachusetts, USA). The cells were maintained horizontally in T-75 flasks (VWR, Radnor, PA) in an incubator at 37° C. with 5% carbon dioxide. On the day of the experiment, the variants were titrated in triplicate at 1:3 dilution directly in a 384-well cell culture treated optical bottom plates (Thermo Fisher Scientific, Waltham, Massachusetts, USA) from 300 pM to 5 fM. JIMT-1 cells were harvested using TrypLE (Thermo Fisher Scientific, Waltham, Massachusetts, USA) washed in media, and counted. Exhausted T cell suspension was mixed with JIMT-1 cells at 5:1 effector to target ratio, washed and resuspended at 0.55 E6 cell/ml. 20 μL of the mixed cell suspension was added to the plate containing the titrated variants. The plates were incubated for 48 h in an incubator at 37° C. with 5% carbon dioxide. The survival of target cells was quantified by high-content assessment. As seen in FIG. 9, the Trispecific (v31929) induced cytotoxicity of target cells with significantly higher potency (n=1 EC₅₀=˜ 50 fM (ambiguous fit), n=2 EC₅₀=21 fM) than the Bispecific (v32497, n=1 EC₅₀=9.4 pM, n=2 EC₅₀)=9.2 pM) and the combination with Atezolizumab (v32497+33449, n=1 EC₅₀=5.6 pM, n=2 EC₅₀=˜ 10.7 pM (ambiguous fit)). The n=2 experiment with additional controls demonstrated that the Trispecific (v31929) has the ability to induce potent cytotoxicity in exhausted T cells as compared to the Bispecific and combination (FIG. 10).

### Trispecific Anti-CD3/PD-L1 Binding on Naive and Exhausted T Cells

It is documented that as T cells become activated and exhausted they downregulate CD3 and begin expressing PD-L1 (Lanzavecchia et al., J Exp Med 1997, Pulko, et al., J Immunol, 2011) in addition to the previously mentioned exhaustion markers. To determine how binding is affected, a side-by-side binding assay to naïve and exhausted T cells from the same donor was performed. A CD3 bispecific benchmark (v35923) was included in the analysis. The variants were serially diluted 1 in 4 in 96-well plates (Thermo Scientific, Waltham, Massachusetts, USA) from 400 nM to 0.38 pM in FACS buffer. Exhausted T cells and frozen naïve T cells from the same donor were washed with FACS buffer, plated with the variants at 40,000 cells/well, and incubated for 1 hr at 4° C. The cells were washed 2× with FACS buffer, and 2 μg/ml of secondary antibody AF647 Goat anti-human IgG Fc (Jackson ImmunoResearch, West Grove, PA) with 1:1000 diluted viability dye FVD eF520 (Thermo Scientific, Waltham, Massachusetts, USA) was added to the wells and incubated for 30 min at room temperature. The cells were washed 2× and resuspended in 50 μL of FACS buffer for flow cytometry analysis on a BD FACS Celesta. Binding to T cells was determined by Geometric Mean from the live population.

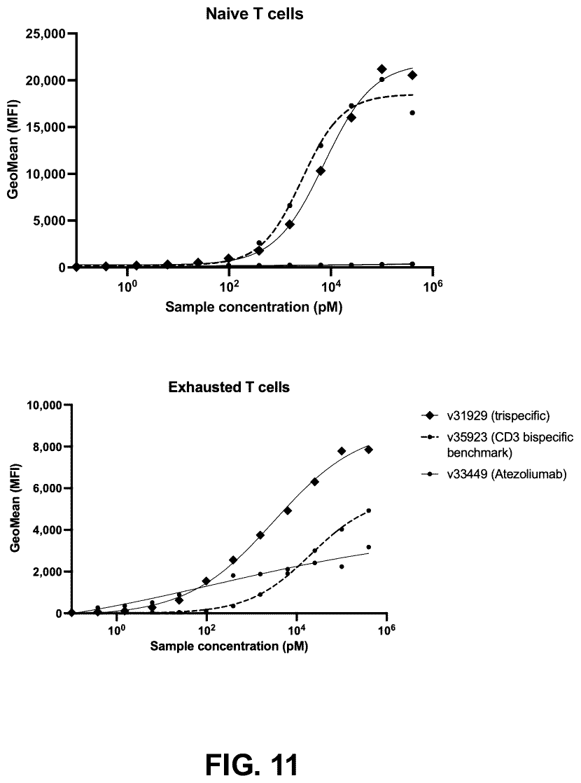

As seen in FIG. 11, on naïve T cells, binding of the trispecific (v31929) and bispecific CD3 benchmark (v35923) is similar both by EC₅₀ (7334 and 2681 respectively) and by maximum binding (21884 and 18520 respectively). However, in the exhausted T cells, the trispecific has a binding advantage (EC₅₀ 3105 and max 8961) over the CD3 bispecific benchmark (EC₅₀ 20073 and max 5611) due to the avidity gain via PD-L1 binding. Atezolizumab (anti-PD-L1 v33449) shows minimal binding to naïve T cells but appreciable binding in exhausted T cells (EC₅₀ 153.2 and max 3991).

### Example 5: Screening of Trispecific PD-1/Anti-CD3/Anti-MSLN Formats in TDCC

The geometry of a T cell engager has been identified as an important parameter to tune efficacy (Chen et al, mAbs, 2020; Ellerman, Methods, 2019). As such, a number of Trispecific PD-1/anti-CD3/anti-MSLN formats exemplified in FIG. 3A-3LL were expressed and purified for in vitro analysis in T cell dependent cellular cytotoxicity assay. Trispecific formats were also assayed for potency across multiple tumor cell lines with varying surface expression levels of MSLN and PD-L1 and for their ability to inhibit PD-1/PD-L1 checkpoint blockade in a checkpoint blockade reporter gene assay.

### TDCC Analysis

A co-culture assay was performed using pan-T cells and tumor cells. H292 (RPMI1640+10% FBS), SNU-216 (RPMI1640+10% FBS), HCT-116 (McCoy's+10% FBS), SKOV-3 (McCoy's+10% FBS), or OVCAR-3 (RPMI1640+20% FBS+0.01 mg/mL insulin) cells were thawed and cultured in 10 cm² dishes (VWR, Radnor, PA) in an incubator at 37° C. with 5% carbon dioxide. On the day of the experiment, the variants were titrated into assay media (RPMI1640+10% FBS+1% Pen/Strep) in triplicate at 1:9 dilution directly in a 384-well cell culture treated optical bottom plates (Thermo Fisher Scientific, Waltham, Massachusetts, USA) from 45000 pM to 0.01 fM. Tumor cells were harvested using TrypLE (Thermo Fisher Scientific, Waltham, Massachusetts, USA) washed in media, and counted. Pan-T cells (BioIVT) were thawed into assay media, washed once in media, and counted on the Cellaca MX cell counter (Perkin Elmer, Waltham, Massachusetts, USA). The pan-T cells were resuspended to 1.0 E6 cells/mL and the tumor cells were resuspended to 2×10⁵ cells/mL. The cell suspensions were mixed at equal volumes to a 5:1 effector to target ratio and added to the assay plates at 20 μL/well. The plates were incubated for 72 hrs in an incubator at 37° C. with 5% carbon dioxide. The survival of target cells was quantified on the Operetta CLS high-content imager (Perkin Elmer, Waltham, Massachusetts, USA).

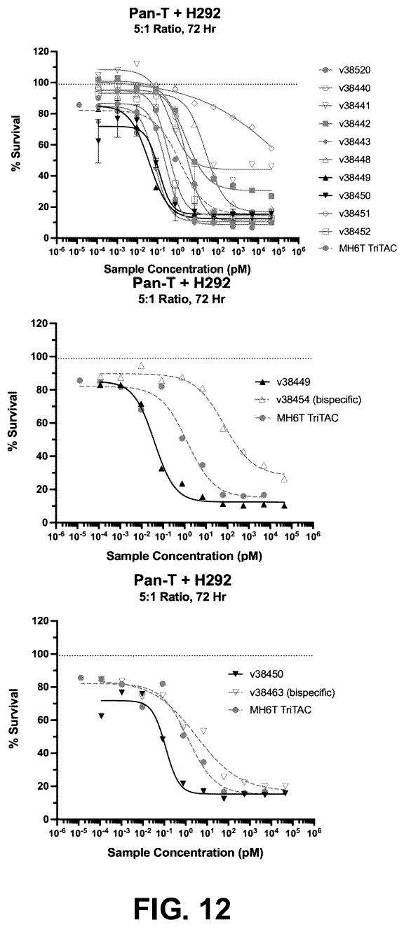

As seen in FIG. 12, ten Trispecific formats were assessed for potency in TDCC assay. All trispecific formats have the same PD-1 domain, anti-CD3 paratope, and either monovalent or bivalent with the same anti-MSLN paratope and differ only in geometry. Some trispecific variants were found to have higher potency than MH6T TriTac and greater potency than the format-matched bispecific control containing attenuated (KO) PD-1 (v38454) or format-matched bispecific control (v38458) (FIG. 12). The calculated EC₅₀ values are shown below in Table X2.

TABLE X2
TDCC EC₅₀ Values for Tested Constructs

| FIG. 12 | Test Articles | EC₅₀ (pM) |
|---|---|---|
| Top | v38520 | 0.254 |
| | v38440 | 2.73 |
| | v38441 | 0.4125 |
| | v38442 | 2.316 |
| | v38443 | 0.07566 |
| | v38448 | 25.24 |
| | v38449 | 0.03884 |
| | v38450 | 0.1163 |
| | v38451 | 599407 |
| | v38452 | 0.348 |
| | MH6T TriTAC | 1.325 |
| Middle | v38449 | 0.03884 |
| | v38454 (bispecific control) | 69.68 |
| | MH6T TriTAC | 1.325 |
| Bottom | v38450 | 0.1163 |
| | v38463 (bispecific control) | 3.152 |
| | MH6T TriTAC | 1.325 |

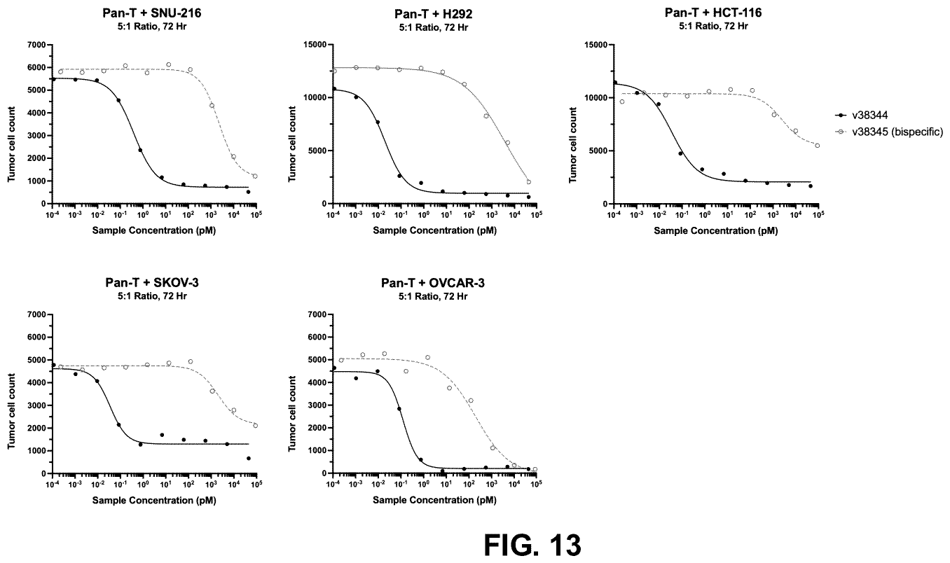

In FIG. 13, an example trispecific format (v38344) compared to the format-matched bispecific with attenuated (KO) PD-1 (v38345) potencies across cell lines SNU-216, H292, HCT-116, SKOV-3, and OVCAR-3. In all tumor cell lines, the Trispecific had superior potency compared to the format-matched bispecific control containing attenuated (KO) PD-1. The calculated EC₅₀ values are shown below in Table X3.

TABLE X3
TDCC EC₅₀ Values (given in pM) for Tested Constructs

| Cell Line | v38344 | v38345 (bispecific) |
|---|---|---|
| SNU-216 | 0.3834 | 2292 |
| H292 | 0.01937 | 3755 |
| HCT-116 | 0.03542 | 2498 |
| SKOV-3 | 0.03474 | 2034 |
| OVCAR-3 | 0.1226 | 199 |

### MSLN and PD-L1 Surface Receptor Quantification for Tumor Cell Lines

Receptor quantification of surface MSLN and PD-L1 was performed on the tumor cell lines tested in TDCC: SNU-216, H292, HCT-116, SKOV-3, and OVCAR-3. PD-L1 receptor quantification was also performed on the tumor cell lines 24 hours following incubation with 20 ng/ml IFNg at 37° C. with 5% CO₂. MSLN and PD-L1 receptor quantification were performed via flow cytometry using Quantum Simply Cellular anti-human and anti-mouse IgG kits respectively (Bangs Laboratories, Fishers, Indiana). Tumor cells were rinsed with PBS (Thermo Fisher Scientific, Waltham, MA), and harvested with TrypLE Express (Thermo Fisher Scientific, Waltham, MA). Cells were counted using Vi-Cell (Beckman Coulter, Indianapolis, IN), washed, and resuspended in FACS buffer-PBS containing 2% FBS (Thermo Fisher Scientific, Waltham, MA) at 4×10\hat{ }6 cells/mL. 25 μL of tumor cell suspension was added in triplicate to a 96-well V-bottom plate (Sarstedt AG, Nümbrecht, Germany). Anti-MSLN-AF647 (RG7787, monovalent antibody, Zymeworks, Vancouver, BC), anti-PD-L1-APC (Clone MIH1, BD Biosciences, San Jose, CA) or irrelevant negative control IgG-AF647 (Zymeworks, Vancouver, BC) antibody at 15 μg/mL was added to the wells and Eppendorf tubes (Thermo Fisher Scientific, Waltham, MA) containing Quantum Simply Cellular IgG beads (anti-human or anti-mouse) and blank beads. Cells and beads were incubated with the antibodies for 1 hr at 4° C. in the dark. Cells and beads were washed, resuspended, and analyzed by flow cytometry. For analysis, a standard curve was generated using the spreadsheet provided by Bangs Laboratories (Fishers, Indiana) for the specific lot of beads, and the surface antigen binding capacity (ABC) was generated by entering the geometric means of the cell populations using the same spreadsheet. ABC values represent the number of molecules of receptor expressed on the cell surface assuming a monovalent binding model.

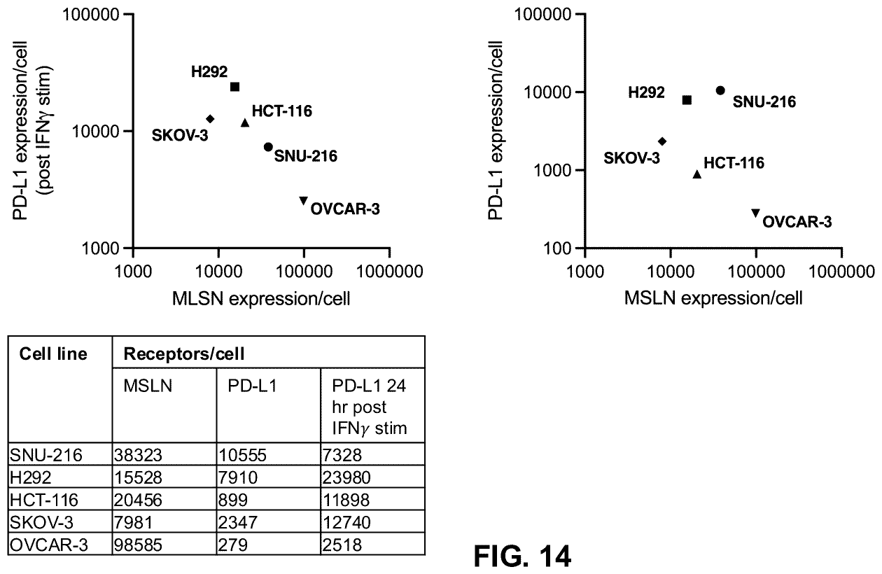

FIG. 14 demonstrates the range of MSLN and PD-L1 surface expression measured for the five cell lines tested in TDCC. This data supports that the Trispecific has superior potency compared to the format-matched bispecific control with attenuated PD-1 (KO, in the figure abbreviated as “bispecific”) across cell lines with differing surface expression levels of PD-L1 and MSLN.

### Example 6: Investigation of Added Functionality of PD-1 Moiety in Hybrid PD-1/PD-L1 Reporter Gene Assay

To investigate blocking of the PD-1:PD-L1 checkpoint engagement by the PD-1 moiety in addition to the T-cell engagement function of the variants, a custom hybrid PD-1/PD-L1 Reporter Gene Assay (RGA) was performed as follows.

### Hybrid PD-1/PD-L1 RGA

HCT-116 (MSLN+, PD-L1+) (ATCC, Manassas, VA) cultured in growth medium consisting of McCoy's medium (Thermo Fisher Scientific, Waltham, MA) supplemented with 10% Fetal Bovine Serum (Thermo Fisher Scientific, Waltham, MA), and Jurkat T cells stably expressing human PD-1 and NFAT-induced luciferase (PD-1/PD-L1 Blockade Bioassay Promega Cat #J1250, Madison, WI) cultured in RPMI-1640 medium with L-glutamine and HEPES supplemented with 10% Fetal Bovine serum, Hygromycin B, Antibiotic G-418 Sulfate solution, Sodium Pyruvate, and MEM nonessential amino acids, were maintained in T-75 or T-175 flasks (Corning, Corning, NY) in an incubator at 37° C. with 5% carbon dioxide prior to assay set-up. Prior to the day of the experiment, tumor cells were treated with 20 ng/ml of Recombinant Human IFN-gamma protein (R&D Systems Cat #285-IF, Minneapolis, MN) for 24 h. On the day of the experiment, the variants were serially titrated 1:8, in duplicate, in a separate titration plate from 150 nM to 0.00014 pM, and then transferred into 384-well Low Flange White Flat Bottom Polystyrene TC-treated Microplates, (Corning Cat #3570, Corning, NY) in 20 μL total volume per well. Tumor cells were dissociated using TrypLE (Thermo Fisher Scientific, Waltham, MA) and mixed with Jurkat cells at a 1:1 ratio in RPMI 1640 supplemented with 1% Fetal Bovine serum. 20 μL of the mixed cell suspension was added to the plate containing the titrated variants. The plates were incubated for 5 h at 37° C. with 5% carbon dioxide. Post incubation, 30 μL of Bio-Glo™ Luciferase Assay reagent (Promega Cat #G7940, Madison, WI) was added to all wells ensuring no bubbles were formed. The plate was read after 10 min in Luminescence mode on the microplate reader (Biotek Synergy H1, Winooski, VT) with a gain of 150.

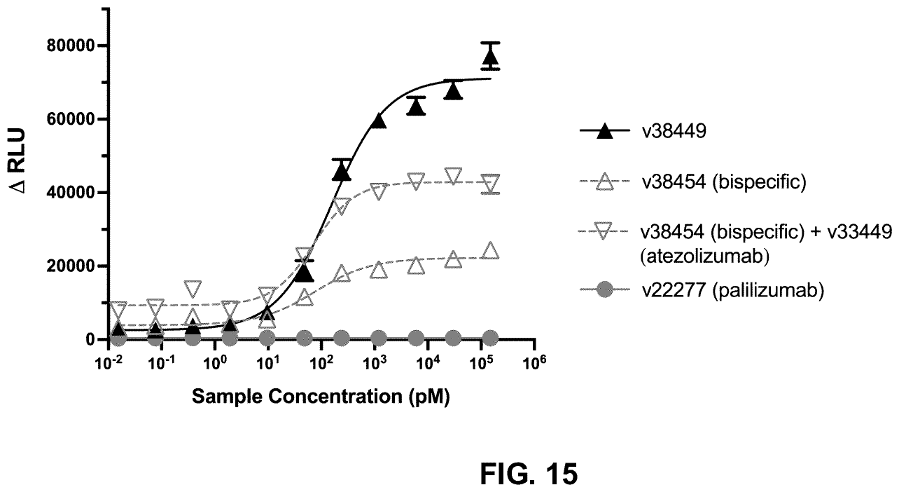

The analysis of the custom RGA to interrogate added functionality of our Trispecific is shown FIG. 15. Tumor cells were treated with 20 ng/ml IFNg for 24 h prior to RGA assay to increase PD-L1 expression on the cell surface. Assay cells treated with a Bispecific variant capable of crosslinking T-cells and tumor cells (v38454) showed CD3 engagement with increasing concentration of variant, visualized by RGA signal. Treatment with the Bispecific variant in combination with a saturating amount (150 nM) of an anti-PD-L1 antibody produced an RGA response compared to Bispecific alone. While the Bispecific CD3×MSLN antibody could productively cross-link T-cells and tumor cells, high concentrations of the anti-PD-L1 antibody robustly blocked the PD-1:PD-L1 checkpoint engagement, leading to a high signal at variant concentrations where CD3 engagement was apparent. Treatment with a Trispecific variant capable of crosslinking T cells and tumor cells as well as PD-L1 blockade (v38449) resulted in a further increase in the RGA signal indicating superior checkpoint blockade with CD3 engagement in the Trispecific compared to combination treatment. An irrelevant anti-RSV antibody (v22277) showed no activity in the RGA.

### Example 7: Screening of Trispecific PD-1/Anti-CD3/Anti-Cldn18.2 Formats in TDCC

The geometry of a trispecific PD-1/anti-CD3/anti-CLDN T cell engager was explored by screening a number of antibody formats exemplified in FIG. 3A-3LL. The variants were expressed and purified for in vitro analysis in T cell dependent cellular cytotoxicity assay. Trispecific formats were also assayed for potency across multiple tumor cell lines with varying surface expression levels of Cldn18.2 and PD-L1 and for their ability to exert PD-1/PD-L1 checkpoint blockade in a checkpoint blockade reporter gene assay.

### TDCC Analysis

A coculture assay was performed using pan-T cells and SNU-620 (RPMI1640+10% FBS, KATO-III (IMDM+20% FBS) and SNI-601 (RPMI1640+10% FBS) tumor cells. Tumor cells were thawed and cultured in 10 cm² dishes (VWR, Radnor, PA) in an incubator at 37° C. with 5% carbon dioxide. On the day of the experiment, the variants were titrated into assay media (RPMI1640+10% FBS+1% Pen/Strep) in triplicate at 1:9 dilution directly in a 384-well cell culture treated optical bottom plates (Thermo Fisher Scientific, Waltham, Massachusetts, USA) from 45000 pM to 0.01 fM. Cells were harvested using TrypLE (Thermo Fisher Scientific, Waltham, Massachusetts, USA) washed in media, and counted. Pan-T cells (Stem Cell Technologies) were thawed into assay media, washed once in media, and counted on the Cellaca MX cell counter (Perkin Elmer, Waltham, Massachusetts, USA). The pan-T cells were resuspended to 1×10⁶ cells/mL and the tumor cells were resuspended to 2×10⁶ cells/mL. The cell suspensions were mixed at equal volumes to a 5:1 effector to target ratio and added to the assay plates at 20 μL/well. The plates were incubated for 72 or 96 hrs in an incubator at 37° C. with 5% carbon dioxide. The survival of target cells was quantified on the Operetta CLS high-content imager (Perkin Elmer, Waltham, Massachusetts, USA)

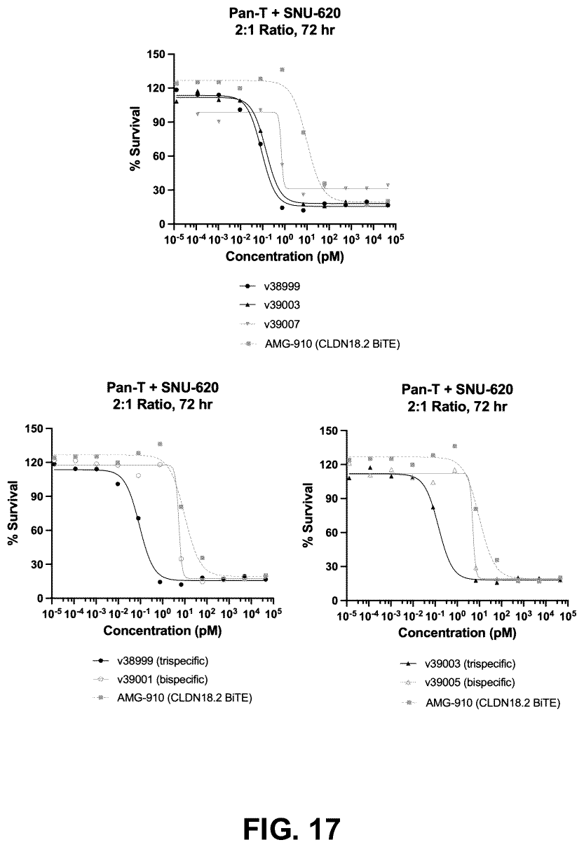

Thirteen PD-1/anti-CD3/anti-CLDN18.2 trispecific T cell engager formats were assessed for potency in a TDCC assay (FIG. 16 and FIG. 17). All trispecific variants tested had the same high affinity (HAC) PD-1 domain, anti-CD3 paratope, and either monovalent or bivalent with the same anti-CLDN18.2 paratope and differed only in format (e.g., total number of antigen binding domains) and geometry (e.g., relative connectivity and location of domains within the fusion protein). Some trispecific variants were found to have higher potency than AMG-910 and greater potency than the format-matched bispecific control containing attenuated PD-1 or format-matched bispecific control (FIG. 16 and FIG. 17).

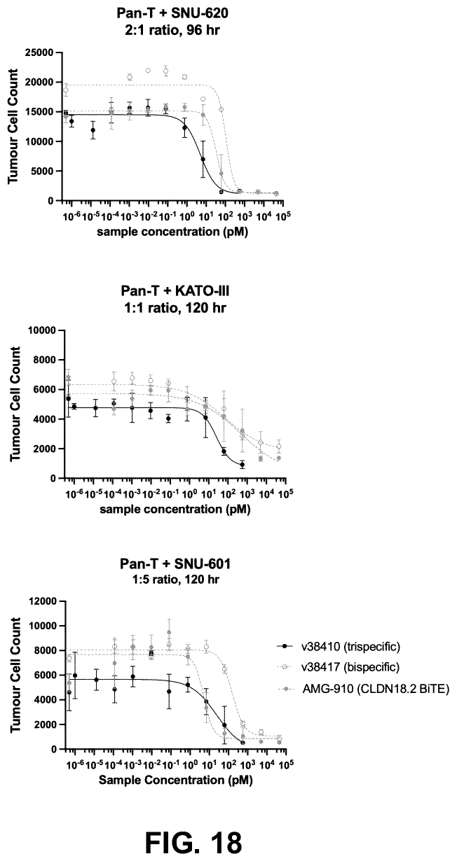

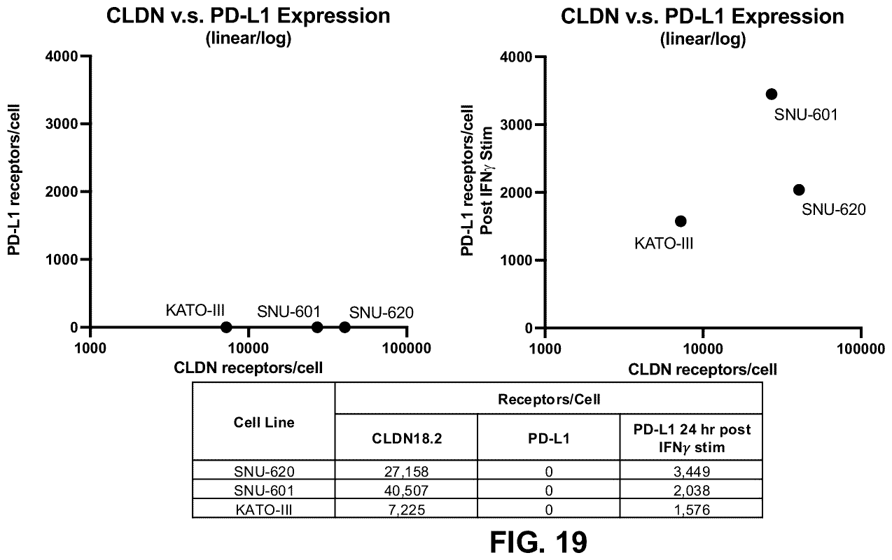

In FIG. 18, an example trispecific format (v38410) was compared to the format-matched bispecific (v38417) and AMG-910 (CLDN18.2 BiTE) potencies across the cell lines SNU-620, KATO-III, and SNU-601. In most tumor cell lines tested, the trispecific appeared to have superior potency compared to the format-matched bispecific control.

The calculated EC₅₀ values (in pM, unless otherwise specified) for the graphs depicted in FIGS. 16-18 are shown below in Table X14.

TABLE X14
TDCC EC₅₀ Values for Tested Constructs and Fusion Proteins

| FIG. 16 | | FIG. 17 | | FIG. 18 | |
|---|---|---|---|---|---|
| Test articles | EC₅₀ | Test article | EC₅₀ | Test article | EC₅₀ |
| v38408 | 0.1963 | v38999 | 0.08886 | v38410 | 4.945 |
| v38409 | 1.108 | v39003 | 0.1416 | v38417 | 108.2 |
| v38410 | 5.079 | v39007 | 0.6891 | | |
| v38411 | n/a | AMG-910 | 9.849 | | |
| v38412 | 0.6458 | | | | |
| v38413 | 724.5 | | | | |
| v38414 | 662.9 | | | | |
| v38415 | Unstable | | | | |
| v38416 | 71603 | | | | |
| v38522 | 0.4877 | | | | |
| AMG-910 | 30.24 | | | | |
| v38408 | 0.1963 | v38999 | 0.08886 | v38410 | 25.2 |
| v38423 | 71.59 | v39001 | 5.255 | v38417 | 71.75 |
| AMG-910 | 30.24 | AMG-910 | 9.849 | AMG-910 | 474.5 |
| v38410 | 5.079 | v39003 | 0.1416 | v38410 | 24.06 |
| v38417 | 128.8 | v39005 | 4.944 | v38417 | 154.5 |
| AMG-910 | 30.24 | AMG-910 | 9.849 | AMG-910 | 5.083 |

### CLDN18.2 and PD-L1 Surface Receptor Quantification for Tumor Cell Lines

Receptor quantification of surface Cldn18.2 and PD-L1 was performed on the tumor cell lines tested in TDCC, e.g., SNU-620, SNU-601, KATO-III. PD-L1 receptor quantification was also performed on the tumor cell lines 24 hours following incubation with 20 ng/ml IFNg at 37° C. with 5% CO₂. CLDN18.2 and PD-L1 receptor quantification were performed via flow cytometry using Quantum Simply Cellular anti-human and anti-mouse IgG kits respectively (Bangs Laboratories, Fishers, Indiana). Tumor cells were rinsed with PBS (Thermo Fisher Scientific, Waltham, MA), and harvested with TrypLE Express (Thermo Fisher Scientific, Waltham, MA). Cells were counted using Vi-Cell (Beckman Coulter, Indianapolis, IN), washed, and resuspended in FACS buffer-PBS containing 2% FBS (Thermo Fisher Scientific, Waltham, MA) at 4×10\hat{ }6 c/mL. 25 μL of tumor cell suspension was added in triplicate to a 96-well V-bottom plate (Sarstedt AG, Nümbrecht, Germany). Anti-Cldn18.2-AF647 (monovalent antibody, Zymeworks, Vancouver, BC), anti-PD-L1-APC (Clone MIH1, BD Biosciences, San Jose, CA) or irrelevant negative control IgG-AF647 (Zymeworks, Vancouver, BC) antibody at 15 μg/mL was added to the wells and Eppendorf tubes (Thermo Fisher Scientific, Waltham, MA) containing Quantum Simply Cellular IgG beads (anti-human or anti-mouse) and blank beads. Cells and beads were incubated with the antibodies for 1 hr at 4° C. in the dark. Cells and beads were washed, resuspended, and analyzed by flow cytometry. For analysis, a standard curve was generated using the spreadsheet provided by Bangs Laboratories (Fishers, Indiana) for the specific lot of beads, and the surface antigen binding capacity (ABC) was generated by entering the geometric means of the cell populations using the same spreadsheet. ABC values represent the number of molecules of receptor expressed on the cell surface assuming a monovalent binding model.

FIG. 19 demonstrates the range of CLDN18.2 and PD-L1 surface expression measured for three cell lines tested in TDCC: SNU-620, SNU-601, and KATO-III.

### Example 8: Investigation of Added Functionality of PD-1 Moiety in Trispecific PD-1/Anti-CD3/Anti-Cldn18.2 Using Hybrid PD-1/PD-L1 Reporter Gene Assay

To investigate blocking of the PD-1:PD-L1 checkpoint engagement by the PD-1 moiety of a fusion protein in addition to the construct's T-cell engagement function, a custom hybrid PD-1/PD-L1 Reporter Gene Assay (RGA) was performed as described herein.

### Hybrid PD-1/PD-L1 RGA

SNU-620 (Cldn18.2+, PD-L1+) cells were cultured in growth medium consisting of RPMI 1640 medium (Thermo Fisher Scientific, Waltham, MA) supplemented with 10% Fetal Bovine Serum (Thermo Fisher Scientific, Waltham, MA), and Jurkat T cells stably expressing human PD-1 and NFAT-induced luciferase (PD-1/PD-L1 Blockade Bioassay Promega Cat #J1250, Madison, WI) were cultured in RPMI 1640 medium with L-glutamine and HEPES supplemented with 10% Fetal Bovine serum, Hygromycin B, Antibiotic G-418 Sulfate solution, Sodium Pyruvate, and MEM nonessential amino acids, were maintained in T-75 or T-175 flasks (Corning, Corning, NY) in an incubator at 37° C. with 5% carbon dioxide prior to assay set-up. Prior to the day of the experiment, tumor cells were treated with 20 ng/ml of Recombinant Human IFN-gamma protein (R&D Systems Cat #285-IF, Minneapolis, MN) for 24 h. On the day of the experiment, the variants were titrated in duplicate 1:8 dilution in a separate titration plate from 150 nM to 0.00014 pM, and then transferred into 384-well Low Flange White Flat Bottom Polystyrene TC-treated Microplates, (Corning Cat #3570, Corning, NY) in 20 μL total volume per well. Tumor cells were dissociated using TrypLE (Thermo Fisher Scientific, Waltham, MA) and mixed with Jurkat cells at a 1:1 ratio in RPMI 1640 supplemented with 1% Fetal Bovine serum. 20 μL of the mixed cell suspension was added to the plate containing the titrated variants. The plates were incubated for 5 h at 37° C. with 5% carbon dioxide. Post incubation, 30 μL of Bio-Glo™ Luciferase Assay reagent (Promega Cat #G7940, Madison, WI) was added to all wells ensuring no bubbles were formed. The plate was read after 10 min in Luminescence mode on the microplate reader (Biotek Synergy H1, Winooski, VT) with a gain of 150.

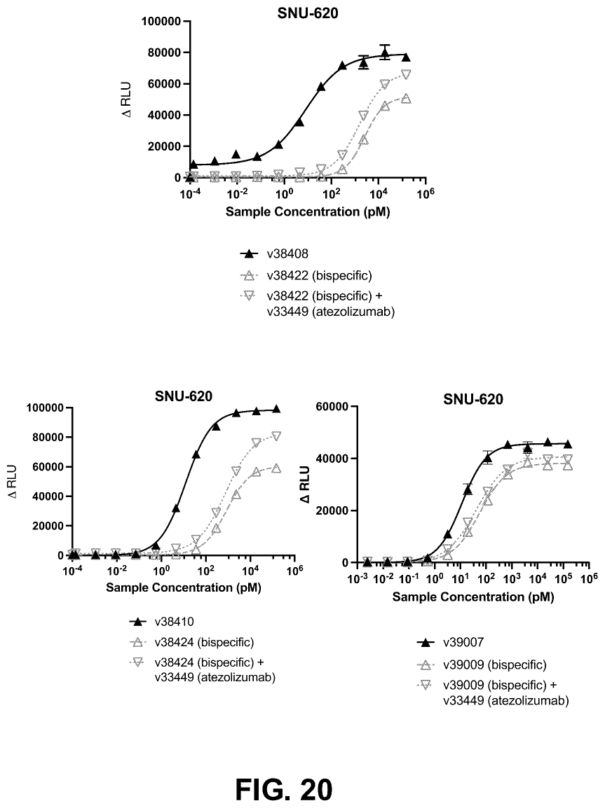

The hybrid PD-1/PD-L1 RGA was used to interrogate the functionality of the high affinity PD-1 moiety in the trispecific variants (FIG. 20). Assay cells treated with a bispecific variant (v38417) capable of crosslinking T cells and tumor cells showed increasing luminescence with increasing concentration of variant. Inclusion of saturating amounts of ant-PD-L1 antibody with the bispecific anti-CD3/anti-CLDN18.2 antibody could productively cross-link T-cells and tumor cells, high concentrations of the anti-PD-L1 antibody robustly blocked the PD-1:PD-L1 checkpoint engagement, leading to higher RLU indicative of increased CD3-mediated T cell signalling. Treatment with a trispecific variant capable of crosslinking T cells and tumor cells as well as PD-L1 blockade (v38408, v38410, v39007) resulted in a further increase in the RGA signal indicating superior checkpoint blockade with CD3 engagement in the trispecific compared to combination treatment.

### Example 9: Screening of Trispecific PD-1/Anti-CD3/Anti-TAA Formats with Affinity-Modulated PD-1 by SPR, TDCC, and Hybrid PD-1/PD-L1 RGA

As PD-L1 can be expressed by many tissues and immune cells under inflammatory conditions, the affinity of the PD-1 moiety in the trispecific fusion protein for PD-L1 was varied relative to the high affinity PD-1 moiety. As such, a panel of PD-1/anti-CD3/anti-TAA trispecific variants were generated containing a PD-1 moiety that was either (i) wildtype in sequence, (ii) contained a single point mutation A132L (see Lazar-Molnar et al., 2017, EBioMedicine, 17 (2017): 30-44), or (iii) 18 mutations in G1 1-4 (scc Maute et al., 2015, Proc. Natl. Acad. Sci., 112 (47):E6506-E6514).). A panel of trispecific PD-1 affinity variants were assayed to measure affinity to PD-L1, CD3, MSLN and PD-L2 by SPR. Potency and PD-1/PD-L1 checkpoint blockade were assayed by TDCC and PD-1/PD-L1 hybrid RGA, respectively. Additionally, cytokine production from TDCC co-culture was also measured.

SPR Binding Affinity of Trispecific PD-1/Anti-CD3/Anti-TAA Formats Containing PD-1 Moiety with a Range of Affinities for PD-L1

Trispecific PD-1/anti-CD3/anti-TAA (e.g., Her2, CLDN18.2, or MSLN) variants were assessed for binding to recombinant human, mouse, and cynomolgus monkey PD-L1, and human PD-L1 by SPR. The variants were also measured for binding affinity to recombinant human CD3. The binding kinetics of select trispecific PD-1/anti-CD3/anti-MSLN variants for recombinant human mesothelin.

### Methods

A Biacore T200 instrument (Cytiva/Danaher, Washington D.C., USA, product #28975001) was used to measure the binding kinetics of trispecifc variants to recombinant PD-L1 and recombinant PD-L2 protein. SPR experiments were conducted at a temperature of 25° C. using HBS EP+ (Cytiva/Danaher, Washington D.C., USA, 10 mM HEPES, 150 mM NaCl, 3 mM EDTA, and 0.05% v/v surfactant P20 as running buffer. Series S Sensor chip CM5, (Cytiva/Danaher, Washington D.C., USA, product #29149603), HBS EP+10× buffer (Cytiva/Danaher, Washington D.C., USA, product #BR100669), 10 mM Sodium Acetate pH 5.5 (Cytiva/Danaher, Washington D.C., USA, product #BR100352), NaOH 50 (Cytiva/Danaher, Washington D.C., USA, product #BR100358), 10 mM Glycine-HCL pH3.0 (Cytiva/Danaher, Washington D.C., USA, product #BR100357), amine coupling kit (Cytiva/Danaher, Washington D.C., USA, product #BR100050) were all purchased from Cytiva. Human PD-L1 (cat #), mouse PD-L1 (cat #), cynomolgus monkey PD-L1 (cat #), human PD-L2 (cat #), human CD3 (cat #), and human mesothelin (cat #) were purchased from R&D Systems (R&D Systems, Minneapolis, MN, USA, cat #10500-CV).

Binding kinetics of trispecific variants for human, mouse, and cynomolgus monkey PD-L1 were measured by covalently immobilizing the PD-L1 on the CM5 Series S sensor chip by amine coupling method. Immediately after EDC/NHS activation, 15 μg/ml human and cynomolgus monkey PD-L1 in 10 mM sodium acetate pH 5.5, and 15 μg/ml mouse PD-L1 in 10 mM sodium acetate pH 5.0 was injected on sample flow cells at 5 ul/min to reach surface ligand densities of ˜890, 1400, 910 resonance units (RU), respectively. Buffer was injected on the reference cell. Ethanolamine was introduced for 7 mins at 10 ul/min to deactivate the surface.

Binding kinetics of trispecific variants for human PD-L2 were measured by covalently immobilizing the PD-L2 on the CM5 Series S sensor chip by amine coupling method. Immediately after EDC/NHS activation, 10 μg/ml PD-L2 in 10 mM sodium acetate pH 5.5 was injected on sample flow cells at 5 ul/min to reach surface ligand densities of ˜450 RU. Buffer was injected on the reference cell. Ethanolamine was introduced for 7 mins at 10 ul/min to deactivate the surface.

Binding kinetics of trispecific variants for human CD3 were measured by covalently immobilizing the CD3 on the CM5 Series S sensor chip by amine coupling method. Immediately after EDC/NHS activation, 5 μg/ml CD3 in 10 mM sodium acetate pH 5.0 was injected on sample flow cells at 5 ul/min to reach surface ligand densities of ˜120 RU. Buffer was injected on the reference cell. Ethanolamine was introduced for 7 mins at 10 ul/min to deactivate the surface.

Binding kinetics of trispecific PD-1/anti-CD3/anti-MSLN variants for human mesothelin were measured by covalently immobilizing the mesothelin on the CM5 Series S sensor chip by amine coupling method. Immediately after EDC/NHS activation, 5 μg/ml mesothelin in 10 mM sodium acetate pH 5.0 was injected on sample flow cells at 5 ul/min to reach surface ligand densities of ˜130 RU. Buffer was injected on the reference cell. Ethanolamine was introduced for 7 mins at 10 ul/min to deactivate the surface.

Trispecific variants were injected on both reference and sample flow cells as analytes. Six concentrations following a three-fold dilution series of trispecific variants were injected at a flow rate of 30 ul/min for 240 s, then dissociate for 500 s. The variant concentration ranges used for human PD-L1, CD3, and mesothelin (MSLN) kinetic measurement were 0.62-50 nM for HAC PD-1 variants, 1.23-100 nM for G1-41 variants, 6.17-500 nM for A132 PD-1 variants, and 12.35-1000 nM for WT PD-1 variants. The concentration ranges used for mouse and cynomolgus monkey PD-L1 kinetic measurement were 0.25-20 nM for HAC PD-1 variants, 1.23-100 nM for G1-41 variants, 6.17-500 nM for A132 PD-1 variants, and 9.88-800 nM for WT PD-1 variants. The concentration ranges used for human PD-L2 kinetic measurement were 7.17-500 nM for HAC PD-1 variants, and 12.35-1000 nM for WT PD-1 variants. 10 mM glycine-HCl pH 1.7 was used to regenerate the human, mouse, and cynomolgus monkey PD-L1 and human PD-L2 surface between each cycle for 30 s at 30 ul/min. 3 M MgCl₂ was used to regenerate the human CD3 and mesothelin surface between each cycle for 30 s at 30 ul/min. The binding kinetics were analyzed using the 1:1 binding model and kinetic analyses were performed using Biacore T200 Evaluation Software v3.0.

### Results

Trispecific variants measured by SPR showed binding to human, mouse, and cynomolgus monkey PD-L1. Trispecific variants with affinity modulated PD-1:wildtype, A132L, G1 4-1, and HAC PD-1 showed binding affinities for human PD-L1 consistent with those that were previously reported for such PD-1 domains. Binding measurements confirm cross-species binding of the different PD-1 moieties to PD-L1 from human, mouse and cynomolgus monkey (Table X4). Furthermore, trispecific variants with PD-1 moiety containing point mutations to knockout binding to PD-L1 did not show measurable binding to human, mouse, or cynomolgus monkey PD-L1 (Table X4).

TABLE X4
Summary table of trispecific variant PD-1 affinity for human,
mouse, or cynomolgus monkey PD-L1 measured by SPR

| | | | | Cynomolgus |
|---|---|---|---|---|
| | | Human | Mouse | monkey |
| | Variant | PD-L1 | PD-L1 | PD-L1 |
| Variant | Description | K_{D} (M) | K_{D} (M) | K_{D} (M) |
| v38400 | CD3 x Her2 Fab x scFv | 1.3E−09 | 9.4E−09 | 1.2E−09 |
| | Fc, high affinity (HAC) | | | |
| | PD-1 attached to CD3 | | | |
| | HC | | | |
| v38401 | CD3 x Her2 Fab x scFv | No | No | No |
| | Fc, attenuated (KO) | binding | binding | binding |
| | PD-1 attached to CD3 | | | |
| | HC | | | |
| v38403 | CD3 x Her2 Fab x scFv | >1.0E−06 | >1.0E−06 | >1.0E−06 |
| | Fc, wildtype (WT) | | | |
| | PD-1 attached to CD3 | | | |
| | HC | | | |
| v38404 | CD3 x Her2 Fab x scFv | 1.1E−07 | 3.0E−07 | 3.3E−07 |
| | Fc, moderate affinity | | | |
| | (A132L) PD-1 attached | | | |
| | to CD3 HC | | | |
| v38405 | CD3 x Her2 Fab x scFv | 3.9E−08 | 1.7E−09 | 6.2E−08 |
| | Fc, moderate affinity | | | |
| | (G1 4-1) PD-1 attached | | | |
| | to CD3 HC | | | |
| v38409 | Cldn18.2 Fab x CD3 | 3.2E−09 | 1.3E−08 | 5.6E−09 |
| | scFv Fc, high affinity | | | |
| | (HAC) PD-1 attached | | | |
| | to HC2 (N-term) | | | |
| v38423 | Cldn18.2 Fab x CD3 | No | No | No |
| | scFv Fc, attenuated | binding | binding | binding |
| | (KO) PD-1 attached to | | | |
| | HC2 (N-term) | | | |
| v38733 | Cldn18.2 Fab x CD3 | 7.5E−08 | 1.9E−08 | 2.3E−08 |
| | scFv Fc, moderate | | | |
| | affinity (G1 4-1) PD-1 | | | |
| | attached to HC2 | | | |
| | (N-term) | | | |
| v38735 | Cldn18.2 Fab x CD3 | 7.4E−08 | 5.7E−08 | 1.1E−07 |
| | scFv Fc, moderate | | | |
| | affinity (A132L) PD-1 | | | |
| | attached to HC2 | | | |
| | (N-term) | | | |
| v38736 | Cldn18.2 Fab x CD3 | 1.1E−07 | 4.6E−08 | 1.4E−07 |
| | scFv Fc, wildtype (WT) | | | |
| | PD-1 attached to HC2 | | | |
| | (N-term) | | | |
| v38408 | CD3 Fab x Cldn18.2 | 3.7E−09 | 8.1E−09 | 3.5E−09 |
| | scFv Fc, high affinity | | | |
| | (HAC) PD-1 attached | | | |
| | to HC1 (N-term) with | | | |
| | GGGGSG linker | | | |
| | sequence | | | |
| v38522 | CD3 Fab x Cldn18.2 | 2.8E−09 | 5.2E−09 | 2.8E−09 |
| | scFv Fc, high affinity | | | |
| | (HAC) PD-1 attached | | | |
| | to HC1 (N-term) with | | | |
| | (EAAAK)₂ linker | | | |
| | sequence | | | |

Trispecific PD-1/anti-CD3/anti-Cldn18.2 variants with different affinity modulated PD-1, but matched format, were measured by SPR to determine the effect of format on binding affinity for human PD-L1 and human CD3. Trispecific variants with PD-1 moiety containing point mutations to knockout binding to PD-L1 did not show measurable binding to human PD-L1 by SPR. The data is summarized in Table X5.

TABLE X5
Binding affinity of format-matched PD-1/anti-CD3/anti-Cldn18.2 trispecific variants for
human PD-L1 and human CD3 measured by SPR

| | | | Human | Human |
|---|---|---|---|---|
| Format | Variant | | PD-L1 | CD3 |
| Schematic^{a} | number | Variant description | K_{D} (M) | K_{D} (M) |
| [symbol] | v38408 v38422 | CD3 Fab x Cldn18.2 scFv Fc, high affinity (HAC) PD-1 attached to HC1 (N-term) with GGGGSG linker CD3 Fab x Cldn18.2 scFv Fc, attenuated (KO) PD-1 attached to HC1 (N-term) with GGGGSG linker | 5.1E−09 No binding | 8.4E−08 4.0E−09 |
| | v38729 | CD3 Fab x Cldn18.2 scFv Fc, moderate | 5.9E−08 | 2.9E−09 |
| | | affinity (G1 4-1) PD-1 attached to HC1 | | |
| | | (N-term) | | |
| | v38731 | CD3 Fab x Cldn18.2 scFv Fc, moderate | 1.2E−07 | 9.4E−08 |
| | | affinity (A132L) PD-1 attached to HC1 | | |
| | | (N-term) | | |
| | v38732 | CD3 Fab x Cldn18.2 scFv Fc, wildtype (WT) | 4.4E−07 | 3.0E−08 |
| | | PD-1 attached to HC1 (N-term) | | |
| [symbol] | v38409 v38423 v38733 | Cldn18.2 Fab x CD3 scFv Fc, high affinity (HAC) PD-1 attached to HC2 (N-term) Cldn18.2 Fab x CD3 scFv Fc, attenuated (KO) PD-1 attached to HC2 (N-term) Cldn18.2 Fab x CD3 scFv Fc, moderate affinity (G1 4-1) PD-1 attached to HC2 (N-term) | 6.5E−09 No binding 8.5E−08 | 4.0E−08 2.2E−08 2.6E−08 |
| | v38735 | Cldn18.2 Fab x CD3 scFv Fc, moderate | 3.0E−07 | 8.2E−08 |
| | | affinity (A132L) PD-1 attached to HC2 | | |
| | | (N-term) | | |
| | v38736 | Cldn18.2 Fab x CD3 scFv Fc, wildtype (WT) | 1.6E−06 | 1.1E−07 |
| | | PD-1 attached to HC2 (N-term) | | |
| [symbol] | v38410 v38424 v38737 | CD3 Fab x Cldn18.2 scFv Fc, high-affinity (HAC) PD-1 attached to HC1 (C-term) CD3 Fab x Cldn18.2 scFv Fc, attenuated (KO) PD-1 attached to HC1 (C-term) CD3 Fab x Cldn18.2 scFv Fc, moderate affinity (G1 4-1) PD-1 attached to HC1 (C-term) | 9.3E−09 No binding 2.8E−07 | 4.5E−08 3.7E−08 3.5E−09 |
| | v38739 | CD3 Fab x Cldn18.2 scFv Fc, moderate | 3.1E−07 | 3.9E−08 |
| | | affinity (A132L) PD-1 attached to HC1 | | |
| | | (C-term) | | |
| | v38740 | CD3 Fab x Cldn18.2 scFv Fc, wildtype (WT) | 5.1E−07 | 3.0E−08 |
| | | PD-1 attached to HC1 (C-term) | | |
| [symbol] | v38412 v38426 | CD3 Fab with C-term Cldn18.2 scFv x Cldn18.2 scFv Fc with high affinity (HAC) PD-1 attached to CD3 LC (N-term) CD3 Fab with C-term Cldn18.2 scFv x Cldn18.2 scFv Fc with attenuated (KO) PD-1 attached to CD3 LC (N-term) | 6.7E−09 No binding | 1.1E−07 7.7E−08 |
| | v38741 | CD3 Fab with C-term Cldn18.2 scFv x | 9.5E−08 | 1.8E−08 |
| | | Cldn18.2 scFv Fc with moderate affinity | | |
| | | (G1 4-1) PD-1 attached to CD3 LC (N-term) | | |
| | v38743 | CD3 Fab with C-term Cldn18.2 scFv x | 2.2E−07 | 1.2E−07 |
| | | Cldn18.2 scFv Fc with moderate affinity | | |
| | | (A132L) PD-1 attached to CD3 LC (N-term) | | |
| | v38744 | CD3 Fab with C-term Cldn18.2 scFv x | 1.9E−06 | 1.9E−07 |
| | | Cldn18.2 scFv Fc with wildtype (WT) PD-1 | | |
| | | attached to CD3 LC (N-term) | | |

^{a}In the schematic, the anti-CD3 polypeptide chain is shaded white/bright grey, the anti-Cldn18.2 polypeptide chain is shaded dark grey, and the PD-1 moiety is striped.

Seven PD-1/anti-CD3/anti-MSLN trispecific formats were measured by SPR to determine the effect of geometry on binding affinity for human PD-L1. human CD3, and human mesothelin. The data is summarized in Table X6.

TABLE X6
Binding affinity of seven PD-1/anti-CD3/anti-MSLN trispecific
formats for human PD-L1, human CD3, and human mesothelin
measured by SPR

| | | Human | Human | Human |
|---|---|---|---|---|
| Variant | | PD-L1 | CD3 | MSLN |
| number | Variant description | K_{D} (M) | K_{D} (M) | K_{D} (M) |
| v38520 | CD3 Fab x MSLN scFv Fc, | 4.4E−09 | 7.9E−08 | 1.0E−09 |
| | high affinity (HAC) PD-1 | | | |
| | attached to CD3 HC | | | |
| | (N-term) with linker | | | |
| | (EAAAK)₂ | | | |
| v38441 | MSLN Fab x CD3 scFv Fc, | 2.9E−09 | 4.0E−08 | 2.5E−10 |
| | high affinity (HAC) PD-1 | | | |
| | attached to MSLN HC | | | |
| | (N-term) | | | |
| v38442 | CD3 Fab x MSLN scFv Fc, | 5.1E−09 | 3.1E−08 | 5.0E−11 |
| | high affinity (HAC) PD-1 | | | |
| | attached to CD3 HC | | | |
| | (C-term) | | | |
| v38443 | CD3 Fab with MSLN scFv | 2.4E−09 | 4.4E−08 | 6.0E−11 |
| | attached to CD3 HC | | | |
| | (N-term) x high affinity | | | |
| | (HAC) PD-1 attached to Fc | | | |
| | hinge | | | |
| v38449 | CD3 Fab with MSLN scFv | 2.8E−09 | 4.1E−08 | 2.5E−10^{a} |
| | attached to CD3 HC | | | |
| | (N-term) x MLSN scFv Fc | | | |
| | with high affinity (HAC) | | | |
| | PD-1 attached (N-term) | | | |
| v38450 | CD3 Fab with MSLN scFv | 6.7E−09 | 4.0E−08 | 1.2E−10^{a} |
| | attached to CD3 HC | | | |
| | (N-term) x MLSN scFv Fc | | | |
| | withhigh affinity (HAC) | | | |
| | PD-1 attached (C-term) | | | |
| v38452 | CD3 Fab with MSLN scFv | 8.8E−09 | 3.8E−08 | 1.2E−10^{a} |
| | attached to CD3 HC | | | |
| | (N-term) x MLSN scFv Fc | | | |
| | with high affinity (HAC) | | | |
| | PD-1 attached to CD3 Fab | | | |
| | LC (C-term) | | | |

^{a}Apparent K_{D} were reported, as these variants are bivalently bound to mesothelin but were fit using a 1:1 kinetic model.

The binding kinetics of the PD-1 moiety within the trispecific variants for human PD-L2 was also measured by SPR. Wildtype PD-1 and affinity modulated A132L PD-1 both were capable of binding PD-L2 with similar affinity as measured for human PD-L1 (Table X7). In contrast, trispecific variants containing high affinity (HAC) PD-1 moiety bound human PD-L1 but not human PD-L2 (Table X7), consistent with literature (see Maute et al., 2015, Proc. Natl. Acad. Sci., 112 (47):E6506-E6514).

TABLE X7
Binding affinity of trispecific variants for human PD-L1 and PD-L2
measured by SPR.

| | | Human | Human |
|---|---|---|---|
| Variant | | PD-L1 | PD-L2 |
| number | Variant description | K_{D} (M) | K_{D} (M) |
| v38410 | CD3 Fab x Cldn18.2 scFv Fc, high- | 3.2E−09 | No |
| | affinity (HAC) PD-1 attached to HC1 | | binding |
| | (C-term) | | |
| v38449 | CD3 Fab with MSLN scFv attached to | 1.7E−08 | No |
| | CD3 HC (N-term) x MLSN scFv Fc with | | binding |
| | high affinity (HAC) PD-1 attached | | |
| | (N-term) | | |
| v38450 | CD3 Fab with MSLN scFv attached to | 3.1E−08 | No |
| | CD3 HC (N-term) x MLSN scFv Fc with | | binding |
| | high affinity (HAC) PD-1 attached | | |
| | (C-term) | | |
| v38739 | CD3 Fab x Cldn18.2 scFv Fc, moderate | 1.1E−07 | 2.3E−07 |
| | affinity (A132L) PD-1 attached to HC1 | | |
| | (C-term) | | |
| v38917 | CD3 Fab with MSLN scFv attached to | 1.1E−07 | 1.2E−07 |
| | CD3 HC (N-term) x MLSN scFv Fc with | | |
| | moderate affinity (A132L) PD-1 attached | | |
| | (N-term) | | |
| v38921 | CD3 Fab x (MSLN scFv)² Fc, moderate | 4.2E−07 | 2.4E−07 |
| | affinity (A132L) PD-1 attached to HC2 | | |
| | (C-term) | | |
| v38918 | CD3 Fab with MSLN scFv attached to | 1.4E−06 | 6.7E−07 |
| | CD3 HC (N-term) x MLSN scFv Fc with | | |
| | wildtype (WT) PD-1 attached (N-term) | | |

### TDCC Analysis of Affinity Modulated PD-1 Trispecific Variants

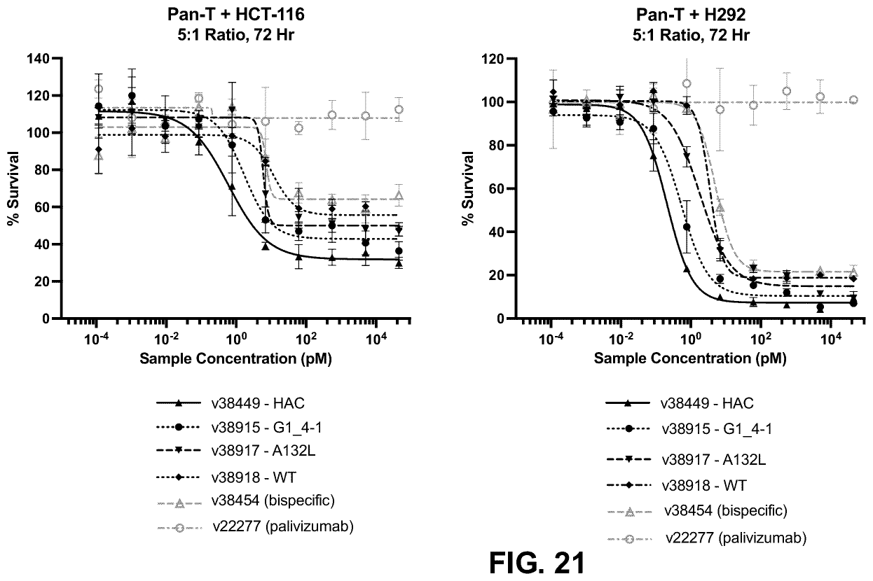

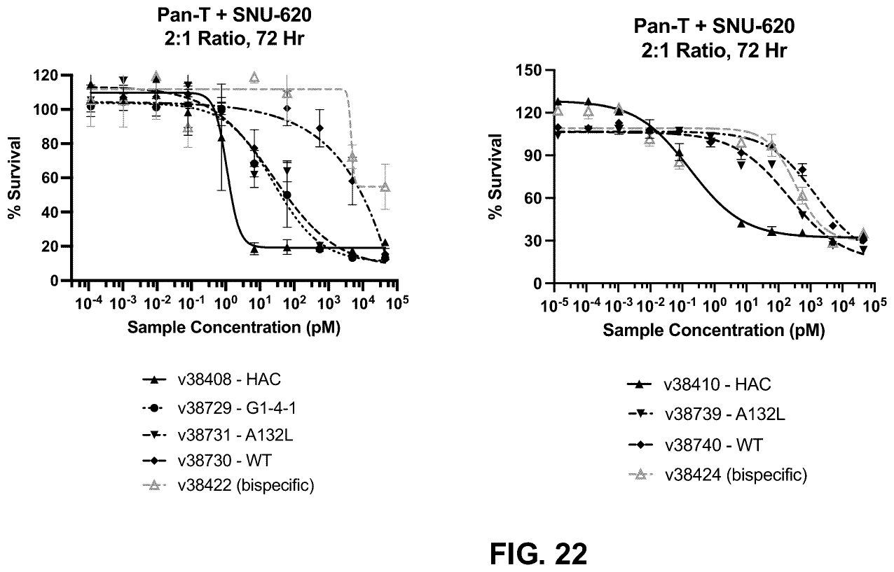

Trispecific fusion protein variants with affinity modulated PD-1 moiety were engineered in trispecific T cell engager formats targeting Cldn18.2 or MSLN. Methods used were as described above. Trispecific T cell engager formats were assessed for potency in TDCC assays (FIG. 21 and FIG. 22). All trispecific formats show PD-1 affinity dependent potency where increasing PD-1 affinity increases potency in TDCC.

### Hybrid PD-1/PD-L1 RGA of Affinity Modulated PD-1 Trispecific Variants

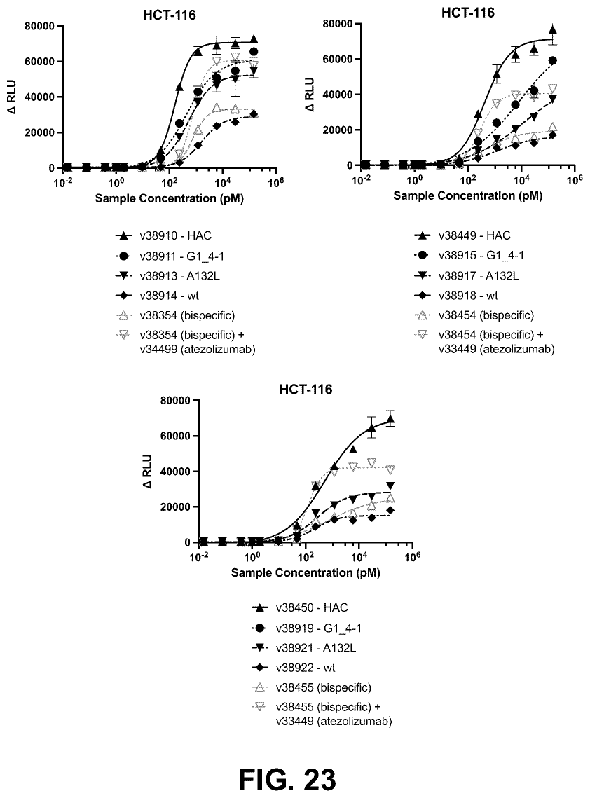

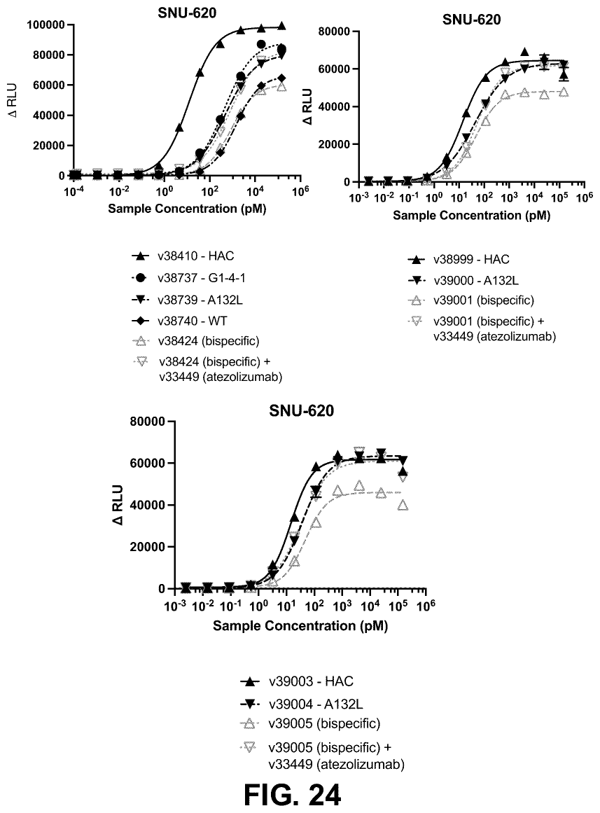

The hybrid PD-1/PD-L1 RGA was used to interrogate the functionality of the PD-1 moiety with different PD-L1 affinities in the trispecific variants (FIG. 23 and FIG. 24). Methods used were as described above. Trispecific formats targeting either MSLN or CLDN18.2 both showed PD-1 moiety affinity for PD-L1-dependent PD-1/PD-L1 blockade by the RGA. Trispecifics containing a PD-1 moiety with higher affinity for PD-L1 had increased PD-1/PD-L1 checkpoint blockade measured by increased RLU (FIG. 23 and FIG. 24).

### Cytokine Analysis

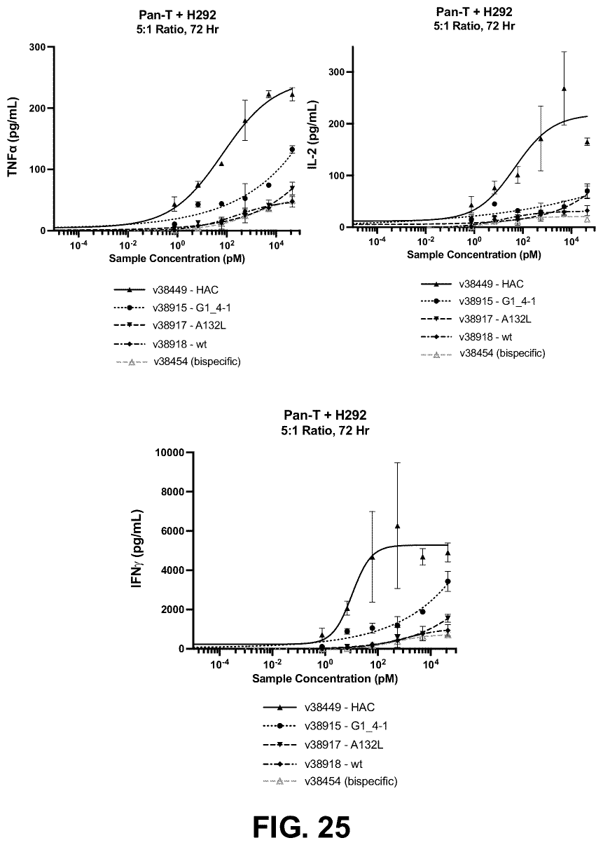

Supernatants from TDCC assays were collected after culture for 72 hour. Supernatants were assayed for the presence of TNF&, IL-2, and IFN-γ by Meso Scale as stipulated by the vendor. The data showed that the trispecific fusion proteins showed increased cytokine production with increasing PD-L1 affinity (FIG. 25).

### Example 10: Trispecific Fusion Protein Variants Engage PD-L1 Expressing Dendritic Cells to Activate T Cells In Vitro

The purpose of this experiment was to determine whether the trispecific (v31929) can engage PD-L1-expressing antigen presenting cells and whether it results in T cell activation beyond that induced by the bispecific antibody alone which does not contain a PD-1 moiety.

### Dendritic Cell (DC) Differentiation

Primary CD14+ monocytes (Stemcell Technologies, Vancouver BC) were differentiated and matured according to manufacturer's protocol for ImmunoCult™ Dendritic Cell Culture Kit (Stemcell Technologies, Vancouver BC). Cells were thawed in pre-warmed DC differentiation media, spun down at 300×g for 10 min, supernatant was discarded, and cells were resuspended at 1×10\hat{ }6 cells/mL. Aliquots were reserved to be re-frozen in FBS+10% DMSO as “Day 0 monocytes”, and differentiation supplement was added to remaining cells at 1:100 final dilution. Cells were distributed into culture plates and left to differentiate for 3 days at 37° C.+5% CO₂. On day 3, media was replaced with fresh DC differentiation media+differentiation supplement. Non-adherent cells from removed media were spun down and re-plated back to culture plate. After 2 days at 37° C.+5% CO₂, DC maturation supplement was added to the plate at 1:100 dilution and left to incubate for an additional 2 days at 37° C.+5% CO₂. On Day 7, the fully mature DCs were harvested with TrypLE™ Express Enzyme (ThermoFisher Scientific, Waltham MA) and resuspended in FACS buffer (PBS+2% FBS) for phenotype assessment and in RPMI+10% FBS for co-culture assay.

### Determination of Differentiation and Maturation Markers (CD14, CD80, CD16, CD11c, and PD-L1) by Flow Cytometry

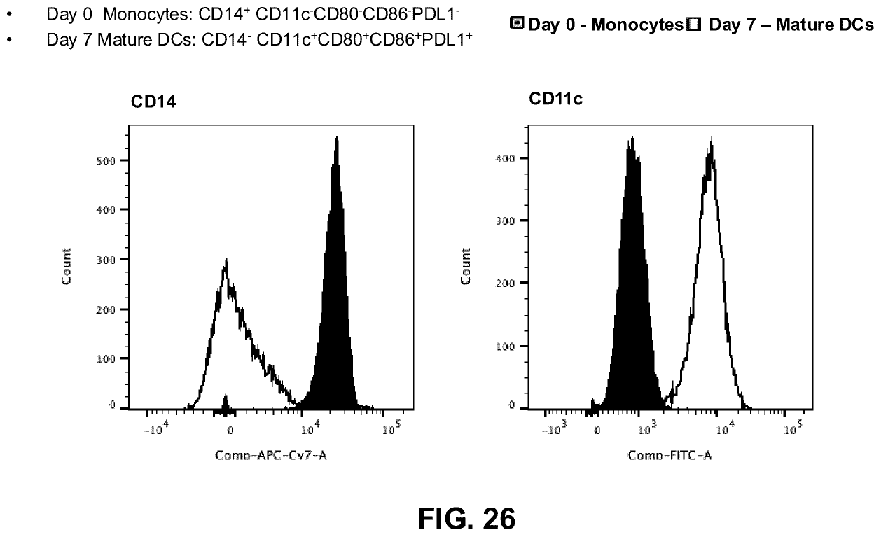

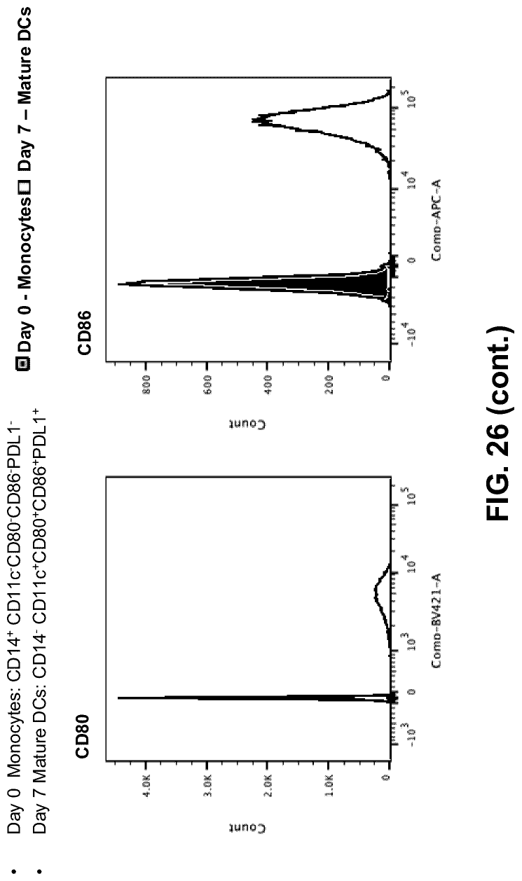

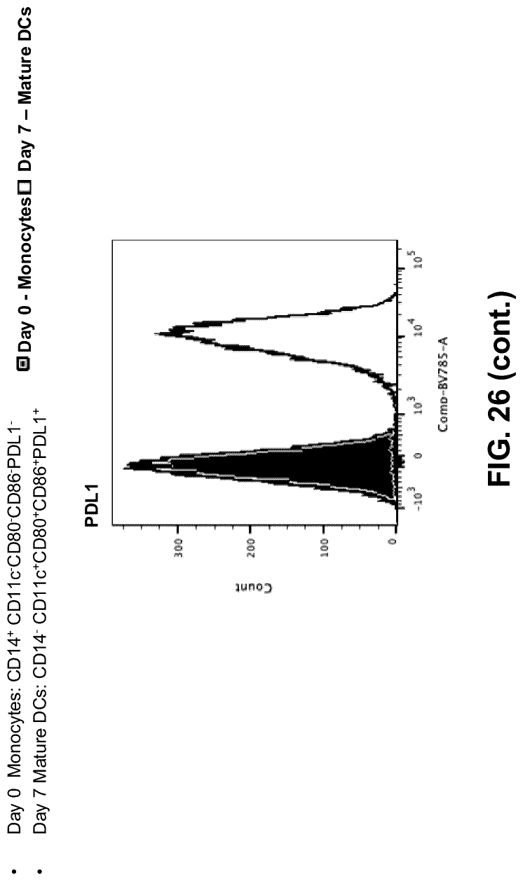

Day 0 monocytes were thawed in RPMI+10% FBS spun down and resuspended in FACS buffer. 4.5×10\hat{ }4 cells each of Day 0 and Day 7 cells were mixed and incubated with equal volume Human TruStain FcX™ (BioLegend, San Diego CA) at 1:10 dilution for 10 min at RT. The cells were then stained with an equal volume of antibody cocktail containing 1:25 dilution of anti-huCD14-APC-Cy7 (BioLegend, San Diego CA), anti-huCD80-BV421 (BD Horizon, San Jose CA), anti-huCD86-APC (BioLegend, San Diego CA), anti-huPDL1-BV786 (BD Horizon, San Jose CA), anti-huCD11c-FITC (BD Pharmingen, San Jose CA), and 1:500 cFluor506 Fixable Viability Dye (ThermoFisher Scientific, Waltham MA) for 30 min at 4° C. The cells were washed 3× and resuspended in 75 μL of FACS buffer for flow cytometry analysis on a BD LSRFortessa™ X-20 Cell Analyzer. Phenotype of Day 0 monocytes (CD14+CD80-CD86-CD11c⁻PD-L1⁻) and mature DCs (CD14-CD80⁺CD86⁺CD11c⁺PD-L1⁺) were confirmed (FIG. 26).

### DC-T Cell Co-Culture Assay

Autologous primary CD3+ T cells (Stemcell Technologies, Vancouver BC) were thawed in pre-warmed RPMI+10% FBS washed 2× with PBS and resuspended at 10×10\hat{ }6 cells/mL in warm PBS containing 5 μM of Cell Proliferation Dye (CPD) eFluor™ 670 (ThermoFisher Scientific, Waltham MA). Cells were stained for 10 mins in a 37° C. water bath in the dark, reaction was quenched by adding 4-5 volumes of cold complete RPMI media (>10% FBS) and left on ice for 5 mins. Cells were spun down at 500×g for 5 mins, supernatant was discarded, and wash was repeated twice. Stained cells were resuspended with RPMI+10% FBS and mixed with autologous matured DCs at a 5:1 ratio. In a round bottom 96-well plate, 30 μL of cell mixture (10,000 cells DCs+50,000 T cells), 40 μL of variant (final concentration 1000, 10, 1, 0.1 or 0.01 pM), and 30 μL of RPMI+10% FBS was added for a total volume of 100 μL. Dynabeads™ human T Activator CD3/CD28 beads (ThermoFisher Scientific, Waltham MA), were added as an activation positive control. Cells were incubated at 37° C.+5% CO₂ for 5 days.

### T Cell Activation Assessment by Flow Cytometry

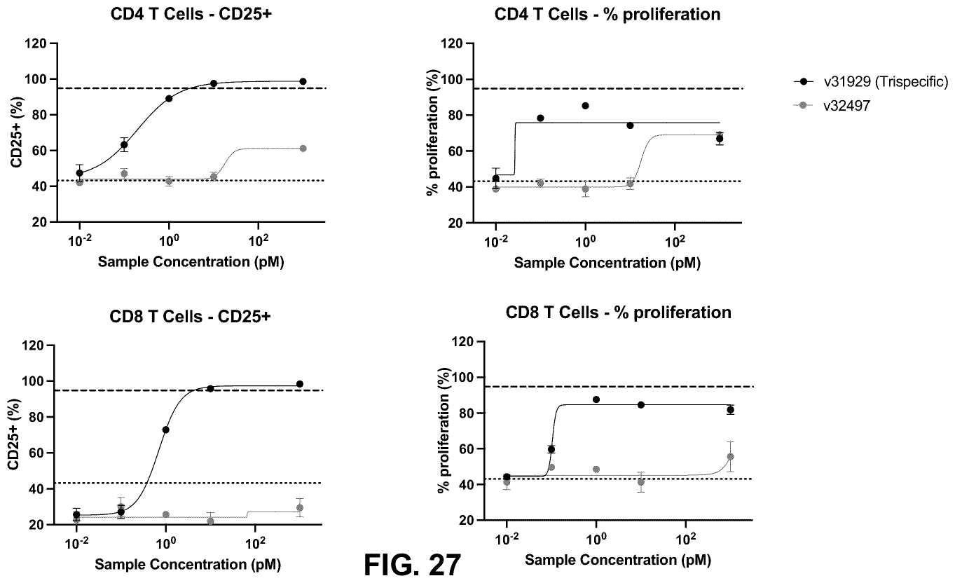

After a 5 day incubation with autologous mature DCs, CPD-labeled T cells were harvested, washed with FACS buffer and transferred to a 96-well v-bottom plate. The cells were stained in an antibody cocktail containing 1:25 dilution of anti-huCD4-BV605 (BioLegend, San Diego CA), anti-huCD8-APC-Cy7 (BD Pharmingen, San Jose CA), and anti-anti-huCD25-PE (BD Horizon, San Jose CA) with 1:1000 dilution eFluor 506 Fixable Viability Dye (ThermoFisher Scientific, Waltham MA) in FACS buffer and incubated for 30 min at 4° C. The cells were then washed 2× with FACS buffer and resuspended in 75 μL FACS buffer+2.5 mM EDTA for flow cytometry analysis on a BD LSRFortessa™ X-20 Cell Analyzer. T cell activation was determined by quantifying proliferation by CPD-dilution and upregulation of CD25 in CD4⁺ and CD8⁺ T cells (FIG. 27).

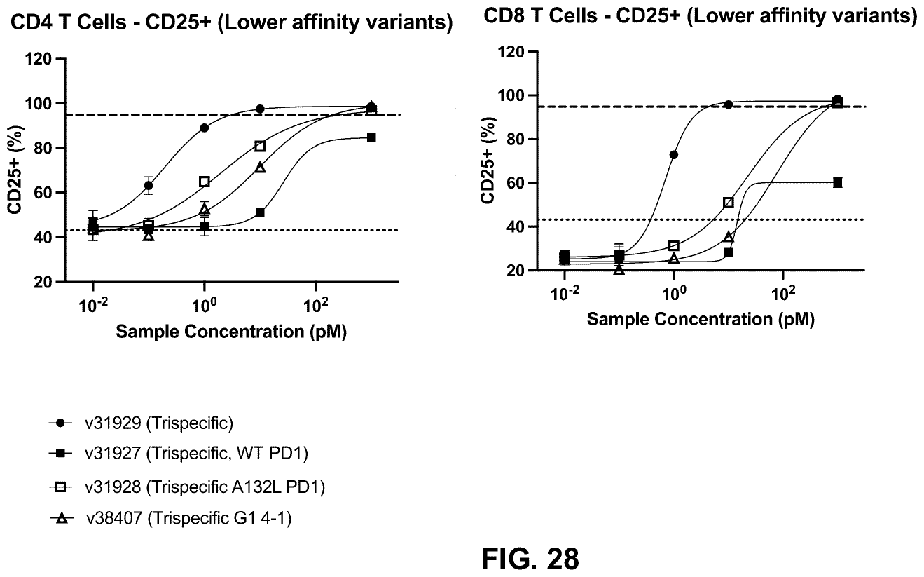

Variants with lower affinity to PD1 moieties v38407, v31928 and v31927 engaged PD-L1-expressing autologous DCs to induce T cell activation as measured by CD25 upregluation in both CD4 and CD8 T cells but to a lesser degree than the high-affinity variant v31929 (FIG. 28) supporting the mechanism for DC-T cell activation being engagement of PD-L1 on DCs via the PD1 protein moiety on the trispecific fusion proteins described herein.

### Example 11: In Vivo Efficacy Study of a Trispecific PD-1/Anti-CD3/Anti-Cldn18.2 Fusion Protein with Affinity-Modulated PD-1 Compared to a Structurally Similar Antibody Construct Having an Attenuated (KO) PD-1 with and without an Anti-PD-L1 Antibody

In order to compare the anti-tumor activity of a PD-1/anti-CD3/anti-CLDN18.2 trispecific fusion protein with affinity-modulated PD-1 (referred to as the “Trispecific”) with an attenuated (“knock-out,” or “KO”) PD-1 (attenuated)/anti-CD3/anti-CLDN18.2 (referred to as the “Bispecific”), an in vivo study is carried out. The efficacy of the Trispecific with varying PD-1 affinity is compared with that of the Bispecific combined with Atezolizumab, an anti-PD-L1 antibody.

To identify suitable donors, in one instance, six PBMC donors were screened using a flow cytometry-based tumor cell killing assay and a mouse model to determine tumor growth kinetics and graft vs host disease. For the tumor cell killing assay, SNU-620 target cells that express CLDN18.2 were labeled with CFSE and co-cultured with PBMC at E:T ratios of 1:1 and 10:1. Test articles were added concurrently with PBMCs with the final concentrations of 100 pM, 10 pM and 1 pM. After 5 days of culture, cells were stained for CD45 and viability dye and analyzed by flow cytometry. For tumor growth kinetics and graft vs host disease assessment, 5×10⁶ SNU-620 tumor cells were injected SC. When tumor volumes reached 100-120 mm³, mice were engrafted with PBMCs IV. Tumors and body weight were monitored twice weekly. Donors that displayed tumor cell killing in a dose dependent manner with addition of test articles and that showed consistent tumor growth for up to four weeks with minimal variation or graft versus host disease were selected for efficacy studies.

For the in vivo efficacy study, NCG (NOD/ShiLtJGpt-Prkd^{em26Cd52}Il2rg^{em26Cd22}/Gpt) immunodeficient mice are orthotopically implanted with 5×10⁶ SNU-620 tumor cells mixed 1:1 with Matrigel in the mammary fat pad. Once tumors reach a mean tumor volume between 100-120 mm³, around 2-3 week after implant, mice are randomized into study groups and 5×10⁶ human PBMCs are administered intravenously and tumor measurements along with body weights are taken twice a week. Test article dosing is initiated 24 hours after randomization. Mice receive intravenous injections of either the Trispecific (v38410, v38739) or Bispecific (v38417) test articles once a week for four weeks at a dose of either 0.1, 0.01 or 0.001 mg/kg. Two group of mice receive 0.01 or 0.001 mg/kg of the Bispecific antibody as well as 5 mg/kg Atezolizumab twice a week for four weeks by intraperitoneal injections to achieve saturation of PD-1 checkpoint blockade. General health and welfare of animals is monitored daily and study concluded 65 days after tumor implant or when the tumor burden of an individual animal reached >2000 mm³.

### Example 12: In Vivo Efficacy Study of a Trispecific PD-1/Anti-CD3/Anti-MSLN Fusion Protein with Affinity-Modulated PD-1 Compared to a Structurally Similar Antibody Construct Having an Attenuated (KO) PD-1 with and without an Anti-PD-L1 Antibody

In order to compare the anti-tumor activity of PD-1/anti-CD3/anti-MSLN fusion protein with varying PD-1 affinity (referred to as the “Trispecific”) with an attenuated (KO) PD-1 (attenuated)/anti-CD3/anti-MSLN (referred to as the “Bispecific”), an in vivo study is carried out. The efficacy of the Trispecific with varying PD-1 affinity is compared with that of the Bispecific combined with Atezolizumab, an anti-PD-L1 antibody.

To identify suitable donors, in one instance, six PBMC donors were screened using a TDCC assay using the method described above and a mouse model to determine tumor growth kinetics and graft vs host disease. For tumor growth kinetics and graft vs host disease assessment, 5×10⁶ HCT116 tumor cells were injected SC and 5×10⁶ PBMCs were engrafted IV on the same day. Tumors and body weight were monitored twice weekly. Donors that displayed tumor cell killing in a dose dependent manner with addition of test articles in our TDCC and that showed demonstrated consistent tumor growth for up to four weeks with minimal variation or graft versus host disease were selected for efficacy studies.

For the in vivo efficacy study, NCG (NOD/ShiLtJGpt-Prkdc^{em26Cd52}Il2rg^{em26Cd22}/Gpt) immunodeficient mice are orthotopically implanted with 5×10⁶ HCT116 tumor cells in PBS in the mammary fat pad. On the same day, 5×10⁶ human PBMCs are administered intravenously. Once tumors reach a mean tumor volume between 150-200 mm³, around 1-2 week after implant, mice are randomized into study groups and test article dosing is initiated. Tumor measurements along with body weights are taken twice a week. Mice receive intravenous injections of either the Trispecific (v38449, v38917) or Bispecific (v38454) test articles once a week for four weeks at a dose of either 3, 0.1 or 0.01 mg/kg. Two group of mice receive 0.1 or 0.01 mg/kg of the Bispecific antibody as well as 5 mg/kg Atezolizumab twice a week for four weeks by intraperitoneal injections to achieve saturation of PD-1 checkpoint blockade. General health and welfare of animals is monitored daily, and study concluded 60 days after tumor implant or when the tumor burden of an individual animal reached >2000 mm³.

### Example 13: Preparation of Anti-CLDN18.2 Antibodies

Antibodies that specifically bind CLDN18.2 were generated by immunizing rabbits with transiently transfected CHO cells expressing human CLDN18.2, as described below. These antibodies, or binding fragments thereof, can be useful for targeting human Cldn18.2, e.g., by using these generated anti-Cldn18.2 binding domains in the trivalent and trispecific antibody constructs of the present disclosure.

CHO-S cells (Invitrogen, Waltham, MA; Cat #R80007) were transiently transfected with a pTT5-based expression plasmid (National Research Council of Canada) encoding human CLDN18.2 according to manufacturer's instructions for the Neon Transfection System (Thermo Fisher Scientific, Waltham, MA). Two New Zealand White rabbits were subcutaneously immunized with transfected CHO cells over 63 days, after which blood was drawn and spleens harvested.

Anti-human CLDN18.2 antibody titers were determined by flow cytometry using HEK293-6E cells (National Research Council of Canada) expressing human CLDN18.2. Both rabbits mounted a significant response against human CLDN18.2.

Immunized rabbits were sacrificed, and the spleens harvested. Splenocytes for each rabbit were used for B cell enrichment and sorted on a FACSAria™ (Becton, Dickinson & Co., Franklin Lakes, NJ) into wells containing lysis buffer with a modified protocol based on the Selected Lymphocyte Antibody Method (SLAM) (Babcook et al., 1996, Proc Natl Acad Sci USA, 93 (15): 7843-7848).

Total RNA from wells containing a single B cell was used as template with SuperScript™ III (Thermo Fisher Scientific Corp., Waltham, MA) and oligo-dT20 (Integrated DNA Technologies, Inc., Coralville, IA) to transcribe cDNA from mRNA. Initial PCR of heavy and light chain antibody-coding sequences was performed using primers and methods modified from Babcook et al., 1996, Proc Natl Acad Sci USA, 93 (15): 7843-7848 and von Boehmer et al., 2016, Nat Protoc., 11 (10): 1908, with cDNA as the nucleic acid template. A subsequent PCR reaction was then performed on these unique sequences using V-segment family and J-segment family-specific primers and the resulting amplicons were cloned into pTT5-based expression plasmids (National Research Council of Canada). Unique heavy chain sequences and light chain sequences emerging from a single well sample were co-expressed in Expi293F cells (Thermo Fisher Scientific, Waltham, MA; Cat #A14527).

Cell supernatants containing secreted antibodies were assessed for human CLDN18.2 specificity by binding on HEK293-6E transfected with the same pTT5 based plasmid used to transfect CHO-S cells. The wells corresponding to human CLDN18.2-binding antibody containing supernatant were selected for sequencing.

Heavy and light chain PCR amplicons were sequenced using NGS-based Amplicon-EZ and analyzed for unique antibody-coding sequences. The following rabbit anti-human CLDN18.2 antibody VH and VL sequences was identified for the antibody referred to below as v35777:

TABLE X8
Rabbit V_{H} and V_{L} Sequences for Anti-Human CLDN18.2 Antibody

| Description | Sequence | SEQ ID NO: |
|---|---|---|
| Rabbit V_{H} | QSLEESGGRLVTPGTPLTLTCTVSGIDLSSNPMIWVRQ | 500 |
| | APGKGLQYIGIIDTDGSTYYASWAKGRFTGSKTSTTV | |
| | DLKITSPTTEDTATYFCARRLHGSSNGYYDDLWGQGT | |
| | LVTISS | |
| Rabbit V_{L} | DGVMTQTPSSVSAAVGGTVTIKCQASQSIYSYLSWYQ | 501 |
| (kappa) | QKPGQRPKLLIYKASTLASGVPSRFKGSGSGTEFTLTIS | |
| | GVQCDDAATYYCQQGYTVTNVDKNTFGGGTEVVVK | |

These rabbit V_{H} and V_{L} sequence was used to prepare a rabbit-human chimeric IgG1/kappa antibody construct, v35777, as follows. Coding sequences for antibody variable regions were cloned in frame into a human IgG1 expression vector or a human C kappa expression vector (based on the pTT5 vector). The human IgG1 constant region starts at alanine Kabat-114 and human C kappa constant region starts at arginine Kabat-108. The activities of the resultant recombinant chimeric antibody construct were confirmed in specificity binding assays.

### Creation of Humanized Candidate Sequences

The rabbit VH and VL sequences from chimeric antibody construct v35777 were aligned against human immunoglobulin germline sequences to select basis germline sequences for humanization. Human germline IGHV3-64*04 with IGHJ4*01 was selected for VH humanization. Human germline IGKV1-39*01 with IGKJ4*01 was selected for VL humanization. The CDR sequences by AbM definition from v35777 were swapped into the selected human germline frameworks to create a basis humanized construct. Several areas of the basis construct were identified for back mutation or deletion to the original rabbit parental sequence to minimize potential disruption to antigen binding. Five new candidate humanized VH sequences, and four new candidate humanized VL sequences were thus created.

### Screening of Humanized Candidate Sequences for Antigen Binding Activity

Recombinant human IgG1-based monoclonal antibodies containing each combination of the candidate V_{H} and V_{L} humanized sequences were expressed in Expi293F and the supernatants screened for binding to SNU-601 cells or HEK293-6E cells transfected with either human CLDN18.2 or human CLDN18.1 by FACS. All antibodies showed no detectable binding to HEK293-6E transfected with CLDN18.1. Five constructs represented by 4 humanized V_{H} and 2 humanized V_{L} sequences (TABLES X9 and X10) demonstrated binding activity similar to the parental v35777 construct (TABLE X13) and were selected for further analysis.

TABLE X9
Humanized V_{H} and V_{L} Domain Sequences

| | | SEQ |
|---|---|---|
| V_{H}/V_{L} | | ID |
| IDS | V_{H}/V_{L} Sequence | NO |
| H2 | QVQLVESGGGLVQPGGSLRLSCSVSGIDLSSNPMIWVRQ | 502 |
| | APGKGLEYIGIIDTDGSTYYADWAKGRFTISKDSSKNTVY | |
| | LQMNSLRAEDTAVYYCARRLHGSSNGYYDDLWGQGTL | |
| | VTVSS | |
| H3 | QVQLVESGGGLVQPGGSLRLSCSVSGIDLSSNPMIWVRQ | 503 |
| | APGKGLQYIGIIDTDGSTYYADWAKGRFTISKDSSKNTVY | |
| | LQMNSLRAEDTAVYYCARRLHGSSNGYYDDLWGQGTL | |
| | VTVSS | |
| H4 | QVQLVESGGGLVQPGGSLRLSCSVSGIDLSSNPMIWVRQ | 504 |
| | APGKGLEYIGIIDTDGSTYYADWAKGRFTISKDSTTVYLQ | |
| | MNSLRAEDTAVYYCARRLHGSSNGYYDDLWGQGTLVT | |
| | VSS | |
| H5 | QVQLVESGGGLVQPGGSLRLSCSVSGIDLSSNPMIWVRQ | 505 |
| | APGKGLQYIGIIDTDGSTYYADWAKGRFTISKDSTTVYLQ | |
| | INSPRAEDTAVYYCARRLHGSSNGYYDDLWGQGTLVTV | |
| | SS | |
| L2 | DGQMTQSPSSVSASVGDRVTITCQASQSIYSYLSWYQQK | 506 |
| | PGQRPKLLIYKASTLASGVPSRFSGSGSGTDFTLTISSVQP | |
| | EDAATYYCQQGYTVTNVDKNTFGGGTKVEVK | |
| L4 | DGQMTQSPSSVSASVGDRVTITCQASQSIYSYLSWYQQK | 507 |
| | PGQRPKLLIYKASTLASGVPSRFSGSGSGTDFTLTISSVQP | |
| | EDFATYYCQQGYTVTNVDKNTFGGGTKVEIK | |

TABLE X10
V_{H} and V_{L} Composition of Final Selected Constructs

| Construct | Humanized V_{H} and V_{L} composition |
|---|---|
| v37407 | H4-L4 |
| v37408 | H4-L2 |
| v37409 | H2-L4 |
| v37410 | H3-L4 |
| v37411 | H5-L4 |

The five selected humanized constructs (v37407, v37408, v37409, v37410, v37411) plus the parental rabbit chimeric construct (v35777) were expressed in 2.5 mL ExpiCHO cultures and purified by Protein A for further characterization. Production titers were significantly increased for the humanized variants compared to the parental chimeric. All humanized constructs expressed to high level and were easily purified by Protein A (mAb SelectSuRe) with high yield and purity (TABLE X11).

TABLE X11
Production of Parental Rabbit Chimeric and Humanized Constructs

| | | Culture | | | |
|---|---|---|---|---|---|
| | | volume | Titer | Yield | UPLC-SEC |
| Construct | Description | (mL) | (mg/L) | (mg) | % purity |
| v35777 | parental rabbit | 510 | 20.6 | 12.1 | 95.0 |
| | chimeric | | | | |
| v37407 | H4-L4 | 380 | 564.4 | 233.1 | >99 |
| v37408 | H4-L2 | 175 | 568.1 | 113.1 | >99 |
| v37409 | H2-L4 | 175 | 442.3 | 131.9 | >99 |
| v37410 | H3-L4 | 175 | 512.8 | 139.7 | >99 |
| v37411 | H5-L4 | 175 | 447.7 | 123.0 | >99 |

The purified rabbit chimeric and humanized constructs were assessed for binding to HEK293-6E cells transfected with human, cynomologus monkey, or mouse CLDN18.2 orthologs (TABLE X12) by flow cytometry. All constructs bound each ortholog-transfected cell with similar single-digit nanomolar or stronger EC₅₀ and similar Bmax to the parental rabbit chimeric construct, suggesting broad cross-reactivity (TABLE X13). Constructs were also assessed for binding to HEK293-6E cells transfected with human CLDN18.1. All constructs were negative for CLDN18.1 binding activity, showing selectivity for the CLDN18.2 splice variant.

TABLE X12
CLDN18.2 Ortholog and CLDN18.1 Sequences

| Name | Sequence | SEQ ID NO |
|---|---|---|
| Human | MAVTACQGLGFVVSLIGIAGIIAATCMDQWSTQDLYNNPV | 508 |
| CLDN18.2 | TAVFNYQGLWRSCVRESSGFTECRGYFTLLGLPAMLQAV | |
| | RALMIVGIVLGAIGLLVSIFALKCIRIGSMEDSAKANMTLTS | |
| | GIMFIVSGLCAIAGVSVFANMLVTNFWMSTANMYTGMGG | |
| | MVQTVQTRYTFGAALFVGWVAGGLTLIGGVMMCIACRG | |
| | LAPEETNYKAVSYHASGHSVAYKPGGFKASTGFGSNTKN | |
| | KKIYDGGARTEDEVQSYPSKHDYV | |
| Cynomolgus | MAVTACQGLGFVVSLIGIAGIIAATCMDQWSTQDLYNNPV | 509 |
| monkey | TAVFNYQGLWRSCVRESSGFTECRGYFTLLGLPAMLQAV | |
| CLDN18 | RALMIVGIVLGAIGLLVSIFALKCIRIGSMEDSAKANMTLTS | |
| | GIMFIVSGLCAIAGVSVFANMLVTNFWMSTANMYTGMGG | |
| | MVQTVQTRYTFGAALFVGWVAGGLTLIGGVMMCIACRG | |
| | LAPEETNYKAVSYHASGHSVAYKPGGFKASTGFGSNTKN | |
| | KKTYDGGAHTEDELQSYPSKHDYV | |
| Mouse | MSVTACQGLGFVVSLIGFAGIIAATCMDQWSTQDLYNNPV | 510 |
| CLDN18 | TAVFNYQGLWRSCVRESSGFTECRGYFTLLGLPAMLQAV | |
| A2.1 | RALMIVGIVLGVIGILVSIFALKCIRIGSMDDSAKAKMTLTS | |
| | GILFIISGICAIIGVSVFANMLVTNFWMSTANMYSGMGGM | |
| | GGMVQTVQTRYTFGAALFVGWVAGGLTLIGGVMMCIAC | |
| | RGLTPDDSNFKAVSYHASGQNVAYRPGGFKASTGFGSNTR | |
| | NKKIYDGGARTEDDEQSHPTKYDYV | |
| Human | MSTTTCQVVAFLLSILGLAGCIAATGMDMWSTQDLYDNP | 511 |
| CLDN18.1 | VTSVFQYEGLWRSCVRQSSGFTECRPYFTILGLPAMLQAV | |
| | RALMIVGIVLGAIGLLVSIFALKCIRIGSMEDSAKANMTLTS | |
| | GIMFIVSGLCAIAGVSVFANMLVTNFWMSTANMYTGMGG | |
| | MVQTVQTRYTFGAALFVGWVAGGLTLIGGVMMCIACRG | |
| | LAPEETNYKAVSYHASGHSVAYKPGGFKASTGFGSNTKN | |
| | KKIYDGGARTEDEVQSYPSKHDYV | |

TABLE X13
FACS Binding Fits (4 parameter logistic) to HEK293-6E
Transfected with Human, Cyno, or Mouse CLDN18.2 Orthologs

| | | Human | | Cyno | | Mouse | |
|---|---|---|---|---|---|---|---|
| | | CLDN18.2 | | CLDN18 | | CLDN18 2.1 | |
| | | EC₅₀ | | EC₅₀ | | EC₅₀ | |
| Construct | Description | (nM) | Bmax | (nM) | Bmax | (nM) | Bmax |
| v35777 | parental rabbit | 1.29 | 2071 | 0.48 | 1658 | 0.87 | 1640 |
| | chimeric | | | | | | |
| v37407 | H4-L4 | 1.54 | 2042 | 0.24 | 1410 | 0.80 | 1543 |
| v37408 | H4-L2 | 1.87 | 2187 | 0.36 | 1520 | 2.65 | 1922 |
| v37409 | H2-L4 | 1.22 | 1962 | 0.35 | 1503 | 0.89 | 1527 |
| v37410 | H3-L4 | 0.97 | 1912 | 0.31 | 1497 | 1.02 | 1690 |
| v37411 | H5-L4 | 1.05 | 2166 | 0.37 | 1731 | 1.03 | 1793 |

### Sequences

TABLE AA
Part 1

| SEQ ID | DESCRIPTION | SEQUENCE |
|---|---|---|
| SEQ ID NO: 1 | CRIS7 CD3 VL | DIQMTQSPSSLSASVGDRVTMTCSASSSVSYMNW |
| | | YQQKPGKAPKRWIYDSSKLASGVPARFSGSGSGT |
| | | DYTLTISSLQPEDFATYYCQQWSRNPPTFGGGTK |
| | | LQIT |
| SEQ ID NO: 2 | CRIS7 CD3 VH | QVQLVESGGGVVQPGRSLRLSCKASGYTFTRST |
| | | MHWVRQAPGQGLEWIGYINPSSAYTNYNQKFKD |
| | | RFTISADKSKSTAFLQMDSLRPEDTGVYFCARPQ |
| | | VHYDYNGFPYWGQGTPVTVSS |
| SEQ ID NO: 3 | Trastuzumab scFv | DIQMTQSPSSLSASVGDRVTITCRASQDVNTAVA |
| | | WYQQKPGKAPKLLIYSASFLYSGVPSRFSGSRSG |
| | | TDFTLTISSLQPEDFATYYCQQHYTTPPTFGQGTK |
| | | VEIKGGSGGGSGGGSGGGSGGGSGEVQLVESGG |
| | | GLVQPGGSLRLSCAASGFNIKDTYIHWVRQAPGK |
| | | GLEWVARIYPTNGYTRYADSVKGRFTISADTSKN |
| | | TAYLQMNSLRAEDTAVYYCSRWGGDGFYAMDY |
| | | WGQGTLVTVSS |
| SEQ ID NO: 4 | Chain A CH3 region | GQPREPQVYVYPPSRDELTKNQVSLTCLVKGFYP |
| | | SDIAVEWESNGQPENNYKTTPPVLDSDGSFALVS |
| | | KLTVDKSRWQQGNVFSCSVMHEALHNHYTQKS |
| | | LSLSPG |
| SEQ ID NO: 5 | Chain B CH3 region | GQPREPQVYVLPPSRDELTKNQVSLLCLVKGFYP |
| | | SDIAVEWESNGQPENNYLTWPPVLDSDGSFFLYS |
| | | KLTVDKSRWQQGNVFSCSVMHEALHNHYTQKS |
| | | LSLSPG |
| SEQ ID NO: 6 | CH2 region with | APEAAGGPSVFLFPPKPKDTLMISRTPEVTCVVVS |
| | L234A- | VSHEDPEVKFNWYVDGVEVHNAKTKPREEQYNS |
| | L235A_D265S | TYRVVSVLTVLHQDWLNGKEYKCKVSNKALPAP |
| | mutations | IEKTISKAK |
| SEQ ID NO: 7 | Wild-type PD-1 | NPPTFSPALLVVTEGDNATFTCSFSNTSESFVLNW |
| | | YRMSPSNQTDKLAAFPEDRSQPGQDCRFRVTQLP |
| | | NGRDFHMSVVRARRNDSGTYLCGAISLAPKAQIK |
| | | ESLRAELRVTE |
| SEQ ID NO: 8 | Wild-type PD-L1 | AFTVTVPKDLYVVEYGSNMTIECKFPVEKQLDLA |
| | | ALIVYWEMEDKNIIQFVHGEEDLKVQHSSYRQRA |
| | | RLLKDQLSLGNAALQITDVKLQDAGVYRCMISY |
| | | GGADYKRITVKVNA |
| SEQ ID NO: 9 | High affinity PD-1 | NPPTFSPALLVVTEGDNATFTCSFSNTSESFHVVW |
| | | HRESPSGQTDTLAAFPEDRSQPGQDARFRVTQLP |
| | | NGRDFHMSVVRARRNDSGTYVCGVISLAPKIQIK |
| | | ESLRAELRVTE |
| SEQ ID NO: 10 | High affinity PD-L1 | AFTVTVPKDLYVVEYGSNMTIECKFPVEKQLDLA |
| | | ALQVFWMMEDKNIIQFVHGEEDLKVQHSSYRQR |
| | | ARLLKDQLSLGNAALQITDVKLQDAGVYTCLIAY |
| | | KGADYKRITVKVNA |
| SEQ ID NO: 11 | WT CPS PD-1 | NPPTFSPALLVVTEGDNATFTCSFSNTSESFVLNW |
| | | YRMSPSNQTDKLAAFPEDRSQPGQDSRFRVTQLP |
| | | NGRDFHMSVVRARRNDSGTYLCGAISLAPKAQIK |
| | | ESLRAELRVTE |
| SEQ ID NO: 530 | Attenuated PD-1 | NPPTFSPALLVVTEGDNATFTCSFSNTSESFRLVW |
| | | HRESPGYETDTLASFPEDRSTPLPDARFRVTQLPN |
| | | GRDFHMSVVRARRNDSGTYVCGAIAFHPVIQIKE |
| | | SLRAELRVTE |
| SEQ ID NO: 531 | Attenuated PD-1 | NPPTFSPALLVVTEGDNATFTCSFSNTSESFRLVW |
| | | HRESPSYQTDTLAAFPEDRSQPGQDARFRVTQLP |
| | | NGRDFHMSVVRARRNDSGTYVCGAISLAPKIQIK |
| | | ESLRAELRVTE |
| SEQ ID NO: 12 | Wild-type CH3 | GQPREPQVYTLPPSRDELTKNQVSLTCLVKGFYP |
| | region | SDIAVEWESNGQPENNYKTTPPVLDSDGSFFLYS |
| | | KLTVDKSRWQQGNVFSCSVMHEALHNHYTQKS |
| | | LSLSPG |
| SEQ ID NO: 13 | Signal peptide | EFATMRPTWAWWLFLVLLLALWAPARG |
| SEQ ID NO: 14 | Human IgG1 Fc | APELLGGPSVFLFPPKPKDTLMISRTPEVTCVVVD |
| | sequence 231-447 | VSHEDPEVKFNWYVDGVEVHNAKTKPREEQYNS |
| | (EU-numbering) | TYRVVSVLTVLHQDWLNGKEYKCKVSNKALPAP |
| | | IEKTISKAKGQPREPQVYTLPPSRDELTKNQVSLT |
| | | CLVKGFYPSDIAVEWESNGQPENNYKTTPPVLDS |
| | | DGSFFLYSKLTVDKSRWQQGNVFSCSVMHEALH |
| | | NHYTQKSLSLSPGK |
| SEQ ID NO: 15 | Linker | EAAAKEAAAK |
| SEQ ID NO: 16 | Linker | EAAAK |
| SEQ ID NO: 17 | Linker | PPPP |
| SEQ ID NO: 18 | Linker | PPP |
| SEQ ID NO: 19 | Linker | GGGGS |
| SEQ ID NO: 20 | Linker | EAAAKEAAAKEAAAK |
| SEQ ID NO: 50 | Linker^{Fc} | EPKSCDKTHTCPPCP |
| SEQ ID NO: 21 | Atezolizumab VH | EVQLVESGGGLVQPGGSLRLSCAASGFTFSDSWI |
| | | HWVRQAPGKGLEWVAWISPYGGSTYYADSVKG |
| | | RFTISADTSKNTAYLQMNSLRAEDTAVYYCARR |
| | | HWPGGFDYWGQGTLVTVSS |
| SEQ ID NO: 22 | Atezolizumab VL | DIQMTQSPSSLSASVGDRVTITCRASQDVSTAVA |
| | | WYQQKPGKAPKLLIYSASFLYSGVPSRFSGSGSG |
| | | TDFTLTISSLQPEDFATYYCQQYLYHPATFGQGT |
| | | KVEIK |
| SEQ ID NO: 23 | Nivolumab VH | QVQLVESGGGVVQPGRSLRLDCKASGITFSNSGM |
| | | HWVRQAPGKGLEWVAVIWYDGSKRYYADSVK |
| | | GRFTISRDNSKNTLFLQMNSLRAEDTAVYYCATN |
| | | DDYWGQGTLVTVSS |
| SEQ ID NO: 24 | Nivolumab VL | EIVLTQSPATLSLSPGERATLSCRASQSVSSYLAW |
| | | YQQKPGQAPRLLIYDASNRATGIPARFSGSGSGT |
| | | DFTLTISSLEPEDFAVYYCQQSSNWPRTFGQGTK |
| | | VEIK |
| SEQ ID NO: 25 | Palivizumab VH | QVTLRESGPALVKPTQTLTLTCTFSGFSLSTSGMS |
| | | VGWIRQPPGKALEWLADIWWDDKKDYNPSLKS |
| | | RLTISKDTSKNQVVLKVTNMDPADTATYYCARS |
| | | MITNWYFDVWGAGTTVTVS |
| SEQ ID NO: 26 | Palivizumab VL | DIQMTQSPSTLSASVGDRVTITCKSQLSVGYMHW |
| | | YQQKPGKAPKLLIYDTSKLASGVPSRFSGSGSGTE |
| | | FTLTISSLQPDDFATYYCFQGSGYPFTFGGGTKLEI |
| | | K |

TABLE AA
Part 2
Clone and Variant sequences*

| | | | | | |
|---|---|---|---|---|---|
| 12985 | Full | DIQMTQSPSSLSASVGDRVTMTCSASS | | | 102 |
| | | SVSYMNWYQQKPGKAPKRWIYDSSKL | | | |
| | | ASGVPARFSGSGSGTDYTLTISSLQPED | | | |
| | | FATYYCQQWSRNPPTFGGGTKLQITRT | | | |
| | | VAAPSVFIFPPSDEQLKSGTASVVCLLN | | | |
| | | NFYPREAKVQWKVDNALQSGNSQESV | | | |
| | | TEQDSKDSTYSLSSTLTLSKADYEKHK | | | |
| | | VYACEVTHQGLSSPVTKSFNRGEC | | | |
| | VL | DIQMTQSPSSLSASVGDRVTMTCSASS | hCris7 | CD3 | 103 |
| | | SVSYMNWYQQKPGKAPKRWIYDSSKL | | | |
| | | ASGVPARFSGSGSGTDYTLTISSLQPED | | | |
| | | FATYYCQQWSRNPPTFGGGTKLQIT | | | |
| | LCDR1 | SASSSVSYMN | | | 211 |
| | LCDR2 | DSSKLAS | | | 105 |
| | LCDR3 | QQWSRNPPT | | | 106 |
| 21490 | Full | DIQMTQSPSSLSASVGDRVTITCRASQD | | | 119 |
| | | VNTAVAWYQQKPGKAPKLLIYSASFL | | | |
| | | YSGVPSRFSGSRSGTDFTLTISSLQPEDF | | | |
| | | ATYYCQQHYTTPPTFGQGTKVEIKGGS | | | |
| | | GGGSGGGSGGGSGGGSGEVQLVESGG | | | |
| | | GLVQPGGSLRLSCAASGFNIKDTYIHW | | | |
| | | VRQAPGKGLEWVARIYPTNGYTRYAD | | | |
| | | SVKGRFTISADTSKNTAYLQMNSLRAE | | | |
| | | DTAVYYCSRWGGDGFYAMDYWGQG | | | |
| | | TLVTVSSEPKSSDKTHTCPPCPAPEAAG | | | |
| | | GPSVFLFPPKPKDTLMISRTPEVTCVVV | | | |
| | | SVSHEDPEVKFNWYVDGVEVHNAKTK | | | |
| | | PREEQYNSTYRVVSVLTVLHQDWLNG | | | |
| | | KEYKCKVSNKALPAPIEKTISKAKGQP | | | |
| | | REPQVYVLPPSRDELTKNQVSLLCLVK | | | |
| | | GFYPSDIAVEWESNGQPENNYLTWPPV | | | |
| | | LDSDGSFFLYSKLTVDKSRWQQGNVFS | | | |
| | | CSVMHEALHNHYTQKSLSLSPG | | | |
| | VH | EVQLVESGGGLVQPGGSLRLSCAASGF | Trastuzumab | HER2 | 120 |
| | | NIKDTYIHWVRQAPGKGLEWVARIYPT | | | |
| | | NGYTRYADSVKGRFTISADTSKNTAYL | | | |
| | | QMNSLRAEDTAVYYCSRWGGDGFYA | | | |
| | | MDYWGQGTLVTVSS | | | |
| | HCDR1 | DTYIH | | | 121 |
| | HCDR2 | RIYPTNGYTRYADSVKG | | | 122 |
| | HCDR3 | WGGDGFYAMDY | | | 123 |
| | VL | DIQMTQSPSSLSASVGDRVTITCRASQD | Trastuzumab | HER2 | 124 |
| | | VNTAVAWYQQKPGKAPKLLIYSASFL | | | |
| | | YSGVPSRFSGSRSGTDFTLTISSLQPEDF | | | |
| | | ATYYCQQHYTTPPTFGQGTKVEIK | | | |
| | LCDR1 | RASQDVNTAVA | | | 125 |
| | LCDR2 | SASFLYS | | | 126 |
| | LCDR3 | QQHYTTPPT | | | 127 |
| 22080 | Full | NPPTFSPALLVVTEGDNATFTCSFSNTS | | | 141 |
| | | ESFHVVWHRESPSGQTDTLAAFPEDRS | | | |
| | | QPGQDARFRVTQLPNGRDFHMSVVRA | | | |
| | | RRNDSGTYVCGVISLAPKIQIKESLRAE | | | |
| | | LRVTEEAAAKEAAAKQVQLVESGGGV | | | |
| | | VQPGRSLRLSCKASGYTFTRSTMHWV | | | |
| | | RQAPGQGLEWIGYINPSSAYTNYNQKF | | | |
| | | KDRFTISADKSKSTAFLQMDSLRPEDT | | | |
| | | GVYFCARPQVHYDYNGFPYWGQGTP | | | |
| | | VTVSSASTKGPSVFPLAPSSKSTSGGTA | | | |
| | | ALGCLVKDYFPEPVTVSWNSGALTSG | | | |
| | | VHTFPAVLQSSGLYSLSSVVTVPSSSLG | | | |
| | | TQTYICNVNHKPSNTKVDKKVEPKSCD | | | |
| | | KTHTCPPCPAPEAAGGPSVFLFPPKPKD | | | |
| | | TLMISRTPEVTCVVVSVSHEDPEVKFN | | | |
| | | WYVDGVEVHNAKTKPREEQYNSTYR | | | |
| | | VVSVLTVLHQDWLNGKEYKCKVSNK | | | |
| | | ALPAPIEKTISKAKGQPREPQVYVYPPS | | | |
| | | RDELTKNQVSLTCLVKGFYPSDIAVEW | | | |
| | | ESNGQPENNYKTTPPVLDSDGSFALVS | | | |
| | | KLTVDKSRWQQGNVFSCSVMHEALH | | | |
| | | NHYTQKSLSLSPG | | | |
| | PD-1 | NPPTFSPALLVVTEGDNATFTCSFSNTS | PD-1 | PD-L1 | 116 |
| | domain | ESFHVVWHRESPSGQTDTLAAFPEDRS | 33-146 | | |
| | | QPGQDARFRVTQLPNGRDFHMSVVRA | | | |
| | | RRNDSGTYVCGVISLAPKIQIKESLRAE | | | |
| | | LRVTE | | | |
| | Linker | EAAAKEAAAK | | | 15 |
| | VH | QVQLVESGGGVVQPGRSLRLSCKASG | hCris7 | CD3 | 2 |
| | | YTFTRSTMHWVRQAPGQGLEWIGYIN | | | |
| | | PSSAYTNYNQKFKDRFTISADKSKSTA | | | |
| | | FLQMDSLRPEDTGVYFCARPQVHYDY | | | |
| | | NGFPYWGQGTPVTVSS | | | |
| | HCDR1 | RSTMH | | | 109 |
| | HCDR2 | YINPSSAYTNYNQKFKD | | | 110 |
| | HCDR3 | PQVHYDYNGFPY | | | 111 |
| 23734 | Full | NPPTFSPALLVVTEGDNATFTCSFSN | | | 190 |
| | | TSESFVLNWYRMSPSNQTDALAAFP | | | |
| | | EDRSQPGQDSRFRVTQLPNGRDFHM | | | |
| | | SVVRARRNDSGTYLCGAASLAPKAQ | | | |
| | | IKESLRAELRVTEEAAAKEAAAKQV | | | |
| | | QLVESGGGVVQPGRSLRLSCKASGY | | | |
| | | TFTRSTMHWVRQAPGQGLEWIGYIN | | | |
| | | PSSAYTNYNQKFKDRFTISADKSKST | | | |
| | | AFLQMDSLRPEDTGVYFCARPQVHY | | | |
| | | DYNGFPYWGQGTPVTVSSASTKGPS | | | |
| | | VFPLAPSSKSTSGGTAALGCLVKDYF | | | |
| | | PEPVTVSWNSGALTSGVHTFPAVLQS | | | |
| | | SGLYSLSSVVTVPSSSLGTQTYICNV | | | |
| | | NHKPSNTKVDKKVEPKSCDKTHTCP | | | |
| | | PCPAPEAAGGPSVFLFPPKPKDTLMIS | | | |
| | | RTPEVTCVVVSVSHEDPEVKFNWYV | | | |
| | | DGVEVHNAKTKPREEQYNSTYRVVS | | | |
| | | VLTVLHQDWLNGKEYKCKVSNKAL | | | |
| | | PAPIEKTISKAKGQPREPQVYVYPPSR | | | |
| | | DELTKNQVSLTCLVKGFYPSDIAVE | | | |
| | | WESNGQPENNYKTTPPVLDSDGSFA | | | |
| | | LVSKLTVDKSRWQQGNVFSCSVMH | | | |
| | | EALHNHYTQKSLSLSPG | | | |
| | PD-1 | NPPTFSPALLVVTEGDNATFTCSFSN | PD-1 | PD-L1 | 191 |
| | domain | TSESFVLNWYRMSPSNQTDALAAFP | 33-146 | | |
| | | EDRSQPGQDSRFRVTQLPNGRDFHM | | | |
| | | SVVRARRNDSGTYLCGAASLAPKAQ | | | |
| | | IKESLRAELRVTE | | | |
| | Linker | EAAAKEAAAK | | | 15 |
| | VH | QVQLVESGGGVVQPGRSLRLSCKAS | hCris7 | CD3 | 2 |
| | | GYTFTRSTMHWVRQAPGQGLEWIG | | | |
| | | YINPSSAYTNYNQKFKDRFTISADKS | | | |
| | | KSTAFLQMDSLRPEDTGVYFCARPQ | | | |
| | | VHYDYNGFPYWGQGTPVTVSS | | | |
| | HCDR1 | RSTMH | | | 109 |
| | HCDR2 | YINPSSAYTNYNQKFKD | | | 110 |
| | HCDR3 | PQVHYDYNGFPY | | | 111 |
| | LCDR2 | RSYQRPS | | | 199 |
| | LCDR3 | ATWDDSLDGWV | | | 200 |

TABLE AA
Part 3: Clone and Variant sequences

| | | | | | SEQ ID |
|---|---|---|---|---|---|
| Clone ID | Region | Amino Acid Sequence | Name | | No: |
| 12932 | Full | QVQLVQSGAEVKKPGASVKVSCKASGYSFTGY | | | 250 |
| | | TMNWVRQAPGQGLEWMGLITPYNGASSYNQKF | | | |
| | | RGKATMTVDTSTSTVYMELSSLRSEDTAVYYCA | | | |
| | | RGGYDGRGFDYWGQGTLVTVSSASTKGPSVFPL | | | |
| | | APSSKSTSGGTAALGCLVKDYFPEPVTVSWNSG | | | |
| | | ALTSGVHTFPAVLQSSGLYSLSSVVTVPSSSLGT | | | |
| | | QTYICNVNHKPSNTKVDKKVEPKSCDKTHTCPP | | | |
| | | CPAPEAAGGPSVFLFPPKPKDTLMISRTPEVTCV | | | |
| | | VVSVSHEDPEVKFNWYVDGVEVHNAKTKPREE | | | |
| | | QYNSTYRVVSVLTVLHQDWLNGKEYKCKVSNK | | | |
| | | ALPAPIEKTISKAKGQPREPQVYVLPPSRDELTK | | | |
| | | NQVSLLCLVKGFYPSDIAVEWESNGQPENNYLT | | | |
| | | WPPVLDSDGSFFLYSKLTVDKSRWQQGNVFSCS | | | |
| | | VMHEALHNHYTQKSLSLSPG | | | |
| | VH | QVQLVQSGAEVKKPGASVKVSCKASGYSFTGY | huRG7787 | MSLN | 297 |
| | | TMNWVRQAPGQGLEWMGLITPYNGASSYNQKF | | | |
| | | RGKATMTVDTSTSTVYMELSSLRSEDTAVYYCA | | | |
| | | RGGYDGRGFDYWGQGTLVTVSS | | | |
| | HCDR1 | GYTMN | | | 286 |
| | HCDR2 | LITPYNGASSYNQKFRG | | | 288 |
| | HCDR3 | GGYDGRGFDY | | | 285 |
| 12933 | Full | DIQMTQSPSSLSASVGDRVTITCSASSSVSYMHW | | | 251 |
| | | YQQKSGKAPKLLIYDTSKLASGVPSRFSGSGSGT | | | |
| | | DFTLTISSLQPEDFATYYCQQWSGYPLTFGQGTK | | | |
| | | LEIKRTVAAPSVFIFPPSDEQLKSGTASVVCLLNN | | | |
| | | FYPREAKVQWKVDNALQSGNSQESVTEQDSKD | | | |
| | | STYSLSSTLTLSKADYEKHKVYACEVTHQGLSSP | | | |
| | | VTKSFNRGEC | | | |
| | VL | DIQMTQSPSSLSASVGDRVTITCSASSSVSYMHW | huRG7787 | MSLN | 276 |
| | | YQQKSGKAPKLLIYDTSKLASGVPSRFSGSGSGT | | | |
| | | DFTLTISSLQPEDFATYYCQQWSGYPLTFGQGTK | | | |
| | | LEIK | | | |
| | LCDR1 | SASSSVSYMH | | | 300 |
| | LCDR2 | DTSKLAS | | | 279 |
| | LCDR3 | QQWSGYPLT | | | 294 |
| 12966 | Full | QVQLVQSGAEVKKPGASVKVSCKASGYSFTGY | | | 252 |
| | | TMNWVRQAPGQGLEWMGLITPYNGASSYNQKF | | | |
| | | RGKATMTVDTSTSTVYMELSSLRSEDTAVYYCA | | | |
| | | RGGYDGRGFDYWGQGTLVTVSSASTKGPSVFPL | | | |
| | | APSSKSTSGGTAALGCLVKDYFPEPVTVSWNSG | | | |
| | | ALTSGVHTFPAVLQSSGLYSLSSVVTVPSSSLGT | | | |
| | | QTYICNVNHKPSNTKVDKKVEPKSCDKTHTCPP | | | |
| | | CPAPEAAGGPSVFLFPPKPKDTLMISRTPEVTCV | | | |
| | | VVSVSHEDPEVKFNWYVDGVEVHNAKTKPREE | | | |
| | | QYNSTYRVVSVLTVLHQDWLNGKEYKCKVSNK | | | |
| | | ALPAPIEKTISKAKGQPREPQVYVYPPSRDELTK | | | |
| | | NQVSLTCLVKGFYPSDIAVEWESNGQPENNYKT | | | |
| | | TPPVLDSDGSFALVSKLTVDKSRWQQGNVFSCS | | | |
| | | VMHEALHNHYTQKSLSLSPG | | | |
| | VH | QVQLVQSGAEVKKPGASVKVSCKASGYSFTGY | huRG7787 | MSLN | 297 |
| | | TMNWVRQAPGQGLEWMGLITPYNGASSYNQKF | | | |
| | | RGKATMTVDTSTSTVYMELSSLRSEDTAVYYCA | | | |
| | | RGGYDGRGFDYWGQGTLVTVSS | | | |
| | HCDR1 | GYTMN | | | 286 |
| | HCDR2 | LITPYNGASSYNQKFRG | | | 288 |
| | HCDR3 | GGYDGRGFDY | | | 285 |
| 12985 | Full | DIQMTQSPSSLSASVGDRVTMTCSASSSVSYMN | | | 253 |
| | | WYQQKPGKAPKRWIYDSSKLASGVPARFSGSGS | | | |
| | | GTDYTLTISSLQPEDFATYYCQQWSRNPPTFGGG | | | |
| | | TKLQITRTVAAPSVFIFPPSDEQLKSGTASVVCLL | | | |
| | | NNFYPREAKVQWKVDNALQSGNSQESVTEQDS | | | |
| | | KDSTYSLSSTLTLSKADYEKHKVYACEVTHQGL | | | |
| | | SSPVTKSFNRGEC | | | |
| | VL | DIQMTQSPSSLSASVGDRVTMTCSASSSVSYMN | hCris7 | CD3 | 278 |
| | | WYQQKPGKAPKRWIYDSSKLASGVPARFSGSGS | | | |
| | | GTDYTLTISSLQPEDFATYYCQQWSRNPPTFGGG | | | |
| | | TKLQIT | | | |
| | LCDR1 | SASSSVSYMN | | | 211 |
| | LCDR2 | DSSKLAS | | | 105 |
| | LCDR3 | QQWSRNPPT | | | 106 |
| 16412 | Full | NFMLTQPHSVSESPGKTVTISCKRNTGNIGSNYV | | | 254 |
| | | NWYQQHEGSSPTTIIYRNDKRPDGVSDRFSGSID | | | |
| | | RSSKSASLTISNLKTEDEADYFCQSYSSGFIFGGG | | | |
| | | TKLTVLGQPKAAPSVTLFPPSSEELQANKATLVC | | | |
| | | LISDFYPGAVTVAWKADSSPVKAGVETTTPSKQ | | | |
| | | SNNKYAASSYLSLTPEQWKSHRSYSCQVTHEGS | | | |
| | | TVEKTVAPTECS | | | |
| | VL | NFMLTQPHSVSESPGKTVTISCKRNTGNIGSNYV | C3E-1 | CD3 | 289 |
| | | NWYQQHEGSSPTTIIYRNDKRPDGVSDRFSGSID | | | |
| | | RSSKSASLTISNLKTEDEADYFCQSYSSGFIFGGG | | | |
| | | TKLTVL | | | |
| | LCDR1 | KRNTGNIGSNYVN | | | 287 |
| | LCDR2 | RNDKRPD | | | 298 |
| | LCDR3 | QSYSSGFI | | | 295 |
| 22080 | Full | NPPTFSPALLVVTEGDNATFTCSFSNTSESFHVV | | | 255 |
| | | WHRESPSGQTDTLAAFPEDRSQPGQDARFRVTQ | | | |
| | | LPNGRDFHMSVVRARRNDSGTYVCGVISLAPKI | | | |
| | | QIKESLRAELRVTEEAAAKEAAAKQVQLVESGG | | | |
| | | GVVQPGRSLRLSCKASGYTFTRSTMHWVRQAP | | | |
| | | GQGLEWIGYINPSSAYTNYNQKFKDRFTISADKS | | | |
| | | KSTAFLQMDSLRPEDTGVYFCARPQVHYDYNGF | | | |
| | | PYWGQGTPVTVSSASTKGPSVFPLAPSSKSTSGG | | | |
| | | TAALGCLVKDYFPEPVTVSWNSGALTSGVHTFP | | | |
| | | AVLQSSGLYSLSSVVTVPSSSLGTQTYICNVNHK | | | |
| | | PSNTKVDKKVEPKSCDKTHTCPPCPAPEAAGGP | | | |
| | | SVFLFPPKPKDTLMISRTPEVTCVVVSVSHEDPE | | | |
| | | VKFNWYVDGVEVHNAKTKPREEQYNSTYRVVS | | | |
| | | VLTVLHQDWLNGKEYKCKVSNKALPAPIEKTIS | | | |
| | | KAKGQPREPQVYVYPPSRDELTKNQVSLTCLVK | | | |
| | | GFYPSDIAVEWESNGQPENNYKTTPPVLDSDGSF | | | |
| | | ALVSKLTVDKSRWQQGNVFSCSVMHEALHNHY | | | |
| | | TQKSLSLSPG | | | |
| | PD-1 | NPPTFSPALLVVTEGDNATFTCSFSNTSESFHVV | PD-1 33- | PD-L1 | 290 |
| | domain | WHRESPSGQTDTLAAFPEDRSQPGQDARFRVTQ | 146 | | |
| | | LPNGRDFHMSVVRARRNDSGTYVCGVISLAPKI | | | |
| | | QIKESLRAELRVTE | | | |
| | Linker | EAAAKEAAAK | | | 15 |
| | VH | QVQLVESGGGVVQPGRSLRLSCKASGYTFTRST | hCris7 | CD3 | 2 |
| | | MHWVRQAPGQGLEWIGYINPSSAYTNYNQKFK | | | |
| | | DRFTISADKSKSTAFLQMDSLRPEDTGVYFCARP | | | |
| | | QVHYDYNGFPYWGQGTPVTVSS | | | |
| | HCDR1 | RSTMH | | | 109 |
| | HCDR2 | YINPSSAYTNYNQKFKD | | | 110 |
| | HCDR3 | PQVHYDYNGFPY | | | 111 |
| 23270 | Full | NPPTFSPALLVVTEGDNATFTCSFSNTSESFHVV | | | 256 |
| | | WHRESPSGQTDTLAAFPEDRSQPGQDARFRVTQ | | | |
| | | LPNGRDFHMSVVRARRNDSGTYVCGVISLAPKI | | | |
| | | QIKESLRAELRVTEAAEPKSSDKTHTCPPCPAPE | | | |
| | | AAGGPSVFLFPPKPKDTLMISRTPEVTCVVVSVS | | | |
| | | HEDPEVKFNWYVDGVEVHNAKTKPREEQYNST | | | |
| | | YRVVSVLTVLHQDWLNGKEYKCKVSNKALPAP | | | |
| | | IEKTISKAKGQPREPQVYVLPPSRDELTKNQVSL | | | |
| | | LCLVKGFYPSDIAVEWESNGQPENNYLTWPPVL | | | |
| | | DSDGSFFLYSKLTVDKSRWQQGNVFSCSVMHE | | | |
| | | ALHNHYTQKSLSLSPG | | | |
| | PD-1 | NPPTFSPALLVVTEGDNATFTCSFSNTSESFHVV | PD-1 33- | PD-L1 | 290 |
| | | WHRESPSGQTDTLAAFPEDRSQPGQDARFRVTQ | 146 | | |
| | domain | LPNGRDFHMSVVRARRNDSGTYVCGVISLAPKI | | | |
| | | QIKESLRAELRVTE | | | |
| | Linker | AA | | | 274 |
| 23570 | Full | DIQMTQSPSSLSASVGDRVTITCSASSSVSYMHW | | | 257 |
| | | YQQKSGKAPKLLIYDTSKLASGVPSRFSGSGSGT | | | |
| | | DFTLTISSLQPEDFATYYCQQWSKHPLTFGQGTK | | | |
| | | LEIKRTVAAPSVFIFPPSDEQLKSGTASVVCLLNN | | | |
| | | FYPREAKVQWKVDNALQSGNSQESVTEQDSKD | | | |
| | | STYSLSSTLTLSKADYEKHKVYACEVTHQGLSSP | | | |
| | | VTKSFNRGEC | | | |
| | VL | DIQMTQSPSSLSASVGDRVTITCSASSSVSYMHW | huRG7787 | MSLN | 277 |
| | | YQQKSGKAPKLLIYDTSKLASGVPSRFSGSGSGT | | | |
| | | DFTLTISSLQPEDFATYYCQQWSKHPLTFGQGTK | | | |
| | | LEIK | | | |
| | LCDR1 | SASSSVSYMH | | | 300 |
| | LCDR2 | DTSKLAS | | | 279 |
| | LCDR3 | QQWSKHPLT | | | 294 |
| 23734 | Full | NPPTFSPALLVVTEGDNATFTCSFSNTSESFVLN | | | 258 |
| | | WYRMSPSNQTDALAAFPEDRSQPGQDSRFRVTQ | | | |
| | | LPNGRDFHMSVVRARRNDSGTYLCGAASLAPK | | | |
| | | AQIKESLRAELRVTEEAAAKEAAAKQVQLVESG | | | |
| | | GGVVQPGRSLRLSCKASGYTFTRSTMHWVRQA | | | |
| | | PGQGLEWIGYINPSSAYTNYNQKFKDRFTISADK | | | |
| | | SKSTAFLQMDSLRPEDTGVYFCARPQVHYDYNG | | | |
| | | FPYWGQGTPVTVSSASTKGPSVFPLAPSSKSTSG | | | |
| | | GTAALGCLVKDYFPEPVTVSWNSGALTSGVHTF | | | |
| | | PAVLQSSGLYSLSSVVTVPSSSLGTQTYICNVNH | | | |
| | | KPSNTKVDKKVEPKSCDKTHTCPPCPAPEAAGG | | | |
| | | PSVFLFPPKPKDTLMISRTPEVTCVVVSVSHEDPE | | | |
| | | VKFNWYVDGVEVHNAKTKPREEQYNSTYRVVS | | | |
| | | VLTVLHQDWLNGKEYKCKVSNKALPAPIEKTIS | | | |
| | | KAKGQPREPQVYVYPPSRDELTKNQVSLTCLVK | | | |
| | | GFYPSDIAVEWESNGQPENNYKTTPPVLDSDGSF | | | |
| | | ALVSKLTVDKSRWQQGNVFSCSVMHEALHNHY | | | |
| | | TQKSLSLSPG | | | |
| | PD-1 | NPPTFSPALLVVTEGDNATFTCSFSNTSESFVLN | PD-1 33- | PD-L1 | 292 |
| | domain | WYRMSPSNQTDALAAFPEDRSQPGQDSRFRVTQ | 146 | | |
| | | LPNGRDFHMSVVRARRNDSGTYLCGAASLAPK | | | |
| | | AQIKESLRAELRVTE | | | |
| | Linker | EAAAKEAAAK | | | 15 |
| | VH | QVQLVESGGGVVQPGRSLRLSCKASGYTFTRST | hCris7 | CD3 | 2 |
| | | MHWVRQAPGQGLEWIGYINPSSAYTNYNQKFK | | | |
| | | DRFTISADKSKSTAFLQMDSLRPEDTGVYFCARP | | | |
| | | QVHYDYNGFPYWGQGTPVTVSS | | | |
| | HCDR1 | RSTMH | | | 109 |
| | HCDR2 | YINPSSAYTNYNQKFKD | | | 110 |
| | HCDR3 | PQVHYDYNGFPY | | | 111 |
| 23867 | Full | DIQMTQSPSSLSASVGDRVTITCSASSSVSYMHW | | | 259 |
| | | YQQKSGKAPKLLIYDTSKLASGVPSRFSGSGSGT | | | |
| | | DFTLTISSLQPEDFATYYCQQWSGYPLTFGQGTK | | | |
| | | LEIKGGGGSGGGGSGGGGSGGGGSQVQLVQSG | | | |
| | | AEVKKPGASVKVSCKASGYSFTGYTMNWVRQA | | | |
| | | PGQGLEWMGLITPYNGASSYNQKFRGKATMTV | | | |
| | | DTSTSTVYMELSSLRSEDTAVYYCARGGYDGRG | | | |
| | | FDYWGQGTLVTVSSAAEPKSSDKTHTCPPCPAP | | | |
| | | EAAGGPSVFLFPPKPKDTLMISRTPEVTCVVVSV | | | |
| | | SHEDPEVKFNWYVDGVEVHNAKTKPREEQYNS | | | |
| | | TYRVVSVLTVLHQDWLNGKEYKCKVSNKALPA | | | |
| | | PIEKTISKAKGQPREPQVYVLPPSRDELTKNQVS | | | |
| | | LLCLVKGFYPSDIAVEWESNGQPENNYLTWPPV | | | |
| | | LDSDGSFFLYSKLTVDKSRWQQGNVFSCSVMHE | | | |
| | | ALHNHYTQKSLSLSPG | | | |
| | VL | DIQMTQSPSSLSASVGDRVTITCSASSSVSYMHW | huRG7787 | MSLN | 276 |
| | | YQQKSGKAPKLLIYDTSKLASGVPSRFSGSGSGT | | | |
| | | DFTLTISSLQPEDFATYYCQQWSGYPLTFGQGTK | | | |
| | | LEIK | | | |
| | VL-VH | GGGGSGGGGSGGGGSGGGGS | | | 284 |
| | linker | | | | |
| | VH | QVQLVQSGAEVKKPGASVKVSCKASGYSFTGY | huRG7787 | MSLN | 297 |
| | | TMNWVRQAPGQGLEWMGLITPYNGASSYNQKF | | | |
| | | RGKATMTVDTSTSTVYMELSSLRSEDTAVYYCA | | | |
| | | RGGYDGRGFDYWGQGTLVTVSS | | | |
| | LCDR1 | SASSSVSYMH | | | 300 |
| | LCDR2 | DTSKLAS | | | 279 |
| | LCDR3 | QQWSGYPLT | | | 294 |
| | HCDR1 | GYTMN | | | 286 |
| | HCDR2 | LITPYNGASSYNQKFRG | | | 288 |
| | HCDR3 | GGYDGRGFDY | | | 285 |
| | Linker | AA | | | 274 |
| 25095 | Full | EVQLVESGGGLVQPGGSLRLSCAASGVTFNYYG | | | 260 |
| | | MSWIRQAPGKGLEWVASITSSGGRIYYPDSVKG | | | |
| | | RFTISRENTQKTLYLQMNSLRAEDTAVYYCTLD | | | |
| | | GRDGWVAYWGQGTLVTVSSGGGGSGGGGSGG | | | |
| | | GGSGGGGSNFMLTQPHSVSESPGKTVTISCKRNT | | | |
| | | GNIGSNYVNWYQQHEGSSPTTIIYRNDKRPDGV | | | |
| | | SDRFSGSIDRSSKSASLTISNLKTEDEADYFCQSY | | | |
| | | SSGFIFGGGTKLTVLAAEPKSSDKTHTCPPCPAPE | | | |
| | | AAGGPSVFLFPPKPKDTLMISRTPEVTCVVVSVS | | | |
| | | HEDPEVKFNWYVDGVEVHNAKTKPREEQYNST | | | |
| | | YRVVSVLTVLHQDWLNGKEYKCKVSNKALPAP | | | |
| | | IEKTISKAKGQPREPQVYVYPPSRDELTKNQVSL | | | |
| | | TCLVKGFYPSDIAVEWESNGQPENNYKTTPPVL | | | |
| | | DSDGSFALVSKLTVDKSRWQQGNVFSCSVMHE | | | |
| | | ALHNHYTQKSLSLSPG | | | |
| | VH | EVQLVESGGGLVQPGGSLRLSCAASGVTFNYYG | C3E-x | CD3 | 281 |
| | | MSWIRQAPGKGLEWVASITSSGGRIYYPDSVKG | | | |
| | | RFTISRENTQKTLYLQMNSLRAEDTAVYYCTLD | | | |
| | | GRDGWVAYWGQGTLVTVSS | | | |
| | VL-VH | GGGGSGGGGSGGGGSGGGGS | | | 284 |
| | linker | | | | |
| | VL | NFMLTQPHSVSESPGKTVTISCKRNTGNIGSNYV | C3E-1 | CD3 | 289 |
| | | NWYQQHEGSSPTTIIYRNDKRPDGVSDRFSGSID | | | |
| | | RSSKSASLTISNLKTEDEADYFCQSYSSGFIFGGG | | | |
| | | TKLTVL | | | |
| | HCDR1 | YYGMS | | | 303 |
| | HCDR2 | SITSSGGRIYYPDSVKG | | | 301 |
| | HCDR3 | DGRDGWVAY | | | 275 |
| | LCDR1 | KRNTGNIGSNYVN | | | 287 |
| | LCDR2 | RNDKRPD | | | 298 |
| | LCDR3 | QSYSSGFI | | | 295 |
| | Linker | AA | | | 274 |
| 29207 | Full | NPPTFSPALLVVTEGDNATFTCSFSNTSESFHVV | | | 261 |
| | | WHRESPSGQTDTLAAFPEDRSQPGQDARFRVTQ | | | |
| | | LPNGRDFHMSVVRARRNDSGTYVCGVISLAPKI | | | |
| | | QIKESLRAELRVTEEAAAKEAAAKEVQLVESGG | | | |
| | | GLVQPGGSLRLSCAASGVTFNYYGMSWIRQAPG | | | |
| | | KGLEWVASITSSGGRIYYPDSVKGRFTISRENTQ | | | |
| | | KTLYLQMNSLRAEDTAVYYCTLDGRDGWVAY | | | |
| | | WGQGTLVTVSSASTKGPSVFPLAPSSKSTSGGTA | | | |
| | | ALGCLVKDYFPEPVTVSWNSGALTSGVHTFPAV | | | |
| | | LQSSGLYSLSSVVTVPSSSLGTQTYICNVNHKPS | | | |
| | | NTKVDKKVEPKSCDKTHTCPPCPAPEAAGGPSV | | | |
| | | FLFPPKPKDTLMISRTPEVTCVVVSVSHEDPEVK | | | |
| | | FNWYVDGVEVHNAKTKPREEQYNSTYRVVSVL | | | |
| | | TVLHQDWLNGKEYKCKVSNKALPAPIEKTISKA | | | |
| | | KGQPREPQVYVYPPSRDELTKNQVSLTCLVKGF | | | |
| | | YPSDIAVEWESNGQPENNYKTTPPVLDSDGSFAL | | | |
| | | VSKLTVDKSRWQQGNVFSCSVMHEALHNHYTQ | | | |
| | | KSLSLSPG | | | |
| | PD-1 | NPPTFSPALLVVTEGDNATFTCSFSNTSESFHVV | PD-1 33- | PD-L1 | 290 |
| | domain | WHRESPSGQTDTLAAFPEDRSQPGQDARFRVTQ | 146 | | |
| | | LPNGRDFHMSVVRARRNDSGTYVCGVISLAPKI | | | |
| | | QIKESLRAELRVTE | | | |
| | Linker | EAAAKEAAAK | | | 15 |
| | VH | EVQLVESGGGLVQPGGSLRLSCAASGVTFNYYG | C3E-4 | CD3 | 281 |
| | | MSWIRQAPGKGLEWVASITSSGGRIYYPDSVKG | | | |
| | | RFTISRENTQKTLYLQMNSLRAEDTAVYYCTLD | | | |
| | | GRDGWVAYWGQGTLVTVSS | | | |
| | HCDR1 | YYGMS | | | 303 |
| | HCDR2 | SITSSGGRIYYPDSVKG | | | 301 |
| | HCDR3 | DGRDGWVAY | | | 275 |
| 29208 | Full | NPPTFSPALLVVTEGDNATFTCSFSNTSESFHVV | | | 262 |
| | | WHRESPSGQTDTLAAFPEDRSQPGQDARFRVTQ | | | |
| | | LPNGRDFHMSVVRARRNDSGTYVCGVISLAPKI | | | |
| | | QIKESLRAELRVTEGGGGSGEVQLVESGGGLVQ | | | |
| | | PGGSLRLSCAASGVTFNYYGMSWIRQAPGKGLE | | | |
| | | WVASITSSGGRIYYPDSVKGRFTISRENTQKTLY | | | |
| | | LQMNSLRAEDTAVYYCTLDGRDGWVAYWGQG | | | |
| | | TLVTVSSASTKGPSVFPLAPSSKSTSGGTAALGC | | | |
| | | LVKDYFPEPVTVSWNSGALTSGVHTFPAVLQSS | | | |
| | | GLYSLSSVVTVPSSSLGTQTYICNVNHKPSNTKV | | | |
| | | DKKVEPKSCDKTHTCPPCPAPEAAGGPSVFLFPP | | | |
| | | KPKDTLMISRTPEVTCVVVSVSHEDPEVKFNWY | | | |
| | | VDGVEVHNAKTKPREEQYNSTYRVVSVLTVLH | | | |
| | | QDWLNGKEYKCKVSNKALPAPIEKTISKAKGQP | | | |
| | | REPQVYVYPPSRDELTKNQVSLTCLVKGFYPSDI | | | |
| | | AVEWESNGQPENNYKTTPPVLDSDGSFALVSKL | | | |
| | | TVDKSRWQQGNVFSCSVMHEALHNHYTQKSLS | | | |
| | | LSPG | | | |
| | PD-1 | NPPTFSPALLVVTEGDNATFTCSFSNTSESFHVV | PD-1 33- | PD-L1 | 290 |
| | domain | WHRESPSGQTDTLAAFPEDRSQPGQDARFRVTQ | 146 | | |
| | | LPNGRDFHMSVVRARRNDSGTYVCGVISLAPKI | | | |
| | | QIKESLRAELRVTE | | | |
| | Linker | GGGGSG | | | 282 |
| | VH | EVQLVESGGGLVQPGGSLRLSCAASGVTFNYYG | C3E-4 | CD3 | 281 |
| | | MSWIRQAPGKGLEWVASITSSGGRIYYPDSVKG | | | |
| | | RFTISRENTQKTLYLQMNSLRAEDTAVYYCTLD | | | |
| | | GRDGWVAYWGQGTLVTVSS | | | |
| | HCDR1 | YYGMS | | | 303 |
| | HCDR2 | SITSSGGRIYYPDSVKG | | | 301 |
| | HCDR3 | DGRDGWVAY | | | 275 |
| 29220 | Full | NPPTFSPALLVVTEGDNATFTCSFSNTSESFHVV | | | 263 |
| | | WHRESPSGQTDTLAAFPEDRSQPGQDARFRVTQ | | | |
| | | LPNGRDFHMSVVRARRNDSGTYVCGVISLAPKI | | | |
| | | QIKESLRAELRVTEGGGGSGNFMLTQPHSVSESP | | | |
| | | GKTVTISCKRNTGNIGSNYVNWYQQHEGSSPTTI | | | |
| | | IYRNDKRPDGVSDRFSGSIDRSSKSASLTISNLKT | | | |
| | | EDEADYFCQSYSSGFIFGGGTKLTVLGQPKAAPS | | | |
| | | VTLFPPSSEELQANKATLVCLISDFYPGAVTVAW | | | |
| | | KADSSPVKAGVETTTPSKQSNNKYAASSYLSLTP | | | |
| | | EQWKSHRSYSCQVTHEGSTVEKTVAPTECS | | | |
| | PD-1 | NPPTFSPALLVVTEGDNATFTCSFSNTSESFHVV | PD-1 33- | PD-L1 | 290 |
| | domain | WHRESPSGQTDTLAAFPEDRSQPGQDARFRVTQ | 146 | | |
| | | LPNGRDFHMSVVRARRNDSGTYVCGVISLAPKI | | | |
| | | QIKESLRAELRVTE | | | |
| | Linker | GGGGSG | | | 282 |
| | VL | NFMLTQPHSVSESPGKTVTISCKRNTGNIGSNYV | C3E-1 | CD3 | 289 |
| | | NWYQQHEGSSPTTIIYRNDKRPDGVSDRFSGSID | | | |
| | | RSSKSASLTISNLKTEDEADYFCQSYSSGFIFGGG | | | |
| | | TKLTVL | | | |
| | LCDR1 | KRNTGNIGSNYVN | | | 287 |
| | LCDR2 | RNDKRPD | | | 298 |
| | LCDR3 | QSYSSGFI | | | 295 |
| 29226 | Full | NFMLTQPHSVSESPGKTVTISCKRNTGNIGSNYV | | | 264 |
| | | NWYQQHEGSSPTTIIYRNDKRPDGVSDRFSGSID | | | |
| | | RSSKSASLTISNLKTEDEADYFCQSYSSGFIFGGG | | | |
| | | TKLTVLGQPKAAPSVTLFPPSSEELQANKATLVC | | | |
| | | LISDFYPGAVTVAWKADSSPVKAGVETTTPSKQ | | | |
| | | SNNKYAASSYLSLTPEQWKSHRSYSCQVTHEGS | | | |
| | | TVEKTVAPTECSGGGGSGDRPWNPPTFSPALLV | | | |
| | | VTEGDNATFTCSFSNTSESFHVVWHRESPSGQTD | | | |
| | | TLAAFPEDRSQPGQDARFRVTQLPNGRDFHMSV | | | |
| | | VRARRNDSGTYVCGVISLAPKIQIKESLRAELRV | | | |
| | | TE | | | |
| | VL | NFMLTQPHSVSESPGKTVTISCKRNTGNIGSNYV | C3E-1 | CD3 | 289 |
| | | NWYQQHEGSSPTTIIYRNDKRPDGVSDRFSGSID | | | |
| | | RSSKSASLTISNLKTEDEADYFCQSYSSGFIFGGG | | | |
| | | TKLTVL | | | |
| | LCDR1 | KRNTGNIGSNYVN | | | 287 |
| | LCDR2 | RNDKRPD | | | 298 |
| | LCDR3 | QSYSSGFI | | | 295 |
| | Linker | GGGGSGDRPW | | | 283 |
| | PD-1 | NPPTFSPALLVVTEGDNATFTCSFSNTSESFHVV | PD-1 33- | PD-L1 | 290 |
| | domain | WHRESPSGQTDTLAAFPEDRSQPGQDARFRVTQ | 146 | | |
| | | LPNGRDFHMSVVRARRNDSGTYVCGVISLAPKI | | | |
| | | QIKESLRAELRVTE | | | |
| 29238 | Full | EVQLVESGGGLVQPGGSLRLSCAASGVTFNYYG | | | 265 |
| | | MSWIRQAPGKGLEWVASITSSGGRIYYPDSVKG | | | |
| | | RFTISRENTQKTLYLQMNSLRAEDTAVYYCTLD | | | |
| | | GRDGWVAYWGQGTLVTVSSASTKGPSVFPLAP | | | |
| | | SSKSTSGGTAALGCLVKDYFPEPVTVSWNSGAL | | | |
| | | TSGVHTFPAVLQSSGLYSLSSVVTVPSSSLGTQT | | | |
| | | YICNVNHKPSNTKVDKKVEPKSCDKTHTCPPCP | | | |
| | | APEAAGGPSVFLFPPKPKDTLMISRTPEVTCVVV | | | |
| | | SVSHEDPEVKFNWYVDGVEVHNAKTKPREEQY | | | |
| | | NSTYRVVSVLTVLHQDWLNGKEYKCKVSNKAL | | | |
| | | PAPIEKTISKAKGQPREPQVYVYPPSRDELTKNQ | | | |
| | | VSLTCLVKGFYPSDIAVEWESNGQPENNYKTTPP | | | |
| | | VLDSDGSFALVSKLTVDKSRWQQGNVFSCSVM | | | |
| | | HEALHNHYTQKSLSLSPGGGGGSGDRPWNPPTF | | | |
| | | SPALLVVTEGDNATFTCSFSNTSESFHVVWHRES | | | |
| | | PSGQTDTLAAFPEDRSQPGQDARFRVTQLPNGR | | | |
| | | DFHMSVVRARRNDSGTYVCGVISLAPKIQIKESL | | | |
| | | RAELRVTE | | | |
| | VH | EVQLVESGGGLVQPGGSLRLSCAASGVTFNYYG | C3E-4 | CD3 | 281 |
| | | MSWIRQAPGKGLEWVASITSSGGRIYYPDSVKG | | | |
| | | RFTISRENTQKTLYLQMNSLRAEDTAVYYCTLD | | | |
| | | GRDGWVAYWGQGTLVTVSS | | | |
| | HCDR1 | YYGMS | | | 303 |
| | HCDR2 | SITSSGGRIYYPDSVKG | | | 301 |
| | HCDR3 | DGRDGWVAY | | | 275 |
| | Linker | GGGGSGDRPW | | | 283 |
| | PD-1 | NPPTFSPALLVVTEGDNATFTCSFSNTSESFHVV | PD-1 33- | PD-L1 | 290 |
| | | WHRESPSGQTDTLAAFPEDRSQPGQDARFRVTQ | 146 | | |
| | domain | LPNGRDFHMSVVRARRNDSGTYVCGVISLAPKI | | | |
| | | QIKESLRAELRVTE | | | |
| 29257 | Full | EVQLVESGGGLVQPGGSLRLSCAASGVTFNYYG | | | 266 |
| | | MSWIRQAPGKGLEWVASITSSGGRIYYPDSVKG | | | |
| | | RFTISRENTQKTLYLQMNSLRAEDTAVYYCTLD | | | |
| | | GRDGWVAYWGQGTLVTVSSASTKGPSVFPLAP | | | |
| | | SSKSTSGGTAALGCLVKDYFPEPVTVSWNSGAL | | | |
| | | TSGVHTFPAVLQSSGLYSLSSVVTVPSSSLGTQT | | | |
| | | YICNVNHKPSNTKVDKKVEPKSCDKTHTCPPCP | | | |
| | | APEAAGGPSVFLFPPKPKDTLMISRTPEVTCVVV | | | |
| | | SVSHEDPEVKFNWYVDGVEVHNAKTKPREEQY | | | |
| | | NSTYRVVSVLTVLHQDWLNGKEYKCKVSNKAL | | | |
| | | PAPIEKTISKAKGQPREPQVYVYPPSRDELTKNQ | | | |
| | | VSLTCLVKGFYPSDIAVEWESNGQPENNYKTTPP | | | |
| | | VLDSDGSFALVSKLTVDKSRWQQGNVFSCSVM | | | |
| | | HEALHNHYTQKSLSLSPGGGGGSGDIQMTQSPS | | | |
| | | SLSASVGDRVTITCSASSSVSYMHWYQQKSGKA | | | |
| | | PKLLIYDTSKLASGVPSRFSGSGSGTDFTLTISSL | | | |
| | | QPEDFATYYCQQWSGYPLTFGQGTKLEIKGGGG | | | |
| | | SGGGGSGGGGSGGGGSQVQLVQSGAEVKKPGA | | | |
| | | SVKVSCKASGYSFTGYTMNWVRQAPGQGLEW | | | |
| | | MGLITPYNGASSYNQKFRGKATMTVDTSTSTVY | | | |
| | | MELSSLRSEDTAVYYCARGGYDGRGFDYWGQG | | | |
| | | TLVTVSS | | | |
| | VH1 | EVQLVESGGGLVQPGGSLRLSCAASGVTFNYYG | C3E-4 | CD3 | 281 |
| | | MSWIRQAPGKGLEWVASITSSGGRIYYPDSVKG | | | |
| | | RFTISRENTQKTLYLQMNSLRAEDTAVYYCTLD | | | |
| | | GRDGWVAYWGQGTLVTVSS | | | |
| | H1CDR1 | YYGMS | | | 303 |
| | H1CDR2 | SITSSGGRIYYPDSVKG | | | 301 |
| | H1CDR3 | DGRDGWVAY | | | 275 |
| | Linker | GGGGSG | | | 282 |
| | VL | DIQMTQSPSSLSASVGDRVTITCSASSSVSYMHW | huRG7787 | MSLN | 276 |
| | | YQQKSGKAPKLLIYDTSKLASGVPSRFSGSGSGT | | | |
| | | DFTLTISSLQPEDFATYYCQQWSGYPLTFGQGTK | | | |
| | | LEIK | | | |
| | VL-VH | GGGGSGGGGSGGGGSGGGGS | | | 284 |
| | linker | | | | |
| | VH2 | QVQLVQSGAEVKKPGASVKVSCKASGYSFTGY | huRG7787 | MSLN | 297 |
| | | TMNWVRQAPGQGLEWMGLITPYNGASSYNQKF | | | |
| | | RGKATMTVDTSTSTVYMELSSLRSEDTAVYYCA | | | |
| | | RGGYDGRGFDYWGQGTLVTVSS | | | |
| | LCDR1 | SASSSVSYMH | | | 300 |
| | LCDR2 | DTSKLAS | | | 279 |
| | LCDR3 | QQWSGYPLT | | | 294 |
| | H2CDR1 | GYTMN | | | 286 |
| | H2CDR2 | LITPYNGASSYNQKFRG | | | 288 |
| | H2CDR3 | GGYDGRGFDY | | | 285 |
| 29258 | Full | NPPTFSPALLVVTEGDNATFTCSFSNTSESFHVV | | | 267 |
| | | WHRESPSGQTDTLAAFPEDRSQPGQDARFRVTQ | | | |
| | | LPNGRDFHMSVVRARRNDSGTYVCGVISLAPKI | | | |
| | | QIKESLRAELRVTEGGGGSGDIQMTQSPSSLSAS | | | |
| | | VGDRVTITCSASSSVSYMHWYQQKSGKAPKLLI | | | |
| | | YDTSKLASGVPSRFSGSGSGTDFTLTISSLQPEDF | | | |
| | | ATYYCQQWSGYPLTFGQGTKLEIKGGGGSGGG | | | |
| | | GSGGGGSGGGGSQVQLVQSGAEVKKPGASVKV | | | |
| | | SCKASGYSFTGYTMNWVRQAPGQGLEWMGLIT | | | |
| | | PYNGASSYNQKFRGKATMTVDTSTSTVYMELSS | | | |
| | | LRSEDTAVYYCARGGYDGRGFDYWGQGTLVTV | | | |
| | | SSAAEPKSSDKTHTCPPCPAPEAAGGPSVFLFPPK | | | |
| | | PKDTLMISRTPEVTCVVVSVSHEDPEVKFNWYV | | | |
| | | DGVEVHNAKTKPREEQYNSTYRVVSVLTVLHQ | | | |
| | | DWLNGKEYKCKVSNKALPAPIEKTISKAKGQPR | | | |
| | | EPQVYVLPPSRDELTKNQVSLLCLVKGFYPSDIA | | | |
| | | VEWESNGQPENNYLTWPPVLDSDGSFFLYSKLT | | | |
| | | VDKSRWQQGNVFSCSVMHEALHNHYTQKSLSL | | | |
| | | SPG | | | |
| | PD-1 | NPPTFSPALLVVTEGDNATFTCSFSNTSESFHVV | PD-1 33- | PD-L1 | 290 |
| | domain | WHRESPSGQTDTLAAFPEDRSQPGQDARFRVTQ | 146 | | |
| | | LPNGRDFHMSVVRARRNDSGTYVCGVISLAPKI | | | |
| | | QIKESLRAELRVTE | | | |
| | Linker | GGGGSG | | | 282 |
| | VL | DIQMTQSPSSLSASVGDRVTITCSASSSVSYMHW | huRG7787 | MSLN | 276 |
| | | YQQKSGKAPKLLIYDTSKLASGVPSRFSGSGSGT | | | |
| | | DFTLTISSLQPEDFATYYCQQWSGYPLTFGQGTK | | | |
| | | LEIK | | | |
| | VL-VH | GGGGSGGGGSGGGGSGGGGS | | | 284 |
| | linker | | | | |
| | VH | QVQLVQSGAEVKKPGASVKVSCKASGYSFTGY | huRG7787 | MSLN | 297 |
| | | TMNWVRQAPGQGLEWMGLITPYNGASSYNQKF | | | |
| | | RGKATMTVDTSTSTVYMELSSLRSEDTAVYYCA | | | |
| | | RGGYDGRGFDYWGQGTLVTVSS | | | |
| | LCDR1 | SASSSVSYMH | | | 300 |
| | LCDR2 | DTSKLAS | | | 279 |
| | LCDR3 | QQWSGYPLT | | | 294 |
| | HCDR1 | GYTMN | | | 286 |
| | HCDR2 | LITPYNGASSYNQKFRG | | | 288 |
| | HCDR3 | GGYDGRGFDY | | | 285 |
| | Linker | AA | | | 274 |
| 29263 | Full | NPPTFSPALLVVTEGDNATFTCSFSNTSESFVLN | | | 268 |
| | | WYRMSPSNQTDALAAFPEDRSQPGQDARFRVT | | | |
| | | QLPNGRDFHMSVVRARRNDSGTYLCGAASLAP | | | |
| | | KAQIKESLRAELRVTEGGGGSGDIQMTQSPSSLS | | | |
| | | ASVGDRVTITCSASSSVSYMHWYQQKSGKAPKL | | | |
| | | LIYDTSKLASGVPSRFSGSGSGTDFTLTISSLQPE | | | |
| | | DFATYYCQQWSGYPLTFGQGTKLEIKGGGGSGG | | | |
| | | GGSGGGGSGGGGSQVQLVQSGAEVKKPGASVK | | | |
| | | VSCKASGYSFTGYTMNWVRQAPGQGLEWMGLI | | | |
| | | TPYNGASSYNQKFRGKATMTVDTSTSTVYMELS | | | |
| | | SLRSEDTAVYYCARGGYDGRGFDYWGQGTLVT | | | |
| | | VSSAAEPKSSDKTHTCPPCPAPEAAGGPSVFLFPP | | | |
| | | KPKDTLMISRTPEVTCVVVSVSHEDPEVKFNWY | | | |
| | | VDGVEVHNAKTKPREEQYNSTYRVVSVLTVLH | | | |
| | | QDWLNGKEYKCKVSNKALPAPIEKTISKAKGQP | | | |
| | | REPQVYVLPPSRDELTKNQVSLLCLVKGFYPSDI | | | |
| | | AVEWESNGQPENNYLTWPPVLDSDGSFFLYSKL | | | |
| | | TVDKSRWQQGNVFSCSVMHEALHNHYTQKSLS | | | |
| | | LSPG | | | |
| | PD-1 | NPPTFSPALLVVTEGDNATFTCSFSNTSESFVLN | PD-1 33- | PD-L1 | 291 |
| | domain | WYRMSPSNQTDALAAFPEDRSQPGQDARFRVT | 146 | | |
| | | QLPNGRDFHMSVVRARRNDSGTYLCGAASLAP | | | |
| | | KAQIKESLRAELRVTE | | | |
| | Linker | GGGGSG | | | 282 |
| | VL | DIQMTQSPSSLSASVGDRVTITCSASSSVSYMHW | huRG7787 | MSLN | 276 |
| | | YQQKSGKAPKLLIYDTSKLASGVPSRFSGSGSGT | | | |
| | | DFTLTISSLQPEDFATYYCQQWSGYPLTFGQGTK | | | |
| | | LEIK | | | |
| | VL-VH | GGGGSGGGGSGGGGSGGGGS | | | 284 |
| | linker | | | | |
| | VH | QVQLVQSGAEVKKPGASVKVSCKASGYSFTGY | huRG7787 | MSLN | 297 |
| | | TMNWVRQAPGQGLEWMGLITPYNGASSYNQKF | | | |
| | | RGKATMTVDTSTSTVYMELSSLRSEDTAVYYCA | | | |
| | | RGGYDGRGFDYWGQGTLVTVSS | | | |
| | LCDR1 | SASSSVSYMH | | | 300 |
| | LCDR2 | DTSKLAS | | | 279 |
| | LCDR3 | QQWSGYPLT | | | 294 |
| | HCDR1 | GYTMN | | | 286 |
| | HCDR2 | LITPYNGASSYNQKFRG | | | 288 |
| | HCDR3 | GGYDGRGFDY | | | 285 |
| | Linker | AA | | | 274 |
| 29264 | Full | DIQMTQSPSSLSASVGDRVTITCSASSSVSYMHW | | | 269 |
| | | YQQKSGKAPKLLIYDTSKLASGVPSRFSGSGSGT | | | |
| | | DFTLTISSLQPEDFATYYCQQWSGYPLTFGQGTK | | | |
| | | LEIKGGGGSGGGGSGGGGSGGGGSQVQLVQSG | | | |
| | | AEVKKPGASVKVSCKASGYSFTGYTMNWVRQA | | | |
| | | PGQGLEWMGLITPYNGASSYNQKFRGKATMTV | | | |
| | | DTSTSTVYMELSSLRSEDTAVYYCARGGYDGRG | | | |
| | | FDYWGQGTLVTVSSAAEPKSSDKTHTCPPCPAP | | | |
| | | EAAGGPSVFLFPPKPKDTLMISRTPEVTCVVVSV | | | |
| | | SHEDPEVKFNWYVDGVEVHNAKTKPREEQYNS | | | |
| | | TYRVVSVLTVLHQDWLNGKEYKCKVSNKALPA | | | |
| | | PIEKTISKAKGQPREPQVYVLPPSRDELTKNQVS | | | |
| | | LLCLVKGFYPSDIAVEWESNGQPENNYLTWPPV | | | |
| | | LDSDGSFFLYSKLTVDKSRWQQGNVFSCSVMHE | | | |
| | | ALHNHYTQKSLSLSPGGGGGSGDRPWNPPTFSP | | | |
| | | ALLVVTEGDNATFTCSFSNTSESFHVVWHRESPS | | | |
| | | GQTDTLAAFPEDRSQPGQDARFRVTQLPNGRDF | | | |
| | | HMSVVRARRNDSGTYVCGVISLAPKIQIKESLRA | | | |
| | | ELRVTE | | | |
| | VL | DIQMTQSPSSLSASVGDRVTITCSASSSVSYMHW | huRG7787 | MSLN | 276 |
| | | YQQKSGKAPKLLIYDTSKLASGVPSRFSGSGSGT | | | |
| | | DFTLTISSLQPEDFATYYCQQWSGYPLTFGQGTK | | | |
| | | LEIK | | | |
| | VL-VH | GGGGSGGGGSGGGGSGGGGS | | | 284 |
| | linker | | | | |
| | VH | QVQLVQSGAEVKKPGASVKVSCKASGYSFTGY | huRG7787 | MSLN | 297 |
| | | TMNWVRQAPGQGLEWMGLITPYNGASSYNQKF | | | |
| | | RGKATMTVDTSTSTVYMELSSLRSEDTAVYYCA | | | |
| | | RGGYDGRGFDYWGQGTLVTVSS | | | |
| | LCDR1 | SASSSVSYMH | | | 300 |
| | LCDR2 | DTSKLAS | | | 279 |
| | LCDR3 | QQWSGYPLT | | | 294 |
| | HCDR1 | GYTMN | | | 286 |
| | HCDR2 | LITPYNGASSYNQKFRG | | | 288 |
| | HCDR3 | GGYDGRGFDY | | | 285 |
| | Linker | AA | | | 274 |
| | C-term | GGGGSGDRPW | | | 283 |
| | Linker | | | | |
| | PD-1 | NPPTFSPALLVVTEGDNATFTCSFSNTSESFHVV | PD-1 33- | PD-L1 | 290 |
| | domain | WHRESPSGQTDTLAAFPEDRSQPGQDARFRVTQ | 146 | | |
| | | LPNGRDFHMSVVRARRNDSGTYVCGVISLAPKI | | | |
| | | QIKESLRAELRVTE | | | |
| 29275 | Full | DIQMTQSPSSLSASVGDRVTITCSASSSVSYMHW | | | 270 |
| | | YQQKSGKAPKLLIYDTSKLASGVPSRFSGSGSGT | | | |
| | | DFTLTISSLQPEDFATYYCQQWSKHPLTFGQGTK | | | |
| | | LEIKGGGGSGGGGSGGGGSGGGGSQVQLVQSG | | | |
| | | AEVKKPGASVKVSCKASGYSFTGYTMNWVRQA | | | |
| | | PGQGLEWMGLITPYNGASSYNQKFRGKATMTV | | | |
| | | DTSTSTVYMELSSLRSEDTAVYYCARGGYDGRG | | | |
| | | FDYWGQGTLVTVSSAAEPKSSDKTHTCPPCPAP | | | |
| | | EAAGGPSVFLFPPKPKDTLMISRTPEVTCVVVSV | | | |
| | | SHEDPEVKFNWYVDGVEVHNAKTKPREEQYNS | | | |
| | | TYRVVSVLTVLHQDWLNGKEYKCKVSNKALPA | | | |
| | | PIEKTISKAKGQPREPQVYVLPPSRDELTKNQVS | | | |
| | | LLCLVKGFYPSDIAVEWESNGQPENNYLTWPPV | | | |
| | | LDSDGSFFLYSKLTVDKSRWQQGNVFSCSVMHE | | | |
| | | ALHNHYTQKSLSLSPG | | | |
| | VL | DIQMTQSPSSLSASVGDRVTITCSASSSVSYMHW | huRG7787 | MSLN | 277 |
| | | YQQKSGKAPKLLIYDTSKLASGVPSRFSGSGSGT | | | |
| | | DFTLTISSLQPEDFATYYCQQWSKHPLTFGQGTK | | | |
| | | LEIK | | | |
| | VL-VH | GGGGSGGGGSGGGGSGGGGS | | | 284 |
| | linker | | | | |
| | VH | QVQLVQSGAEVKKPGASVKVSCKASGYSFTGY | huRG7787 | MSLN | 297 |
| | | TMNWVRQAPGQGLEWMGLITPYNGASSYNQKF | | | |
| | | RGKATMTVDTSTSTVYMELSSLRSEDTAVYYCA | | | |
| | | RGGYDGRGFDYWGQGTLVTVSS | | | |
| | LCDR1 | SASSSVSYMH | | | 300 |
| | LCDR2 | DTSKLAS | | | 279 |
| | LCDR3 | QQWSGYPLT | | | 294 |
| | HCDR1 | GYTMN | | | 286 |
| | HCDR2 | LITPYNGASSYNQKFRG | | | 288 |
| | HCDR3 | GGYDGRGFDY | | | 285 |
| | Linker | AA | | | 274 |
| 29276 | Full | NPPTFSPALLVVTEGDNATFTCSFSNTSESFHVV | | | 271 |
| | | WHRESPSGQTDTLAAFPEDRSQPGQDARFRVTQ | | | |
| | | LPNGRDFHMSVVRARRNDSGTYVCGVISLAPKI | | | |
| | | QIKESLRAELRVTEGGGGSGQVQLVQSGAEVKK | | | |
| | | PGASVKVSCKASGYSFTGYTMNWVRQAPGQGL | | | |
| | | EWMGLITPYNGASSYNQKFRGKATMTVDTSTST | | | |
| | | VYMELSSLRSEDTAVYYCARGGYDGRGFDYWG | | | |
| | | QGTLVTVSSASTKGPSVFPLAPSSKSTSGGTAAL | | | |
| | | GCLVKDYFPEPVTVSWNSGALTSGVHTFPAVLQ | | | |
| | | SSGLYSLSSVVTVPSSSLGTQTYICNVNHKPSNT | | | |
| | | KVDKKVEPKSCDKTHTCPPCPAPEAAGGPSVFL | | | |
| | | FPPKPKDTLMISRTPEVTCVVVSVSHEDPEVKFN | | | |
| | | WYVDGVEVHNAKTKPREEQYNSTYRVVSVLTV | | | |
| | | LHQDWLNGKEYKCKVSNKALPAPIEKTISKAKG | | | |
| | | QPREPQVYVLPPSRDELTKNQVSLLCLVKGFYPS | | | |
| | | DIAVEWESNGQPENNYLTWPPVLDSDGSFFLYS | | | |
| | | KLTVDKSRWQQGNVFSCSVMHEALHNHYTQKS | | | |
| | | LSLSPG | | | |
| | PD-1 | NPPTFSPALLVVTEGDNATFTCSFSNTSESFHVV | PD-1 33- | PD-L1 | 290 |
| | | WHRESPSGQTDTLAAFPEDRSQPGQDARFRVTQ | 146 | | |
| | domain | LPNGRDFHMSVVRARRNDSGTYVCGVISLAPKI | | | |
| | | QIKESLRAELRVTE | | | 282 |
| | Linker | GGGGSG | | | |
| | VH | QVQLVQSGAEVKKPGASVKVSCKASGYSFTGY | huRG7787 | MSLN | 297 |
| | | TMNWVRQAPGQGLEWMGLITPYNGASSYNQKF | | | |
| | | RGKATMTVDTSTSTVYMELSSLRSEDTAVYYCA | | | |
| | | RGGYDGRGFDYWGQGTLVTVSS | | | |
| | HCDR1 | GYTMN | | | 286 |
| | HCDR2 | LITPYNGASSYNQKFRG | | | 288 |
| | HCDR3 | GGYDGRGFDY | | | 285 |
| 29282 | Full | DIQMTQSPSSLSASVGDRVTITCSASSSVSYMHW | | | 272 |
| | | YQQKSGKAPKLLIYDTSKLASGVPSRFSGSGSGT | | | |
| | | DFTLTISSLQPEDFATYYCQQWSKHPLTFGQGTK | | | |
| | | LEIKGGGGSGGGGSGGGGSGGGGSQVQLVQSG | | | |
| | | AEVKKPGASVKVSCKASGYSFTGYTMNWVRQA | | | |
| | | PGQGLEWMGLITPYNGASSYNQKFRGKATMTV | | | |
| | | DTSTSTVYMELSSLRSEDTAVYYCARGGYDGRG | | | |
| | | FDYWGQGTLVTVSSGGGGSGEVQLVESGGGLV | | | |
| | | QPGGSLRLSCAASGVTFNYYGMSWIRQAPGKGL | | | |
| | | EWVASITSSGGRIYYPDSVKGRFTISRENTQKTL | | | |
| | | YLQMNSLRAEDTAVYYCTLDGRDGWVAYWGQ | | | |
| | | GTLVTVSSASTKGPSVFPLAPSSKSTSGGTAALG | | | |
| | | CLVKDYFPEPVTVSWNSGALTSGVHTFPAVLQS | | | |
| | | SGLYSLSSVVTVPSSSLGTQTYICNVNHKPSNTK | | | |
| | | VDKKVEPKSCDKTHTCPPCPAPEAAGGPSVFLFP | | | |
| | | PKPKDTLMISRTPEVTCVVVSVSHEDPEVKFNW | | | |
| | | YVDGVEVHNAKTKPREEQYNSTYRVVSVLTVL | | | |
| | | HQDWLNGKEYKCKVSNKALPAPIEKTISKAKGQ | | | |
| | | PREPQVYVYPPSRDELTKNQVSLTCLVKGFYPSD | | | |
| | | IAVEWESNGQPENNYKTTPPVLDSDGSFALVSK | | | |
| | | LTVDKSRWQQGNVFSCSVMHEALHNHYTQKSL | | | |
| | | SLSPG | | | |
| | VL | DIQMTQSPSSLSASVGDRVTITCSASSSVSYMHW | huRG7787 | MSLN | 277 |
| | | YQQKSGKAPKLLIYDTSKLASGVPSRFSGSGSGT | | | |
| | | DFTLTISSLQPEDFATYYCQQWSKHPLTFGQGTK | | | |
| | | LEIK | | | |
| | VL-VH | GGGGSGGGGSGGGGSGGGGS | | | 284 |
| | linker | | | | |
| | VH1 | QVQLVQSGAEVKKPGASVKVSCKASGYSFTGY | huRG7787 | MSLN | 297 |
| | | TMNWVRQAPGQGLEWMGLITPYNGASSYNQKF | | | |
| | | RGKATMTVDTSTSTVYMELSSLRSEDTAVYYCA | | | |
| | | RGGYDGRGFDYWGQGTLVTVSS | | | |
| | LCDR1 | SASSSVSYMH | | | 300 |
| | LCDR2 | DTSKLAS | | | 279 |
| | LCDR3 | QQWSKHPLT | | | 294 |
| | H1CDR1 | GYTMN | | | 286 |
| | H1CDR2 | LITPYNGASSYNQKFRG | | | 288 |
| | H1CDR3 | GGYDGRGFDY | | | 285 |
| | Linker | GGGGSG | | | 282 |
| | VH2 | EVQLVESGGGLVQPGGSLRLSCAASGVTFNYYG | C3E-4 | CD3 | 281 |
| | | MSWIRQAPGKGLEWVASITSSGGRIYYPDSVKG | | | |
| | | RFTISRENTQKTLYLQMNSLRAEDTAVYYCTLD | | | |
| | | GRDGWVAYWGQGTLVTVSS | | | |
| | H2CDR1 | YYGMS | | | 303 |
| | H2CDR2 | SITSSGGRIYYPDSVKG | | | 301 |
| | H2CDR3 | DGRDGWVAY | | | 275 |
| 29283 | Full | DIQMTQSPSSLSASVGDRVTITCSASSSVSYMHW | | | 273 |
| | | YQQKSGKAPKLLIYDTSKLASGVPSRFSGSGSGT | | | |
| | | DFTLTISSLQPEDFATYYCQQWSGYPLTFGQGTK | | | |
| | | LEIKGGGGSGGGGSGGGGSGGGGSQVQLVQSG | | | |
| | | AEVKKPGASVKVSCKASGYSFTGYTMNWVRQA | | | |
| | | PGQGLEWMGLITPYNGASSYNQKFRGKATMTV | | | |
| | | DTSTSTVYMELSSLRSEDTAVYYCARGGYDGRG | | | |
| | | FDYWGQGTLVTVSSGGGGSGEVQLVESGGGLV | | | |
| | | QPGGSLRLSCAASGVTFNYYGMSWIRQAPGKGL | | | |
| | | EWVASITSSGGRIYYPDSVKGRFTISRENTQKTL | | | |
| | | YLQMNSLRAEDTAVYYCTLDGRDGWVAYWGQ | | | |
| | | GTLVTVSSASTKGPSVFPLAPSSKSTSGGTAALG | | | |
| | | CLVKDYFPEPVTVSWNSGALTSGVHTFPAVLQS | | | |
| | | SGLYSLSSVVTVPSSSLGTQTYICNVNHKPSNTK | | | |
| | | VDKKVEPKSCDKTHTCPPCPAPEAAGGPSVFLFP | | | |
| | | PKPKDTLMISRTPEVTCVVVSVSHEDPEVKFNW | | | |
| | | YVDGVEVHNAKTKPREEQYNSTYRVVSVLTVL | | | |
| | | HQDWLNGKEYKCKVSNKALPAPIEKTISKAKGQ | | | |
| | | PREPQVYVYPPSRDELTKNQVSLTCLVKGFYPSD | | | |
| | | IAVEWESNGQPENNYKTTPPVLDSDGSFALVSK | | | |
| | | LTVDKSRWQQGNVFSCSVMHEALHNHYTQKSL | | | |
| | | SLSPG | | | |
| | VL | DIQMTQSPSSLSASVGDRVTITCSASSSVSYMHW | huRG7787 | MSLN | 276 |
| | | YQQKSGKAPKLLIYDTSKLASGVPSRFSGSGSGT | | | |
| | | DFTLTISSLQPEDFATYYCQQWSGYPLTFGQGTK | | | |
| | | LEIK | | | |
| | VL-VH | GGGGSGGGGSGGGGSGGGGS | | | 284 |
| | linker | | | | |
| | (or | | | | |
| | linker- | | | | |
| | scFv) | | | | |
| | VH1 | QVQLVQSGAEVKKPGASVKVSCKASGYSFTGY | huRG7787 | MSLN | 297 |
| | | TMNWVRQAPGQGLEWMGLITPYNGASSYNQKF | | | |
| | | RGKATMTVDTSTSTVYMELSSLRSEDTAVYYCA | | | |
| | | RGGYDGRGFDYWGQGTLVTVSS | | | |
| | LCDR1 | SASSSVSYMH | | | 300 |
| | LCDR2 | DTSKLAS | | | 279 |
| | LCDR3 | QQWSGYPLT | | | 294 |
| | H1CDR1 | GYTMN | | | 286 |
| | H1CDR2 | LITPYNGASSYNQKFRG | | | 288 |
| | H1CDR3 | GGYDGRGFDY | | | 285 |
| | Linker | GGGGSG | | | 282 |
| | VH2 | EVQLVESGGGLVQPGGSLRLSCAASGVTFNYYG | C3E-4 | CD3 | 281 |
| | | MSWIRQAPGKGLEWVASITSSGGRIYYPDSVKG | | | |
| | | RFTISRENTQKTLYLQMNSLRAEDTAVYYCTLD | | | |
| | | GRDGWVAYWGQGTLVTVSS | | | |
| | H2CDR1 | YYGMS | | | 303 |
| | H2CDR2 | SITSSGGRIYYPDSVKG | | | 301 |
| | H2CDR3 | DGRDGWVAY | | | 275 |
| 29206 | | NPPTFSPALLVVTEGDNATFTCSFSNTSESFVLN | | CD3/PD- | 400 |
| | | WYRMSPSNQTDALAAFPEDRSQPGQDARFRVT | | L1 | |
| | | QLPNGRDFHMSVVRARRNDSGTYLCGAASLAP | | | |
| | | KAQIKESLRAELRVTEEAAAKEAAAKEVQLVES | | | |
| | | GGGLVQPGGSLRLSCAASGVTFNYYGMSWIRQ | | | |
| | | APGKGLEWVASITSSGGRIYYPDSVKGRFTISRE | | | |
| | | NTQKTLYLQMNSLRAEDTAVYYCTLDGRDGW | | | |
| | | VAYWGQGTLVTVSSASTKGPSVFPLAPSSKSTSG | | | |
| | | GTAALGCLVKDYFPEPVTVSWNSGALTSGVHTF | | | |
| | | PAVLQSSGLYSLSSVVTVPSSSLGTQTYICNVNH | | | |
| | | KPSNTKVDKKVEPKSCDKTHTCPPCPAPEAAGG | | | |
| | | PSVFLFPPKPKDTLMISRTPEVTCVVVSVSHEDPE | | | |
| | | VKFNWYVDGVEVHNAKTKPREEQYNSTYRVVS | | | |
| | | VLTVLHQDWLNGKEYKCKVSNKALPAPIEKTIS | | | |
| | | KAKGQPREPQVYVYPPSRDELTKNQVSLTCLVK | | | |
| | | GFYPSDIAVEWESNGQPENNYKTTPPVLDSDGSF | | | |
| | | ALVSKLTVDKSRWQQGNVFSCSVMHEALHNHY | | | |
| | | TQKSLSLSPG | | | |
| 29204 | | NPPTFSPALLVVTEGDNATFTCSFSNTSESFVLN | | CD3/PD- | 401 |
| | | WYRMSPSNQTDKLAAFPEDRSQPGQDARFRVT | | L1 | |
| | | QLPNGRDFHMSVVRARRNDSGTYLCGAISLAPK | | | |
| | | AQIKESLRAELRVTEEAAAKEAAAKEVQLVESG | | | |
| | | GGLVQPGGSLRLSCAASGVTFNYYGMSWIRQAP | | | |
| | | GKGLEWVASITSSGGRIYYPDSVKGRFTISRENT | | | |
| | | QKTLYLQMNSLRAEDTAVYYCTLDGRDGWVA | | | |
| | | YWGQGTLVTVSSASTKGPSVFPLAPSSKSTSGGT | | | |
| | | AALGCLVKDYFPEPVTVSWNSGALTSGVHTFPA | | | |
| | | VLQSSGLYSLSSVVTVPSSSLGTQTYICNVNHKP | | | |
| | | SNTKVDKKVEPKSCDKTHTCPPCPAPEAAGGPS | | | |
| | | VFLFPPKPKDTLMISRTPEVTCVVVSVSHEDPEV | | | |
| | | KFNWYVDGVEVHNAKTKPREEQYNSTYRVVSV | | | |
| | | LTVLHQDWLNGKEYKCKVSNKALPAPIEKTISK | | | |
| | | AKGQPREPQVYVYPPSRDELTKNQVSLTCLVKG | | | |
| | | FYPSDIAVEWESNGQPENNYKTTPPVLDSDGSFA | | | |
| | | LVSKLTVDKSRWQQGNVFSCSVMHEALHNHYT | | | |
| | | QKSLSLSPG | | | |
| 29203 | | NPPTFSPALLVVTEGDNATFTCSFSNTSESFVLN | | CD3/PD- | 402 |
| | | WYRMSPSNQTDKLAAFPEDRSQPGQDARFRVT | | L1 | |
| | | QLPNGRDFHMSVVRARRNDSGTYLCGAISLAPK | | | |
| | | LQIKESLRAELRVTEEAAAKEAAAKEVQLVESG | | | |
| | | GGLVQPGGSLRLSCAASGVTFNYYGMSWIRQAP | | | |
| | | GKGLEWVASITSSGGRIYYPDSVKGRFTISRENT | | | |
| | | QKTLYLQMNSLRAEDTAVYYCTLDGRDGWVA | | | |
| | | YWGQGTLVTVSSASTKGPSVFPLAPSSKSTSGGT | | | |
| | | AALGCLVKDYFPEPVTVSWNSGALTSGVHTFPA | | | |
| | | VLQSSGLYSLSSVVTVPSSSLGTQTYICNVNHKP | | | |
| | | SNTKVDKKVEPKSCDKTHTCPPCPAPEAAGGPS | | | |
| | | VFLFPPKPKDTLMISRTPEVTCVVVSVSHEDPEV | | | |
| | | KFNWYVDGVEVHNAKTKPREEQYNSTYRVVSV | | | |
| | | LTVLHQDWLNGKEYKCKVSNKALPAPIEKTISK | | | |
| | | AKGQPREPQVYVYPPSRDELTKNQVSLTCLVKG | | | |
| | | FYPSDIAVEWESNGQPENNYKTTPPVLDSDGSFA | | | |
| | | LVSKLTVDKSRWQQGNVFSCSVMHEALHNHYT | | | |
| | | QKSLSLSPG | | | |
| 29202 | | NPPTFSPALLVVTEGDNATFTCSFSNTSESFRLV | | CD3/PD- | 403 |
| | | WHRESPGYETDTLASFPEDRSTPLPDARFRVTQL | | L1 | |
| | | PNGRDFHMSVVRARRNDSGTYVCGAIAFHPVIQ | | | |
| | | IKESLRAELRVTEEAAAKEAAAKEVQLVESGGG | | | |
| | | LVQPGGSLRLSCAASGVTFNYYGMSWIRQAPGK | | | |
| | | GLEWVASITSSGGRIYYPDSVKGRFTISRENTQK | | | |
| | | TLYLQMNSLRAEDTAVYYCTLDGRDGWVAYW | | | |
| | | GQGTLVTVSSASTKGPSVFPLAPSSKSTSGGTAA | | | |
| | | LGCLVKDYFPEPVTVSWNSGALTSGVHTFPAVL | | | |
| | | QSSGLYSLSSVVTVPSSSLGTQTYICNVNHKPSN | | | |
| | | TKVDKKVEPKSCDKTHTCPPCPAPEAAGGPSVF | | | |
| | | LFPPKPKDTLMISRTPEVTCVVVSVSHEDPEVKF | | | |
| | | NWYVDGVEVHNAKTKPREEQYNSTYRVVSVLT | | | |
| | | VLHQDWLNGKEYKCKVSNKALPAPIEKTISKAK | | | |
| | | GQPREPQVYVYPPSRDELTKNQVSLTCLVKGFY | | | |
| | | PSDIAVEWESNGQPENNYKTTPPVLDSDGSFALV | | | |
| | | SKLTVDKSRWQQGNVFSCSVMHEALHNHYTQK | | | |
| | | SLSLSPG | | | |
| 29214 | | NPPTFSPALLVVTEGDNATFTCSFSNTSESFHVV | | Cldn18.2/ | 404 |
| | | WHRESPSGQTDTLAAFPEDRSQPGQDARFRVTQ | | PD-L1 | |
| | | LPNGRDFHMSVVRARRNDSGTYVCGVISLAPKI | | | |
| | | QIKESLRAELRVTEGGGGSGQVQLVESGGGLVQ | | | |
| | | PGGSLRLSCSVSGIDLSSNPMIWVRQAPGKGLQY | | | |
| | | IGIIDTDGSTYYADWAKGRFTISKDSTTVYLQINS | | | |
| | | PRAEDTAVYYCARRLHGSSNGYYDDLWGQGTL | | | |
| | | VTVSSASTKGPSVFPLAPSSKSTSGGTAALGCLV | | | |
| | | KDYFPEPVTVSWNSGALTSGVHTFPAVLQSSGL | | | |
| | | YSLSSVVTVPSSSLGTQTYICNVNHKPSNTKVDK | | | |
| | | KVEPKSCDKTHTCPPCPAPEAAGGPSVFLFPPKP | | | |
| | | KDTLMISRTPEVTCVVVSVSHEDPEVKFNWYVD | | | |
| | | GVEVHNAKTKPREEQYNSTYRVVSVLTVLHQD | | | |
| | | WLNGKEYKCKVSNKALPAPIEKTISKAKGQPRE | | | |
| | | PQVYVLPPSRDELTKNQVSLLCLVKGFYPSDIAV | | | |
| | | EWESNGQPENNYLTWPPVLDSDGSFFLYSKLTV | | | |
| | | DKSRWQQGNVFSCSVMHEALHNHYTQKSLSLS | | | |
| | | PG | | | |
| 28196 | LC | DGQMTQSPSSVSASVGDRVTITCQASQSIYSYLS | | Cldn18.2 | 405 |
| | | WYQQKPGQRPKLLIYKASTLASGVPSRFSGSGS | | | |
| | | GTDFTLTISSVQPEDFATYYCQQGYTVTNVDKN | | | |
| | | TFGGGTKVEIKRTVAAPSVFIFPPSDEQLKSGTAS | | | |
| | | VVCLLNNFYPREAKVQWKVDNALQSGNSQESV | | | |
| | | TEQDSKDSTYSLSSTLTLSKADYEKHKVYACEV | | | |
| | | THQGLSSPVTKSFNRGEC | | | |
| 29237 | | QVQLVESGGGLVQPGGSLRLSCSVSGIDLSSNPM | | Cldn18.2/ | 406 |
| | | IWVRQAPGKGLQYIGIIDTDGSTYYADWAKGRF | | CD3 | |
| | | TISKDSTTVYLQINSPRAEDTAVYYCARRLHGSS | | | |
| | | NGYYDDLWGQGTLVTVSSGGGGSGGGGSGGG | | | |
| | | GSGGGGSDGQMTQSPSSVSASVGDRVTITCQAS | | | |
| | | QSIYSYLSWYQQKPGQRPKLLIYKASTLASGVPS | | | |
| | | RFSGSGSGTDFTLTISSVQPEDFATYYCQQGYTV | | | |
| | | TNVDKNTFGGGTKVEIKGGGGSGEVQLVESGG | | | |
| | | GLVQPGGSLRLSCAASGVTFNYYGMSWIRQAPG | | | |
| | | KGLEWVASITSSGGRIYYPDSVKGRFTISRENTQ | | | |
| | | KTLYLQMNSLRAEDTAVYYCTLDGRDGWVAY | | | |
| | | WGQGTLVTVSSASTKGPSVFPLAPSSKSTSGGTA | | | |
| | | ALGCLVKDYFPEPVTVSWNSGALTSGVHTFPAV | | | |
| | | LQSSGLYSLSSVVTVPSSSLGTQTYICNVNHKPS | | | |
| | | NTKVDKKVEPKSCDKTHTCPPCPAPEAAGGPSV | | | |
| | | FLFPPKPKDTLMISRTPEVTCVVVSVSHEDPEVK | | | |
| | | FNWYVDGVEVHNAKTKPREEQYNSTYRVVSVL | | | |
| | | TVLHQDWLNGKEYKCKVSNKALPAPIEKTISKA | | | |
| | | KGQPREPQVYVYPPSRDELTKNQVSLTCLVKGF | | | |
| | | YPSDIAVEWESNGQPENNYKTTPPVLDSDGSFAL | | | |
| | | VSKLTVDKSRWQQGNVFSCSVMHEALHNHYTQ | | | |
| | | KSLSLSPG | | | |
| 28273 | | QVQLVESGGGLVQPGGSLRLSCSVSGIDLSSNPM | | Cldn18.2 | 407 |
| | | IWVRQAPGKGLQYIGIIDTDGSTYYADWAKGRF | | | |
| | | TISKDSTTVYLQINSPRAEDTAVYYCARRLHGSS | | | |
| | | NGYYDDLWGQGTLVTVSSGGGGSGGGGSGGG | | | |
| | | GSGGGGSDGQMTQSPSSVSASVGDRVTITCQAS | | | |
| | | QSIYSYLSWYQQKPGQRPKLLIYKASTLASGVPS | | | |
| | | RFSGSGSGTDFTLTISSVQPEDFATYYCQQGYTV | | | |
| | | TNVDKNTFGGGTKVEIKAAEPKSSDKTHTCPPCP | | | |
| | | APEAAGGPSVFLFPPKPKDTLMISRTPEVTCVVV | | | |
| | | SVSHEDPEVKFNWYVDGVEVHNAKTKPREEQY | | | |
| | | NSTYRVVSVLTVLHQDWLNGKEYKCKVSNKAL | | | |
| | | PAPIEKTISKAKGQPREPQVYVLPPSRDELTKNQ | | | |
| | | VSLLCLVKGFYPSDIAVEWESNGQPENNYLTWP | | | |
| | | PVLDSDGSFFLYSKLTVDKSRWQQGNVFSCSVM | | | |
| | | HEALHNHYTQKSLSLSPG | | | |
| 25094 | | EVQLVESGGGLVQPGGSLRLSCAASGVTFNYYG | | CD3 | 408 |
| | | MSWIRQAPGKGLEWVASITSSGGRIYYPDSVKG | | | |
| | | RFTISRENTQKTLYLQMNSLRAEDTAVYYCTLD | | | |
| | | GRDGWVAYWGQGTLVTVSSASTKGPSVFPLAP | | | |
| | | SSKSTSGGTAALGCLVKDYFPEPVTVSWNSGAL | | | |
| | | TSGVHTFPAVLQSSGLYSLSSVVTVPSSSLGTQT | | | |
| | | YICNVNHKPSNTKVDKKVEPKSCDKTHTCPPCP | | | |
| | | APEAAGGPSVFLFPPKPKDTLMISRTPEVTCVVV | | | |
| | | SVSHEDPEVKFNWYVDGVEVHNAKTKPREEQY | | | |
| | | NSTYRVVSVLTVLHQDWLNGKEYKCKVSNKAL | | | |
| | | PAPIEKTISKAKGQPREPQVYVYPPSRDELTKNQ | | | |
| | | VSLTCLVKGFYPSDIAVEWESNGQPENNYKTTPP | | | |
| | | VLDSDGSFALVSKLTVDKSRWQQGNVFSCSVM | | | |
| | | HEALHNHYTQKSLSLSPG | | | |
| 29213 | | NPPTFSPALLVVTEGDNATFTCSFSNTSESFVLN | | PD- | 409 |
| | | WYRMSPSNQTDALAAFPEDRSQPGQDARFRVT | | L1/CD3 | |
| | | QLPNGRDFHMSVVRARRNDSGTYLCGAASLAP | | | |
| | | KAQIKESLRAELRVTEGGGGSGEVQLVESGGGL | | | |
| | | VQPGGSLRLSCAASGVTFNYYGMSWIRQAPGK | | | |
| | | GLEWVASITSSGGRIYYPDSVKGRFTISRENTQK | | | |
| | | TLYLQMNSLRAEDTAVYYCTLDGRDGWVAYW | | | |
| | | GQGTLVTVSSASTKGPSVFPLAPSSKSTSGGTAA | | | |
| | | LGCLVKDYFPEPVTVSWNSGALTSGVHTFPAVL | | | |
| | | QSSGLYSLSSVVTVPSSSLGTQTYICNVNHKPSN | | | |
| | | TKVDKKVEPKSCDKTHTCPPCPAPEAAGGPSVF | | | |
| | | LFPPKPKDTLMISRTPEVTCVVVSVSHEDPEVKF | | | |
| | | NWYVDGVEVHNAKTKPREEQYNSTYRVVSVLT | | | |
| | | VLHQDWLNGKEYKCKVSNKALPAPIEKTISKAK | | | |
| | | GQPREPQVYVYPPSRDELTKNQVSLTCLVKGFY | | | |
| | | PSDIAVEWESNGQPENNYKTTPPVLDSDGSFALV | | | |
| | | SKLTVDKSRWQQGNVFSCSVMHEALHNHYTQK | | | |
| | | SLSLSPG | | | |
| 29219 | | NPPTFSPALLVVTEGDNATFTCSFSNTSESFVLN | | PD-L1/ | 410 |
| | | WYRMSPSNQTDALAAFPEDRSQPGQDARFRVT | | Cldn18.2 | |
| | | QLPNGRDFHMSVVRARRNDSGTYLCGAASLAP | | | |
| | | KAQIKESLRAELRVTEGGGGSGQVQLVESGGGL | | | |
| | | VQPGGSLRLSCSVSGIDLSSNPMIWVRQAPGKGL | | | |
| | | QYIGIIDTDGSTYYADWAKGRFTISKDSTTVYLQ | | | |
| | | INSPRAEDTAVYYCARRLHGSSNGYYDDLWGQ | | | |
| | | GTLVTVSSASTKGPSVFPLAPSSKSTSGGTAALG | | | |
| | | CLVKDYFPEPVTVSWNSGALTSGVHTFPAVLQS | | | |
| | | SGLYSLSSVVTVPSSSLGTQTYICNVNHKPSNTK | | | |
| | | VDKKVEPKSCDKTHTCPPCPAPEAAGGPSVFLFP | | | |
| | | PKPKDTLMISRTPEVTCVVVSVSHEDPEVKFNW | | | |
| | | YVDGVEVHNAKTKPREEQYNSTYRVVSVLTVL | | | |
| | | HQDWLNGKEYKCKVSNKALPAPIEKTISKAKGQ | | | |
| | | PREPQVYVLPPSRDELTKNQVSLLCLVKGFYPSD | | | |
| | | IAVEWESNGQPENNYLTWPPVLDSDGSFFLYSK | | | |
| | | LTVDKSRWQQGNVFSCSVMHEALHNHYTQKSL | | | |
| | | SLSPG | | | |
| 29243 | | EVQLVESGGGLVQPGGSLRLSCAASGVTFNYYG | | CD3/PD- | 411 |
| | | MSWIRQAPGKGLEWVASITSSGGRIYYPDSVKG | | L1 | |
| | | RFTISRENTQKTLYLQMNSLRAEDTAVYYCTLD | | | |
| | | GRDGWVAYWGQGTLVTVSSASTKGPSVFPLAP | | | |
| | | SSKSTSGGTAALGCLVKDYFPEPVTVSWNSGAL | | | |
| | | TSGVHTFPAVLQSSGLYSLSSVVTVPSSSLGTQT | | | |
| | | YICNVNHKPSNTKVDKKVEPKSCDKTHTCPPCP | | | |
| | | APEAAGGPSVFLFPPKPKDTLMISRTPEVTCVVV | | | |
| | | SVSHEDPEVKFNWYVDGVEVHNAKTKPREEQY | | | |
| | | NSTYRVVSVLTVLHQDWLNGKEYKCKVSNKAL | | | |
| | | PAPIEKTISKAKGQPREPQVYVYPPSRDELTKNQ | | | |
| | | VSLTCLVKGFYPSDIAVEWESNGQPENNYKTTPP | | | |
| | | VLDSDGSFALVSKLTVDKSRWQQGNVFSCSVM | | | |
| | | HEALHNHYTQKSLSLSPGGGGGSGDRPWNPPTF | | | |
| | | SPALLVVTEGDNATFTCSFSNTSESFVLNWYRM | | | |
| | | SPSNQTDALAAFPEDRSQPGQDARFRVTQLPNG | | | |
| | | RDFHMSVVRARRNDSGTYLCGAASLAPKAQIKE | | | |
| | | SLRAELRVTE | | | |
| 29225 | | NPPTFSPALLVVTEGDNATFTCSFSNTSESFVLN | | PD- | 412 |
| | | WYRMSPSNQTDALAAFPEDRSQPGQDARFRVT | | L1/CD3 | |
| | | QLPNGRDFHMSVVRARRNDSGTYLCGAASLAP | | | |
| | | KAQIKESLRAELRVTEGGGGSGNFMLTQPHSVS | | | |
| | | ESPGKTVTISCKRNTGNIGSNYVNWYQQHEGSS | | | |
| | | PTTIIYRNDKRPDGVSDRFSGSIDRSSKSASLTISN | | | |
| | | LKTEDEADYFCQSYSSGFIFGGGTKLTVLGQPKA | | | |
| | | APSVTLFPPSSEELQANKATLVCLISDFYPGAVT | | | |
| | | VAWKADSSPVKAGVETTTPSKQSNNKYAASSY | | | |
| | | LSLTPEQWKSHRSYSCQVTHEGSTVEKTVAPTE | | | |
| | | CS | | | |
| 29209 | | NPPTFSPALLVVTEGDNATFTCSFSNTSESFRLV | | CD3 | 413 |
| | | WHRESPGYETDTLASFPEDRSTPLPDARFRVTQL | | | |
| | | PNGRDFHMSVVRARRNDSGTYVCGAIAFHPVIQ | | | |
| | | IKESLRAELRVTEGGGGSGEVQLVESGGGLVQP | | | |
| | | GGSLRLSCAASGVTFNYYGMSWIRQAPGKGLE | | | |
| | | WVASITSSGGRIYYPDSVKGRFTISRENTQKTLY | | | |
| | | LQMNSLRAEDTAVYYCTLDGRDGWVAYWGQG | | | |
| | | TLVTVSSASTKGPSVFPLAPSSKSTSGGTAALGC | | | |
| | | LVKDYFPEPVTVSWNSGALTSGVHTFPAVLQSS | | | |
| | | GLYSLSSVVTVPSSSLGTQTYICNVNHKPSNTKV | | | |
| | | DKKVEPKSCDKTHTCPPCPAPEAAGGPSVFLFPP | | | |
| | | KPKDTLMISRTPEVTCVVVSVSHEDPEVKFNWY | | | |
| | | VDGVEVHNAKTKPREEQYNSTYRVVSVLTVLH | | | |
| | | QDWLNGKEYKCKVSNKALPAPIEKTISKAKGQP | | | |
| | | REPQVYVYPPSRDELTKNQVSLTCLVKGFYPSDI | | | |
| | | AVEWESNGQPENNYKTTPPVLDSDGSFALVSKL | | | |
| | | TVDKSRWQQGNVFSCSVMHEALHNHYTQKSLS | | | |
| | | LSPG | | | |
| 29212 | | NPPTFSPALLVVTEGDNATFTCSFSNTSESFVLN | | PD- | 414 |
| | | WYRMSPSNQTDKLAAFPEDRSQPGQDARFRVT | | L1/CD3 | |
| | | QLPNGRDFHMSVVRARRNDSGTYLCGAISLAPK | | | |
| | | AQIKESLRAELRVTEGGGGSGEVQLVESGGGLV | | | |
| | | QPGGSLRLSCAASGVTFNYYGMSWIRQAPGKGL | | | |
| | | EWVASITSSGGRIYYPDSVKGRFTISRENTQKTL | | | |
| | | YLQMNSLRAEDTAVYYCTLDGRDGWVAYWGQ | | | |
| | | GTLVTVSSASTKGPSVFPLAPSSKSTSGGTAALG | | | |
| | | CLVKDYFPEPVTVSWNSGALTSGVHTFPAVLQS | | | |
| | | SGLYSLSSVVTVPSSSLGTQTYICNVNHKPSNTK | | | |
| | | VDKKVEPKSCDKTHTCPPCPAPEAAGGPSVFLFP | | | |
| | | PKPKDTLMISRTPEVTCVVVSVSHEDPEVKFNW | | | |
| | | YVDGVEVHNAKTKPREEQYNSTYRVVSVLTVL | | | |
| | | HQDWLNGKEYKCKVSNKALPAPIEKTISKAKGQ | | | |
| | | PREPQVYVYPPSRDELTKNQVSLTCLVKGFYPSD | | | |
| | | IAVEWESNGQPENNYKTTPPVLDSDGSFALVSK | | | |
| | | LTVDKSRWQQGNVFSCSVMHEALHNHYTQKSL | | | |
| | | SLSPG | | | |
| 29215 | | NPPTFSPALLVVTEGDNATFTCSFSNTSESFRLV | | Cldn18.2 | 415 |
| | | WHRESPGYETDTLASFPEDRSTPLPDARFRVTQL | | | |
| | | PNGRDFHMSVVRARRNDSGTYVCGAIAFHPVIQ | | | |
| | | IKESLRAELRVTEGGGGSGQVQLVESGGGLVQP | | | |
| | | GGSLRLSCSVSGIDLSSNPMIWVRQAPGKGLQYI | | | |
| | | GIIDTDGSTYYADWAKGRFTISKDSTTVYLQINS | | | |
| | | PRAEDTAVYYCARRLHGSSNGYYDDLWGQGTL | | | |
| | | VTVSSASTKGPSVFPLAPSSKSTSGGTAALGCLV | | | |
| | | KDYFPEPVTVSWNSGALTSGVHTFPAVLQSSGL | | | |
| | | YSLSSVVTVPSSSLGTQTYICNVNHKPSNTKVDK | | | |
| | | KVEPKSCDKTHTCPPCPAPEAAGGPSVFLFPPKP | | | |
| | | KDTLMISRTPEVTCVVVSVSHEDPEVKFNWYVD | | | |
| | | GVEVHNAKTKPREEQYNSTYRVVSVLTVLHQD | | | |
| | | WLNGKEYKCKVSNKALPAPIEKTISKAKGQPRE | | | |
| | | PQVYVLPPSRDELTKNQVSLLCLVKGFYPSDIAV | | | |
| | | EWESNGQPENNYLTWPPVLDSDGSFFLYSKLTV | | | |
| | | DKSRWQQGNVFSCSVMHEALHNHYTQKSLSLS | | | |
| | | PG | | | |
| 28423 | | DGQMTQSPSSVSASVGDRVTITCQASQSIYSYLS | | Cldn18.2 | 416 |
| | | WYQQKPGQRPKLLIYKASTLASGVPSRFSGSGS | | | |
| | | GTDFTLTISSVQPEDFATYYCQQGYTVTNVDKN | | | |
| | | TFGGGTKVEIKRTVAAPSVFIFPPSDEQLKSGTAS | | | |
| | | VVCLLNNFYPREAKVQWKVDNALQSGNSQESV | | | |
| | | TEQDSKDSTYSLSSTLTLSKADYEKHKVYACEV | | | |
| | | THQGLSSPVTKSFNRGEC | | | |
| 29217 | | NPPTFSPALLVVTEGDNATFTCSFSNTSESFVLN | | PD-L1/ | 417 |
| | | WYRMSPSNQTDKLAAFPEDRSQPGQDARFRVT | | Cldn18.2 | |
| | | QLPNGRDFHMSVVRARRNDSGTYLCGAISLAPK | | | |
| | | LQIKESLRAELRVTEGGGGSGQVQLVESGGGLV | | | |
| | | QPGGSLRLSCSVSGIDLSSNPMIWVRQAPGKGLQ | | | |
| | | YIGIIDTDGSTYYADWAKGRFTISKDSTTVYLQI | | | |
| | | NSPRAEDTAVYYCARRLHGSSNGYYDDLWGQG | | | |
| | | TLVTVSSASTKGPSVFPLAPSSKSTSGGTAALGC | | | |
| | | LVKDYFPEPVTVSWNSGALTSGVHTFPAVLQSS | | | |
| | | GLYSLSSVVTVPSSSLGTQTYICNVNHKPSNTKV | | | |
| | | DKKVEPKSCDKTHTCPPCPAPEAAGGPSVFLFPP | | | |
| | | KPKDTLMISRTPEVTCVVVSVSHEDPEVKFNWY | | | |
| | | VDGVEVHNAKTKPREEQYNSTYRVVSVLTVLH | | | |
| | | QDWLNGKEYKCKVSNKALPAPIEKTISKAKGQP | | | |
| | | REPQVYVLPPSRDELTKNQVSLLCLVKGFYPSDI | | | |
| | | AVEWESNGQPENNYLTWPPVLDSDGSFFLYSKL | | | |
| | | TVDKSRWQQGNVFSCSVMHEALHNHYTQKSLS | | | |
| | | LSPG | | | |
| 29218 | | NPPTFSPALLVVTEGDNATFTCSFSNTSESFVLN | | PD-L1/ | 418 |
| | | WYRMSPSNQTDKLAAFPEDRSQPGQDARFRVT | | Cldn18.2 | |
| | | QLPNGRDFHMSVVRARRNDSGTYLCGAISLAPK | | | |
| | | AQIKESLRAELRVTEGGGGSGQVQLVESGGGLV | | | |
| | | QPGGSLRLSCSVSGIDLSSNPMIWVRQAPGKGLQ | | | |
| | | YIGIIDTDGSTYYADWAKGRFTISKDSTTVYLQI | | | |
| | | NSPRAEDTAVYYCARRLHGSSNGYYDDLWGQG | | | |
| | | TLVTVSSASTKGPSVFPLAPSSKSTSGGTAALGC | | | |
| | | LVKDYFPEPVTVSWNSGALTSGVHTFPAVLQSS | | | |
| | | GLYSLSSVVTVPSSSLGTQTYICNVNHKPSNTKV | | | |
| | | DKKVEPKSCDKTHTCPPCPAPEAAGGPSVFLFPP | | | |
| | | KPKDTLMISRTPEVTCVVVSVSHEDPEVKFNWY | | | |
| | | VDGVEVHNAKTKPREEQYNSTYRVVSVLTVLH | | | |
| | | QDWLNGKEYKCKVSNKALPAPIEKTISKAKGQP | | | |
| | | REPQVYVLPPSRDELTKNQVSLLCLVKGFYPSDI | | | |
| | | AVEWESNGQPENNYLTWPPVLDSDGSFFLYSKL | | | |
| | | TVDKSRWQQGNVFSCSVMHEALHNHYTQKSLS | | | |
| | | LSPG | | | |
| 29239 | | EVQLVESGGGLVQPGGSLRLSCAASGVTFNYYG | | CD3 | 419 |
| | | MSWIRQAPGKGLEWVASITSSGGRIYYPDSVKG | | | |
| | | RFTISRENTQKTLYLQMNSLRAEDTAVYYCTLD | | | |
| | | GRDGWVAYWGQGTLVTVSSASTKGPSVFPLAP | | | |
| | | SSKSTSGGTAALGCLVKDYFPEPVTVSWNSGAL | | | |
| | | TSGVHTFPAVLQSSGLYSLSSVVTVPSSSLGTQT | | | |
| | | YICNVNHKPSNTKVDKKVEPKSCDKTHTCPPCP | | | |
| | | APEAAGGPSVFLFPPKPKDTLMISRTPEVTCVVV | | | |
| | | SVSHEDPEVKFNWYVDGVEVHNAKTKPREEQY | | | |
| | | NSTYRVVSVLTVLHQDWLNGKEYKCKVSNKAL | | | |
| | | PAPIEKTISKAKGQPREPQVYVYPPSRDELTKNQ | | | |
| | | VSLTCLVKGFYPSDIAVEWESNGQPENNYKTTPP | | | |
| | | VLDSDGSFALVSKLTVDKSRWQQGNVFSCSVM | | | |
| | | HEALHNHYTQKSLSLSPGGGGGSGDRPWNPPTF | | | |
| | | SPALLVVTEGDNATFTCSFSNTSESFRLVWHRES | | | |
| | | PGYETDTLASFPEDRSTPLPDARFRVTQLPNGRD | | | |
| | | FHMSVVRARRNDSGTYVCGAIAFHPVIQIKESLR | | | |
| | | AELRVTE | | | |
| 29242 | | EVQLVESGGGLVQPGGSLRLSCAASGVTFNYYG | | CD3/PD- | 420 |
| | | MSWIRQAPGKGLEWVASITSSGGRIYYPDSVKG | | L1 | |
| | | RFTISRENTQKTLYLQMNSLRAEDTAVYYCTLD | | | |
| | | GRDGWVAYWGQGTLVTVSSASTKGPSVFPLAP | | | |
| | | SSKSTSGGTAALGCLVKDYFPEPVTVSWNSGAL | | | |
| | | TSGVHTFPAVLQSSGLYSLSSVVTVPSSSLGTQT | | | |
| | | YICNVNHKPSNTKVDKKVEPKSCDKTHTCPPCP | | | |
| | | APEAAGGPSVFLFPPKPKDTLMISRTPEVTCVVV | | | |
| | | SVSHEDPEVKFNWYVDGVEVHNAKTKPREEQY | | | |
| | | NSTYRVVSVLTVLHQDWLNGKEYKCKVSNKAL | | | |
| | | PAPIEKTISKAKGQPREPQVYVYPPSRDELTKNQ | | | |
| | | VSLTCLVKGFYPSDIAVEWESNGQPENNYKTTPP | | | |
| | | VLDSDGSFALVSKLTVDKSRWQQGNVFSCSVM | | | |
| | | HEALHNHYTQKSLSLSPGGGGGSGDRPWNPPTF | | | |
| | | SPALLVVTEGDNATFTCSFSNTSESFVLNWYRM | | | |
| | | SPSNQTDKLAAFPEDRSQPGQDARFRVTQLPNG | | | |
| | | RDFHMSVVRARRNDSGTYLCGAISLAPKAQIKE | | | |
| | | SLRAELRVTE | | | |
| 29221 | | NPPTFSPALLVVTEGDNATFTCSFSNTSESFRLV | | CD3 | 421 |
| | | WHRESPGYETDTLASFPEDRSTPLPDARFRVTQL | | | |
| | | PNGRDFHMSVVRARRNDSGTYVCGAIAFHPVIQ | | | |
| | | IKESLRAELRVTEGGGGSGNFMLTQPHSVSESPG | | | |
| | | KTVTISCKRNTGNIGSNYVNWYQQHEGSSPTTII | | | |
| | | YRNDKRPDGVSDRESGSIDRSSKSASLTISNLKTE | | | |
| | | DEADYFCQSYSSGFIFGGGTKLTVLGQPKAAPSV | | | |
| | | TLFPPSSEELQANKATLVCLISDFYPGAVTVAWK | | | |
| | | ADSSPVKAGVETTTPSKQSNNKYAASSYLSLTPE | | | |
| | | QWKSHRSYSCQVTHEGSTVEKTVAPTECS | | | |
| 29223 | | NPPTFSPALLVVTEGDNATFTCSFSNTSESFVLN | | PD- | 422 |
| | | WYRMSPSNQTDKLAAFPEDRSQPGQDARFRVT | | L1/CD3 | |
| | | QLPNGRDFHMSVVRARRNDSGTYLCGAISLAPK | | | |
| | | LQIKESLRAELRVTEGGGGSGNFMLTQPHSVSES | | | |
| | | PGKTVTISCKRNTGNIGSNYVNWYQQHEGSSPT | | | |
| | | TIIYRNDKRPDGVSDRFSGSIDRSSKSASLTISNLK | | | |
| | | TEDEADYFCQSYSSGFIFGGGTKLTVLGQPKAAP | | | |
| | | SVTLFPPSSEELQANKATLVCLISDFYPGAVTVA | | | |
| | | WKADSSPVKAGVETTTPSKQSNNKYAASSYLSL | | | |
| | | TPEQWKSHRSYSCQVTHEGSTVEKTVAPTECS | | | |
| 29224 | | NPPTFSPALLVVTEGDNATFTCSFSNTSESFVLN | | PD- | 423 |
| | | WYRMSPSNQTDKLAAFPEDRSQPGQDARFRVT | | L1/CD3 | |
| | | QLPNGRDFHMSVVRARRNDSGTYLCGAISLAPK | | | |
| | | AQIKESLRAELRVTEGGGGSGNFMLTQPHSVSES | | | |
| | | PGKTVTISCKRNTGNIGSNYVNWYQQHEGSSPT | | | |
| | | TIIYRNDKRPDGVSDRFSGSIDRSSKSASLTISNLK | | | |
| | | TEDEADYFCQSYSSGFIFGGGTKLTVLGQPKAAP | | | |
| | | SVTLFPPSSEELQANKATLVCLISDFYPGAVTVA | | | |
| | | WKADSSPVKAGVETTTPSKQSNNKYAASSYLSL | | | |
| | | TPEQWKSHRSYSCQVTHEGSTVEKTVAPTECS | | | |
| 29262 | | NPPTFSPALLVVTEGDNATFTCSFSNTSESFVLN | | PD-L1/ | 424 |
| | | WYRMSPSNQTDKLAAFPEDRSQPGQDARFRVT | | MSLN | |
| | | QLPNGRDFHMSVVRARRNDSGTYLCGAISLAPK | | | |
| | | AQIKESLRAELRVTEGGGGSGDIQMTQSPSSLSA | | | |
| | | SVGDRVTITCSASSSVSYMHWYQQKSGKAPKLL | | | |
| | | IYDTSKLASGVPSRFSGSGSGTDFTLTISSLQPEDF | | | |
| | | ATYYCQQWSGYPLTFGQGTKLEIKGGGGSGGG | | | |
| | | GSGGGGSGGGGSQVQLVQSGAEVKKPGASVKV | | | |
| | | SCKASGYSFTGYTMNWVRQAPGQGLEWMGLIT | | | |
| | | PYNGASSYNQKFRGKATMTVDTSTSTVYMELSS | | | |
| | | LRSEDTAVYYCARGGYDGRGFDYWGQGTLVTV | | | |
| | | SSAAEPKSSDKTHTCPPCPAPEAAGGPSVFLFPPK | | | |
| | | PKDTLMISRTPEVTCVVVSVSHEDPEVKFNWYV | | | |
| | | DGVEVHNAKTKPREEQYNSTYRVVSVLTVLHQ | | | |
| | | DWLNGKEYKCKVSNKALPAPIEKTISKAKGQPR | | | |
| | | EPQVYVLPPSRDELTKNQVSLLCLVKGFYPSDIA | | | |
| | | VEWESNGQPENNYLTWPPVLDSDGSFFLYSKLT | | | |
| | | VDKSRWQQGNVFSCSVMHEALHNHYTQKSLSL | | | |
| | | SPG | | | |
| 29250 | | NPPTFSPALLVVTEGDNATFTCSFSNTSESFVLN | | PD-L1/ | 425 |
| | | WYRMSPSNQTDALAAFPEDRSQPGQDARFRVT | | Cldn18.2 | |
| | | QLPNGRDFHMSVVRARRNDSGTYLCGAASLAP | | | |
| | | KAQIKESLRAELRVTEGGGGSGQVQLVESGGGL | | | |
| | | VQPGGSLRLSCSVSGIDLSSNPMIWVRQAPGKGL | | | |
| | | QYIGIIDTDGSTYYADWAKGRFTISKDSTTVYLQ | | | |
| | | INSPRAEDTAVYYCARRLHGSSNGYYDDLWGQ | | | |
| | | GTLVTVSSGGGGSGGGGSGGGGSGGGGSDGQM | | | |
| | | TQSPSSVSASVGDRVTITCQASQSIYSYLSWYQQ | | | |
| | | KPGQRPKLLIYKASTLASGVPSRFSGSGSGTDFT | | | |
| | | LTISSVQPEDFATYYCQQGYTVTNVDKNTFGGG | | | |
| | | TKVEIKAAEPKSSDKTHTCPPCPAPEAAGGPSVF | | | |
| | | LFPPKPKDTLMISRTPEVTCVVVSVSHEDPEVKF | | | |
| | | NWYVDGVEVHNAKTKPREEQYNSTYRVVSVLT | | | |
| | | VLHQDWLNGKEYKCKVSNKALPAPIEKTISKAK | | | |
| | | GQPREPQVYVLPPSRDELTKNQVSLLCLVKGFY | | | |
| | | PSDIAVEWESNGQPENNYLTWPPVLDSDGSFFL | | | |
| | | YSKLTVDKSRWQQGNVFSCSVMHEALHNHYTQ | | | |
| | | KSLSLSPG | | | |
| 29256 | | QVQLVESGGGLVQPGGSLRLSCSVSGIDLSSNPM | | Cldn18.2/ | 426 |
| | | IWVRQAPGKGLQYIGIIDTDGSTYYADWAKGRF | | PD-L1 | |
| | | TISKDSTTVYLQINSPRAEDTAVYYCARRLHGSS | | | |
| | | NGYYDDLWGQGTLVTVSSGGGGSGGGGSGGG | | | |
| | | GSGGGGSDGQMTQSPSSVSASVGDRVTITCQAS | | | |
| | | QSIYSYLSWYQQKPGQRPKLLIYKASTLASGVPS | | | |
| | | RFSGSGSGTDFTLTISSVQPEDFATYYCQQGYTV | | | |
| | | TNVDKNTFGGGTKVEIKAAEPKSSDKTHTCPPCP | | | |
| | | APEAAGGPSVFLFPPKPKDTLMISRTPEVTCVVV | | | |
| | | SVSHEDPEVKFNWYVDGVEVHNAKTKPREEQY | | | |
| | | NSTYRVVSVLTVLHQDWLNGKEYKCKVSNKAL | | | |
| | | PAPIEKTISKAKGQPREPQVYVLPPSRDELTKNQ | | | |
| | | VSLLCLVKGFYPSDIAVEWESNGQPENNYLTWP | | | |
| | | PVLDSDGSFFLYSKLTVDKSRWQQGNVFSCSVM | | | |
| | | HEALHNHYTQKSLSLSPGGGGGSGDRPWNPPTF | | | |
| | | SPALLVVTEGDNATFTCSFSNTSESFVLNWYRM | | | |
| | | SPSNQTDALAAFPEDRSQPGQDARFRVTQLPNG | | | |
| | | RDFHMSVVRARRNDSGTYLCGAASLAPKAQIKE | | | |
| | | SLRAELRVTE | | | |
| 12989 | | QVQLVESGGGVVQPGRSLRLSCKASGYTFTRST | | CD3 | 1427 |
| | | MHWVRQAPGQGLEWIGYINPSSAYTNYNQKFK | | | |
| | | DRFTISADKSKSTAFLQMDSLRPEDTGVYFCARP | | | |
| | | QVHYDYNGFPYWGQGTPVTVSSASTKGPSVFPL | | | |
| | | APSSKSTSGGTAALGCLVKDYFPEPVTVSWNSG | | | |
| | | ALTSGVHTFPAVLQSSGLYSLSSVVTVPSSSLGT | | | |
| | | QTYICNVNHKPSNTKVDKKVEPKSCDKTHTCPP | | | |
| | | CPAPEAAGGPSVFLFPPKPKDTLMISRTPEVTCV | | | |
| | | VVSVSHEDPEVKFNWYVDGVEVHNAKTKPREE | | | |
| | | QYNSTYRVVSVLTVLHQDWLNGKEYKCKVSNK | | | |
| | | ALPAPIEKTISKAKGQPREPQVYVYPPSRDELTK | | | |
| | | NQVSLTCLVKGFYPSDIAVEWESNGQPENNYKT | | | |
| | | TPPVLDSDGSFALVSKLTVDKSRWQQGNVFSCS | | | |
| | | VMHEALHNHYTQKSLSLSPG | | | |
| 22082 | | NPPTFSPALLVVTEGDNATFTCSFSNTSESFVLN | | PD- | 428 |
| | | WYRMSPSNQTDKLAAFPEDRSQPGQDSRFRVTQ | | L1/CD3 | |
| | | LPNGRDFHMSVVRARRNDSGTYLCGAISLAPKA | | | |
| | | QIKESLRAELRVTEEAAAKEAAAKQVQLVESGG | | | |
| | | GVVQPGRSLRLSCKASGYTFTRSTMHWVRQAP | | | |
| | | GQGLEWIGYINPSSAYTNYNQKFKDRFTISADKS | | | |
| | | KSTAFLQMDSLRPEDTGVYFCARPQVHYDYNGF | | | |
| | | PYWGQGTPVTVSSASTKGPSVFPLAPSSKSTSGG | | | |
| | | TAALGCLVKDYFPEPVTVSWNSGALTSGVHTFP | | | |
| | | AVLQSSGLYSLSSVVTVPSSSLGTQTYICNVNHK | | | |
| | | PSNTKVDKKVEPKSCDKTHTCPPCPAPEAAGGP | | | |
| | | SVFLFPPKPKDTLMISRTPEVTCVVVSVSHEDPE | | | |
| | | VKFNWYVDGVEVHNAKTKPREEQYNSTYRVVS | | | |
| | | VLTVLHQDWLNGKEYKCKVSNKALPAPIEKTIS | | | |
| | | KAKGQPREPQVYVYPPSRDELTKNQVSLTCLVK | | | |
| | | GFYPSDIAVEWESNGQPENNYKTTPPVLDSDGSF | | | |
| | | ALVSKLTVDKSRWQQGNVFSCSVMHEALHNHY | | | |
| | | TQKSLSLSPG | | | |
| 22081 | | NPPTFSPALLVVTEGDNATFTCSFSNTSESFVLN | | PD- | 429 |
| | | WYRMSPSNQTDKLAAFPEDRSQPGQDSRFRVTQ | | L1/CD3 | |
| | | LPNGRDFHMSVVRARRNDSGTYLCGAISLAPKL | | | |
| | | QIKESLRAELRVTEEAAAKEAAAKQVQLVESGG | | | |
| | | GVVQPGRSLRLSCKASGYTFTRSTMHWVRQAP | | | |
| | | GQGLEWIGYINPSSAYTNYNQKFKDRFTISADKS | | | |
| | | KSTAFLQMDSLRPEDTGVYFCARPQVHYDYNGF | | | |
| | | PYWGQGTPVTVSSASTKGPSVFPLAPSSKSTSGG | | | |
| | | TAALGCLVKDYFPEPVTVSWNSGALTSGVHTFP | | | |
| | | AVLQSSGLYSLSSVVTVPSSSLGTQTYICNVNHK | | | |
| | | PSNTKVDKKVEPKSCDKTHTCPPCPAPEAAGGP | | | |
| | | SVFLFPPKPKDTLMISRTPEVTCVVVSVSHEDPE | | | |
| | | VKFNWYVDGVEVHNAKTKPREEQYNSTYRVVS | | | |
| | | VLTVLHQDWLNGKEYKCKVSNKALPAPIEKTIS | | | |
| | | KAKGQPREPQVYVYPPSRDELTKNQVSLTCLVK | | | |
| | | GFYPSDIAVEWESNGQPENNYKTTPPVLDSDGSF | | | |
| | | ALVSKLTVDKSRWQQGNVFSCSVMHEALHNHY | | | |
| | | TQKSLSLSPG | | | |
| 29200 | | NPPTFSPALLVVTEGDNATFTCSFSNTSESFRLV | | PD- | 430 |
| | | WHRESPGYETDTLASFPEDRSTPLPDARFRVTQL | | L1/CD3 | |
| | | PNGRDFHMSVVRARRNDSGTYVCGAIAFHPVIQ | | | |
| | | IKESLRAELRVTEEAAAKEAAAKQVQLVESGGG | | | |
| | | VVQPGRSLRLSCKASGYTFTRSTMHWVRQAPG | | | |
| | | QGLEWIGYINPSSAYTNYNQKFKDRFTISADKSK | | | |
| | | STAFLQMDSLRPEDTGVYFCARPQVHYDYNGFP | | | |
| | | YWGQGTPVTVSSASTKGPSVFPLAPSSKSTSGGT | | | |
| | | AALGCLVKDYFPEPVTVSWNSGALTSGVHTFPA | | | |
| | | VLQSSGLYSLSSVVTVPSSSLGTQTYICNVNHKP | | | |
| | | SNTKVDKKVEPKSCDKTHTCPPCPAPEAAGGPS | | | |
| | | VFLFPPKPKDTLMISRTPEVTCVVVSVSHEDPEV | | | |
| | | KFNWYVDGVEVHNAKTKPREEQYNSTYRVVSV | | | |
| | | LTVLHQDWLNGKEYKCKVSNKALPAPIEKTISK | | | |
| | | AKGQPREPQVYVYPPSRDELTKNQVSLTCLVKG | | | |
| | | FYPSDIAVEWESNGQPENNYKTTPPVLDSDGSFA | | | |
| | | LVSKLTVDKSRWQQGNVFSCSVMHEALHNHYT | | | |
| | | QKSLSLSPG | | | |
| 29269 | | DIQMTQSPSSLSASVGDRVTITCSASSSVSYMHW | | PD- | 431 |
| | | YQQKSGKAPKLLIYDTSKLASGVPSRFSGSGSGT | | L1/MSLN | |
| | | DFTLTISSLQPEDFATYYCQQWSGYPLTFGQGTK | | | |
| | | LEIKGGGGSGGGGSGGGGSGGGGSQVQLVQSG | | | |
| | | AEVKKPGASVKVSCKASGYSFTGYTMNWVRQA | | | |
| | | PGQGLEWMGLITPYNGASSYNQKFRGKATMTV | | | |
| | | DTSTSTVYMELSSLRSEDTAVYYCARGGYDGRG | | | |
| | | FDYWGQGTLVTVSSAAEPKSSDKTHTCPPCPAP | | | |
| | | EAAGGPSVFLFPPKPKDTLMISRTPEVTCVVVSV | | | |
| | | SHEDPEVKFNWYVDGVEVHNAKTKPREEQYNS | | | |
| | | TYRVVSVLTVLHQDWLNGKEYKCKVSNKALPA | | | |
| | | PIEKTISKAKGQPREPQVYVLPPSRDELTKNQVS | | | |
| | | LLCLVKGFYPSDIAVEWESNGQPENNYLTWPPV | | | |
| | | LDSDGSFFLYSKLTVDKSRWQQGNVFSCSVMHE | | | |
| | | ALHNHYTQKSLSLSPGGGGGSGDRPWNPPTFSP | | | |
| | | ALLVVTEGDNATFTCSFSNTSESFVLNWYRMSP | | | |
| | | SNQTDALAAFPEDRSQPGQDARFRVTQLPNGRD | | | |
| | | FHMSVVRARRNDSGTYLCGAASLAPKAQIKESL | | | |
| | | RAELRVTE | | | |
| 29232 | | NPPTFSPALLVVTEGDNATFTCSFSNTSESFRLV | | PD-L1 | 432 |
| | | WHRESPGYETDTLASFPEDRSTPLPDARFRVTQL | | | |
| | | PNGRDFHMSVVRARRNDSGTYVCGAIAFHPVIQ | | | |
| | | IKESLRAELRVTEAAEPKSSDKTHTCPPCPAPEA | | | |
| | | AGGPSVFLFPPKPKDTLMISRTPEVTCVVVSVSH | | | |
| | | EDPEVKFNWYVDGVEVHNAKTKPREEQYNSTY | | | |
| | | RVVSVLTVLHQDWLNGKEYKCKVSNKALPAPIE | | | |
| | | KTISKAKGQPREPQVYVLPPSRDELTKNQVSLLC | | | |
| | | LVKGFYPSDIAVEWESNGQPENNYLTWPPVLDS | | | |
| | | DGSFFLYSKLTVDKSRWQQGNVFSCSVMHEAL | | | |
| | | HNHYTQKSLSLSPG | | | |
| 29234 | | NPPTFSPALLVVTEGDNATFTCSFSNTSESFVLN | | PD-L1 | 433 |
| | | WYRMSPSNQTDKLAAFPEDRSQPGQDARFRVT | | | |
| | | QLPNGRDFHMSVVRARRNDSGTYLCGAISLAPK | | | |
| | | LQIKESLRAELRVTEAAEPKSSDKTHTCPPCPAPE | | | |
| | | AAGGPSVFLFPPKPKDTLMISRTPEVTCVVVSVS | | | |
| | | HEDPEVKFNWYVDGVEVHNAKTKPREEQYNST | | | |
| | | YRVVSVLTVLHQDWLNGKEYKCKVSNKALPAP | | | |
| | | IEKTISKAKGQPREPQVYVLPPSRDELTKNQVSL | | | |
| | | LCLVKGFYPSDIAVEWESNGQPENNYLTWPPVL | | | |
| | | DSDGSFFLYSKLTVDKSRWQQGNVFSCSVMHE | | | |
| | | ALHNHYTQKSLSLSPG | | | |
| 29235 | | NPPTFSPALLVVTEGDNATFTCSESNTSESFVLN | | PD-L1 | 1434 |
| | | WYRMSPSNQTDKLAAFPEDRSQPGQDARFRVT | | | |
| | | QLPNGRDFHMSVVRARRNDSGTYLCGAISLAPK | | | |
| | | AQIKESLRAELRVTEAAEPKSSDKTHTCPPCPAP | | | |
| | | EAAGGPSVFLFPPKPKDTLMISRTPEVTCVVVSV | | | |
| | | SHEDPEVKFNWYVDGVEVHNAKTKPREEQYNS | | | |
| | | TYRVVSVLTVLHQDWLNGKEYKCKVSNKALPA | | | |
| | | PIEKTISKAKGQPREPQVYVLPPSRDELTKNQVS | | | |
| | | LLCLVKGFYPSDIAVEWESNGQPENNYLTWPPV | | | |
| | | LDSDGSFFLYSKLTVDKSRWQQGNVFSCSVMHE | | | |
| | | ALHNHYTQKSLSLSPG | | | |
| 29259 | | NPPTFSPALLVVTEGDNATFTCSFSNTSESFRLV | | PD-L1/ | 435 |
| | | WHRESPGYETDTLASFPEDRSTPLPDARFRVTQL | | MSLN | |
| | | PNGRDFHMSVVRARRNDSGTYVCGAIAFHPVIQ | | | |
| | | IKESLRAELRVTEGGGGSGDIQMTQSPSSLSASV | | | |
| | | GDRVTITCSASSSVSYMHWYQQKSGKAPKLLIY | | | |
| | | DTSKLASGVPSRFSGSGSGTDFTLTISSLQPEDFA | | | |
| | | TYYCQQWSGYPLTFGQGTKLEIKGGGGSGGGGS | | | |
| | | GGGGSGGGGSQVQLVQSGAEVKKPGASVKVSC | | | |
| | | KASGYSFTGYTMNWVRQAPGQGLEWMGLITPY | | | |
| | | NGASSYNQKFRGKATMTVDTSTSTVYMELSSLR | | | |
| | | SEDTAVYYCARGGYDGRGFDYWGQGTLVTVSS | | | |
| | | AAEPKSSDKTHTCPPCPAPEAAGGPSVFLFPPKP | | | |
| | | KDTLMISRTPEVTCVVVSVSHEDPEVKFNWYVD | | | |
| | | GVEVHNAKTKPREEQYNSTYRVVSVLTVLHQD | | | |
| | | WLNGKEYKCKVSNKALPAPIEKTISKAKGQPRE | | | |
| | | PQVYVLPPSRDELTKNQVSLLCLVKGFYPSDIAV | | | |
| | | EWESNGQPENNYLTWPPVLDSDGSFFLYSKLTV | | | |
| | | DKSRWQQGNVFSCSVMHEALHNHYTQKSLSLS | | | |
| | | PG | | | |
| 29265 | | DIQMTQSPSSLSASVGDRVTITCSASSSVSYMHW | | PD-L1/ | 436 |
| | | YQQKSGKAPKLLIYDTSKLASGVPSRFSGSGSGT | | MSLN | |
| | | DFTLTISSLQPEDFATYYCQQWSGYPLTFGQGTK | | | |
| | | LEIKGGGGSGGGGSGGGGSGGGGSQVQLVQSG | | | |
| | | AEVKKPGASVKVSCKASGYSFTGYTMNWVRQA | | | |
| | | PGQGLEWMGLITPYNGASSYNQKFRGKATMTV | | | |
| | | DTSTSTVYMELSSLRSEDTAVYYCARGGYDGRG | | | |
| | | FDYWGQGTLVTVSSAAEPKSSDKTHTCPPCPAP | | | |
| | | EAAGGPSVFLFPPKPKDTLMISRTPEVTCVVVSV | | | |
| | | SHEDPEVKFNWYVDGVEVHNAKTKPREEQYNS | | | |
| | | TYRVVSVLTVLHQDWLNGKEYKCKVSNKALPA | | | |
| | | PIEKTISKAKGQPREPQVYVLPPSRDELTKNQVS | | | |
| | | LLCLVKGFYPSDIAVEWESNGQPENNYLTWPPV | | | |
| | | LDSDGSFFLYSKLTVDKSRWQQGNVFSCSVMHE | | | |
| | | ALHNHYTQKSLSLSPGGGGGSGDRPWNPPTFSP | | | |
| | | ALLVVTEGDNATFTCSFSNTSESFRLVWHRESPG | | | |
| | | YETDTLASFPEDRSTPLPDARFRVTQLPNGRDFH | | | |
| | | MSVVRARRNDSGTYVCGAIAFHPVIQIKESLRAE | | | |
| | | LRVTE | | | |
| 29268 | | DIQMTQSPSSLSASVGDRVTITCSASSSVSYMHW | | PD-L1/ | 437 |
| | | YQQKSGKAPKLLIYDTSKLASGVPSRFSGSGSGT | | MSLN | |
| | | DFTLTISSLQPEDFATYYCQQWSGYPLTFGQGTK | | | |
| | | LEIKGGGGSGGGGSGGGGSGGGGSQVQLVQSG | | | |
| | | AEVKKPGASVKVSCKASGYSFTGYTMNWVRQA | | | |
| | | PGQGLEWMGLITPYNGASSYNQKFRGKATMTV | | | |
| | | DTSTSTVYMELSSLRSEDTAVYYCARGGYDGRG | | | |
| | | FDYWGQGTLVTVSSAAEPKSSDKTHTCPPCPAP | | | |
| | | EAAGGPSVFLFPPKPKDTLMISRTPEVTCVVVSV | | | |
| | | SHEDPEVKFNWYVDGVEVHNAKTKPREEQYNS | | | |
| | | TYRVVSVLTVLHQDWLNGKEYKCKVSNKALPA | | | |
| | | PIEKTISKAKGQPREPQVYVLPPSRDELTKNQVS | | | |
| | | LLCLVKGFYPSDIAVEWESNGQPENNYLTWPPV | | | |
| | | LDSDGSFFLYSKLTVDKSRWQQGNVFSCSVMHE | | | |
| | | ALHNHYTQKSLSLSPGGGGGSGDRPWNPPTFSP | | | |
| | | ALLVVTEGDNATFTCSFSNTSESFVLNWYRMSP | | | |
| | | SNQTDKLAAFPEDRSQPGQDARFRVTQLPNGRD | | | |
| | | FHMSVVRARRNDSGTYLCGAISLAPKAQIKESLR | | | |
| | | AELRVTE | | | |
| 29231 | | NFMLTQPHSVSESPGKTVTISCKRNTGNIGSNYV | | CD3/PD- | 438 |
| | | NWYQQHEGSSPTTIIYRNDKRPDGVSDRFSGSID | | L1 | |
| | | RSSKSASLTISNLKTEDEADYFCQSYSSGFIFGGG | | | |
| | | TKLTVLGQPKAAPSVTLFPPSSEELQANKATLVC | | | |
| | | LISDFYPGAVTVAWKADSSPVKAGVETTTPSKQ | | | |
| | | SNNKYAASSYLSLTPEQWKSHRSYSCQVTHEGS | | | |
| | | TVEKTVAPTECSGGGGSGDRPWNPPTFSPALLV | | | |
| | | VTEGDNATFTCSFSNTSESFVLNWYRMSPSNQT | | | |
| | | DALAAFPEDRSQPGQDARFRVTQLPNGRDFHMS | | | |
| | | VVRARRNDSGTYLCGAASLAPKAQIKESLRAEL | | | |
| | | RVTE | | | |
| 23866 | | DIQMTQSPSSLSASVGDRVTITCSASSSVSYMHW | | MSLN/ | 439 |
| | | YQQKSGKAPKLLIYDTSKLASGVPSRFSGSGSGT | | CD3 | |
| | | DFTLTISSLQPEDFATYYCQQWSGYPLTFGQGTK | | | |
| | | LEIKGGGGSGGGGSGGGGSGGGGSQVQLVQSG | | | |
| | | AEVKKPGASVKVSCKASGYSFTGYTMNWVRQA | | | |
| | | PGQGLEWMGLITPYNGASSYNQKFRGKATMTV | | | |
| | | DTSTSTVYMELSSLRSEDTAVYYCARGGYDGRG | | | |
| | | FDYWGQGTLVTVSSGGGGSEVQLVESGGGLVQ | | | |
| | | PGGSLRLSCAASGVTFNYYGMSWIRQAPGKGLE | | | |
| | | WVASITSSGGRIYYPDSVKGRFTISRENTQKTLY | | | |
| | | LQMNSLRAEDTAVYYCTLDGRDGWVAYWGQG | | | |
| | | TLVTVSSASTKGPSVFPLAPSSKSTSGGTAALGC | | | |
| | | LVKDYFPEPVTVSWNSGALTSGVHTFPAVLQSS | | | |
| | | GLYSLSSVVTVPSSSLGTQTYICNVNHKPSNTKV | | | |
| | | DKKVEPKSCDKTHTCPPCPAPEAAGGPSVFLFPP | | | |
| | | KPKDTLMISRTPEVTCVVVSVSHEDPEVKENWY | | | |
| | | VDGVEVHNAKTKPREEQYNSTYRVVSVLTVLH | | | |
| | | QDWLNGKEYKCKVSNKALPAPIEKTISKAKGQP | | | |
| | | REPQVYVYPPSRDELTKNQVSLTCLVKGFYPSDI | | | |
| | | AVEWESNGQPENNYKTTPPVLDSDGSFALVSKL | | | |
| | | TVDKSRWQQGNVFSCSVMHEALHNHYTQKSLS | | | |
| | | LSPG | | | |
| 12153 | | EPKSSDKTHTCPPCPAPEAAGGPSVFLFPPKPKD | | n/a (Fc | 440 |
| | | TLMISRTPEVTCVVVSVSHEDPEVKFNWYVDGV | | only, | |
| | | EVHNAKTKPREEQYNSTYRVVSVLTVLHQDWL | | incl. | |
| | | NGKEYKCKVSNKALPAPIEKTISKAKGQPREPQV | | hinge) | |
| | | YVLPPSRDELTKNQVSLLCLVKGFYPSDIAVEWE | | | |
| | | SNGQPENNYLTWPPVLDSDGSFFLYSKLTVDKS | | | |
| | | RWQQGNVFSCSVMHEALHNHYTQKSLSLSPG | | | |
| 29201 | | NPPTFSPALLVVTEGDNATFTCSFSNTSESFRLV | | PD-L1 | 441 |
| | | WHRESPSYQTDTLAAFPEDRSQPGQDARFRVTQ | | (low | |
| | | LPNGRDFHMSVVRARRNDSGTYVCGAISLAPKI | | aff.)/ | |
| | | QIKESLRAELRVTEEAAAKEAAAKQVQLVESGG | | CD3 | |
| | | GVVQPGRSLRLSCKASGYTFTRSTMHWVRQAP | | | |
| | | GQGLEWIGYINPSSAYTNYNQKFKDRFTISADKS | | | |
| | | KSTAFLQMDSLRPEDTGVYFCARPQVHYDYNGF | | | |
| | | PYWGQGTPVTVSSASTKGPSVFPLAPSSKSTSGG | | | |
| | | TAALGCLVKDYFPEPVTVSWNSGALTSGVHTFP | | | |
| | | AVLQSSGLYSLSSVVTVPSSSLGTQTYICNVNHK | | | |
| | | PSNTKVDKKVEPKSCDKTHTCPPCPAPEAAGGP | | | |
| | | SVFLFPPKPKDTLMISRTPEVTCVVVSVSHEDPE | | | |
| | | VKFNWYVDGVEVHNAKTKPREEQYNSTYRVVS | | | |
| | | VLTVLHQDWLNGKEYKCKVSNKALPAPIEKTIS | | | |
| | | KAKGQPREPQVYVYPPSRDELTKNQVSLTCLVK | | | |
| | | GFYPSDIAVEWESNGQPENNYKTTPPVLDSDGSF | | | |
| | | ALVSKLTVDKSRWQQGNVFSCSVMHEALHNHY | | | |
| | | TQKSLSLSPG | | | |
| v38450 | H1 | DIQMTQSPSSLSASVGDRVTITCSASSSVSYMHW | | MSLN | 150 |
| | (29283) | YQQKSGKAPKLLIYDTSKLASGVPSRFSGSGSGT | | (scFv), | |
| | | DFTLTISSLQPEDFATYYCQQWSGYPLTFGQGTK | | CD3 | |
| | | LEIKGGGGSGGGGSGGGGSGGGGSQVQLVQSG | | (Fab) | |
| | | AEVKKPGASVKVSCKASGYSFTGYTMNWVRQA | | | |
| | | PGQGLEWMGLITPYNGASSYNQKFRGKATMTV | | | |
| | | DTSTSTVYMELSSLRSEDTAVYYCARGGYDGRG | | | |
| | | FDYWGQGTLVTVSSGGGGSGEVQLVESGGGLV | | | |
| | | QPGGSLRLSCAASGVTFNYYGMSWIRQAPGKGL | | | |
| | | EWVASITSSGGRIYYPDSVKGRFTISRENTQKTL | | | |
| | | YLQMNSLRAEDTAVYYCTLDGRDGWVAYWGQ | | | |
| | | GTLVTVSSASTKGPSVFPLAPSSKSTSGGTAALG | | | |
| | | CLVKDYFPEPVTVSWNSGALTSGVHTFPAVLQS | | | |
| | | SGLYSLSSVVTVPSSSLGTQTYICNVNHKPSNTK | | | |
| | | VDKKVEPKSCDKTHTCPPCPAPEAAGGPSVFLFP | | | |
| | | PKPKDTLMISRTPEVTCVVVSVSHEDPEVKFNW | | | |
| | | YVDGVEVHNAKTKPREEQYNSTYRVVSVLTVL | | | |
| | | HQDWLNGKEYKCKVSNKALPAPIEKTISKAKGQ | | | |
| | | PREPQVYVYPPSRDELTKNQVSLTCLVKGFYPSD | | | |
| | | IAVEWESNGQPENNYKTTPPVLDSDGSFALVSK | | | |
| | | LTVDKSRWQQGNVFSCSVMHEALHNHYTQKSL | | | |
| | | SLSPG | | | |
| | L1 | NFMLTQPHSVSESPGKTVTISCKRNTGNIGSNYV | | CD3 | 254 |
| | (16412) | NWYQQHEGSSPTTIIYRNDKRPDGVSDRFSGSID | | | |
| | | RSSKSASLTISNLKTEDEADYFCQSYSSGFIFGGG | | | |
| | | TKLTVLGQPKAAPSVTLFPPSSEELQANKATLVC | | | |
| | | LISDFYPGAVTVAWKADSSPVKAGVETTTPSKQ | | | |
| | | SNNKYAASSYLSLTPEQWKSHRSYSCQVTHEGS | | | |
| | | TVEKTVAPTECS | | | |
| | H2 | DIQMTQSPSSLSASVGDRVTITCSASSSVSYMHW | | MSLN | 151 |
| | (29264) | YQQKSGKAPKLLIYDTSKLASGVPSRFSGSGSGT | | (scFv), | |
| | | DFTLTISSLQPEDFATYYCQQWSGYPLTFGQGTK | | PD-L1 | |
| | | LEIKGGGGSGGGGSGGGGSGGGGSQVQLVQSG | | | |
| | | AEVKKPGASVKVSCKASGYSFTGYTMNWVRQA | | | |
| | | PGQGLEWMGLITPYNGASSYNQKFRGKATMTV | | | |
| | | DTSTSTVYMELSSLRSEDTAVYYCARGGYDGRG | | | |
| | | FDYWGQGTLVTVSSAAEPKSSDKTHTCPPCPAP | | | |
| | | EAAGGPSVFLFPPKPKDTLMISRTPEVTCVVVSV | | | |
| | | SHEDPEVKFNWYVDGVEVHNAKTKPREEQYNS | | | |
| | | TYRVVSVLTVLHQDWLNGKEYKCKVSNKALPA | | | |
| | | PIEKTISKAKGQPREPQVYVLPPSRDELTKNQVS | | | |
| | | LLCLVKGFYPSDIAVEWESNGQPENNYLTWPPV | | | |
| | | LDSDGSFFLYSKLTVDKSRWQQGNVFSCSVMHE | | | |
| | | ALHNHYTQKSLSLSPGGGGGSGDRPWNPPTFSP | | | |
| | | ALLVVTEGDNATFTCSFSNTSESFHVVWHRESPS | | | |
| | | GQTDTLAAFPEDRSQPGQDARFRVTQLPNGRDF | | | |
| | | HMSVVRARRNDSGTYVCGVISLAPKIQIKESLRA | | | |
| | | ELRVTE | | | |
| v38917 | H1 | DIQMTQSPSSLSASVGDRVTITCSASSSVSYMHW | | MSLN | 150 |
| | (29283) | YQQKSGKAPKLLIYDTSKLASGVPSRFSGSGSGT | | (scFv), | |
| | | DFTLTISSLQPEDFATYYCQQWSGYPLTFGQGTK | | CD3 | |
| | | LEIKGGGGSGGGGSGGGGSGGGGSQVQLVQSG | | (Fab) | |
| | | AEVKKPGASVKVSCKASGYSFTGYTMNWVRQA | | | |
| | | PGQGLEWMGLITPYNGASSYNQKFRGKATMTV | | | |
| | | DTSTSTVYMELSSLRSEDTAVYYCARGGYDGRG | | | |
| | | FDYWGQGTLVTVSSGGGGSGEVQLVESGGGLV | | | |
| | | QPGGSLRLSCAASGVTFNYYGMSWIRQAPGKGL | | | |
| | | EWVASITSSGGRIYYPDSVKGRFTISRENTQKTL | | | |
| | | YLQMNSLRAEDTAVYYCTLDGRDGWVAYWGQ | | | |
| | | GTLVTVSSASTKGPSVFPLAPSSKSTSGGTAALG | | | |
| | | CLVKDYFPEPVTVSWNSGALTSGVHTFPAVLQS | | | |
| | | SGLYSLSSVVTVPSSSLGTQTYICNVNHKPSNTK | | | |
| | | VDKKVEPKSCDKTHTCPPCPAPEAAGGPSVFLFP | | | |
| | | PKPKDTLMISRTPEVTCVVVSVSHEDPEVKFNW | | | |
| | | YVDGVEVHNAKTKPREEQYNSTYRVVSVLTVL | | | |
| | | HQDWLNGKEYKCKVSNKALPAPIEKTISKAKGQ | | | |
| | | PREPQVYVYPPSRDELTKNQVSLTCLVKGFYPSD | | | |
| | | IAVEWESNGQPENNYKTTPPVLDSDGSFALVSK | | | |
| | | LTVDKSRWQQGNVFSCSVMHEALHNHYTQKSL | | | |
| | | SLSPG | | | |
| | L1 | NFMLTQPHSVSESPGKTVTISCKRNTGNIGSNYV | | CD3 | 254 |
| | (16412) | NWYQQHEGSSPTTIIYRNDKRPDGVSDRFSGSID | | | |
| | | RSSKSASLTISNLKTEDEADYFCQSYSSGFIFGGG | | | |
| | | TKLTVLGQPKAAPSVTLFPPSSEELQANKATLVC | | | |
| | | LISDFYPGAVTVAWKADSSPVKAGVETTTPSKQ | | | |
| | | SNNKYAASSYLSLTPEQWKSHRSYSCQVTHEGS | | | |
| | | TVEKTVAPTECS | | | |
| | H2 | NPPTFSPALLVVTEGDNATFTCSFSNTSESFVLN | | PD-L1, | 152 |
| | (29261) | WYRMSPSNQTDKLAAFPEDRSQPGQDARFRVT | | MSLN | |
| | | QLPNGRDFHMSVVRARRNDSGTYLCGAISLAPK | | | |
| | | LQIKESLRAELRVTEGGGGSGDIQMTQSPSSLSA | | | |
| | | SVGDRVTITCSASSSVSYMHWYQQKSGKAPKLL | | | |
| | | IYDTSKLASGVPSRFSGSGSGTDFTLTISSLQPEDF | | | |
| | | ATYYCQQWSGYPLTFGQGTKLEIKGGGGSGGG | | | |
| | | GSGGGGSGGGGSQVQLVQSGAEVKKPGASVKV | | | |
| | | SCKASGYSFTGYTMNWVRQAPGQGLEWMGLIT | | | |
| | | PYNGASSYNQKFRGKATMTVDTSTSTVYMELSS | | | |
| | | LRSEDTAVYYCARGGYDGRGFDYWGQGTLVTV | | | |
| | | SSAAEPKSSDKTHTCPPCPAPEAAGGPSVFLFPPK | | | |
| | | PKDTLMISRTPEVTCVVVSVSHEDPEVKFNWYV | | | |
| | | DGVEVHNAKTKPREEQYNSTYRVVSVLTVLHQ | | | |
| | | DWLNGKEYKCKVSNKALPAPIEKTISKAKGQPR | | | |
| | | EPQVYVLPPSRDELTKNQVSLLCLVKGFYPSDIA | | | |
| | | VEWESNGQPENNYLTWPPVLDSDGSFFLYSKLT | | | |
| | | VDKSRWQQGNVFSCSVMHEALHNHYTQKSLSL | | | |
| | | SPG | | | |
| v38921 | H1 | DIQMTQSPSSLSASVGDRVTITCSASSSVSYMHW | | MSLN | 150 |
| | (29283) | YQQKSGKAPKLLIYDTSKLASGVPSRFSGSGSGT | | (scFv), | |
| | | DFTLTISSLQPEDFATYYCQQWSGYPLTFGQGTK | | CD3 | |
| | | LEIKGGGGSGGGGSGGGGSGGGGSQVQLVQSG | | (Fab) | |
| | | AEVKKPGASVKVSCKASGYSFTGYTMNWVRQA | | | |
| | | PGQGLEWMGLITPYNGASSYNQKFRGKATMTV | | | |
| | | DTSTSTVYMELSSLRSEDTAVYYCARGGYDGRG | | | |
| | | FDYWGQGTLVTVSSGGGGSGEVQLVESGGGLV | | | |
| | | QPGGSLRLSCAASGVTFNYYGMSWIRQAPGKGL | | | |
| | | EWVASITSSGGRIYYPDSVKGRFTISRENTQKTL | | | |
| | | YLQMNSLRAEDTAVYYCTLDGRDGWVAYWGQ | | | |
| | | GTLVTVSSASTKGPSVFPLAPSSKSTSGGTAALG | | | |
| | | CLVKDYFPEPVTVSWNSGALTSGVHTFPAVLQS | | | |
| | | SGLYSLSSVVTVPSSSLGTQTYICNVNHKPSNTK | | | |
| | | VDKKVEPKSCDKTHTCPPCPAPEAAGGPSVFLFP | | | |
| | | PKPKDTLMISRTPEVTCVVVSVSHEDPEVKFNW | | | |
| | | YVDGVEVHNAKTKPREEQYNSTYRVVSVLTVL | | | |
| | | HQDWLNGKEYKCKVSNKALPAPIEKTISKAKGQ | | | |
| | | PREPQVYVYPPSRDELTKNQVSLTCLVKGFYPSD | | | |
| | | IA VEWESNGQPENNYKTTPPVLDSDGSFALVSK | | | |
| | | LTVDKSRWQQGNVFSCSVMHEALHNHYTQKSL | | | |
| | | SLSPG | | | |
| | L1 | NFMLTQPHSVSESPGKTVTISCKRNTGNIGSNYV | | CD3 | 254 |
| | (16412) | NWYQQHEGSSPTTIIYRNDKRPDGVSDRFSGSID | | | |
| | | RSSKSASLTISNLKTEDEADYFCQSYSSGFIFGGG | | | |
| | | TKLTVLGQPKAAPSVTLFPPSSEELQANKATLVC | | | |
| | | LISDFYPGAVTVAWKADSSPVKAGVETTTPSKQ | | | |
| | | SNNKYAASSYLSLTPEQWKSHRSYSCQVTHEGS | | | |
| | | TVEKTVAPTECS | | | |
| | H2 | DIQMTQSPSSLSASVGDRVTITCSASSSVSYMHW | | MSLN, | 153 |
| | (29267) | YQQKSGKAPKLLIYDTSKLASGVPSRFSGSGSGT | | PD-L1 | |
| | | DFTLTISSLQPEDFATYYCQQWSGYPLTFGQGTK | | | |
| | | LEIKGGGGSGGGGSGGGGSGGGGSQVQLVQSG | | | |
| | | AEVKKPGASVKVSCKASGYSFTGYTMNWVRQA | | | |
| | | PGQGLEWMGLITPYNGASSYNQKFRGKATMTV | | | |
| | | DTSTSTVYMELSSLRSEDTAVYYCARGGYDGRG | | | |
| | | FDYWGQGTLVTVSSAAEPKSSDKTHTCPPCPAP | | | |
| | | EAAGGPSVFLFPPKPKDTLMISRTPEVTCVVVSV | | | |
| | | SHEDPEVKFNWYVDGVEVHNAKTKPREEQYNS | | | |
| | | TYRVVSVLTVLHQDWLNGKEYKCKVSNKALPA | | | |
| | | PIEKTISKAKGQPREPQVYVLPPSRDELTKNQVS | | | |
| | | LLCLVKGFYPSDIAVEWESNGQPENNYLTWPPV | | | |
| | | LDSDGSFFLYSKLTVDKSRWQQGNVFSCSVMHE | | | |
| | | ALHNHYTQKSLSLSPGGGGGSGDRPWNPPTFSP | | | |
| | | ALLVVTEGDNATFTCSFSNTSESFVLNWYRMSP | | | |
| | | SNQTDKLAAFPEDRSQPGQDARFRVTQLPNGRD | | | |
| | | FHMSVVRARRNDSGTYLCGAISLAPKLQIKESLR | | | |
| | | AELRVTE | | | |
| 38739 | HCDR1 | SNPMI | | Cldn18.2 | 310 |
| | HCDR2 | LIDTDGSTYYADWAKG | | Cldn18.2 | 311 |
| | HCDR3 | RLHGSSNGYYDDL | | Cldn18.2 | 312 |
| | LCDR1 | QASQSIYSYLS | | Cldn18.2 | 313 |
| | LCDR2 | KASTLAS | | Cldn18.2 | 314 |
| | LCDR3 | QQGYTVTNVDKNT | | Cldn18.2 | 315 |
| | VH | QVQLVESGGGLVQPGGSLRLSCSVSGIDLSSNPM | | Cldn18.2 | 316 |
| | | IWVRQAPGKGLQYIGIIDTDGSTYYADWAKGRF | | | |
| | | TISKDSTTVYLQINSPRAEDTAVYYCARRLHGSS | | | |
| | | NGYYDDLWGQGTLVTVSS | | | |
| | VL | DGQMTQSPSSVSASVGDRVTITCQASQSIYSYLS | | Cldn18.2 | 317 |
| | | WYQQKPGQRPKLLIYKASTLASGVPSRFSGSGS | | | |
| | | GTDFTLTISSVQPEDFATYYCQQGYTVTNVDKN | | | |
| | | TFGGGTKVEIK | | | |
| | H2 | QVQLVESGGGLVQPGGSLRLSCSVSGIDLSSNPM | | Cldn18.2 | 318 |
| | (28373) | IWVRQAPGKGLQYIGIIDTDGSTYYADWAKGRF | | | |
| | | TISKDSTTVYLQINSPRAEDTAVYYCARRLHGSS | | | |
| | | NGYYDDLWGQGTLVTVSSGGGGSGGGGSGGG | | | |
| | | GSGGGGSDGQMTQSPSSVSASVGDRVTITCQAS | | | |
| | | QSIYSYLSWYQQKPGQRPKLLIYKASTLASGVPS | | | |
| | | RFSGSGSGTDFTLTISSVQPEDFATYYCQQGYTV | | | |
| | | TNVDKNTFGGGTKVEIKAAEPKSSDKTHTCPPCP | | | |
| | | APEAAGGPSVFLFPPKPKDTLMISRTPEVTCVVV | | | |
| | | SVSHEDPEVKFNWYVDGVEVHNAKTKPREEQY | | | |
| | | NSTYRVVSVLTVLHQDWLNGKEYKCKVSNKAL | | | |
| | | PAPIEKTISKAKGQPREPQVYVLPPSRDELTKNQ | | | |
| | | VSLLCLVKGFYPSDIAVEWESNGQPENNYLTWP | | | |
| | | PVLDSDGSFFLYSKLTVDKSRWQQGNVFSCSVM | | | |
| | | HEALHNHYTQKSLSLSPG | | | |
| | H1 | EVQLVESGGGLVQPGGSLRLSCAASGVTFNYYG | | CD3/PD- | 319 |
| | (29241) | MSWIRQAPGKGLEWVASITSSGGRIYYPDSVKG | | L1 | |
| | | RFTISRENTQKTLYLQMNSLRAEDTAVYYCTLD | | | |
| | | GRDGWVAYWGQGTLVTVSSASTKGPSVFPLAP | | | |
| | | SSKSTSGGTAALGCLVKDYFPEPVTVSWNSGAL | | | |
| | | TSGVHTFPAVLQSSGLYSLSSVVTVPSSSLGTQT | | | |
| | | YICNVNHKPSNTKVDKKVEPKSCDKTHTCPPCP | | | |
| | | APEAAGGPSVFLFPPKPKDTLMISRTPEVTCVVV | | | |
| | | SVSHEDPEVKFNWYVDGVEVHNAKTKPREEQY | | | |
| | | NSTYRVVSVLTVLHQDWLNGKEYKCKVSNKAL | | | |
| | | PAPIEKTISKAKGQPREPQVYVYPPSRDELTKNQ | | | |
| | | VSLTCLVKGFYPSDIAVEWESNGQPENNYKTTPP | | | |
| | | VLDSDGSFALVSKLTVDKSRWQQGNVFSCSVM | | | |
| | | HEALHNHYTQKSLSLSPGGGGGSGDRPWNPPTF | | | |
| | | SPALLVVTEGDNATFTCSFSNTSESFVLNWYRM | | | |
| | | SPSNQTDKLAAFPEDRSQPGQDARFRVTQLPNG | | | |
| | | RDFHMSVVRARRNDSGTYLCGAISLAPKLQIKES | | | |
| | | LRAELRVTE | | | |
| | L1 | NFMLTQPHSVSESPGKTVTISCKRNTGNIGSNYV | | CD3 | 254 |
| | (16412) | NWYQQHEGSSPTTIIYRNDKRPDGVSDRFSGSID | | | |
| | | RSSKSASLTISNLKTEDEADYFCQSYSSGFIFGGG | | | |
| | | TKLTVLGQPKAAPSVTLFPPSSEELQANKATLVC | | | |
| | | LISDFYPGAVTVAWKADSSPVKAGVETTTPSKQ | | | |
| | | SNNKYAASSYLSLTPEQWKSHRSYSCQVTHEGS | | | |
| | | TVEKTVAPTECS | | | |
| v38410 | H2 | QVQLVESGGGLVQPGGSLRLSCSVSGIDLSSNPM | | Cldn18.2 | 318 |
| | (28373) | IWVRQAPGKGLQYIGIIDTDGSTYYADWAKGRF | | | |
| | | TISKDSTTVYLQINSPRAEDTAVYYCARRLHGSS | | | |
| | | NGYYDDLWGQGTLVTVSSGGGGSGGGGSGGG | | | |
| | | GSGGGGSDGQMTQSPSSVSASVGDRVTITCQAS | | | |
| | | QSIYSYLSWYQQKPGQRPKLLIYKASTLASGVPS | | | |
| | | RFSGSGSGTDFTLTISSVQPEDFATYYCQQGYTV | | | |
| | | TNVDKNTFGGGTKVEIKAAEPKSSDKTHTCPPCP | | | |
| | | APEAAGGPSVFLFPPKPKDTLMISRTPEVTCVVV | | | |
| | | SVSHEDPEVKFNWYVDGVEVHNAKTKPREEQY | | | |
| | | NSTYRVVSVLTVLHQDWLNGKEYKCKVSNKAL | | | |
| | | PAPIEKTISKAKGQPREPQVYVLPPSRDELTKNQ | | | |
| | | VSLLCLVKGFYPSDIAVEWESNGQPENNYLTWP | | | |
| | | PVLDSDGSFFLYSKLTVDKSRWQQGNVFSCSVM | | | |
| | | HEALHNHYTQKSLSLSPG | | | |
| | H1 | EVQLVESGGGLVQPGGSLRLSCAASGVTFNYYG | | CD3/PD- | 320 |
| | (29238) | MSWIRQAPGKGLEWVASITSSGGRIYYPDSVKG | | L1 | |
| | | RFTISRENTQKTLYLQMNSLRAEDTAVYYCTLD | | | |
| | | GRDGWVAYWGQGTLVTVSSASTKGPSVFPLAP | | | |
| | | SSKSTSGGTAALGCLVKDYFPEPVTVSWNSGAL | | | |
| | | TSGVHTFPAVLQSSGLYSLSSVVTVPSSSLGTQT | | | |
| | | YICNVNHKPSNTKVDKKVEPKSCDKTHTCPPCP | | | |
| | | APEAAGGPSVFLFPPKPKDTLMISRTPEVTCVVV | | | |
| | | SVSHEDPEVKFNWYVDGVEVHNAKTKPREEQY | | | |
| | | NSTYRVVSVLTVLHQDWLNGKEYKCKVSNKAL | | | |
| | | PAPIEKTISKAKGQPREPQVYVYPPSRDELTKNQ | | | |
| | | VSLTCLVKGFYPSDIAVEWESNGQPENNYKTTPP | | | |
| | | VLDSDGSFALVSKLTVDKSRWQQGNVFSCSVM | | | |
| | | HEALHNHYTQKSLSLSPGGGGGSGDRPWNPPTF | | | |
| | | SPALLVVTEGDNATFTCSFSNTSESFHVVWHRES | | | |
| | | PSGQTDTLAAFPEDRSQPGQDARFRVTQLPNGR | | | |
| | | DFHMSVVRARRNDSGTYVCGVISLAPKIQIKESL | | | |
| | | RAELRVTE | | | |
| | L1 | NFMLTQPHSVSESPGKTVTISCKRNTGNIGSNYV | | CD3 | 254 |
| | (16412) | NWYQQHEGSSPTTIIYRNDKRPDGVSDRFSGSID | | | |
| | | RSSKSASLTISNLKTEDEADYFCQSYSSGFIFGGG | | | |
| | | TKLTVLGQPKAAPSVTLFPPSSEELQANKATLVC | | | |
| | | LISDFYPGAVTVAWKADSSPVKAGVETTTPSKQ | | | |
| | | SNNKYAASSYLSLTPEQWKSHRSYSCQVTHEGS | | | |
| | | TVEKTVAPTECS | | | |
| v38408 | H1 | NPPTFSPALLVVTEGDNATFTCSFSNTSESFHVV | | PD-L1, | 321 |
| | (29208) | WHRESPSGQTDTLAAFPEDRSQPGQDARFRVTQ | | CD3 | |
| | | LPNGRDFHMSVVRARRNDSGTYVCGVISLAPKI | | | |
| | | QIKESLRAELRVTEGGGGSGEVQLVESGGGLVQ | | | |
| | | PGGSLRLSCAASGVTFNYYGMSWIRQAPGKGLE | | | |
| | | WVASITSSGGRIYYPDSVKGRFTISRENTQKTLY | | | |
| | | LQMNSLRAEDTAVYYCTLDGRDGWVAYWGQG | | | |
| | | TLVTVSSASTKGPSVFPLAPSSKSTSGGTAALGC | | | |
| | | LVKDYFPEPVTVSWNSGALTSGVHTFPAVLQSS | | | |
| | | GLYSLSSVVTVPSSSLGTQTYICNVNHKPSNTKV | | | |
| | | DKKVEPKSCDKTHTCPPCPAPEAAGGPSVFLFPP | | | |
| | | KPKDTLMISRTPEVTCVVVSVSHEDPEVKFNWY | | | |
| | | VDGVEVHNAKTKPREEQYNSTYRVVSVLTVLH | | | |
| | | QDWLNGKEYKCKVSNKALPAPIEKTISKAKGQP | | | |
| | | REPQVYVYPPSRDELTKNQVSLTCLVKGFYPSDI | | | |
| | | AVEWESNGQPENNYKTTPPVLDSDGSFALVSKL | | | |
| | | TVDKSRWQQGNVFSCSVMHEALHNHYTQKSLS | | | |
| | | LSPG | | | |
| | L1 | NFMLTQPHSVSESPGKTVTISCKRNTGNIGSNYV | | CD3 | 254 |
| | (16412) | NWYQQHEGSSPTTIIYRNDKRPDGVSDRFSGSID | | | |
| | | RSSKSASLTISNLKTEDEADYFCQSYSSGFIFGGG | | | |
| | | TKLTVLGQPKAAPSVTLFPPSSEELQANKATLVC | | | |
| | | LISDFYPGAVTVAWKADSSPVKAGVETTTPSKQ | | | |
| | | SNNKYAASSYLSLTPEQWKSHRSYSCQVTHEGS | | | |
| | | TVEKTVAPTECS | | | |
| | H2 | QVQLVESGGGLVQPGGSLRLSCSVSGIDLSSNPM | | Cldn18.2 | 318 |
| | (28373) | IWVRQAPGKGLQYIGIIDTDGSTYYADWAKGRF | | | |
| | | TISKDSTTVYLQINSPRAEDTAVYYCARRLHGSS | | | |
| | | NGYYDDLWGQGTLVTVSSGGGGSGGGGSGGG | | | |
| | | GSGGGGSDGQMTQSPSSVSASVGDRVTITCQAS | | | |
| | | QSIYSYLSWYQQKPGQRPKLLIYKASTLASGVPS | | | |
| | | RFSGSGSGTDFTLTISSVQPEDFATYYCQQGYTV | | | |
| | | TNVDKNTFGGGTKVEIKAAEPKSSDKTHTCPPCP | | | |
| | | APEAAGGPSVFLFPPKPKDTLMISRTPEVTCVVV | | | |
| | | SVSHEDPEVKFNWYVDGVEVHNAKTKPREEQY | | | |
| | | NSTYRVVSVLTVLHQDWLNGKEYKCKVSNKAL | | | |
| | | PAPIEKTISKAKGQPREPQVYVLPPSRDELTKNQ | | | |
| | | VSLLCLVKGFYPSDIAVEWESNGQPENNYLTWP | | | |
| | | PVLDSDGSFFLYSKLTVDKSRWQQGNVFSCSVM | | | |
| | | HEALHNHYTQKSLSLSPG | | | |
| v38731 | H1 | NPPTFSPALLVVTEGDNATFTCSFSNTSESFVLN | | PD-L1, | 322 |
| | (29211) | WYRMSPSNQTDKLAAFPEDRSQPGQDARFRVT | | CD3 | |
| | | QLPNGRDFHMSVVRARRNDSGTYLCGAISLAPK | | | |
| | | LQIKESLRAELRVTEGGGGSGEVQLVESGGGLV | | | |
| | | QPGGSLRLSCAASGVTFNYYGMSWIRQAPGKGL | | | |
| | | EWVASITSSGGRIYYPDSVKGRFTISRENTQKTL | | | |
| | | YLQMNSLRAEDTAVYYCTLDGRDGWVAYWGQ | | | |
| | | GTLVTVSSASTKGPSVFPLAPSSKSTSGGTAALG | | | |
| | | CLVKDYFPEPVTVSWNSGALTSGVHTFPAVLQS | | | |
| | | SGLYSLSSVVTVPSSSLGTQTYICNVNHKPSNTK | | | |
| | | VDKKVEPKSCDKTHTCPPCPAPEAAGGPSVFLFP | | | |
| | | PKPKDTLMISRTPEVTCVVVSVSHEDPEVKFNW | | | |
| | | YVDGVEVHNAKTKPREEQYNSTYRVVSVLTVL | | | |
| | | HQDWLNGKEYKCKVSNKALPAPIEKTISKAKGQ | | | |
| | | PREPQVYVYPPSRDELTKNQVSLTCLVKGFYPSD | | | |
| | | IAVEWESNGQPENNYKTTPPVLDSDGSFALVSK | | | |
| | | LTVDKSRWQQGNVFSCSVMHEALHNHYTQKSL | | | |
| | | SLSPG | | | |
| | L1 | NFMLTQPHSVSESPGKTVTISCKRNTGNIGSNYV | | CD3 | 254 |
| | (16412) | NWYQQHEGSSPTTIIYRNDKRPDGVSDRFSGSID | | | |
| | | RSSKSASLTISNLKTEDEADYFCQSYSSGFIFGGG | | | |
| | | TKLTVLGQPKAAPSVTLFPPSSEELQANKATLVC | | | |
| | | LISDFYPGAVTVAWKADSSPVKAGVETTTPSKQ | | | |
| | | SNNKYAASSYLSLTPEQWKSHRSYSCQVTHEGS | | | |
| | | TVEKTVAPTECS | | | |
| | H2 | QVQLVESGGGLVQPGGSLRLSCSVSGIDLSSNPM | | Cldn18.2 | 318 |
| | (28373) | IWVRQAPGKGLQYIGIIDTDGSTYYADWAKGRF | | | |
| | | TISKDSTTVYLQINSPRAEDTAVYYCARRLHGSS | | | |
| | | NGYYDDLWGQGTLVTVSSGGGGSGGGGSGGG | | | |
| | | GSGGGGSDGQMTQSPSSVSASVGDRVTITCQAS | | | |
| | | QSIYSYLSWYQQKPGQRPKLLIYKASTLASGVPS | | | |
| | | RFSGSGSGTDFTLTISSVQPEDFATYYCQQGYTV | | | |
| | | TNVDKNTFGGGTKVEIKAAEPKSSDKTHTCPPCP | | | |
| | | APEAAGGPSVFLFPPKPKDTLMISRTPEVTCVVV | | | |
| | | SVSHEDPEVKFNWYVDGVEVHNAKTKPREEQY | | | |
| | | NSTYRVVSVLTVLHQDWLNGKEYKCKVSNKAL | | | |
| | | PAPIEKTISKAKGQPREPQVYVLPPSRDELTKNQ | | | |
| | | VSLLCLVKGFYPSDIAVEWESNGQPENNYLTWP | | | |
| | | PVLDSDGSFFLYSKLTVDKSRWQQGNVFSCSVM | | | |
| | | HEALHNHYTQKSLSLSPG | | | |
| v38999 | H1 | EVQLVESGGGLVQPGGSLRLSCAASGVTFNYYG | | CD3, | 323 |
| | (29244) | MSWIRQAPGKGLEWVASITSSGGRIYYPDSVKG | | Cldn18.2 | |
| | | RFTISRENTQKTLYLQMNSLRAEDTAVYYCTLD | | | |
| | | GRDGWVAYWGQGTLVTVSSASTKGPSVFPLAP | | | |
| | | SSKSTSGGTAALGCLVKDYFPEPVTVSWNSGAL | | | |
| | | TSGVHTFPAVLQSSGLYSLSSVVTVPSSSLGTQT | | | |
| | | YICNVNHKPSNTKVDKKVEPKSCDKTHTCPPCP | | | |
| | | APEAAGGPSVFLFPPKPKDTLMISRTPEVTCVVV | | | |
| | | SVSHEDPEVKFNWYVDGVEVHNAKTKPREEQY | | | |
| | | NSTYRVVSVLTVLHQDWLNGKEYKCKVSNKAL | | | |
| | | PAPIEKTISKAKGQPREPQVYVYPPSRDELTKNQ | | | |
| | | VSLTCLVKGFYPSDIAVEWESNGQPENNYKTTPP | | | |
| | | VLDSDGSFALVSKLTVDKSRWQQGNVFSCSVM | | | |
| | | HEALHNHYTQKSLSLSPGGGGGSGQVQLVESGG | | | |
| | | GLVQPGGSLRLSCSVSGIDLSSNPMIWVRQAPGK | | | |
| | | GLQYIGIIDTDGSTYYADWAKGRFTISKDSTTVY | | | |
| | | LQINSPRAEDTAVYYCARRLHGSSNGYYDDLW | | | |
| | | GQGTLVTVSSGGGGSGGGGSGGGGSGGGGSDG | | | |
| | | QMTQSPSSVSASVGDRVTITCQASQSIYSYLSWY | | | |
| | | QQKPGQRPKLLIYKASTLASGVPSRFSGSGSGTD | | | |
| | | FTLTISSVQPEDFATYYCQQGYTVTNVDKNTFG | | | |
| | | GGTKVEIK | | | |
| | L1 | NFMLTQPHSVSESPGKTVTISCKRNTGNIGSNYV | | CD3 | 254 |
| | (16412) | NWYQQHEGSSPTTIIYRNDKRPDGVSDRFSGSID | | | |
| | | RSSKSASLTISNLKTEDEADYFCQSYSSGFIFGGG | | | |
| | | TKLTVLGQPKAAPSVTLFPPSSEELQANKATLVC | | | |
| | | LISDFYPGAVTVAWKADSSPVKAGVETTTPSKQ | | | |
| | | SNNKYAASSYLSLTPEQWKSHRSYSCQVTHEGS | | | |
| | | TVEKTVAPTECS | | | |
| | H2 | NPPTFSPALLVVTEGDNATFTCSFSNTSESFHVV | | PD-L1, | 324 |
| | (29245) | WHRESPSGQTDTLAAFPEDRSQPGQDARFRVTQ | | Cldn18.2 | |
| | | LPNGRDFHMSVVRARRNDSGTYVCGVISLAPKI | | | |
| | | QIKESLRAELRVTEGGGGSGQVQLVESGGGLVQ | | | |
| | | PGGSLRLSCSVSGIDLSSNPMIWVRQAPGKGLQY | | | |
| | | IGIIDTDGSTYYADWAKGRFTISKDSTTVYLQINS | | | |
| | | PRAEDTAVYYCARRLHGSSNGYYDDLWGQGTL | | | |
| | | VTVSSGGGGSGGGGSGGGGSGGGGSDGQMTQS | | | |
| | | PSSVSASVGDRVTITCQASQSIYSYLSWYQQKPG | | | |
| | | QRPKLLIYKASTLASGVPSRFSGSGSGTDFTLTIS | | | |
| | | SVQPEDFATYYCQQGYTVTNVDKNTFGGGTKV | | | |
| | | EIKAAEPKSSDKTHTCPPCPAPEAAGGPSVFLFPP | | | |
| | | KPKDTLMISRTPEVTCVVVSVSHEDPEVKENWY | | | |
| | | VDGVEVHNAKTKPREEQYNSTYRVVSVLTVLH | | | |
| | | QDWLNGKEYKCKVSNKALPAPIEKTISKAKGQP | | | |
| | | REPQVYVLPPSRDELTKNQVSLLCLVKGFYPSDI | | | |
| | | AVEWESNGQPENNYLTWPPVLDSDGSFFLYSKL | | | |
| | | TVDKSRWQQGNVFSCSVMHEALHNHYTQKSLS | | | |
| | | LSPG | | | |
| v39000 | H1 | EVQLVESGGGLVQPGGSLRLSCAASGVTFNYYG | | CD3, | 323 |
| | (29244) | MSWIRQAPGKGLEWVASITSSGGRIYYPDSVKG | | Cldn18.2 | |
| | | RFTISRENTQKTLYLQMNSLRAEDTAVYYCTLD | | | |
| | | GRDGWVAYWGQGTLVTVSSASTKGPSVFPLAP | | | |
| | | SSKSTSGGTAALGCLVKDYFPEPVTVSWNSGAL | | | |
| | | TSGVHTFPAVLQSSGLYSLSSVVTVPSSSLGTQT | | | |
| | | YICNVNHKPSNTKVDKKVEPKSCDKTHTCPPCP | | | |
| | | APEAAGGPSVFLFPPKPKDTLMISRTPEVTCVVV | | | |
| | | SVSHEDPEVKFNWYVDGVEVHNAKTKPREEQY | | | |
| | | NSTYRVVSVLTVLHQDWLNGKEYKCKVSNKAL | | | |
| | | PAPIEKTISKAKGQPREPQVYVYPPSRDELTKNQ | | | |
| | | VSLTCLVKGFYPSDIAVEWESNGQPENNYKTTPP | | | |
| | | VLDSDGSFALVSKLTVDKSRWQQGNVFSCSVM | | | |
| | | HEALHNHYTQKSLSLSPGGGGGSGQVQLVESGG | | | |
| | | GLVQPGGSLRLSCSVSGIDLSSNPMIWVRQAPGK | | | |
| | | GLQYIGIIDTDGSTYYADWAKGRFTISKDSTTVY | | | |
| | | LQINSPRAEDTAVYYCARRLHGSSNGYYDDLW | | | |
| | | GQGTLVTVSSGGGGSGGGGSGGGGSGGGGSDG | | | |
| | | QMTQSPSSVSASVGDRVTITCQASQSIYSYLSWY | | | |
| | | QQKPGQRPKLLIYKASTLASGVPSRFSGSGSGTD | | | |
| | | FTLTISSVQPEDFATYYCQQGYTVTNVDKNTFG | | | |
| | | GGTKVEIK | | | |
| | L1 | NFMLTQPHSVSESPGKTVTISCKRNTGNIGSNYV | | CD3 | 254 |
| | (16412) | INWYQQHEGSSPTTIIYRNDKRPDGVSDRFSGSID | | | |
| | | RSSKSASLTISNLKTEDEADYFCQSYSSGFIFGGG | | | |
| | | TKLTVLGQPKAAPSVTLFPPSSEELQANKATLVC | | | |
| | | LISDFYPGAVTVAWKADSSPVKAGVETTTPSKQ | | | |
| | | SNNKYAASSYLSLTPEQWKSHRSYSCQVTHEGS | | | |
| | | TVEKTVAPTECS | | | |
| | H2 | NPPTFSPALLVVTEGDNATFTCSFSNTSESFVLN | | PD-L1, | 325 |
| | (29248) | WYRMSPSNQTDKLAAFPEDRSQPGQDARFRVT | | Cldn18.2 | |
| | | QLPNGRDFHMSVVRARRNDSGTYLCGAISLAPK | | | |
| | | LQIKESLRAELRVTEGGGGSGQVQLVESGGGLV | | | |
| | | QPGGSLRLSCSVSGIDLSSNPMIWVRQAPGKGLQ | | | |
| | | YIGIIDTDGSTYYADWAKGRFTISKDSTTVYLQI | | | |
| | | NSPRAEDTAVYYCARRLHGSSNGYYDDLWGQG | | | |
| | | TLVTVSSGGGGSGGGGSGGGGSGGGGSDGQMT | | | |
| | | QSPSSVSASVGDRVTITCQASQSIYSYLSWYQQK | | | |
| | | PGQRPKLLIYKASTLASGVPSRFSGSGSGTDFTLT | | | |
| | | ISSVQPEDFATYYCQQGYTVTNVDKNTFGGGTK | | | |
| | | VEIKAAEPKSSDKTHTCPPCPAPEAAGGPSVFLFP | | | |
| | | PKPKDTLMISRTPEVTCVVVSVSHEDPEVKFNW | | | |
| | | YVDGVEVHNAKTKPREEQYNSTYRVVSVLTVL | | | |
| | | HQDWLNGKEYKCKVSNKALPAPIEKTISKAKGQ | | | |
| | | PREPQVYVLPPSRDELTKNQVSLLCLVKGFYPSD | | | |
| | | IAVEWESNGQPENNYLTWPPVLDSDGSFFLYSK | | | |
| | | LTVDKSRWQQGNVFSCSVMHEALHNHYTQKSL | | | |
| | | SLSPG | | | |
| v39003 | H1 | EVQLVESGGGLVQPGGSLRLSCAASGVTFNYYG | | CD3, | 323 |
| | (29244) | MSWIRQAPGKGLEWVASITSSGGRIYYPDSVKG | | Cldn18.2 | |
| | | RFTISRENTQKTLYLQMNSLRAEDTAVYYCTLD | | | |
| | | GRDGWVAYWGQGTLVTVSSASTKGPSVFPLAP | | | |
| | | SSKSTSGGTAALGCLVKDYFPEPVTVSWNSGAL | | | |
| | | TSGVHTFPAVLQSSGLYSLSSVVTVPSSSLGTQT | | | |
| | | YICNVNHKPSNTKVDKKVEPKSCDKTHTCPPCP | | | |
| | | APEAAGGPSVFLFPPKPKDTLMISRTPEVTCVVV | | | |
| | | SVSHEDPEVKFNWYVDGVEVHNAKTKPREEQY | | | |
| | | NSTYRVVSVLTVLHQDWLNGKEYKCKVSNKAL | | | |
| | | PAPIEKTISKAKGQPREPQVYVYPPSRDELTKNQ | | | |
| | | VSLTCLVKGFYPSDIAVEWESNGQPENNYKTTPP | | | |
| | | VLDSDGSFALVSKLTVDKSRWQQGNVFSCSVM | | | |
| | | HEALHNHYTQKSLSLSPGGGGGSGQVQLVESGG | | | |
| | | GLVQPGGSLRLSCSVSGIDLSSNPMIWVRQAPGK | | | |
| | | GLQYIGIIDTDGSTYYADWAKGRFTISKDSTTVY | | | |
| | | LQINSPRAEDTAVYYCARRLHGSSNGYYDDLW | | | |
| | | GQGTLVTVSSGGGGSGGGGSGGGGSGGGGSDG | | | |
| | | QMTQSPSSVSASVGDRVTITCQASQSIYSYLSWY | | | |
| | | QQKPGQRPKLLIYKASTLASGVPSRFSGSGSGTD | | | |
| | | FTLTISSVQPEDFATYYCQQGYTVTNVDKNTFG | | | |
| | | GGTKVEIK | | | |
| | L1 | NFMLTQPHSVSESPGKTVTISCKRNTGNIGSNYV | | CD3 | 254 |
| | (16412) | NWYQQHEGSSPTTIIYRNDKRPDGVSDRESGSID | | | |
| | | RSSKSASLTISNLKTEDEADYFCQSYSSGFIFGGG | | | |
| | | TKLTVLGQPKAAPSVTLFPPSSEELQANKATLVC | | | |
| | | LISDFYPGAVTVAWKADSSPVKAGVETTTPSKQ | | | |
| | | SNNKYAASSYLSLTPEQWKSHRSYSCQVTHEGS | | | |
| | | TVEKTVAPTECS | | | |
| | H2 | QVQLVESGGGLVQPGGSLRLSCSVSGIDLSSNPM | | Cldn18.2, | 326 |
| | (29251) | IWVRQAPGKGLQYIGIIDTDGSTYYADWAKGRF | | PD-L1 | |
| | | TISKDSTTVYLQINSPRAEDTAVYYCARRLHGSS | | | |
| | | NGYYDDLWGQGTLVTVSSGGGGSGGGGSGGG | | | |
| | | GSGGGGSDGQMTQSPSSVSASVGDRVTITCQAS | | | |
| | | QSIYSYLSWYQQKPGQRPKLLIYKASTLASGVPS | | | |
| | | RFSGSGSGTDFTLTISSVQPEDFATYYCQQGYTV | | | |
| | | TNVDKNTFGGGTKVEIKAAEPKSSDKTHTCPPCP | | | |
| | | APEAAGGPSVFLFPPKPKDTLMISRTPEVTCVVV | | | |
| | | SVSHEDPEVKFNWYVDGVEVHNAKTKPREEQY | | | |
| | | NSTYRVVSVLTVLHQDWLNGKEYKCKVSNKAL | | | |
| | | PAPIEKTISKAKGQPREPQVYVLPPSRDELTKNQ | | | |
| | | VSLLCLVKGFYPSDIAVEWESNGQPENNYLTWP | | | |
| | | PVLDSDGSFFLYSKLTVDKSRWQQGNVFSCSVM | | | |
| | | HEALHNHYTQKSLSLSPGGGGGSGDRPWNPPTF | | | |
| | | SPALLVVTEGDNATFTCSFSNTSESFHVVWHRES | | | |
| | | PSGQTDTLAAFPEDRSQPGQDARFRVTQLPNGR | | | |
| | | DFHMSVVRARRNDSGTYVCGVISLAPKIQIKESL | | | |
| | | RAELRVTE | | | |
| v39004 | H1 | EVQLVESGGGLVQPGGSLRLSCAASGVTFNYYG | | CD3, | 323 |
| | (29244) | MSWIRQAPGKGLEWVASITSSGGRIYYPDSVKG | | Cldn18.2 | |
| | | RFTISRENTQKTLYLQMNSLRAEDTAVYYCTLD | | | |
| | | GRDGWVAYWGQGTLVTVSSASTKGPSVFPLAP | | | |
| | | SSKSTSGGTAALGCLVKDYFPEPVTVSWNSGAL | | | |
| | | TSGVHTFPAVLQSSGLYSLSSVVTVPSSSLGTQT | | | |
| | | YICNVNHKPSNTKVDKKVEPKSCDKTHTCPPCP | | | |
| | | APEAAGGPSVFLFPPKPKDTLMISRTPEVTCVVV | | | |
| | | SVSHEDPEVKFNWYVDGVEVHNAKTKPREEQY | | | |
| | | NSTYRVVSVLTVLHQDWLNGKEYKCKVSNKAL | | | |
| | | PAPIEKTISKAKGQPREPQVYVYPPSRDELTKNQ | | | |
| | | VSLTCLVKGFYPSDIAVEWESNGQPENNYKTTPP | | | |
| | | VLDSDGSFALVSKLTVDKSRWQQGNVFSCSVM | | | |
| | | HEALHNHYTQKSLSLSPGGGGGSGQVQLVESGG | | | |
| | | GLVQPGGSLRLSCSVSGIDLSSNPMIWVRQAPGK | | | |
| | | GLQYIGIIDTDGSTYYADWAKGRFTISKDSTTVY | | | |
| | | LQINSPRAEDTAVYYCARRLHGSSNGYYDDLW | | | |
| | | GQGTLVTVSSGGGGSGGGGSGGGGSGGGGSDG | | | |
| | | QMTQSPSSVSASVGDRVTITCQASQSIYSYLSWY | | | |
| | | QQKPGQRPKLLIYKASTLASGVPSRFSGSGSGTD | | | |
| | | FTLTISSVQPEDFATYYCQQGYTVTNVDKNTFG | | | |
| | | GGTKVEIK | | | |
| | L1 | NFMLTQPHSVSESPGKTVTISCKRNTGNIGSNYV | | CD3 | 254 |
| | (16412) | NWYQQHEGSSPTTIIYRNDKRPDGVSDRFSGSID | | | |
| | | RSSKSASLTISNLKTEDEADYFCQSYSSGFIFGGG | | | |
| | | TKLTVLGQPKAAPSVTLFPPSSEELQANKATLVC | | | |
| | | LISDFYPGAVTVAWKADSSPVKAGVETTTPSKQ | | | |
| | | SNNKYAASSYLSLTPEQWKSHRSYSCQVTHEGS | | | |
| | | TVEKTVAPTECS | | | |
| | H2 | QVQLVESGGGLVQPGGSLRLSCSVSGIDLSSNPM | | Cldn18.2, | 327 |
| | (29254) | IWVRQAPGKGLQYIGIIDTDGSTYYADWAKGRF | | PD-L1 | |
| | | TISKDSTTVYLQINSPRAEDTAVYYCARRLHGSS | | | |
| | | NGYYDDLWGQGTLVTVSSGGGGSGGGGSGGG | | | |
| | | GSGGGGSDGQMTQSPSSVSASVGDRVTITCQAS | | | |
| | | QSIYSYLSWYQQKPGQRPKLLIYKASTLASGVPS | | | |
| | | RFSGSGSGTDFTLTISSVQPEDFATYYCQQGYTV | | | |
| | | TNVDKNTFGGGTKVEIKAAEPKSSDKTHTCPPCP | | | |
| | | APEAAGGPSVFLFPPKPKDTLMISRTPEVTCVVV | | | |
| | | SVSHEDPEVKFNWYVDGVEVHNAKTKPREEQY | | | |
| | | NSTYRVVSVLTVLHQDWLNGKEYKCKVSNKAL | | | |
| | | PAPIEKTISKAKGQPREPQVYVLPPSRDELTKNQ | | | |
| | | VSLLCLVKGFYPSDIAVEWESNGQPENNYLTWP | | | |
| | | PVLDSDGSFFLYSKLTVDKSRWQQGNVFSCSVM | | | |
| | | HEALHNHYTQKSLSLSPGGGGGSGDRPWNPPTF | | | |
| | | SPALLVVTEGDNATFTCSFSNTSESFVLNWYRM | | | |
| | | SPSNQTDKLAAFPEDRSQPGQDARFRVTQLPNG | | | |
| | | RDFHMSVVRARRNDSGTYLCGAISLAPKLQIKES | | | |
| | | LRAELRVTE | | | |

*Any heavy (H1, H2, etc.) and light (L) chain clone sequences shown to be part of a specific variant (e.g., trispecific fusion protein) can also be used and combined to produce other variants than those recited in this table. For example, the clone sequences set forth in clone numbers 29244, 16412, and 29254 which can be combined to yield variant 39004 can also be used separately and be combined with any other clone sequence(s) to produce additional trivalent and/or tetravalent trispecific fusion proteins as described herein.

TABLE BB
Anti-CD3 Domain Sequences

| Anti- | | | |
|---|---|---|---|
| CD3 | | | Seq |
| para- | Sequence | | ID |
| tope | Type | Sequence | No. |
| 1 | VH | EVQLVESGGGLVQPGGSLRLSCAASGVT | 206 |
| | | FNYYGMSWIRQAPGKGLEWVASITSSGG | |
| | | RIYYPDSVKGRFTISRENTQKTLYLQMN | |
| | | SLRAEDTAVYYCTLDGRDGWVAYWGQGT | |
| | | LVTVSS | |
| | Kabat HCDR1 | YYGMS | 303 |
| | Kabat HCDR2 | SITSSGGRIYYPDSVKG | 301 |
| | Kabat HCDR3 | DGRDGWVAY | 275 |
| | VL | NFMLTQPHSVSESPGKTVTISCKRNTGN | 210 |
| | | IGSNYVNWYQQHEGSSPTTIIYRNDKRP | |
| | | DGVSDRFSGSIDRSSKSASLTISNLKTE | |
| | | DEADYFCQSYSSGFIFGGGTKLTVL | |
| | Kabat LCDR1 | KRNTGNIGSNYVN | 287 |
| | Kabat LCDR2 | RNDKRPD | 298 |
| | Kabat LCDR3 | QSYSSGFI | 295 |
| 2 | VH | EVOLVESGGGLVQPGGSLRLSCAASGVT | 215 |
| | | FNYYGMSWIRQAPGKGLEWVASITRSGG | |
| | | RIYYPDSVKGRFTISRENTQKTLYLQMN | |
| | | SLRAEDTAVYYCTLDGRDGWVAYWGQGT | |
| | | LVTVSS | |
| | Kabat HCDR1 | YYGMS | 303 |
| | Kabat HCDR2 | SITRSGGRIYYPDSVKG | 217 |
| | Kabat HCDR3 | DGRDGWVAY | 275 |
| | VL | NFMLTQPSSVSGVPGQRVTISCTGNTGN | 219 |
| | | IGSNYVNWYQQLPGTAPKLLIYRDDKRP | |
| | | SGVPDRFSGSKSGTSASLAITGFQAEDE | |
| | | ADYYCQSYSSGFIFGGGTKLTVL | |
| | Kabat LCDR1 | TGNTGNIGSNYVN | 220 |
| | Kabat LCDR2 | RDDKRPS | 221 |
| | Kabat LCDR3 | QSYSSGFI | 295 |
| 3 | VH | EVOLVESGGGLVQPGGSLKLSCAASGFT | 223 |
| | | FNKYAMNWVRQAPGKGLEWVARIRSKYN | |
| | | NYATYYADSVKDRFTISRDDSKNTAYLQ | |
| | | MNNLKTEDTAVYYCVRHGNFGNSYISYW | |
| | | AYWGQGTLVTVSS | |
| | Kabat HCDR1 | KYAMN | 224 |
| | Kabat HCDR2 | RIRSKYNNYATYYADSVKD | 225 |
| | Kabat HCDR3 | HGNFGNSYISYWAY | 226 |
| | VL | QTVVTQEPSLTVSPGGTVTLTCGSSTGA | 227 |
| | | VTSGNYPNWVQQKPGQAPRGLIGGTKFL | |
| | | APGTPARFSGSLLGGKAALTLSGVQPED | |
| | | EAEYYCVLWYSNRWVFGGGTKLTVL | |
| | Kabat LCDR1 | GSSTGAVTSGNYPN | 228 |
| | Kabat LCDR2 | GTKFLAP | 229 |
| | Kabat LCDR3 | VLWYSNRWV | 230 |
| 4 | VH | EVQLLESGGGLVQPGGSLRLSCAASGFT | 231 |
| | | FSTYAMNWVRQAPGKGLEWVSRIRSKYN | |
| | | NYATYYADSVKGRFTISRDDSKNTLYLQ | |
| | | MNSLRAEDTAVYYCVRHGNFGNSYVSWF | |
| | | AYWGQGTLVTVSS | |
| | Kabat HCDR1 | TYAMN | 232 |
| | Kabat HCDR2 | RIRSKYNNYATYYADSVKG | 233 |
| | Kabat HCDR3 | HGNFGNSYVSWFAY | 234 |
| | VL | QAVVTQEPSLTVSPGGTVTLTCGSSTGA | 235 |
| | | VTTSNYANWVQEKPGQAFRGLIGGTNKR | |
| | | APGTPARFSGSLLGGKAALTLSGAQPED | |
| | | EAEYYCALWYSNLWVFGGGTKLTVL | |
| | Kabat LCDR1 | GSSTGAVTTSNYAN | 236 |
| | Kabat LCDR2 | GTNKRAP | 237 |
| | Kabat LCDR3 | ALWYSNLWV | 238 |
| 5 | VH | QVQLVQSGAEVKKPGASVKVSCKASGYT | 239 |
| | | FTRSTMHWVRQAPGQGLEWIGYINPSSA | |
| | | YTNYNQKFKDRVTITADKSTSTAYMELS | |
| | | SLRSEDTAVYYCASPQVHYDYNGFPYWG | |
| | | QGTLVTVSS | |
| | Kabat HCDR1 | RSTMH | 207 |
| | Kabat HCDR2 | YINPSSAYTNYNQKFKD | 208 |
| | Kabat HCDR3 | PQVHYDYNGFPY | 209 |
| | VL | DIQMTQSPSSLSASVGDRVTITCSASSS | 243 |
| | | VSYMNWYQQKPGKAPKRLIYDSSKLASG | |
| | | VPSRFSGSGSGTEFTLTISSLQPEDFAT | |
| | | YYCQQWSRNPPTFGGGTKVEIK | |
| | Kabat LCDR1 | SASSSVSYMN | 211 |
| | Kabat LCDR2 | DSSKLAS | 212 |
| | Kabat LCDR3 | QQWSRNPPT | 214 |

1. A trispecific fusion protein comprising:
(i) a first binding domain capable of binding CD3 on the surface of a cytotoxic effector cell;
(ii) a second binding domain capable of binding a tumor associated antigen (TAA) on the surface of a tumor cell;
(iii) a third binding domain capable of binding PD-L1 on the surface of a tumor cell; and
(iv) a scaffold,
wherein the first binding domain, the second binding domain and the third binding domain are operably linked to the scaffold.

2. (canceled)

3. The trispecific fusion protein of claim 1, wherein the third binding domain is linked to (i) the first binding domain, (ii) the second binding domain, or (iii) the scaffold.

4. (canceled)

5. (canceled)

6. (canceled)

7. The trispecific fusion protein of claim 1, wherein:
a) the first binding domain is a Fab and the second binding domain is an scFv; or
b) the first binding domain is an scFv and the second binding domain is a Fab; or
c) the first binding domain is a Fab and the second binding domain is a Fab; or
d) the first binding domain is an scFv and the second binding domain is an scFv.

8. A trispecific fusion protein comprising:
(i) a first binding domain capable of binding CD3 on the surface of a cytotoxic effector cell, wherein the first binding domain is a Fab domain;
(ii) a second binding domain capable of binding a tumor-associated antigen (TAA) on the surface of a tumor cell;
(iii) a third binding domain capable of binding PD-L1 on the surface of a tumor cell; and
(iv) a scaffold,
wherein the first binding domain, the second binding domain and the third binding domain are operably linked to the scaffold, and
wherein the third binding domain is linked to (i) the N- or C-terminus of the light chain of the Fab domain, (ii) the second binding domain, or (iii) the scaffold.

9. The trispecific fusion protein of claim 1, wherein the scaffold comprises a dimeric Fc domain comprising a first Fc polypeptide and a second Fc polypeptide.

10. The trispecific fusion protein of claim 9, wherein the dimeric Fc domain is a heterodimeric Fc domain, and wherein the amino acid sequence of the first Fc polypeptide differs in at least one amino acid residue from the amino acid sequence of the second Fc polypeptide.

11. The trispecific fusion protein of claim 9, wherein the first binding domain is linked to the first Fc polypeptide via a first linker^{Fc} and the second binding domain is linked to the second Fc polypeptide via a second linker^{Fc}.

12. (canceled)

13. The trispecific fusion protein of claim 11, wherein the first linker^{Fc}, the second linker^{Fc}, or both, comprise or consist of an IgG hinge region, or a portion or variant thereof.

14. The trispecific fusion protein of claim 11, wherein the first linker^{Fc}, the second linker^{Fc}, or both, comprise an amino acid sequence having at least 80%, 90%, or 95% sequence identity to the amino acid sequence set forth in SEQ ID NO: 50 or a fragment thereof.

15. The trispecific fusion protein of claim 9, wherein the first binding domain is a Fab domain and is linked to the N-terminus of the first Fc polypeptide via the C-terminus of the Fab heavy chain.

16. The trispecific fusion protein of claim 9, wherein the second binding domain is an scFv domain and is linked via its C-terminus to the N-terminus of the second Fc polypeptide.

17. The trispecific fusion protein of claim 9,
wherein the trispecific fusion protein comprises three polypeptide chains comprising two immunoglobulin G heavy chains and one immunoglobulin light chain,
wherein the first heavy chain comprises, from N- to C-terminus, Fab VH and CH1 domains linked to CH2 and CH3 domains of the first Fc polypeptide, the second heavy chain comprises, from N- to C-terminus, scFv VH and VL or VL and VH domains linked to CH2 and CH3 domains of the second Fc polypeptide, and the light chain comprises, from N- to C-terminus, Fab VL and CL domains, and
wherein the light chain is capable of forming a Fab domain with the Fab VH and CH1 domains of the first heavy chain.

18. The trispecific fusion protein of claim 1, wherein the third binding domain comprises a PD-1 polypeptide.

19. (canceled)

20. The trispecific fusion protein of claim 18, wherein the PD-1 polypeptide is a wildtype PD-1 polypeptide comprising or consisting of the amino acid sequence set forth in SEQ ID NO: 7, or a portion of fragment thereof.

21. The trispecific fusion protein of claim 18, wherein the PD-1 polypeptide comprises one or more amino acid modifications compared to a corresponding wildtype PD-1 polypeptide that increase or decrease the binding affinity of the PD-1 polypeptide to PD-L1 when compared to the binding affinity of the corresponding wildtype PD-1 polypeptide to PD-L1.

22. (canceled)

23. The trispecific fusion protein of claim 21, wherein the PD-1 polypeptide has a binding affinity for PD-L1 of from about 100 μM to about 10 pM, from about 10 μM to about 150 pM, from about 100 nM and 150 pM, or from about 5 nM to about 90 nM.

24. The trispecific fusion protein of claim 18, wherein the PD-1 polypeptide comprises an amino acid sequence having at least about 80%, 90%, 95%, 99%, or 100% sequence identity to the amino acid sequence set forth in SEQ ID NO: 9 or 10.

25. The trispecific fusion protein of claim 1, wherein the first binding domain comprises:
a VH domain comprising a HCDR1 sequence selected from the group consisting of RSTMH (SEQ ID NO: 207), YYGMS (SEQ ID NO: 303), KYAMN (SEQ ID NO: 224) and TYAMN (SEQ ID NO: 232), a HCDR2 sequence selected from the group consisting of YINPSSAYTNYNQKFKD (SEQ ID NO: 208), SITSSGGRIYYPDSVKG (SEQ ID NO: 301), SITRSGGRIYYPDSVKG (SEQ ID NO: 217), RIRSKYNNYATYYADSVKD (SEQ ID NO: 225) and RIRSKYNNYATYYADSVKG (SEQ ID NO: 233), and a HCDR3 sequence selected from the group consisting of PQVHYDYNGFPY (SEQ ID NO: 209), DGRDGWVAY (SEQ ID NO: 275), HGNFGNSYISYWAY (SEQ ID NO: 226) and HGNFGNSYVSWFAY (SEQ ID NO: 234); and
a VL domain comprising an LCDR1 sequence selected from the group consisting of SASSSVSYMN (SEQ ID NO: 211), KRNTGNIGSNYVN (SEQ ID NO: 287), TGNTGNIGSNYVN (SEQ ID NO: 220), GSSTGAVTSGNYPN (SEQ ID NO: 228) and GSSTGAVTTSNYAN (SEQ ID NO: 236), an LCDR2 sequence selected from the group consisting of DSSKLAS (SEQ ID NO: 212), RNDKRPD (SEQ ID NO: 298), RDDKRPS (SEQ ID NO: 221), GTKFLAP (SEQ ID NO: 229), RSYQRPS (SEQ ID NO: 199) and GTNKRAP (SEQ ID NO: 237), and an LCDR3 sequence selected from the group consisting of QQWSRNPPT (SEQ ID NO: 214), QSYSSGFI (SEQ ID NO: 295), VLWYSNRWV (SEQ ID NO: 230), ATWDDSLDGWV (SEQ ID NO: 200) and ALWYSNLWV (SEQ ID NO: 238).

26. The trispecific fusion protein of claim 1, wherein the first binding domain comprises an anti-CD3 domain comprising:
a VH domain comprising an amino acid sequence having at least about 90%, 95%, 97%, 99%, or 100% sequence identity to the sequence set forth in any one of SEQ ID NOs: 2, 215, 223, and 231, and
a VL domain comprising an amino acid sequence having at least about 90%, 95%, 97%, 99%, or 100% sequence identity to the sequence set forth in any one of SEQ ID NOs: 1, 219, 227, and 235.

27. (canceled)

28. The trispecific fusion protein of claim 1, wherein the TAA is Cldn18.2 or MSLN.

29. The trispecific fusion protein of claim 1, wherein the TAA is HER2 and the second binding domain comprises an anti-HER2 domain comprising:
a VH domain comprising a HCDR1 sequence comprising DTYIH (SEQ ID NO: 121), a HCDR2 sequence comprising RIYPTNGYTRYADSVKG (SEQ ID NO: 122), and a HCDR3 sequence comprising WGGDGFYAMDY (SEQ ID NO: 123), and
a VL domain comprising an LCDR1 sequence comprising RASQDVNTAVA (SEQ ID NO: 125), an LCDR2 sequence comprising SASFLYS (SEQ ID NO: 126), and an LCDR3 sequence comprising QQHYTTPPT (SEQ ID NO: 127).

30. The trispecific fusion protein of claim 1, wherein the TAA is MSLN the second binding domain comprises an anti-MSLN domain comprising:
a VH domain comprising a HCDR1 sequence comprising GYTMN (SEQ ID NO: 286), a HCDR2 sequence comprising LITPYNGASSYNQKFRG (SEQ ID NO: 288) and a HCDR3 sequence comprising GGYDGRGFDY (SEQ ID NO: 285), and
a VL domain comprising an LCDR1 sequence comprising SASSSVSYMH (SEQ ID NO: 300), an LCDR2 sequence comprising DTSKLAS (SEQ ID NO: 279) and an LCDR3 sequence comprising QQWSGYPLT (SEQ ID NO: 294).

31. The trispecific fusion protein of claim 1, wherein the TAA is Cldn18.2 and the second binding domain comprises an anti-Cldn18.2 domain comprising:
a VH domain comprising a HCDR1 sequence comprising SNPMI (SEQ ID NO: 310), a HCDR2 sequence comprising IIDTDGSTYYADWAKG (SEQ ID NO: 311) and a HCDR3 sequence comprising RLHGSSNGYYDDL (SEQ ID NO: 312), and
a VL domain comprising an LCDR1 sequence comprising QASQSIYSYLS (SEQ ID NO: 313), an LCDR2 sequence comprising KASTLAS (SEQ ID NO: 314) and an LCDR3 sequence comprising QQGYTVTNVDKNT (SEQ ID NO: 315).

32. The trispecific fusion protein of claim 31, wherein the anti-Cldn18.2 VH sequence of the second binding domain comprises an amino acid sequence having at least about 90%, 95%, 97%, 99%, or 100% sequence identity to the sequence set forth in SEQ ID NO: 316, and the anti Cldn18.2 VL sequence of the second binding domain comprises an amino acid sequence having at least about 90%, 95%, 97%, 99%, or 100% sequence identity to the sequence set forth in SEQ ID NO: 317.

33. The trispecific fusion protein of claim 9, wherein the first Fc polypeptide and the second Fc polypeptide each comprise a CH2 domain comprising an amino acid sequence having at least about 90%, 95%, 97%, 99%, or 100% sequence identity to the sequence set forth in SEQ ID NO: 6.

34. The trispecific fusion protein of claim 9, wherein one of the first Fc polypeptide or the second Fc polypeptide comprises a CH3 domain comprising an amino acid sequence having at least about 90%, 95%, 97%, 99%, or 100% sequence identity to the sequence set forth in SEQ ID NO: 4, and the other Fc polypeptide comprises a CH3 domain comprising an amino acid sequence having at least about 90%, 95%, 97%, 99%, or 100% sequence identity to the sequence set forth in SEQ ID NO: 5.

35. The trispecific fusion protein of claim 1, wherein the TAA is HER2 and wherein the trispecific fusion protein comprises (i) a first heavy chain having at least 90%, 95%, 97%, 99% or 100% sequence identity to the sequence set forth in SEQ ID NO: 190, (ii) a second heavy chain having at least 90%, 95%, 97%, 99% or 100% sequence identity to the sequence set forth in SEQ ID NO: 119, and (iii) a light chain having at least 90%, 95%, 97%, 99% or 100% sequence identity to the sequence set forth in SEQ ID NO: 253.

36. The trispecific fusion protein of claim 1, wherein the TAA is MSLN and the trispecific fusion protein comprises (i) a first heavy chain having at least 90%, 95%, 97%, 99% or 100% sequence identity to the sequence set forth in SEQ ID NOs: 141, 190, 261, 262, 265, 271, or 272, (ii) a second heavy chain having at least 90%, 95%, 97%, 99% or 100% sequence identity to the sequence set forth in SEQ ID NOs: 256, 259, 260, or 270 and (iii) a light chain having at least 90%, 95%, 97%, 99% or 100% sequence identity to the sequence set forth in SEQ ID NOs: 253, 254, or 257.

37. The trispecific fusion protein of claim 1, wherein the TAA is Cldn18.2 and the trispecific fusion protein comprises (i) a first heavy chain having at least 90%, 95%, 97%, 99% or 100% sequence identity to the sequence set forth in SEQ ID NOs: 262, 265, 319, or 322, (ii) a second heavy chain having at least 90%, 95%, 97%, 99% or 100% sequence identity to the sequence set forth in SEQ ID NOs: 152, 153, 269 or 318 and (iii) a light chain having at least 90%, 95%, 97%, 99% or 100% sequence identity to the sequence set forth in SEQ ID NO: 254.

38. (canceled)

39. A tetravalent and trispecific fusion protein comprising:
(i) a first binding domain capable of binding CD3 on the surface of a cytotoxic effector cell;
(ii) a second binding domain and a third binding domain capable of binding a tumor-associated antigen (TAA) on the surface of a tumor cell;
(iii) a fourth binding domain capable of binding PD-L1 on the surface of a tumor cell; and
(iv) a scaffold,
wherein the first binding domain, the second binding domain, the third binding domain and the fourth binding domain are operably linked to the scaffold.

40. The tetravalent and trispecific fusion protein of claim 39, wherein the scaffold comprises a dimeric Fc domain comprising a first Fc polypeptide and a second Fc polypeptide.

41. The tetravalent and trispecific fusion protein of claim 40, wherein the dimeric Fc domain is a heterodimeric Fc domain, and wherein the amino acid sequence of the first Fc polypeptide differs in at least one amino acid residue from the amino acid sequence of the second Fc polypeptide.

42. The tetravalent and trispecific fusion protein of claim 39, wherein:
a) the first binding domain is a Fab and the second and third binding domains are both scFv domains;
b) the first binding domain is an scFv domain and the second and third binding domains are both Fab domains;
c) the first binding domain, the second binding domain and the third binding domain are Fab domains; or
d) the first binding domain, the second binding domain and the third binding domain are scFv domains.

43. The tetravalent and trispecific fusion protein of claim 42, wherein (i) the first binding domain is a Fab domain, (ii) the second binding domain is a first scFv domain, and (iii) the third binding domain is a second scFv domain.

44. The tetravalent and trispecific fusion protein of claim 40, wherein the first binding domain is linked to the N-terminus of the first Fc polypeptide, and the second binding domain is linked to the N-terminus of the second Fc polypeptide.

45. The tetravalent and trispecific fusion protein of claim 39, wherein the third binding domain is linked to (i) the first binding domain, (ii) the second binding domain, or (iii) the scaffold.

46. The tetravalent and trispecific fusion protein of claim 40, wherein:
(a) the first binding domain is a Fab domain comprising a Fab heavy chain and a Fab light chain, wherein the C-terminus of the Fab heavy chain is linked to the N-terminus of the first Fc polypeptide;
(b) the second binding domain is a first scFv domain comprising the structure, from N- to C-terminus, VH-Linker-VL or VL-Linker-VH, wherein the C-terminus of the first scFv domain is linked to the N-terminus of the second Fc polypeptide; and
(c) the third binding domain is a second scFv domain comprising the structure, from N- to C-terminus, VH-Linker-VL or VL-Linker-VH, wherein the C-terminus of the second scFv domain is linked to (i) the N-terminus of the Fab heavy or light chain, or (ii) the C-terminus of the first or second Fc polypeptide.

47. The tetravalent and trispecific fusion protein of claim 39, wherein the second binding domain and the third binding domain are capable of binding the same TAA.

48. The tetravalent and trispecific fusion protein of claim 39, wherein the TAA is MSLN and the trispecific fusion protein comprises (i) a first heavy chain having at least 90%, 95%, 97%, 99% or 100% sequence identity to the clone sequence set forth in SEQ ID NOs: 266 or 273, 29257 or 29283, (ii) a second heavy chain having at least 90%, 95%, 97%, 99% or 100% sequence identity to the sequence set forth in SEQ ID NOs: 256, 267, 152, 268, 153, 152 and (iii) a light chain having at least 90%, 95%, 97%, 99% or 100% sequence identity to the sequence set forth in SEQ ID NOs: 254, 264 or 263.

49. The tetravalent and trispecific fusion protein of claim 39, wherein the TAA is Cldn18.2 and the trispecific fusion protein comprises (i) a first heavy chain having at least 90%, 95%, 97%, 99% or 100% sequence identity to the clone sequence set forth in SEQ ID NO: 323, (ii) a second heavy chain having at least 90%, 95%, 97%, 99% or 100% sequence identity to the sequence set forth in SEQ ID NOs: 324, 325, 326 or 327 and (iii) a light chain having at least 90%, 95%, 97%, 99% or 100% sequence identity to the sequence set forth in SEQ ID NO: 254.

50. (canceled)

51. (canceled)

52. (canceled)

53. (canceled)

54. The trispecific fusion protein of claim 1, wherein the trispecific fusion protein:
(i) is capable of binding HER2 and is selected from the group consisting of v38400, v38403, v38404, v38405, v31927, v31928, v31929 and v38407;
(ii) is capable of binding MSLN and (ii-a) is trivalent and selected from the group consisting of v38520, v38440, v38441, v38442, v38443, v38344, v38345, v38910, v38911, v38913 and v38914, or (ii-b) is tetravalent and selected from the group consisting of v38448, v38449, v38450, v38451, v38452, v38915, v38919, v38921 and v38922; or
(iii) is capable of binding Cldn18.2 and (iii-a) is trivalent and selected from the group consisting of v38408, v38409, v38410, v38411, v38522, v38729, v38731, v38732, v38733, v38735, v38736, v38737, v38739 and v38740, or (iii-b) is tetravalent and selected from the group consisting of v38412, v38413, v38414, v38415, v38416, v38741, v38743, v38744, v38917, v38918, v38999, v39000, v39003, v39004 and v39007.

55. A pharmaceutical composition comprising the trispecific fusion protein of claim 1, and a pharmaceutically acceptable carrier, excipient, diluent, or combination thereof.

56. A nucleic acid molecule or a set of nucleic acid molecules encoding the trispecific fusion protein of claim 1.

57. A vector or a set of vectors comprising the nucleic acid molecule or the set of nucleic acid molecules of claim 56.

58. A cell comprising the nucleic acid molecule or the set of nucleic acid molecules of claim 56, or the vector or the set of vectors of claim 57.

59. A method of producing the trispecific fusion protein of claim 1, comprising:
(a) obtaining a host cell culture comprising at least one host cell comprising one or more nucleic acid molecules encoding the trispecific fusion protein; and
(b) recovering the trispecific fusion protein from the host cell culture.

60. The method of claim 59, further comprising purifying the trispecific fusion protein.

61. A method of eliciting an anti-tumor immune response in a cell population comprising cytotoxic effector cells and tumor cells, the method comprising contacting the cell population with an effective amount of the trispecific fusion protein of claim 1, wherein the cytotoxic effector cells express CD3, and the tumor cells express the TAA and PD-L1.

62. A method of inhibiting the proliferation of tumor cells expressing the TAA and PD-L1, the method comprising contacting a cell population comprising the tumor cells and cytotoxic effector cells with an effective amount of the trispecific fusion protein of claim 1, wherein the cytotoxic effector cells express CD3.

63. A method of killing tumor cells expressing the TAA and PD-L1, the method comprising contacting a cell population comprising the tumor cells and cytotoxic effector cells with an effective amount of the trispecific fusion protein of claim 1, wherein the cytotoxic effector cells express CD3.

64. The method of claim 61, wherein the TAA and PD-L1 are located on the same tumor cell or different tumor cells.

65. (canceled)

66. (canceled)

67. (canceled)

68. A method for treating a cancer in a subject in need thereof, the method comprising administering to the subject the trispecific fusion protein of claim 1.

69. (canceled)

70. The method of claim 68, wherein the subject is a rodent, a non-human primate, or a human.

71. (canceled)

72. (canceled)

73. (canceled)

74. (canceled)

75. (canceled)
